SEQUENCE LISTING (199 sequences)

SEQ ID NO: 1 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 2 - RNA, 100 nt, synthetic construct
  source 1..100: mol_type = other RNA
  modified_base 1..4: mod_base = OTHER; note = Phosphorothioate bond
  modified_base 94..100: mod_base = OTHER; note = Phosphorothioate bond
ggtgagtgagtgtgtgcgtggttttagagctagaaatagcaagttaaaataaggctagtccgttatcaacttgaaaaagtggcaccgagtcggtgctttt

SEQ ID NO: 3 - RNA, 70 nt, synthetic construct
  source 1..70: mol_type = other RNA
  modified_base 1..4: mod_base = OTHER; note = Phosphorothioate bond
  modified_base 64..70: mod_base = OTHER; note = Phosphorothioate bond
ggtgagtgagtgtgtgcgtggttttagagctagaaatagcaagttaaaataaggctagtccgttatcaac

SEQ ID NO: 4 - RNA, 65 nt, synthetic construct
  source 1..65: mol_type = other RNA
  modified_base 1..4: mod_base = OTHER; note = Phosphorothioate bond
  modified_base 59..65: mod_base = OTHER; note = Phosphorothioate bond
ggtgagtgagtgtgtgcgtggttttagaggaaacaagttaaaataaggctagtccgttatcaact

SEQ ID NO: 5 - RNA, 100 nt, synthetic construct; identical to SEQ ID NO: 2
  source 1..100: mol_type = other RNA
  modified_base 1..4: mod_base = OTHER; note = Phosphorothioate bond
  modified_base 97..100: mod_base = OTHER; note = Phosphorothioate bond
  modified_base 1..3: mod_base = OTHER; note = 2-prime-O-methyl RNA nucleotides
  modified_base 97..99: mod_base = um

SEQ ID NO: 6 - RNA, 70 nt, synthetic construct; identical to SEQ ID NO: 3
  source 1..70: mol_type = other RNA
  modified_base 1..4: mod_base = OTHER; note = Phosphorothioate bond
  modified_base 67..70: mod_base = OTHER; note = Phosphorothioate bond
  modified_base 1..3: mod_base = OTHER; note = 2-prime-O-methyl RNA nucleotides
  modified_base 67..69: mod_base = OTHER; note = 2-prime-O-methyl RNA nucleotides

SEQ ID NO: 7 - RNA, 65 nt, synthetic construct; identical to SEQ ID NO: 4
  source 1..65: mol_type = other RNA
  modified_base 1..4: mod_base = OTHER; note = Phosphorothioate bond
  modified_base 1..3: mod_base = OTHER; note = 2-prime-O-methyl RNA nucleotides
  modified_base 62..64: mod_base = OTHER; note = 2-prime O-methyl RNA nucleotides
  modified_base 62..65: mod_base = OTHER; note = Phosphorothioate bond

SEQ ID NO: 8 - RNA, 100 nt, synthetic construct
  source 1..100: mol_type = other RNA
  modified_base 1..4: mod_base = OTHER; note = Phosphorothioate bond
  modified_base 97..100: mod_base = OTHER; note = Phosphorothioate bond
  modified_base 1..3: mod_base = OTHER; note = 2-prime-O-methyl RNA nucleotides
  modified_base 97..99: mod_base = um
aattatggggattactaggagttttagagctagaaatagcaagttaaaataaggctagtccgttatcaacttgaaaaagtggcaccgagtcggtgctttt

SEQ ID NO: 9 - RNA, 70 nt, synthetic construct
  source 1..70: mol_type = other RNA
  modified_base 1..3: mod_base = OTHER; note = 2-prime-O-methyl RNA nucleotides
  modified_base 67..69: mod_base = OTHER; note = 2-prime-O-methyl RNA nucleotides
  modified_base 1..4: mod_base = OTHER; note = Phosphorothioate bond
  modified_base 67..70: mod_base = OTHER; note = Phosphorothioate bond
aattatggggattactaggagttttagagctagaaatagcaagttaaaataaggctagtccgttatcaac

SEQ ID NO: 10 - RNA, 65 nt, synthetic construct
  source 1..65: mol_type = other RNA
  modified_base 1..4: mod_base = OTHER; note = Phosphorothioate bond
  modified_base 62..65: mod_base = OTHER; note = Phosphorothioate bond
  modified_base 62..64: mod_base = OTHER; note = 2-prime-O-methyl RNA nucleotides
  modified_base 1..3: mod_base = OTHER; note = 2-prime-O-methyl RNA nucleotides
aattatggggattactaggagttttagaggaaacaagttaaaataaggctagtccgttatcaact

SEQ ID NO: 11 - RNA, 100 nt, synthetic construct; identical to SEQ ID NO: 8
  source 1..100: mol_type = other RNA

SEQ ID NO: 12 - RNA, 70 nt, synthetic construct; identical to SEQ ID NO: 9
  source 1..70: mol_type = other RNA

SEQ ID NO: 13 - RNA, 65 nt, synthetic construct; identical to SEQ ID NO: 10
  source 1..65: mol_type = other RNA

SEQ ID NO: 14 - RNA, 20 nt, synthetic construct
  source 1..20: mol_type = other RNA
ggtgagtgagtgtgtgcgtg

SEQ ID NO: 15 - RNA, 20 nt, synthetic construct
  source 1..20: mol_type = other RNA
aattatggggattactagga

SEQ ID NO: 16 - RNA, 44 nt, synthetic construct
  source 1..44: mol_type = other RNA
agtccgttatcaacttgaaaaagtggcaccgagtcggtgctttt

SEQ ID NO: 17 - RNA, 50 nt, synthetic construct
  source 1..50: mol_type = other RNA
gttttagagctagaaatagcaagttaaaataaggctagtccgttatcaac

SEQ ID NO: 18 - RNA, 45 nt, synthetic construct
  source 1..45: mol_type = other RNA
gttttagaggaaacaagttaaaataaggctagtccgttatcaact

SEQ ID NO: 19 - DNA, 21 bp, synthetic construct
  source 1..21: mol_type = other DNA
cctcagttccctggcaacatc

SEQ ID NO: 20 - DNA, 27 bp, synthetic construct
  source 1..27: mol_type = other DNA
aagaatgttgtgataaaaggtgatgct

SEQ ID NO: 21 - DNA, 57 bp, synthetic construct
  source 1..57: mol_type = other DNA
acactctttccctacacgacgctcttccgatctagatggcaaaagtcgatgactcgt

SEQ ID NO: 22 - DNA, 52 bp, synthetic construct
  source 1..52: mol_type = other DNA
acactctttccctacacgacgctcttccgatctcgcctcttagtgccagcat

SEQ ID NO: 23 - DNA, 63 bp, synthetic construct
  source 1..63: mol_type = other DNA
acactctttccctacacgacgctcttccgatctcaatattgttaagaaaaccgaagtgcagac

SEQ ID NO: 24 - DNA, 60 bp, synthetic construct
  source 1..60: mol_type = other DNA
acactctttccctacacgacgctcttccgatctcctgaaggataaccgggagaaaattga

SEQ ID NO: 25 - DNA, 59 bp, synthetic construct
  source 1..59: mol_type = other DNA
acactctttccctacacgacgctcttccgatctccatttaccacttacgcaaaaagctg

SEQ ID NO: 26 - DNA, 60 bp, synthetic construct
  source 1..60: mol_type = other DNA
acactctttccctacacgacgctcttccgatctatcgccattcaatcaaaaagaacttga

SEQ ID NO: 27 - DNA, 64 bp, synthetic construct
  source 1..64: mol_type = other DNA
acactctttccctacacgacgctcttccgatctttgtagcgaaagtagaaaagggtaaatccaa

SEQ ID NO: 28 - DNA, 65 bp, synthetic construct
  source 1..65: mol_type = other DNA
acactctttccctacacgacgctcttccgatctgatgactccctgacttttaaagaggatattca

SEQ ID NO: 29 - DNA, 54 bp, synthetic construct
  source 1..54: mol_type = other DNA
acactctttccctacacgacgctcttccgatctactggcgaaattgtttgggac

SEQ ID NO: 30 - DNA, 54 bp, synthetic construct
  source 1..54: mol_type = other DNA
acactctttccctacacgacgctcttccgatctagcgtagctccttcgagaaga

SEQ ID NO: 31 - DNA, 59 bp, synthetic construct
  source 1..59: mol_type = other DNA
acactctttccctacacgacgctcttccgatctatggacaaaaagtactctattggcct

SEQ ID NO: 32 - DNA, 55 bp, synthetic construct
  source 1..55: mol_type = other DNA
acactctttccctacacgacgctcttccgatctcgtaagcgctataccagtacga

SEQ ID NO: 33 - DNA, 74 bp, synthetic construct
  source 1..74: mol_type = other DNA
acactctttccctacacgacgctcttccgatctagaaataattttgtttaactttaataaggagatataccatg

SEQ ID NO: 34 - DNA, 49 bp, synthetic construct
  source 1..49: mol_type = other DNA
acactctttccctacacgacgctcttccgatctagcacgtcgccggtac

SEQ ID NO: 35 - DNA, 52 bp, synthetic construct
  source 1..52: mol_type = other DNA
acactctttccctacacgacgctcttccgatctggacagcactgacaaagct

SEQ ID NO: 36 - DNA, 52 bp, synthetic construct
  source 1..52: mol_type = other DNA
acactctttccctacacgacgctcttccgatctagcaagtcccgtcgcttag

SEQ ID NO: 37 - DNA, 52 bp, synthetic construct
  source 1..52: mol_type = other DNA
acactctttccctacacgacgctcttccgatctcgacgccatcttgctttcg

SEQ ID NO: 38 - DNA, 63 bp, synthetic construct
  source 1..63: mol_type = other DNA
acactctttccctacacgacgctcttccgatctcaagaggaattctacaaatttatcaagcct

SEQ ID NO: 39 - DNA, 51 bp, synthetic construct
  source 1..51: mol_type = other DNA
acactctttccctacacgacgctcttccgatctgtaggccccttagcacgg

SEQ ID NO: 40 - DNA, 56 bp, synthetic construct
  source 1..56: mol_type = other DNA
acactctttccctacacgacgctcttccgatctaccaaggttaaatacgtgacgga

SEQ ID NO: 41 - DNA, 69 bp, synthetic construct
  source 1..69: mol_type = other DNA
acactctttccctacacgacgctcttccgatctatcattaaggataaagacttcttagataatgaagag

SEQ ID NO: 42 - DNA, 59 bp, synthetic construct
  source 1..59: mol_type = other DNA
acactctttccctacacgacgctcttccgatctcgattctggatttcttgaagtcggac

SEQ ID NO: 43 - DNA, 56 bp, synthetic construct
  source 1..56: mol_type = other DNA
acactctttccctacacgacgctcttccgatctaaaacatcgtgattgaaatggcg

SEQ ID NO: 44 - DNA, 56 bp, synthetic construct
  source 1..56: mol_type = other DNA
acactctttccctacacgacgctcttccgatctgctgagtgactacgacgttgatc

SEQ ID NO: 45 - DNA, 55 bp, synthetic construct
  source 1..55: mol_type = other DNA
acactctttccctacacgacgctcttccgatctgagcgagttagataaggcagga

SEQ ID NO: 46 - DNA, 53 bp, synthetic construct
  source 1..53: mol_type = other DNA
acactctttccctacacgacgctcttccgatctacgcgtacctgaatgcagtg

SEQ ID NO: 47 - DNA, 55 bp, synthetic construct
  source 1..55: mol_type = other DNA
acactctttccctacacgacgctcttccgatctcgtccgttgatcgaaacaaacg

SEQ ID NO: 48 - DNA, 54 bp, synthetic construct
  source 1..54: mol_type = other DNA
acactctttccctacacgacgctcttccgatctccgtagcctactctgttctcg

SEQ ID NO: 49 - DNA, 51 bp, synthetic construct
  source 1..51: mol_type = other DNA
acactctttccctacacgacgctcttccgatctccggcgagttgcagaagg

SEQ ID NO: 50 - DNA, 56 bp, synthetic construct
  source 1..56: mol_type = other DNA
acactctttccctacacgacgctcttccgatctctgagcgcttacaataagcatcg

SEQ ID NO: 51 - DNA, 23 bp, synthetic construct
  source 1..23: mol_type = other DNA
caccaaggttcacagcctgaaaa

SEQ ID NO: 52 - DNA, 24 bp, synthetic construct
  source 1..24: mol_type = other DNA
acacatccatgggacttctgcctc

SEQ ID NO: 53 - DNA, 57 bp, synthetic construct
  source 1..57: mol_type = other DNA
gtgactggagttcagacgtgtgctcttccgatctgcgtaagtggtaaatggttgggt

SEQ ID NO: 54 - DNA, 65 bp, synthetic construct
  source 1..65: mol_type = other DNA
gtgactggagttcagacgtgtgctcttccgatctaggcttgataaatttgtagaattcctcttgg

SEQ ID NO: 55 - DNA, 60 bp, synthetic construct
  source 1..60: mol_type = other DNA
gtgactggagttcagacgtgtgctcttccgatcttctactttcgctacaacgagaacaga

SEQ ID NO: 56 - DNA, 57 bp, synthetic construct
  source 1..57: mol_type = other DNA
gtgactggagttcagacgtgtgctcttccgatctcttgtcgactacctcttcgaagt

SEQ ID NO: 57 - DNA, 64 bp, synthetic construct
  source 1..64: mol_type = other DNA
gtgactggagttcagacgtgtgctcttccgatcttggacaagctgaataaacaatttatcaaca

SEQ ID NO: 58 - DNA, 56 bp, synthetic construct
  source 1..56: mol_type = other DNA
gtgactggagttcagacgtgtgctcttccgatctggcggtggaaaaacgagtcatc

SEQ ID NO: 59 - DNA, 64 bp, synthetic construct
  source 1..64: mol_type = other DNA
gtgactggagttcagacgtgtgctcttccgatctagcttaatgataagatctttcttgacctct

SEQ ID NO: 60 - DNA, 58 bp, synthetic construct
  source 1..58: mol_type = other DNA
gtgactggagttcagacgtgtgctcttccgatcttgtcctttctgggtcgtttgattc

SEQ ID NO: 61 - DNA, 63 bp, synthetic construct
  source 1..63: mol_type = other DNA
gtgactggagttcagacgtgtgctcttccgatctttatctgaattacgtttgggtaagatgct

SEQ ID NO: 62 - DNA, 51 bp, synthetic construct
  source 1..51: mol_type = other DNA
gtgactggagttcagacgtgtgctcttccgatcttgcaactcgccggcaga

SEQ ID NO: 63 - DNA, 56 bp, synthetic construct
  source 1..56: mol_type = other DNA
gtgactggagttcagacgtgtgctcttccgatctgtttccccggagtcaaacaaca

SEQ ID NO: 64 - DNA, 64 bp, synthetic construct
  source 1..64: mol_type = other DNA
gtgactggagttcagacgtgtgctcttccgatcttacgattactttctgttcgacttaagcatt

SEQ ID NO: 65 - DNA, 53 bp, synthetic construct
  source 1..53: mol_type = other DNA
gtgactggagttcagacgtgtgctcttccgatcttgcgattcttacgccgagt

SEQ ID NO: 66 - DNA, 63 bp, synthetic construct
  source 1..63: mol_type = other DNA
gtgactggagttcagacgtgtgctcttccgatctatcatatgtgccagggctaaatagataag

SEQ ID NO: 67 - DNA, 59 bp, synthetic construct
  source 1..59: mol_type = other DNA
gtgactggagttcagacgtgtgctcttccgatctccaaacaaaccattctttttctcgc

SEQ ID NO: 68 - DNA, 58 bp, synthetic construct
  source 1..58: mol_type = other DNA
gtgactggagttcagacgtgtgctcttccgatctttcggtgttaacgcggagaatatc

SEQ ID NO: 69 - DNA, 58 bp, synthetic construct
  source 1..58: mol_type = other DNA
gtgactggagttcagacgtgtgctcttccgatctccaataactcttcggtaccatcca

SEQ ID NO: 70 - DNA, 54 bp, synthetic construct
  source 1..54: mol_type = other DNA
gtgactggagttcagacgtgtgctcttccgatctcacgcgaaacggctattacc

SEQ ID NO: 71 - DNA, 52 bp, synthetic construct
  source 1..52: mol_type = other DNA
gtgactggagttcagacgtgtgctcttccgatctaagaaaagcgggcttgcg

SEQ ID NO: 72 - DNA, 60 bp, synthetic construct
  source 1..60: mol_type = other DNA
gtgactggagttcagacgtgtgctcttccgatctacgatgtcctcgagaatatcttcgtt

SEQ ID NO: 73 - DNA, 55 bp, synthetic construct
  source 1..55: mol_type = other DNA
gtgactggagttcagacgtgtgctcttccgatctctgcatgaagttgcgattagc

SEQ ID NO: 74 - DNA, 59 bp, synthetic construct
  source 1..59: mol_type = other DNA
gtgactggagttcagacgtgtgctcttccgatctgtcctttctgggtcgtttgattctc

SEQ ID NO: 75 - DNA, 59 bp, synthetic construct
  source 1..59: mol_type = other DNA
gtgactggagttcagacgtgtgctcttccgatcttagaatcgtctttgaggaagctctg

SEQ ID NO: 76 - DNA, 60 bp, synthetic construct
  source 1..60: mol_type = other DNA
gtgactggagttcagacgtgtgctcttccgatctgtgatttgacgagtctccactaactg

SEQ ID NO: 77 - DNA, 53 bp, synthetic construct
  source 1..53: mol_type = other DNA
gtgactggagttcagacgtgtgctcttccgatcttgataagggcggtcccaac

SEQ ID NO: 78 - DNA, 55 bp, synthetic construct
  source 1..55: mol_type = other DNA
gtgactggagttcagacgtgtgctcttccgatctacgccctttgtcccaaacaat

SEQ ID NO: 79 - DNA, 60 bp, synthetic construct
  source 1..60: mol_type = other DNA
gtgactggagttcagacgtgtgctcttccgatctttggatttacccttttctactttcgc

SEQ ID NO: 80 - DNA, 63 bp, synthetic construct
  source 1..63: mol_type = other DNA
gtgactggagttcagacgtgtgctcttccgatctacgtactttgatggaagtgctaattcatt

SEQ ID NO: 81 - DNA, 61 bp, synthetic construct
  source 1..61: mol_type = other DNA
gtgactggagttcagacgtgtgctcttccgatctggtgaatgatattttcagcctgctcac

SEQ ID NO: 82 - DNA, 53 bp, synthetic construct
  source 1..53: mol_type = other DNA
gtgactggagttcagacgtgtgctcttccgatctcaagcttgtcgacctgcag

SEQ ID NO: 83 - DNA, 8065 bp, synthetic construct
  source 1..8065: mol_type = other DNA
tgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatggacaaaaagtactctattggcctggatatcgggaccaacagcgtcgggtgggctgttatcaccgacgagtataaagtaccttcgaaaaagttcaaagtgctgggcaacaccgatcgccattcaatcaaaaagaacttgattggtgcgctgttgtttgactccggggaaaccgccgaggcgactcgccttaaacgtacagcacgtcgccggtacactcggcgtaagaatcgcatttgctatttgcaggaaatctttagcaacgagatggcaaaagtcgatgactcgtttttccaccgcctcgaggaaagctttctggtggaggaagacaaaaagcatgagcgtcacccgatcttcggcaacattgtcgatgaagtagcgtatcatgaaaaatacccaaccatttaccacttacgcaaaaagctggtggacagcactgacaaagctgatttgcgccttatctatttagccctggcacatatgattaagtttcgtggtcacttcctgatcgaaggagacttaaatcccgacaacagtgatgttgataaattgtttattcagcttgtccaaacttacaatcaactgttcgaggaaaacccgatcaatgcctccggtgtggatgcaaaagccattttaagtgcacgccttagcaagtcccgtcgcttagaaaaccttatcgcgcagctgcccggcgagaaaaagaatggtttgtttgggaaccttattgccttgagcttaggcctcaccccgaatttcaaaagtaatttcgatcttgcagaagacgccaaattacaactgtcgaaggatacttatgatgacgatctcgataatctgttagcgcagattggtgaccaatacgccgatctttttctggcggctaaaaatctgagcgacgccatcttgctttcggatattctccgcgttaacaccgaaatcacgaaagcgcctcttagtgccagcatgattaaacgttatgatgaacaccaccaggacctgaccttactcaaagcgttggttcgccagcaactgccagagaagtacaaagaaatcttctttgatcagtcaaagaatggttatgccggctatattgacgggggtgcaagccaagaggaattctacaaatttatcaagcctattctggagaaaatggatggtaccgaagagttattggtgaagcttaaccgtgaagacctcctgcggaaacagcgcacattcgataatggttcgatcccacaccaaatccatttgggggagttacacgctattttgcgtcgccaggaagacttttaccctttcctgaaggataaccgggagaaaattgagaagatccttacctttcgtattccgtattacgtaggccccttagcacggggtaatagccgtttcgcgtggatgacacggaagtcggaagagacgatcaccccgtggaacttcgaagaggtagtcgacaagggcgcatcagcgcagtcttttattgaacgtatgacgaatttcgataaaaacttgcccaatgagaaggtgcttccgaaacattccttgttatatgaatattttacagtttacaacgagctgaccaaggttaaatacgtgacggaaggaatgcgcaagcccgcttttcttagcggtgagcaaaaaaaggcgatcgtcgacctgttattcaaaacgaatcgtaaggtgactgtaaagcaactcaaagaagattacttcaaaaagattgagtgcttcgacagcgtcgaaatctctggggtagaggatcggtttaacgcaagtttaggtacctaccatgacctgcttaaaatcattaaggataaagacttcttagataatgaagagaacgaagatattctcgaggacatcgtcttgacgttaaccttatttgaggatcgtgaaatgattgaggaacgcctcaaaacttatgcccacctgttcgacgataaggtgatgaagcagctgaaacgtcggcgctacacaggatggggccgcttgagtcgcaaacttattaacggaatccgtgacaagcaatccggcaaaacgattctggatttcttgaagtcggacggatttgctaatcgcaacttcatgcagttgatccatgatgactccctgacttttaaagaggatattcaaaaggcgcaggttagtggtcaaggcgacagcttacacgaacacatcgcaaatttggctggttcgccggccattaaaaaggggatcctccagaccgtgaaagttgtagatgagcttgttaaggtcatgggtcgtcataagcccgaaaacatcgtgattgaaatggcgcgggagaatcaaacgacccagaaaggacaaaagaatagccgtgaacggatgaagcggatcgaggaaggcattaaagagctggggtctcaaatcttgaaggaacaccctgtggagaacactcagctccaaaatgaaaaactttacctgtactatttgcagaacggacgcgatatgtacgtggaccaagagttggatattaatcggctgagtgactacgacgttgatcatatcgtcccgcagagcttcctcaaagacgattctattgacaataaggtactgacgcgctctgataaaaaccgtggtaagtcggacaacgtgccctccgaagaggttgtgaaaaagatgaaaaattattggcgccagcttttaaacgcgaagctgatcacacaacgtaaattcgataatttgaccaaggctgaacggggtggcctgagcgagttagataaggcaggatttattaaacgccagttagtggagactcgtcaaatcaccaaacatgtcgcgcagattttggacagccggatgaacaccaagtacgatgaaaatgacaaactgatccgtgaggtgaaagtcattactctgaagtccaaattagttagtgatttccggaaggactttcaattctacaaagtccgtgaaattaataactatcatcacgcacatgacgcgtacctgaatgcagtggttgggaccgcccttatcaagaaatatcctaagctggagtcggagtttgtctatggcgactataaggtatacgatgttcgcaaaatgattgcgaaatctgagcaggagatcggtaaggcaaccgcaaaatatttcttttactcaaacattatgaatttctttaagacagaaatcactctggccaacggggagattcgcaaacgtccgttgatcgaaacaaacggcgagactggcgaaattgtttgggacaaagggcgtgatttcgcgacggtgcgcaaggtactgagcatgcctcaagtcaatattgttaagaaaaccgaagtgcagacgggcgggttttccaaggaaagcatcttacccaaacgtaattcagataaacttattgcacgcaaaaaggactgggatccgaaaaagtatggaggcttcgacagtccaaccgtagcctactctgttctcgttgtagcgaaagtagaaaagggtaaatccaagaaactgaaatctgtcaaggagttgcttggaatcaccattatggagcgtagctccttcgagaagaacccgattgactttctggaagccaaaggatataaagaggtcaagaaagatcttatcattaagctgcctaagtattcactcttcgagctggaaaatggtcgtaaacgcatgctcgcttctgccggcgagttgcagaagggcaatgaattagcacttccatcaaagtacgttaacttcctgtatttggccagccattacgagaaactgaaggggtctccagaggacaacgaacagaaacaattatttgtagagcagcacaagcattatcttgatgaaatcattgagcaaatttccgaattcagtaaacgcgtaatcctggccgatgcaaacctcgacaaggtgctgagcgcttacaataagcatcgcgacaaacctatccgtgagcaggctgaaaatatcattcacctgttcacattaacgaacctgggcgctccggccgcttttaaatatttcgacacgacaatcgaccgtaagcgctataccagtacgaaagaagtgttggatgcgacccttattcaccagtcaattacaggattatatgagacccgtatcgaccttagccaattaggtggggatgagctcggcgcgcctgcaggtcgacaagcttgcggccgcataatgcttaagtcgaacagaaagtaatcgtatcctcagcggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcag

SEQ ID NO: 84 - DNA, 6011 bp, synthetic construct
  source 1..6011: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccaggctttacactttatgcttccggctcgtataatgtgtggaatcacacaggaaacagaattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgttgcctcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcaccggtttccagggcgcttatctggtgctgggtctggtggcgctgggcttcaccttaatttccgtgttcacgcttagcggccccggcccgctttccctgctgcgtcgtcaggtgaatgaagtcgcttaagcaatcaatgtcggatgcggaccgggagcgctgtgaatacagtgctccctttttttattgagctcggtaccttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgattgaaaacgttgcagttcaaggtttacacctataaaagagagagccgctatcgcctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccacccagatggtcagtgtgccggtctccgtcatcggagaagaagtggctgatctcagccaccgcgaaaatgacatcaaaaacgccattaatctgatgttctggggaatataatctagaggtgagtgagtgtgtgcgtgtggaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 85 - RNA, 100 nt, synthetic construct; identical to SEQ ID NO: 8
  source 1..100: mol_type = other RNA

SEQ ID NO: 86 - RNA, 74 nt, synthetic construct
  source 1..74: mol_type = other RNA
aattatggggattactaggagttttagagctagaaatagcaagttaaaataaggctagtccgttatcaactttt

SEQ ID NO: 87 - RNA, 68 nt, synthetic construct
  source 1..68: mol_type = other RNA
aattatggggattactaggagttttagaggaaacaagttaaaataaggctagtccgttatcaactttt

SEQ ID NO: 88 - RNA, 71 nt, synthetic construct
  source 1..71: mol_type = other RNA
aattatggggattactaggagttttagagctagaaatagcaagttaaaataaggctagtccgttatctttt

SEQ ID NO: 89 - RNA, 66 nt, synthetic construct
  source 1..66: mol_type = other RNA
aattatggggattactaggagttttagagctagaaatagcaagttaaaataaggctagtccgtttt

SEQ ID NO: 90 - RNA, 65 nt, synthetic construct
  source 1..65: mol_type = other RNA
aattatggggattactaggagttttagagctagaaatagcaagttaaaataaggctagtcctttt

SEQ ID NO: 91 - RNA, 62 nt, synthetic construct
  source 1..62: mol_type = other RNA
aattatggggattactaggagttttagagctagaaatagcaagttaaaataaggctagtttt

SEQ ID NO: 92 - RNA, 69 nt, synthetic construct
  source 1..69: mol_type = other RNA
aattatggggattactaggagttttagagctagaaatagcaagttaaaataaggctagtccgttatttt

SEQ ID NO: 93 - RNA, 65 nt, synthetic construct
  source 1..65: mol_type = other RNA
aattatggggattactaggagttttagaggaaacaagttaaaataaggctagtccgttatctttt

SEQ ID NO: 94 - RNA, 63 nt, synthetic construct
  source 1..63: mol_type = other RNA
aattatggggattactaggagttttagaggaaacaagttaaaataaggctagtccgttatttt

SEQ ID NO: 95 - RNA, 60 nt, synthetic construct
  source 1..60: mol_type = other RNA
aattatggggattactaggagttttagaggaaacaagttaaaataaggctagtccgtttt

SEQ ID NO: 96 - RNA, 59 nt, synthetic construct
  source 1..59: mol_type = other RNA
aattatggggattactaggagttttagaggaaacaagttaaaataaggctagtcctttt

SEQ ID NO: 97 - RNA, 100 nt, synthetic construct; identical to SEQ ID NO: 2
  source 1..100: mol_type = other RNA

SEQ ID NO: 98 - RNA, 74 nt, synthetic construct
  source 1..74: mol_type = other RNA
ggtgagtgagtgtgtgcgtggttttagagctagaaatagcaagttaaaataaggctagtccgttatcaactttt

SEQ ID NO: 99 - RNA, 68 nt, synthetic construct
  source 1..68: mol_type = other RNA
ggtgagtgagtgtgtgcgtggttttagaggaaacaagttaaaataaggctagtccgttatcaactttt

SEQ ID NO: 100 - RNA, 71 nt, synthetic construct
  source 1..71: mol_type = other RNA
ggtgagtgagtgtgtgcgtggttttagagctagaaatagcaagttaaaataaggctagtccgttatctttt

SEQ ID NO: 101 - RNA, 66 nt, synthetic construct
  source 1..66: mol_type = other RNA
ggtgagtgagtgtgtgcgtggttttagagctagaaatagcaagttaaaataaggctagtccgtttt

SEQ ID NO: 102 - RNA, 65 nt, synthetic construct
  source 1..65: mol_type = other RNA
ggtgagtgagtgtgtgcgtggttttagagctagaaatagcaagttaaaataaggctagtcctttt

SEQ ID NO: 103 - RNA, 62 nt, synthetic construct
  source 1..62: mol_type = other RNA
ggtgagtgagtgtgtgcgtggttttagagctagaaatagcaagttaaaataaggctagtttt

SEQ ID NO: 104 - RNA, 69 nt, synthetic construct
  source 1..69: mol_type = other RNA
ggtgagtgagtgtgtgcgtggttttagagctagaaatagcaagttaaaataaggctagtccgttatttt

SEQ ID NO: 105 - RNA, 65 nt, synthetic construct
  source 1..65: mol_type = other RNA
ggtgagtgagtgtgtgcgtggttttagaggaaacaagttaaaataaggctagtccgttatctttt

SEQ ID NO: 106 - RNA, 63 nt, synthetic construct
  source 1..63: mol_type = other RNA
ggtgagtgagtgtgtgcgtggttttagaggaaacaagttaaaataaggctagtccgttatttt

SEQ ID NO: 107 - RNA, 60 nt, synthetic construct
  source 1..60: mol_type = other RNA
ggtgagtgagtgtgtgcgtggttttagaggaaacaagttaaaataaggctagtccgtttt

SEQ ID NO: 108 - RNA, 59 nt, synthetic construct
  source 1..59: mol_type = other RNA
ggtgagtgagtgtgtgcgtggttttagaggaaacaagttaaaataaggctagtcctttt

SEQ ID NO: 109 - DNA, 358 bp, synthetic construct
  source 1..358: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagtccgttatcaacttgaaaaagtggcaccgagtcggtgcttttttt

SEQ ID NO: 110 - DNA, 332 bp, synthetic construct
  source 1..332: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagtccgttatcaacttttttt

SEQ ID NO: 111 - DNA, 326 bp, synthetic construct
  source 1..326: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagaggaaacaagttaaaataaggctagtccgttatcaacttttttt

SEQ ID NO: 112 - DNA, 329 bp, synthetic construct
  source 1..329: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagtccgttatcttttttt

SEQ ID NO: 113 - DNA, 324 bp, synthetic construct
  source 1..324: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagtccgttttttt

SEQ ID NO: 114 - DNA, 323 bp, synthetic construct
  source 1..323: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagtccttttttt

SEQ ID NO: 115 - DNA, 320 bp, synthetic construct
  source 1..320: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagttttttt

SEQ ID NO: 116 - DNA, 327 bp, synthetic construct
  source 1..327: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagagctagaaatagcaagttaaaataaggctagtccgttattttttt

SEQ ID NO: 117 - DNA, 323 bp, synthetic construct
  source 1..323: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagaggaaacaagttaaaataaggctagtccgttatcttttttt

SEQ ID NO: 118 - DNA, 321 bp, synthetic construct
  source 1..321: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagaggaaacaagttaaaataaggctagtccgttattttttt

SEQ ID NO: 119 - DNA, 318 bp, synthetic construct
  source 1..318: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagaggaaacaagttaaaataaggctagtccgttttttt

SEQ ID NO: 120 - DNA, 317 bp, synthetic construct
  source 1..317: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaattatggggattactaggagttttagaggaaacaagttaaaataaggctagtccttttttt

SEQ ID NO: 121 - DNA, 358 bp, synthetic construct
  source 1..358: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagtccgttatcaacttgaaaaagtggcaccgagtcggtgcttttttt

SEQ ID NO: 122 - DNA, 332 bp, synthetic construct
  source 1..332: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagtccgttatcaacttttttt

SEQ ID NO: 123 - DNA, 326 bp, synthetic construct
  source 1..326: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagaggaaacaagttaaaataaggctagtccgttatcaacttttttt

SEQ ID NO: 124 - DNA, 329 bp, synthetic construct
  source 1..329: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagtccgttatcttttttt

SEQ ID NO: 125 - DNA, 324 bp, synthetic construct
  source 1..324: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagtccgttttttt

SEQ ID NO: 126 - DNA, 323 bp, synthetic construct
  source 1..323: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagtccttttttt

SEQ ID NO: 127 - DNA, 320 bp, synthetic construct
  source 1..320: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagttttttt

SEQ ID NO: 128 - DNA, 327 bp, synthetic construct
  source 1..327: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagagctagaaatagcaagttaaaataaggctagtccgttattttttt

SEQ ID NO: 129 - DNA, 323 bp, synthetic construct
  source 1..323: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagaggaaacaagttaaaataaggctagtccgttatcttttttt

SEQ ID NO: 130 - DNA, 321 bp, synthetic construct
  source 1..321: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagaggaaacaagttaaaataaggctagtccgttattttttt

SEQ ID NO: 131 - DNA, 318 bp, synthetic construct
  source 1..318: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagaggaaacaagttaaaataaggctagtccgttttttt

SEQ ID NO: 132 - DNA, 317 bp, synthetic construct
  source 1..317: mol_type = other DNA
acgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgagtgagtgtgtgcgtggttttagaggaaacaagttaaaataaggctagtccttttttt

SEQ ID NO: 133 - protein, 1368 aa, synthetic construct [sequence omitted]

SEQ ID NO: 134 - DNA, 4104 bp, synthetic construct
  source 1..4104: mol_type = other DNA
atggacaaaaagtactctattggcctggatatcgggaccaacagcgtcgggtgggctgttatcaccgacgagtataaagtaccttcgaaaaagttcaaagtgctgggcaacaccgatcgccattcaatcaaaaagaacttgattggtgcgctgttgtttgactccggggaaaccgccgaggcgactcgccttaaacgtacagcacgtcgccggtacactcggcgtaagaatcgcatttgctatttgcaggaaatctttagcaacgagatggcaaaagtcgatgactcgtttttccaccgcctcgaggaaagctttctggtggaggaagacaaaaagcatgagcgtcacccgatcttcggcaacattgtcgatgaagtagcgtatcatgaaaaatacccaaccatttaccacttacgcaaaaagctggtggacagcactgacaaagctgatttgcgccttatctatttagccctggcacatatgattaagtttcgtggtcacttcctgatcgaaggagacttaaatcccgacaacagtgatgttgataaattgtttattcagcttgtccaaacttacaatcaactgttcgaggaaaacccgatcaatgcctccggtgtggatgcaaaagccattttaagtgcacgccttagcaagtcccgtcgcttagaaaaccttatcgcgcagctgcccggcgagaaaaagaatggtttgtttgggaaccttattgccttgagcttaggcctcaccccgaatttcaaaagtaatttcgatcttgcagaagacgccaaattacaactgtcgaaggatacttatgatgacgatctcgataatctgttagcgcagattggtgaccaatacgccgatctttttctggcggctaaaaatctgagcgacgccatcttgctttcggatattctccgcgttaacaccgaaatcacgaaagcgcctcttagtgccagcatgattaaacgttatgatgaacaccaccaggacctgaccttactcaaagcgttggttcgccagcaactgccagagaagtacaaagaaatcttctttgatcagtcaaagaatggttatgccggctatattgacgggggtgcaagccaagaggaattctacaaatttatcaagcctattctggagaaaatggatggtaccgaagagttattggtgaagcttaaccgtgaagacctcctgcggaaacagcgcacattcgataatggttcgatcccacaccaaatccatttgggggagttacacgctattttgcgtcgccaggaagacttttaccctttcctgaaggataaccgggagaaaattgagaagatccttacctttcgtattccgtattacgtaggccccttagcacggggtaatagccgtttcgcgtggatgacacggaagtcggaagagacgatcaccccgtggaacttcgaagaggtagtcgacaagggcgcatcagcgcagtcttttattgaacgtatgacgaatttcgataaaaacttgcccaatgagaaggtgcttccgaaacattccttgttatatgaatattttacagtttacaacgagctgaccaaggttaaatacgtgacggaaggaatgcgcaagcccgcttttcttagcggtgagcaaaaaaaggcgatcgtcgacctgttattcaaaacgaatcgtaaggtgactgtaaagcaactcaaagaagattacttcaaaaagattgagtgcttcgacagcgtcgaaatctctggggtagaggatcggtttaacgcaagtttaggtacctaccatgacctgcttaaaatcattaaggataaagacttcttagataatgaagagaacgaagatattctcgaggacatcgtcttgacgttaaccttatttgaggatcgtgaaatgattgaggaacgcctcaaaacttatgcccacctgttcgacgataaggtgatgaagcagctgaaacgtcggcgctacacaggatggggccgcttgagtcgcaaacttattaacggaatccgtgacaagcaatccggcaaaacgattctggatttcttgaagtcggacggatttgctaatcgcaacttcatgcagttgatccatgatgactccctgacttttaaagaggatattcaaaaggcgcaggttagtggtcaaggcgacagcttacacgaacacatcgcaaatttggctggttcgccggccattaaaaaggggatcctccagaccgtgaaagttgtagatgagcttgttaaggtcatgggtcgtcataagcccgaaaacatcgtgattgaaatggcgcgggagaatcaaacgacccagaaaggacaaaagaatagccgtgaacggatgaagcggatcgaggaaggcattaaagagctggggtctcaaatcttgaaggaacaccctgtggagaacactcagctccaaaatgaaaaactttacctgtactatttgcagaacggacgcgatatgtacgtggaccaagagttggatattaatcggctgagtgactacgacgttgatcatatcgtcccgcagagcttcctcaaagacgattctattgacaataaggtactgacgcgctctgataaaaaccgtggtaagtcggacaacgtgccctccgaagaggttgtgaaaaagatgaaaaattattggcgccagcttttaaacgcgaagctgatcacacaacgtaaattcgataatttgaccaaggctgaacggggtggcctgagcgagttagataaggcaggatttattaaacgccagttagtggagactcgtcaaatcaccaaacatgtcgcgcagattttggacagccggatgaacaccaagtacgatgaaaatgacaaactgatccgtgaggtgaaagtcattactctgaagtccaaattagttagtgatttccggaaggactttcaattctacaaagtccgtgaaattaataactatcatcacgcacatgacgcgtacctgaatgcagtggttgggaccgcccttatcaagaaatatcctaagctggagtcggagtttgtctatggcgactataaggtatacgatgttcgcaaaatgattgcgaaatctgagcaggagatcggtaaggcaaccgcaaaatatttcttttactcaaacattatgaatttctttaagacagaaatcactctggccaacggggagattcgcaaacgtccgttgatcgaaacaaacggcgagactggcgaaattgtttgggacaaagggcgtgatttcgcgacggtgcgcaaggtactgagcatgcctcaagtcaatattgttaagaaaaccgaagtgcagacgggcgggttttccaaggaaagcatcttacccaaacgtaattcagataaacttattgcacgcaaaaaggactgggatccgaaaaagtatggaggcttcgacagtccaaccgtagcctactctgttctcgttgtagcgaaagtagaaaagggtaaatccaagaaactgaaatctgtcaaggagttgcttggaatcaccattatggagcgtagctccttcgagaagaacccgattgactttctggaagccaaaggatataaagaggtcaagaaagatcttatcattaagctgcctaagtattcactcttcgagctggaaaatggtcgtaaacgcatgctcgcttctgccggcgagttgcagaagggcaatgaattagcacttccatcaaagtacgttaacttcctgtatttggccagccattacgagaaactgaaggggtctccagaggacaacgaacagaaacaattatttgtagagcagcacaagcattatcttgatgaaatcattgagcaaatttccgaattcagtaaacgcgtaatcctggccgatgcaaacctcgacaaggtgctgagcgcttacaataagcatcgcgacaaacctatccgtgagcaggctgaaaatatcattcacctgttcacattaacgaacctgggcgctccggccgcttttaaatatttcgacacgacaatcgaccgtaagcgctataccagtacgaaagaagtgttggatgcgacccttattcaccagtcaattacaggattatatgagacccgtatcgaccttagccaattaggtggggat

SEQ ID NO: 135 - protein, 1368 aa, synthetic construct
MDKKYSIGLDIGTNSVGWAVITDEYKVPSKEFKVLGNTDRHSIKKNLIGALLFDSAKTAEATRLKRTKRRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHKQDLRLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETIRPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFYKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 136 - DNA, 4104 bp, synthetic construct
  source 1..4104: mol_type = other DNA
atggacaagaagtacagcatcggcctggacatcggcaccaattctgttggctgggccgtgatcaccgacgagtacaaggtgcccagcaaggagttcaaggtgctgggcaacaccgaccggcacagcatcaagaagaatctgatcggcgccctgctgttcgactctgccaagacagccgaagccaccagactgaagagaaccaagagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcacaagcaggatctgcggctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatccggccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcggccacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttctacaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgac

SEQ ID NO: 137 - protein, 1368 aa, synthetic construct
MDKKYSIGLDIGTNSVGWAVITDEYKVPSKKFKVLGNTDRHSIKKNLIGALLFDSGETAEATRLKRTKRRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETIRPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFYKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 138 - DNA, 4104 bp, synthetic construct
  source 1..4104: mol_type = other DNA
atggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccaagagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatccggccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttctacaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgac

SEQ ID NO: 139 - protein, 1368 aa, synthetic construct
MDKKYSIGLDIGTNSVGWAVITDEYKVPSKEFKVLGNTDRHSIKKNLIGALLFDSAKTAEATRLKRTKRRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETIRPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFYKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 140 - DNA, 4104 bp, synthetic construct
  source 1..4104: mol_type = other DNA
atggacaagaagtacagcatcggcctggacatcggcaccaattctgttggctgggccgtgatcaccgacgagtacaaggtgcccagcaaggagttcaaggtgctgggcaacaccgaccggcacagcatcaagaagaatctgatcggcgccctgctgttcgactctgccaagacagccgaagccaccagactgaagagaaccaagagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatccggccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttctacaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgac

SEQ ID NO: 141 - protein, 1368 aa, synthetic construct
MDKKYSIGLDIGTNSVGWAVITDEYKVPSKEFKVLGNTDRHSIKKNLIGALLFDSAKTAEATRLKRTKRRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETIRPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVREVLSMPQVNIVKKTEVQTGGFYKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 142 - DNA, 4104 bp, synthetic construct
  source 1..4104: mol_type = other DNA
atggacaagaagtacagcatcggcctggacatcggcaccaattctgttggctgggccgtgatcaccgacgagtacaaggtgcccagcaaggagttcaaggtgctgggcaacaccgaccggcacagcatcaagaagaatctgatcggcgccctgctgttcgactctgccaagacagccgaagccaccagactgaagagaaccaagagacggcggtacaccagaagaaagaaccggatctgctacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatccggccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcgggaggtgctgagcatgccccaagtgaatatcgtgaagaaaaccgaggtgcagacaggcggcttctacaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagcttggaggcgac

SEQ ID NO: 143 - RNA, 70 nt, synthetic construct
  source 1..70: mol_type = other RNA
nnnnnnnnnnnnnnnnnnnngttttagagctagaaatagcaagttaaaataaggctagtccgttatcaac

SEQ ID NO: 144 - RNA, 69 nt, synthetic construct
  source 1..69: mol_type = other RNA
nnnnnnnnnnnnnnnnnnnngttttagagctagaaatagcaagttaaaataaggctagtccgttatcaa

SEQ ID NO: 145 - RNA, 68 nt, synthetic construct
  source 1..68: mol_type = other RNA
nnnnnnnnnnnnnnnnnnnngttttagagctagaaatagcaagttaaaataaggctagtccgttatca

SEQ ID NO: 146 - RNA, 67 nt, synthetic construct
  source 1..67: mol_type = other RNA
nnnnnnnnnnnnnnnnnnnngttttagagctagaaatagcaagttaaaataaggctagtccgttatc

SEQ ID NO: 147 - RNA, 66 nt, synthetic construct
  source 1..66: mol_type = other RNA
nnnnnnnnnnnnnnnnnnnngttttagagctagaaatagcaagttaaaataaggctagtccgttat

SEQ ID NO: 148 - RNA, 65 nt, synthetic construct
  source 1..65: mol_type = other RNA
nnnnnnnnnnnnnnnnnnnngttttagagctagaaatagcaagttaaaataaggctagtccgtta

SEQ ID NO: 149 - RNA, 64 nt, synthetic construct
  source 1..64: mol_type = other RNA
nnnnnnnnnnnnnnnnnnnngttttagagctagaaatagcaagttaaaataaggctagtccgtt

SEQ ID NO: 150 - RNA, 63 nt, synthetic construct
  source 1..63: mol_type = other RNA
nnnnnnnnnnnnnnnnnnnngttttagagctagaaatagcaagttaaaataaggctagtccgt

SEQ ID NO: 151 - RNA, 62 nt, synthetic construct
  source 1..62: mol_type = other RNA
nnnnnnnnnnnnnnnnnnnngttttagagctagaaatagcaagttaaaataaggctagtccg

SEQ ID NO: 152 - RNA, 61 nt, synthetic construct
  source 1..61: mol_type = other RNA
nnnnnnnnnnnnnnnnnnnngttttagagctagaaatagcaagttaaaataaggctagtcc

SEQ ID NO: 153 - RNA, 60 nt, synthetic construct
  source 1..60: mol_type = other RNA
nnnnnnnnnnnnnnnnnnnngttttagagctagaaatagcaagttaaaataaggctagtc

SEQ ID NO: 154 - RNA, 59 nt, synthetic construct
  source 1..59: mol_type = other RNA
nnnnnnnnnnnnnnnnnnnngttttagagctagaaatagcaagttaaaataaggctagt

SEQ ID NO: 155 - RNA, 58 nt, synthetic construct
  source 1..58: mol_type = other RNA
nnnnnnnnnnnnnnnnnnnngttttagagctagaaatagcaagttaaaataaggctag

SEQ ID NO: 156 - RNA, 65 nt, synthetic construct
  source 1..65: mol_type = other RNA
nnnnnnnnnnnnnnnnnnnngttttagaggaaacaagttaaaataaggctagtccgttatcaact

SEQ ID NO: 157 - RNA, 64 nt, synthetic construct
  source 1..64: mol_type = other RNA
nnnnnnnnnnnnnnnnnnnngttttagaggaaacaagttaaaataaggctagtccgttatcaac

SEQ ID NO: 158 - RNA, 63 nt, synthetic construct
  source 1..63: mol_type = other RNA
nnnnnnnnnnnnnnnnnnnngttttagaggaaacaagttaaaataaggctagtccgttatcaa

SEQ ID NO: 159 - RNA, 62 nt, synthetic construct
  source 1..62: mol_type = other RNA
nnnnnnnnnnnnnnnnnnnngttttagaggaaacaagttaaaataaggctagtccgttatca

SEQ ID NO: 160 - RNA, 61 nt, synthetic construct
  source 1..61: mol_type = other RNA
nnnnnnnnnnnnnnnnnnnngttttagaggaaacaagttaaaataaggctagtccgttatc

SEQ ID NO: 161 - RNA, 60 nt, synthetic construct
  source 1..60: mol_type = other RNA
nnnnnnnnnnnnnnnnnnnngttttagaggaaacaagttaaaataaggctagtccgttat

SEQ ID NO: 162 - RNA, 59 nt, synthetic construct
  source 1..59: mol_type = other RNA
nnnnnnnnnnnnnnnnnnnngttttagaggaaacaagttaaaataaggctagtccgtta

SEQ ID NO: 163 - RNA, 58 nt, synthetic construct
  source 1..58: mol_type = other RNA
nnnnnnnnnnnnnnnnnnnngttttagaggaaacaagttaaaataaggctagtccgtt

SEQ ID NO: 164 - RNA, 57 nt, synthetic construct
  source 1..57: mol_type = other RNA
nnnnnnnnnnnnnnnnnnnngttttagaggaaacaagttaaaataaggctagtccgt

SEQ ID NO: 165 - RNA, 56 nt, synthetic construct
  source 1..56: mol_type = other RNA
nnnnnnnnnnnnnnnnnnnngttttagaggaaacaagttaaaataaggctagtccg

SEQ ID NO: 166 - RNA, 55 nt, synthetic construct
  source 1..55: mol_type = other RNA
nnnnnnnnnnnnnnnnnnnngttttagaggaaacaagttaaaataaggctagtcc

SEQ ID NO: 167 - RNA, 54 nt, synthetic construct
  source 1..54: mol_type = other RNA
nnnnnnnnnnnnnnnnnnnngttttagaggaaacaagttaaaataaggctagtc

SEQ ID NO: 168 - RNA, 53 nt, synthetic construct
  source 1..53: mol_type = other RNA
nnnnnnnnnnnnnnnnnnnngttttagaggaaacaagttaaaataaggctagt

SEQ ID NO: 169 - protein, 26 aa, synthetic construct
KRRWKKNFIAVSAANRFKKISSSGAL

SEQ ID NO: 170 - protein, 15 aa, synthetic construct
GLNDIFEAQKIEWHE

SEQ ID NO: 171 - protein, 6 aa, synthetic construct
EEEEEE

SEQ ID NO: 172 - protein, 13 aa, synthetic construct
GAPVPYPDPLEPR

SEQ ID NO: 173 - protein, 8 aa, synthetic construct
DYKDDDDK

SEQ ID NO: 174 - protein, 9 aa, synthetic construct
YPYDVPDYA

SEQ ID NO: 175 - protein, 6 aa, synthetic construct
HHHHHH

SEQ ID NO: 176 - protein, 10 aa, synthetic construct
EQKLISEEDL

SEQ ID NO: 177 - protein, 18 aa, synthetic construct
TKENPRSNQEESYDDNES

SEQ ID NO: 178 - protein, 15 aa, synthetic construct
KETAAAKFERQHMDS

SEQ ID NO: 179 - protein, 38 aa, synthetic construct
MDEKTTGWRGGHVVEGLAGELEQLRARLEHHPQGQREP

SEQ ID NO: 180 - protein, 13 aa, synthetic construct
SLAELLNAGLGGS

SEQ ID NO: 181 - protein, 8 aa, synthetic construct
TQDPSRVG

SEQ ID NO: 182 - protein, 8 aa, synthetic construct
WSHPQFEK

SEQ ID NO: 183 - protein, 6 aa, synthetic construct
CCPGCC

SEQ ID NO: 184 - protein, 14 aa, synthetic construct
GKPIPNPLLGLDST

SEQ ID NO: 185 - protein, 11 aa, synthetic construct
YTDIEMNRLGK

SEQ ID NO: 186 - protein, 8 aa, synthetic construct
DLYDDDDK

SEQ ID NO: 187 - protein, 16 aa, synthetic construct
TDKDMTITFTNKKDAE

SEQ ID NO: 188 - protein, 13 aa, synthetic construct
AHIVMVDAYKPTK

SEQ ID NO: 189 - protein, 12 aa, synthetic construct
KLGDIEFIKVNK

SEQ ID NO: 190 - RNA, 29 nt, synthetic construct
  source 1..29: mol_type = other RNA
ggtgagtgagtgtgtgcgtggttttagag

SEQ ID NO: 191 - RNA, 35 nt, synthetic construct
  source 1..35: mol_type = other RNA
caagttaaaataaggctagtccgttatcaactttt

SEQ ID NO: 192 - RNA, 32 nt, synthetic construct
  source 1..32: mol_type = other RNA
caagttaaaataaggctagtccgttatctttt

SEQ ID NO: 193 - RNA, 65 nt, synthetic construct; identical to SEQ ID NO: 105
  source 1..65: mol_type = other RNA

SEQ ID NO: 194 - RNA, 30 nt, synthetic construct
  source 1..30: mol_type = other RNA
caagttaaaataaggctagtccgttatttt

SEQ ID NO: 195 - RNA, 63 nt, synthetic construct; identical to SEQ ID NO: 106
  source 1..63: mol_type = other RNA

SEQ ID NO: 196 - RNA, 27 nt, synthetic construct
  source 1..27: mol_type = other RNA
caagttaaaataaggctagtccgtttt

SEQ ID NO: 197 - RNA, 60 nt, synthetic construct; identical to SEQ ID NO: 107
  source 1..60: mol_type = other RNA

SEQ ID NO: 198 - RNA, 26 nt, synthetic construct
  source 1..26: mol_type = other RNA
caagttaaaataaggctagtcctttt

SEQ ID NO: 199 - RNA, 59 nt, synthetic construct; identical to SEQ ID NO: 108
  source 1..59: mol_type = other RNA

(19) United States
(12) Patent Application Publication
(10) Pub. No.: US 2025/0297237 A1
(43) Pub. Date: Sep. 25, 2025

(54) Engineering of CAS9 Variants That Possess Targeted Nuclease Activity When Paired with Short SGRNAS

(71) Applicant: Integrated DNA Technologies, Inc., Coralville, IA (US)

(72) Inventors: Steve Ehren GLENN, North Liberty, IA (US); Christopher Anthony VAKULSKAS, North Liberty, IA (US)

(73) Assignee: Integrated DNA Technologies, Inc., Coralville, IA (US)

(21) Appl. No.: 19/085,189

(22) Filed: Mar. 20, 2025

Related U.S. Application Data
(60) Provisional application No. 63/567,739, filed on Mar. 20, 2024.

(51) Int. Cl. C12N 9/22; C12N 15/11; C12N 15/86; C12N 15/88

(52) U.S. Cl. CPC C12N 9/22; C12N 15/111; C12N 15/86; C12N 15/88; C12N 2310/20; C12N 2310/531

(57) ABSTRACT

The present invention pertains to isolated variants of the Cas9 protein that, when complexed with a guide RNA with a scaffold sequence shorter than 76 nucleotides to form a CRISPR/Cas9 endonuclease, the resultant CRISPR/Cas9 endonuclease cleaves a double-stranded DNA target in living cells with greater efficiency than a CRISPR/Cas9 endonuclease comprising the wild-type Cas9 protein complexed with the guide RNA with a scaffold sequence shorter than 76 nucleotides.

This application claims the benefit of U.S. Ser. No. 63/567,739, filed Mar. 20, 2024, the entirety of which is incorporated herein by reference.

### SEQUENCE LISTING

The instant application contains a Sequence Listing which has been submitted in XML format via Patent Center and is hereby incorporated by reference in its entirety. Said XML copy, created on Mar. 20, 2025, is named 63910022US01.xml, and is 241 kbytes in size.

### FIELD OF THE INVENTION

This invention pertains to the ability of CRISPR/Cas9 to cleave double-stranded DNA in a targeted manner in living cells when complexed with sgRNAs containing scaffold regions shorter than 76 nucleotides.

### BACKGROUND OF THE INVENTION

Cas9 is an RNA guided endonuclease from the Clustered Regularly Interspaced Short Palindromic Repeat (CRISPR)-Cas (CRISPR-associated) bacterial adaptive immune system of Streptococcus pyogenes¹. Cas9 is guided to a 23 nt DNA target sequence by a target site-specific 20 nt complementary RNA (part of the 44 nt crRNA) and a universal 89 nt tracrRNA, collectively referred to as the guide RNA (gRNA) complex. The Cas9-gRNA ribonucleoprotein (RNP) complex mediates double-stranded DNA breaks (DSBs) which are then typically repaired by the non-homologous end joining (NHEJ), microhomology mediated end joining, or homology-directed repair (HDR) system if a suitable template nucleic acid is present.

Cas9 can also be directed to cleave DNA using a chimeric single guide RNA (sgRNA) consisting of a fusion of the crRNA and tracrRNA using a flexible linker¹. One of the earliest chimeric guide designs consisted of a 20 nt spacer sequence+12 nt from the crRNA constant region+a 4 nt flexible linker+26 nt from the tracrRNA (62 nucleotides total). While this design was active in vitro, it resulted in very poor though detectible activity at a subset of sites tested in human cells^{1, 2}. A different guide RNA design incorporating a much more extensive (60 nt) segment of the tracrRNA was later found to facilitate much higher levels of editing in human cells³. This design consisted of a 96 nt guide expressed using a U6 promoter in human cells, resulting in a ˜100 nt single guide RNA when the average ˜4 U's are added as part of the terminator sequence for the U6 promoter³⁻⁵. This is the design typically used today for Cas9 mediated genome editing.

Efficient editing can be achieved when Cas9 is delivered as a ribonucleoprotein complex (RNP). Early publications using Cas9 to edit in human cells expressed Cas9 and the guide RNAs off of plasmids, however this approach can lead to high levels of off target editing and risks plasmid integration into the genome^{2, 3, 6, 7}. Alternatively, high levels of editing can be achieved through delivery of Cas9 protein purified from E. coli in complex with sgRNA that has been produced through in vitro transcription or chemical synthesis^{6, 8, 9}. While chemical synthesis provides the advantage of being able to use chemical modifications to improve guide stability and editing levels, this comes with the challenge of a tradeoff between guide length and full-length yield during synthesis^{8.}

The present disclosure pertains to the ability of a variant of CRISPR/Cas9 to cleave double-stranded DNA in a targeted manner in living cells when complexed with sgRNAs with scaffold sequences shorter than 76 nucleotides

### BRIEF SUMMARY OF THE INVENTION

In a first aspect, an isolated variant of Cas9 protein is disclosed. When the isolated variant of Cas9 protein is complexed with an RNA guide with a scaffold sequence shorter than 76 nucleotides to form a CRISPR/Cas9 endonuclease, the resultant CRISPR/Cas9 endonuclease cleaves a double-stranded DNA target in living cells with greater efficiency than a CRISPR/Cas9 endonuclease comprising the wild-type Cas9 protein complexed with an RNA guide with a scaffold sequence shorter than 76 nucleotides.

In a second aspect, an isolated nucleic acid encoding a variant of Cas9 protein is disclosed.

In some embodiments, the variant of Cas9 protein as disclosed herein comprises at least one amino acid substitution selected from the group consisting of the following relative to the wild-type Cas9 amino acid sequence of SEQ ID NO: 133:
at least one amino acid substitution selected from the group consisting of D2T, D2Q, D2V, D2Y, D2S, D2C, D2L, D2F, D2N, D2R, D2M, D2P, D2E, D2A, K3N, K3I, K3R, K3D, K3V, K3Y, K3S, K3F, K4S, S6T, G8P, G8T, L9V, T13S, T13V, N14D, N14S, S15T, V16I, V16L, W18Y, A19V, A19L, A19C, T22E, T22D, T22V, T22Q, D23S, D23T, D23P, D23C, E24T, E24H, E24D, E24G, E24S, E24A, E24R, Y25W, Y25F, Y25I, K26N, K26H, K26R, K26E, K26D, K26V, K26Y, K26C, K26S, V27D, P28I, P28A, P28V, P28Y, P28F, P28Q, P28H, S29R, S29L, S29A, S29K, S29Q, S29E, S29V, S29N, S29I, K30T, K30M, K30I, K30L, K30A, K30G, K30S, K30F, K30C, K30R, K30D, K30V, K30Y, K30W, K30Q, K30E, K31N, K31R, K31M, K31Q, K31P, K31E, K31V, K31S, K31G, K31I, K31L, K31D, K31A, K31T, K31W, F32T, F32M, F32L, F32A, F32Y, F32I, F32P, F32C, F32V, K33N, K33T, K33S, K33I, K33Q, K33H, K33P, K33R, K33L, K33E, K33D, K33A, K33G, K33V, K33Y, K33C, K33F, K33W, K33M, V34L, V34R, V34I, V34A, V34C, V34T, L35P, L35R, L35E, L35S, L35V, L35T, L35A, L35K, L35N, G36A, G36S, G36N, G36Q, N37K, N37G, N37T, N37M, N37R, N37A, T38R, T38D, T38G, T38A, T38V, T38M, T38S, D39Q, D39E, D39G, D39V, D39S, D39F, D39L, D39A, D39R, D39W, R40N, R40T, R40S, R40H, R40P, R40L, R40E, R40G, R40V, R40W, R40C, R40F, R40M, R40A, H41T, H41M, H41R, H41E, H41D, H41Y, H41C, H41N, H41I, H41P, H41L, H41S, H41F, S42M, S42H, S42R, S42E, S42A, S42L, S42Q, S42P, S42I, S42D, S42Y, I43M, I43H, I43L, I43D, I43A, I43G, I43V, I43Y, I43W, I43T, I43R, I43E, I43C, I43S, I43Q, K44S, K44I, K44Q, K44D, K44A, K44Y, K44L, K44G, K44V, K44C, K44M, K44E, K44P, K45R, K45A, K45M, K45S, N46S, N46H, N46P, N46L, N46E, N46G, N46Y, N46C, N46K, N46M, N46R, N46D, N46A, N46F, N46I, N46Q, N46W, L47S, L47V, 148M, I48W, I48L, A50I, A50L, A50V, A50C, A50T, A50S, A50N, A50Y, A50F, A50H, L51T, L51R, L51A, L51V, L51C, L51Y, L51F, L51W, L52I, L52F, L52V, F53L, F53Y, D54K, D54N, D54T, D54R, D54S, D54P, D54A, D54H, D54G, D54L, D54E, S55T, S55E, S55P, S55F, S55G, S55M, S55R, G56R, G56A, G56V, G56P, G56S, E57K, E57T, E57R, E57M, E57Q, E57L, E57V, E57S, E57C, E57A, E57D, E57I, E57H, T58S, T58G, T58R, A59L, E60K, E60T, E60R, E60S, E60Q, E60H, E60A, E60V, E60Y, E60L, E60D, E60G, A61K, A61R, A61L, A61V, A61F, A61N, A61T, T62R, L64K, L64R, L64M, L64I, K65H, K65R, K65L, K65Y, K65F, K65M, K65A, R66P, T67S, T67G, T67R, T67A, T67Q, T67C, T67L, A68K, A68T, A68R, A68M, A68I, A68Q, A68L, A68V, A68H, A68S, R70M, Y72N, Y72T, Y721, Y72L, Y72G, Y72V, Y72C, Y72F, Y72M, Y72A, Y72K, T73R, T73A, K76R, K76S, K76H, K76T, N77K, N77R, N77C, N77A, N77M, N77Y, R78W, R78P, I79L, C80K, C80N, C80R, C80I, C80H, C80L, C80V, C80S, C80G, Q83I, E84K, E84S, E84G, E84F, E84D, 185M, I85L, S87T, S87M, S87G, S87V, S87K, S87R, S87H, S87E, S87A, S87W, N88I, N88E, N88V, N88S, N88Q, N88A, N88F, E89P, E89T, E89S, E89A, M90L, A91K, A91T, A91H, A91E, A91S, A91C, A91L, A91N, A91V, K92N, K92S, K92I, K92L, K92V, K92T, K92M, K92P, K92A, K92G, V93T, V93E, V93M, D95T, D95M, D95P, D95G, D95V, D95C, D95L, D95K, D95I, D95Q, D95R, S96T, S96H, S96A, S96K, S96N, S96R, S96L, S96E, S96V, S96Y, F98W, H99K, H99R, H99E, H99A, H99Y, H99C, H99F, H99M, H99D, H99Q, H99S, H99W, H99L, H99N, H99T, H99G, H99V, L101R, E102A, E102G, E102N, E102T, E102Q, E102R, E102S, F105S, V107I, V107T, E108I, E108P, E108A, E108S, E108C, E108T, E108M, E108R, E108H, E108V, E108N, K112T, K112P, K112A, H113Y, H113C, H113F, E114T, E114S, E114Q, E114N, E114A, E114P, R115K, H116W, I118F, I118M, I118S, F119Y, G120S, G120P, I122V, I122L, 1122R, V123T, V123H, V123S, V123L, V123N, V123M, V123D, V123G, D124K, D124E, D124M, D124R, D124S, D124A, E125D, V126I, V126D, V126A, A127K, A127S, A127N, A127R, A127L, Y128H, H129L, E130K, E130R, E130I, E130Q, E130A, E130M, E130D, E130L, E130N, E130G, E130T, E130H, K131R, K131M, K131Q, K131L, K131E, K131A, K131T, K131N, K131D, K131H, K131S, Y132S, I135S, Y136F, K140N, K140T, K140Q, K140H, K140L, K140A, K140G, K140Y, K140S, K140C, K140F, K140W, K141R, K141M, K141Q, K141H, K141E, K141A, K141V, K141W, K141F, K141D, L142Q, V143A, V143T, V143C, V143L, D144T, D144I, S145G, S145E, S145A, S145C, T146R, T146P, T146A, T146Y, T146S, T146N, T146I, T146G, T146L, D147K, D147N, D147H, D147A, D147F, D147P, D147R, K148R, K148I, K148A, K148S, K148C, A149G, A149L, A149E, A149S, L151A, L151C, L151P, I154V, I154L, L156S, L156N, L156C, L156M, L156H, L156V, A157I, L158V, L158S, L158A, L158T, L158C, A159S, A159H, M161S, M161L, M161C, I162L, F164Y, F168L, L169N, L169R, L169H, L169F, L169K, L169G, D173E, D173P, N175T, N175M, N175E, N175A, N175V, N175C, N175R, N175S, P176N, P176S, P176D, P176A, P176C, D177Y, D177M, D177E, D177C, N178R, D180T, D180S, D180M, D180H, D180L, D180E, D180A, D180G, D180Y, D180W, D180C, D180F, V181N, V181P, V181E, V181D, V181S, V181R, V181L, V181A, D182H, D182A, D182G, D182E, D182W, K183N, K183T, K183S, K183M, K183I, K183Q, K183H, K183P, K183C, K183F, K183R, K183L, K183E, K183A, K183G, K183V, L184I, F185T, F185Q, F185L, F185E, F185A, F185S, F185I, F185G, F185V, F185Y, I186K, I186E, I186D, I186S, 1186W, I186T, 1186Q, 1186P, I186G, I186C, I186F, Q187P, Q187D, Q187Y, Q187W, Q187T, Q187R, Q187S, Q187H, Q187C, L188I, L188Q, L188P, L188E, L188V, L188Y, L188S, L188F, L188D, L188G, V189N, V189Y, V189W, V189C, V189T, V189R, V189L, V189F, Q190T, Q190E, Q190A, Q190V, Q190W, Q190C, Q190P, Q190R, Q190G, T191S, T191P, T191L, T191E, T191D, T191A, T191V, T191K, T191G, Y192H, Y192V, Y192F, Y192S, Y192L, Y192W, N193Q, N193A, N193G, N193W, N193L, N193V, Q194I, Q194H, Q194L, Q194E, Q194G, Q194Y, Q194S, Q194W, Q194C, Q194T, Q194D, Q194V, L195T, L195M, L195I, L195Q, L195H, L195E, L195D, L195A, L195G, L195V, L195S, L195W, L195F, L195C, F196M, F196L, F196E, F196D, F196G, F196V, F196W, F196T, F196S, F196A, F196C, E197D, E197V, E197S, E197P, E198T, E198D, E198C, E198R, E198H, E198P, E198G, E198Y, E198S, E198L, N199K, N199R, N199E, N199A, N199G, N199S, N1991, N199Q, N199P, N199L, N199D, N199C, P200K, P200T, P200V, P200F, P200N, P200R, P200G, P200C, P200L, I201T, I201S, I201M, I201L, I201E, I201G, I201V, I201Y, I201C, I201F, I201K, I201R, I201D, I201W, N202K, N202R, N202S, N202P, N202D, N202A, N202Y, N202F, N202M, N202I, N202L, N202G, N202V, A203I, A203Q, A203L, A203E, A203W, A203F, A203T, A203M, A203H, A203R, A203V, A203S, S204N, S204M, S204R, S204L, S204A, S204V, S204C, S204T, S204P, S204G, S204W, S204F, G205T, G205I, G205Q, G205E, G205V, G205W, G205N, G205M, G205R, G205L, G205D, G205Y, G205S, V206K, V206N, V206R, V206S, V206M, V206H, V206P, V206L, V206E, V206D, V206A, V206G, V206Y, V206W, V206F, V206Q, D207T, D207R, D207P, D207L, D207A, D207G, D207V, D207Y, D207S, D207I, D207W, A208T, A208S, A208H, A208P, A208L, A208E, A208D, A208G, A208V, A208Y, A208W, A208C, A208F, A208M, A208I, K209N, K209G, A210N, A210T, A210M, A210P, A210G, A210S, A210K, A210R, A210Q, A210V, I211S, I211M, 1211H, I211P, I211L, I211E, I211A, I211G, I211V, 1211F, I211T, I211R, I211D, L212I, L212V, L212F, S213N, S213T, S213M, S213L, S213E, S213C, S213R, S213I, S213Q, S213A, S213V, A214S, A214D, A214W, A214G, R215T, R215S, R215M, R215I, R215Q, R215P, R215L, R215E, R215A, R215V, R215Y, R215W, R215C, R215F, R215N, L216E, L216D, L216Y, L216H, S217R, S217M, S217Q, S217E, S217D, S217A, S217C, S217N, S217I, S217P, S217G, S217V, S217Y, S219T, S219I, S219Q, S219H, S219L, S219E, S219D, S219A, S219G, S219W, S219C, S219F, S219P, S219V, R220K, R220T, R220S, R220M, R220Q, R220H, R220P, R220L, R220E, R220D, R220A, R220G, R220V, R220C, R220F, R220I, R220W, R221E, R221G, R221Y, L222N, L222T, L222S, L222M, L222I, L222Q, L222H, L222E, L222A, L222G, L222Y, L222W, L222C, L222F, E223S, E223P, E223D, N224T, N224P, N224E, N224D, N224A, N224V, N224S, N224W, N224L, L225K, L225S, L225M, L225Q, L225G, L225V, L225Y, L225W, L225A, L225C, I226K, 1226N, I226T, I226S, I226M, 1226Q, 1226P, 1226L, 1226D, 1226G, 1226V, I226R, I226H, 1226E, I226A, I226W, I226F, A227T, A227S, A227L, A227D, A227W, A227C, A227F, A227H, A227P, A227G, A227Y, Q228N, Q228T, Q228P, Q228E, Q228G, Q228V, Q228S, Q228W, Q228L, Q228A, Q228Y, L229K, L229M, L229H, L229P, L229D, L229Y, L229W, L229F, L229T, L229R, L229S, L229A, P230M, P230H, P230G, P230S, P230L, P230V, P230Y, P230W, G231K, G231N, G231T, G231R, G231M, G231I, G231Q, G231E, G231D, G231V, G231S, G231A, G231L, G231F, E232H, E232R, E232L, E232V, E232N, E232M, E232I, E232P, E232D, E232A, E232G, E232W, E232C, K233T, K233S, K233Q, K233R, K233L, K233E, K233G, K233V, K233N, K233P, K233A, K233C, K234M, K234I, K234Q, K234L, K234D, K234A, K234G, K234Y, K234W, K234S, K234H, K234R, N235M, N235P, N235E, N235V, N235Y, N235S, N235F, N235T, N235I, N235L, N235A, N235W, G236T, G236I, G236H, G236P, G236L, G236A, G236V, G236S, G236Q, G236E, G236D, L237N, L237T, L237S, L237H, L237W, L237F, L237R, L237M, L237I, L237P, L237G, L237Y, L237C, F238K, F238N, F238S, F238Q, F238H, F238P, F238L, F238G, F238R, F238M, F238E, F238A, F238V, F238C, G239K, G239T, G239R, G239I, G239Q, G239P, G239L, G239A, G239V, G239Y, G239W, G239F, G239M, G239H, G239E, G239D, G239C, N240L, N240G, N240S, N240Q, N240P, N240V, N240W, N240F, L241T, L241I, L241Q, L241V, L241Y, L241W, L241F, L241S, L241H, L241A, L241G, 1242K, I242N, I242T, I242S, I242Q, I242H, I242L, I242E, I242A, I242G, I242V, I242Y, I242W, I242C, I242F, I242M, A243N, A243T, A243I, A243H, A243D, A243V, A243C, A243Q, A243R, A243L, A243G, A243Y, A243S, L244M, L244S, L244W, L244R, L244Q, L244D, L244Y, L244C, S245M, S245I, S245L, S245W, S245Y, S245F, L246N, L246S, L246M, L246H, L246G, L246Y, L246F, L246T, L246C, G247N, G247T, G247S, G247Q, G247L, G247W, G247C, G247M, G247R, G247V, L248N, L248T, L248H, L248A, L248G, L248V, L248F, L248K, L248M, L248Q, L248E, L248C, T249N, T249S, T249Q, T249P, T249A, T249W, T249F, T249R, T249V, P250N, P250S, P250M, P250H, P250G, P250T, P250Q, P250L, P250D, P250A, P250W, P250C, N251T, N251I, N251H, N251R, N251E, N251L, N251A, N251G, N251V, N251S, F252T, F252A, F252V, F252Q, F252P, F252L, K253N, K253T, K253R, K253S, K253M, K253I, K253L, K253G, K253Q, K253E, K253V, K253Y, K253W, K253C, K253F, S254T, S254A, S254G, S254F, S254P, S254V, N255T, N255S, N255P, N255E, N255G, N255V, N255Y, N255C, N255I, N255D, N255A, F256N, F256Q, F256H, F256L, F256E, F256A, F256G, F256V, F256Y, F256T, F256S, F256R, F256C, D257S, D257Q, D257L, D257V, L258R, L258S, L258M, L258G, L258Y, L258F, L258T, L258P, L258E, L258D, A259N, A259M, A259I, A259Q, A259P, A259G, A259V, A259S, A259W, A259C, A259F, A259L, A259E, E260D, E260A, E260G, E260C, E260I, E260L, E260V, D261E, D261R, D261L, D261A, A262N, A262R, A262L, A262D, A262C, A262M, A262E, A262G, A262V, K263R, K263E, K263G, K263A, K263S, L264K, L264Y, L264C, L264S, L264M, L264Q, L264H, L264P, L264G, L264V, L264W, L264F, Q265T, Q265M, Q265W, Q265S, Q265L, L266T, L266V, L266C, L266F, L266S, L266I, L266E, L266Y, S267I, S267P, S267L, S267A, S267V, S267K, S267T, S267Y, K268N, K268T, K268E, K268P, K268R, K268L, D269L, D269V, T270R, T270S, T270Q, T270E, T270A, T270G, T270V, T270C, Y271K, Y271N, Y271P, Y271L, Y271S, Y271R, Y271Q, Y271F, D272K, D272R, D272H, D272L, D272A, D272G, D272Y, D272W, D272C, D272F, D272T, D272S, D272M, D272I, D272Q, D272P, D272V, D273K, D273N, D273T, D273R, D273S, D273Q, D273H, D273P, D273L, D273A, D273V, D273Y, D273C, D273E, D273G, D273W, D274Q, D274P, D274L, D274E, D274A, D274G, D274V, D274Y, D274N, D274T, D274S, D274M, D274I, D274W, L275K, L275S, L275H, L275A, L275Y, L275F, L275N, L275T, L275M, L275I, L275E, L275D, L275G, L275W, L275C, D276K, D276N, D276T, D276R, D276S, D276M, D276I, D276Q, D276H, D276P, D276L, D276A, D276G, D276V, D276Y, D276W, D276C, D276F, D276E, N277K, N277I, N277E, N277Y, N277R, N277M, N277Q, N277H, N277G, N277F, L278Q, L278A, L278F, L278W, L279N, L279S, L2791, L279Q, L279H, L279E, L279D, L279A, L279G, L279Y, L279F, L279K, L279T, L279R, L279M, L279P, L279V, L279W, A280K, A280N, A280R, A280S, A280I, A280Q, A280H, A280P, A280L, A280D, A280G, A280V, A280Y, A280W, A280C, A280F, A280T, A280M, Q281T, Q281P, Q281L, Q281E, Q281V, Q281S, Q281M, Q281R, Q281A, Q281G, Q281Y, Q281F, I282T, I282H, I282E, I282D, I282A, I282G, I282V, I282W, 1282F, I282N, I282Q, I282R, 1282S, G283T, G283H, G283D, G283Y, G283W, G283C, G283L, G283F, G283N, G283S, G283M, G283I, G283Q, G283P, G283V, D284S, D284H, D284T, D284A, D284Y, D284W, Q285T, Q285S, Q285M, Q285L, Q285E, Q285D, Q285Y, Q285W, Q285C, Q285N, Q285H, Q285P, Q285R, Q285A, Q285F, Y286N, Y286T, Y286Q, Y286E, Y286D, Y286V, Y286C, Y286R, Y286H, Y286A, Y286G, Y286S, A287N, A287T, A287R, A287S, A287M, A287I, A287Q, A287H, A287P, A287E, A287D, A287G, A287V, A287Y, A287W, A287F, A287K, A287L, D288M, D288W, D288S, D288Q, D288P, D288L, D288E, D288A, D288G, D288V, D288Y, D288F, L289N, L289S, L289M, L289Q, L289H, L289P, L289D, L289G, L289V, L289Y, L289C, L289T, L289A, L289W, F290N, F290T, F290Y, F290W, F290S, F290I, F290Q, F290H, F290P, F290E, F290A, F290G, F290C, L291K, L291N, L291R, L291H, L291D, L291A, L291Y, L291W, L291F, L291T, L291S, L291M, L291I, L291Q, L291P, L291E, L291G, L291V, L291C, A292N, A292R, A292P, A292S, A292E, A293K, A293N, A293S, A293I, A293L, A293E, A293D, A293G, A293V, A293C, A293T, A293H, A293F, K294R, K294E, K294W, K294T, K294G, K294Y, K294S, K294F, N295K, N295T, N295R, N295I, N295Q, N295P, N295E, N295V, N295Y, N295W, N295C, N295F, N295S, N295A, N295G, L296A, L296V, L296Y, L296S, L296W, L296C, L296F, L296T, L296D, S297N, S297M, S297G, S297Y, S297W, S297K, S297L, D298M, D298H, D298A, D298Y, D298N, D298R, D298S, D298Q, D298G, A299T, A299R, A299E, A299V, A299C, A299S, 1300R, 1300Q, 1300A, 1300H, 1300V, L301K, L301T, L301S, L301I, L301R, L301A, L301G, L301V, L301C, L301N, L301Q, S303M, S303H, S303E, S303A, S303W, S303L, S303F, S303K, S303R, S303G, D304N, D304M, D304I, D304Q, D304L, D304V, D304W, D304C, R307N, R307T, R307S, R307A, R307V, R307C, R307Q, R307G, V308Q, V308D, V308G, V308W, V308L, N309K, N309R, N309E, N309A, N309G, N309S, N309P, N309D, T310K, T310S, T310P, T310A, T310G, T310W, T310L, E311D, E311K, E311G, I312M, I312E, I312D, I312V, I312C, I312N, I312T, I312L, I312A, I312G, I312S, T313S, T313E, T313D, T313C, K314N, K314T, K314S, K314H, K314P, K314L, K314E, K314D, K314A, K314G, K314V, K314Y, K314C, A319S, A319Y, A319N, A319T, S320A, S320G, M321Y, M321F, I322T, I322M, 1322V, 1322S, 1322C, K323N, K323T, K323R, K323S, K323M, K323I, K323Q, K323H, K323L, K323E, K323D, K323A, K323G, K323V, K323C, K323F, K323Y, Y325V, Y325H, D326E, D326N, D326Q, D326A, E327T, E327S, E327I, H329A, H329G, H329M, H329S, H329K, H329R, H329C, Q330N, Q330T, Q330R, Q330L, Q330E, Q330D, Q330G, Q330V, Q330C, Q330S, Q330F, Q330A, D331E, L332K, L332R, L332N, L332C, T333R, T333L, T333S, T333C, T333I, T333A, T333G, T333V, T333W, T333K, T333N, T333Q, T333H, T333M, L334T, L334S, L334M, L334I, L334Q, L334H, L334R, L334E, L334A, L334G, L334V, L334W, L334D, L334C, L334K, L335V, L335C, L335W, L335A, L335M, L335I, A337K, A337T, A337R, A337S, A337M, A337L, A337Y, A337C, A337Q, A337G, A337V, A337N, A337H, A337W, L338N, L338R, L338S, L338V, L338Y, L338C, L338A, L338T, L338F, L338Q, V339T, V339I, V339C, R340K, Q341T, Q341R, Q341M, Q341L, Q341D, Q341A, Q341G, Q341S, Q341K, Q341C, Q341V, Q341H, Q341F, Q342K, Q342S, Q342E, Q342W, Q342C, Q342F, Q342N, Q342A, Q342G, Q342R, Q342L, Q342M, L343R, L343A, L343V, L343K, L343M, L343D, L343W, L343T, L343G, L343S, P344N, P344T, P344R, P344G, P344S, P344K, P344H, E345K, E345N, E345T, E345R, E345S, E345M, E345Q, E345H, E345L, E345A, E345G, E345V, E345Y, E345C, E345F, E345I, E345P, K346R, K346N, K346C, K346M, K346Q, K346E, K346V, K346S, K346D, Y347K, Y347R, K348R, K348I, K348A, E349K, E349N, E349T, E349R, E349S, E349M, E349I, E349Q, E349H, E349P, E349L, E349D, E349A, E349G, E349V, E349Y, E349W, E349C, I350T, I350L, I350V, 1350M, F352Y, F352A, F352C, F352T, F352S, F352R, F352L, F352V, F352I, D353M, D353L, D353E, D353V, D353S, D353C, D353N, D353T, D353Q, D353H, D353A, Q354R, Q354S, Q354A, Q354G, Q354Y, Q354T, Q354H, Q354P, Q354L, Q354E, Q354D, Q354K, Q354V, S355N, S355M, S355I, S355H, S355R, S355A, S355K, S355G, S355T, S355C, S355L, S355D, S355V, S355Y, K356S, K356R, K356F, K356L, K356D, N357R, N357K, N357S, N357E, N357I, N357Y, N357W, Y359N, Y359F, A360G, Y362F, Y362N, 1363V, 1363S, 1363P, D364K, D364T, D364R, D364S, D364M, D364H, D364L, D364A, D364G, D364V, D364C, D364F, D364N, D364W, D364Q, G365S, G365A, G366S, G366H, G366P, G366Y, G366L, G366F, G366K, G366T, G366A, A367K, A367T, A367S, A367R, A367C, S368N, S368T, S368R, S368L, S368H, E370S, E370H, E370D, E370V, E370K, E370M, E370P, E370A, E370G, E370W, E371K, E371N, E371R, E371M, E371Q, E371L, E371D, E371S, E371A, E371G, F372L, Y373Q, K374N, F375T, F375S, F375H, F375L, F375V, F375Y, F375A, F375W, 1376V, K377N, K377M, K377Q, K377H, K377R, K377A, K377S, P378K, P378T, P378H, P378R, P378V, P378S, P378L, P378F, 1379K, 1379N, 1379T, 1379Q, 1379Y, 1379S, 1379W, 1379C, 1379H, 1379P, 1379R, 1379F, L380V, E381S, E381I, E381Q, E381H, E381R, E381L, E381D, E381G, E381V, E381N, K382Q, K382R, K382L, K382P, M383H, M383L, D384I, D384P, D384G, D384V, D384Y, D384S, D384N, D384Q, D384R, D384L, D384A, D384C, G385Q, G385R, G385L, G385E, G385A, G385S, G385C, G385K, G385H, G385D, T386N, T386A, T386R, T386V, E387K, E387T, E387D, E387C, E387R, E387V, E388D, E388P, E388G, L389V, L389F, L390T, L390R, L390Q, L390E, L390V, L390S, L390M, L390I, V391N, V391T, V391S, V391I, V391H, V391E, V391D, V391F, V391K, V391P, V391L, V391A, K392S, K392A, K392R, L393I, L393V, L393C, N394M, N394Q, N394L, N394E, N394D, N394G, N394W, N394H, N394A, N394V, N394Y, R395I, R395L, R395A, R395C, R395M, L399M, L399C, T404M, T404V, T404S, F405T, F405I, F405L, F405W, F405S, F405Q, F405R, F405A, F405V, D406H, D406E, D406Q, D406A, N407Y, S409A, S409V, S409C, S409F, L416R, L416E, L416A, L416Y, L416W, L416F, L416M, G417Q, G417E, G417K, G417T, L419M, H420Q, H420A, H420S, H420G, H420L, A421P, A421E, A421D, A421G, A421V, A421S, A421N, I422M, I422V, I422T, I422L, L423M, L423I, R424M, R424Q, R424E, R424V, R424S, R424K, R424I, R424L, R424A, R424C, R425K, R425S, R425A, R425C, E427T, E427K, E427R, E427S, D428M, D428Q, D428R, D428L, D428E, D428G, D428V, D428S, D428W, D428C, D428T, D428I, F429H, F429S, F429W, F429C, F429Y, Y430W, Y430F, P431Q, P431G, P431V, P431K, P431D, F432V, L433R, K434N, K434R, K434S, K434E, K434D, K434A, K434F, D435T, D435S, D435A, D435V, D435Y, D435C, D435M, D435Q, D435W, N436Q, N436H, N436E, N436G, N436T, N436A, R437M, R437L, R437V, R437C, R437K, R437N, R437A, E438K, E438Q, E438L, E438V, E438S, E438G, E438W, 1440F, 1440L, I440V, E441D, E441V, E441R, K442L, K442T, K442Q, T445S, T445H, T445E, T445A, T445Y, T445W, T445R, T445N, T445G, F446C, R447T, R447I, R447V, Y450H, Y450C, Y450S, Y451W, R457K, R457N, R457M, R457A, R457S, N459H, N459T, F462W, A463S, M465L, M465V, T466I, T466V, T466K, T466H, K468E, K468L, S469T, S469R, S469M, S469K, S469L, E470N, E470D, E470A, E470T, E470M, E470V, E471Q, T472K, T472R, T472S, T472H, T472P, T472A, T472N, T474R, T474Y, T474H, P475N, E479N, E479W, E479D, E480S, E480F, E480A, K484L, K484V, K484M, G485K, G485P, G485R, G485L, G485E, G485D, G485A, G485W, G485H, G485V, S487C, A488L, A488V, A488S, Q489N, Q489T, Q489R, Q489L, Q489D, Q489A, S490N, S490T, S490A, S490G, S490L, F491Y, F491S, I492V, 1492Y, E493S, E493D, E493Y, E493F, R494T, R494V, R494L, M495H, M495F, T496Q, T496G, T496N, T496V, T496S, N497Q, N497P, N497R, N497M, N497I, N497W, F498N, F498T, F498S, F498H, F498L, F498D, F498A, F498V, F498C, F498R, F498E, F498G, D499R, K500R, K500S, K500P, K500E, K500A, K500G, K500V, K500W, K500C, K500F, K500T, K500Q, K500H, N501T, N501R, N501H, N501L, N501E, N501S, N501C, L502V, L502A, P503R, P503V, N504K, N504P, N504E, N504A, N504Y, N504L, N504T, N504Q, N504C, E505Q, E505D, E505V, E505T, K506T, K506M, K506A, K506V, V507S, V507L, V507I, V507E, V507A, L508H, P509S, P509A, K510R, K510T, H511K, H511R, H511S, H511Q, S512R, S512L, L513K, L513T, L513P, L513M, L513I, L513V, L513W, L514H, L514T, L514V, L514S, Y515N, Y515L, Y515A, Y515F, Y515K, Y515Q, Y515R, Y515V, E516S, E516D, E516G, E516L, Y517N, Y517L, Y517D, Y517G, Y517S, Y517F, Y517E, Y517V, F518Y, T519D, T519Y, T519S, T519I, T519Q, T519P, V520T, V520G, V520A, Y521L, Y521V, E523S, E523V, E523C, E523A, L524T, L524I, L524S, L524F, T525N, T525A, T525V, T525Y, T525S, T525L, K526H, K526P, K526R, K526S, K526L, K526V, K526F, V527T, V527I, V527F, V527S, V527M, V527A, K528R, K528I, K528Q, K528H, K528A, K528G, K528V, K528W, K528P, K528S, Y529V, Y529S, Y529R, Y529W, V530I, V530H, V530L, V530E, V530D, V530G, V530W, V530T, V530S, V530M, V530Q, V530A, V530Y, V530F, T531R, T531Q, T531L, T531E, T531D, T531A, T531V, T531Y, T531N, T531S, T531F, E532M, E532G, E532W, E532K, E532I, E532Q, E532D, E532S, G533T, G533Q, G533L, G533A, G533N, G533R, G533E, G533D, M534R, M534S, M534L, M534N, M534T, M534I, M534P, M534G, M534V, R535T, R535I, R535L, R535D, R535V, R535S, R535C, R535F, R535A, R535G, R535W, K536T, K536I, K536H, K536P, K536L, K536E, K536D, K536A, K536G, K536V, K536Y, K536S, K536W, K536C, K536F, K536N, K536R, K536M, P537I, P537Q, P537H, P537D, P537A, P537V, P537C, P537F, P537E, A538N, A538Q, A538L, A538Y, A538W, A538T, A538M, A538H, A538P, A538D, A538G, F539R, F539S, F539P, F539L, F539D, F539V, F539W, F539K, F539T, F539C, L540T, L540I, L540V, S541I, S541P, S541R, S541V, S541Y, S541D, S541A, S541G, S541C, G542N, G542Q, G542L, G542S, G542C, G542K, G542R, G542M, G542A, E543S, E543P, E543L, E543G, E543V, E543C, E543N, E543T, E543D, E543F, Q544T, Q544R, Q544S, Q544L, Q544A, Q544G, Q544N, Q544V, K545N, K545R, K546R, K546E, K546A, K546V, K546T, K546I, K546Q, K546L, K546G, K546Y, K546S, K546C, A547N, A547M, A547D, A547G, A547H, A547P, A547V, A547L, A547F, 1548T, 1548M, 1548V, 1548L, 1548C, V549L, V549E, V549D, V549S, V549C, D550S, D550M, D550G, D550V, D550C, D550R, L551K, L551E, L551D, L551N, L551S, L551H, L551A, L551Y, L551F, L552H, L552A, L552S, F553L, F553K, F553Y, K554R, K554Y, K554T, T555R, T555L, T555E, T555D, T555G, T555Y, T555S, T555C, T555F, T555N, T555I, T555P, N556T, N556Q, N556S, N556I, N556L, N556D, N556A, R557T, R557I, R557L, R557A, R557V, R557W, R557C, R557Y, R557F, K558N, K558T, K558R, K558L, K558Y, K558C, K558F, K558S, K558V, K558W, V559N, V559L, V559A, V559P, V559G, V559W, T560I, T560Q, T560L, T560G, T560V, T560F, T560K, T560N, T560S, T560W, V561P, V561H, V561D, V561A, K562N, K562Q, K562H, K562P, K562R, K562L, K562A, K562T, K562S, Q563I, Q563L, Q563V, Q563W, L564T, L564I, L564V, L564C, L564P, L564G, K565H, K565D, K565A, K565Y, K565C, K565M, K565Q, K565P, K565R, K565L, K565G, K565F, E566D, E566Y, E566L, E566A, D567M, D567V, D567S, D567C, D567H, D567E, D567A, Y568I, Y568L, Y568H, F569M, F569W, F569L, K570M, K570A, K570G, K570S, K570W, K570F, K570L, K570V, K570C, K571H, K571P, K571G, K571L, K571T, K571M, K571R, K571D, K571S, K571C, 1572T, 1572A, 1572S, 1572C, I572F, I572L, E573R, E573S, E573Q, E573H, E573D, E573G, E573Y, E573L, E573F, E573W, C574K, C574I, C574P, C574R, C574S, C574A, C574G, C574V, F575K, F575T, F575I, F575D, F575G, F575S, F575M, F575H, F575L, F575E, F575A, D576N, D576I, D576G, D576L, D576H, D576R, D576E, D576V, D576C, S577T, S577R, S577P, S577L, S577M, S577V, V578S, V578M, V578D, V578I, V578P, V578L, E579T, E579R, E579D, E579A, E579Y, E579S, E579I, E579L, I580T, I580M, I580L, 1580V, 1580F, S581T, S581M, S581Q, S581K, S581R, S581L, S581G, S581V, S581Y, S581C, G582N, G582T, G582S, G582Q, G582L, G582V, G582Y, G582F, G582R, G582P, G582A, G582W, V583R, V583M, V583P, V583W, V583F, V583S, V583Y, E584T, E584W, E584M, E584L, E584D, E584G, D585Q, D585N, D585T, D585L, R586S, R586H, R586L, R586G, R586Y, R586C, R586P, R586F, F587M, F587Y, F587L, N588K, N588T, N588R, N588M, N588E, N588G, N588S, N588H, N588C, A589K, A589T, A589Y, A589C, A589N, A589R, A589F, S590R, S590H, S590A, S590V, S590C, S590F, S590M, S590P, S590L, L591C, L591H, G592M, G592L, G592V, G592Y, G592S, G592W, G592F, G592T, G592H, T593G, T593S, T593C, Y594M, Y594F, H595R, H595G, H595F, H595S, L597V, L597Q, L598S, L598H, L598R, L598V, L598Y, L598W, L598A, L598G, K599T, K599I, K599Q, K599H, K599L, K599A, K599V, K599Y, K599W, K599R, K599G, K599S, 1600E, 1600F, 1600P, I600G, I601S, I601F, I601T, I601M, I601G, 1601V, I601C, K602N, K602M, K602I, K602Q, K602H, K602P, K602R, K602L, K602E, K602A, K602G, K602W, K602F, K602S, K602V, D603T, D603L, D603V, D603Y, D603H, D603E, K604R, K604G, K604Y, K604E, D605T, D605E, D605W, D605G, F606K, F606R, F606V, F606L, L607F, L607T, L607I, L607Y, L607W, D608S, D608Q, D608V, D608H, N609S, N609M, N609P, N609R, N609L, N609E, N609G, N609Y, N609K, N609H, N609D, N609V, N609W, E610N, E610R, E610I, E610L, E610K, E610D, E610G, E611Q, E611L, E611A, E611C, E611T, E611H, E611R, E611G, E611V, E611Y, E611S, E611F, N612T, N612V, N612L, N612A, N612W, N612C, E613K, E613M, E613Q, E613R, E613L, E613S, E613A, E613V, D614N, D614S, D614E, D614P, D614V, D614Y, D614L, I615R, I615S, I615M, I615H, I615P, I615L, I615D, 1615G, 1615K, I615T, 1615C, 1615F, L616V, L616F, L616C, E617T, D618P, D618L, 1619T, 1619M, I619L, 1619V, 1619F, 1619A, I619W, V620A, V620T, V620L, V620G, L621N, L621H, L621E, L621Y, L621W, L621F, L621T, L621M, L621V, L621S, L623N, L623M, L623V, L623T, T624S, T624C, L625R, E627M, E627I, E627Q, E627H, E627P, E627L, E627V, E627Y, E627C, E627R, R629N, R629T, R629M, R629Q, R629H, R629L, R629A, R629Y, R629W, R629C, R629F, R629I, 1632L, 1632V, E633H, E633D, E633S, E633G, E634M, E634Q, E634S, E634T, E634L, E634G, E634V, L636T, L636M, L636Y, L636S, L636F, K637N, K637T, K637S, K637Q, K637D, K637G, K637W, K637R, K637L, K637A, K637V, T638K, T638N, T638S, T638Q, T638H, T638E, T638D, T638A, T638G, T638C, T638P, T638L, T638V, Y639I, Y639L, Y639A, Y639V, Y639S, Y639K, Y639T, Y639P, Y639C, A640M, A640I, A640G, A640V, A640L, A640S, A640R, A640E, H641K, H641S, H641I, H641R, H641L, H641E, H641A, H641C, H641N, H641Q, H641V, L642T, L642R, L642S, L642Q, L642A, L642V, L642Y, F643I, F643L, F643G, F643W, D644T, D644P, D644R, D644L, D645W, K646N, K646T, K646S, K646M, K646Q, K646H, K646P, K646R, K646L, K646E, K646D, K646A, K646G, K646V, K646Y, K646W, K646C, K646F, K646I, V647L, V647D, V647G, V647S, V647T, V647M, V647H, M648T, M648S, M648D, M648Y, M648W, M648L, M648F, K649T, K649S, K649M, K649I, K649Q, K649H, K649P, K649L, K649E, K649D, K649A, K649G, K649V, K649Y, K649W, K649C, K649F, K649N, K649R, Q650T, Q650I, Q650E, Q650D, Q650V, Q650Y, Q650F, Q650N, Q650M, Q650P, Q650A, Q650C, Q650L, L651I, L651V, K652T, K652M, K652I, K652Q, K652H, K652P, K652E, K652D, K652G, K652Y, K652W, K652F, K652N, K652R, K652L, K652V, K652S, R653T, R653S, R653M, R653I, R653H, R653P, R653L, R653E, R653D, R653A, R653G, R653V, R653Y, R653C, R653F, R653N, R653W, R654K, R654T, R654M, R654I, R654A, R654V, R655T, R655M, R655I, R655L, R655A, R655G, R655Y, R655W, R655K, R655V, R655S, R655F, Y656W, Y656L, T657K, T657R, T657M, T657L, T657A, T657W, T657E, T657G, G658K, G658T, G658R, G658H, G658N, G658S, G658Q, G658A, W659C, G660S, G660Q, G660C, G660A, R661G, R661Y, R661S, R661L, L662K, L662N, L662T, L662M, L662D, L662G, L662I, L662R, L662E, L662A, L662V, R664Q, R664L, R664A, R664Y, R664C, R664S, R664P, R664G, R664V, K665T, K665S, K665M, K665Q, K665P, K665E, K665Y, K665H, K665A, K665V, K665C, K665L, K665F, L666T, L666I, 1667L, 1667V, 1667C, N668T, N668M, N668L, N668D, N668C, G669S, I670L, 1670M, 1670H, I670V, R671K, R671T, R671M, R671E, R671V, R671C, R671L, R671F, R671H, R671P, R671S, K673S, K673H, K673A, K673Y, K673C, K673T, K673M, K673R, K673L, K673G, K673V, Q674T, Q674S, Q674E, Q674A, Q674G, Q674Y, Q674K, Q674N, Q674R, Q674L, S675Q, S675E, S675T, S675R, S675A, G676K, G676N, G676R, G676Q, G676L, G676Y, G676W, G676E, G676S, G676F, K677R, K677M, K677Q, K677H, K677Y, K677F, K677T, K677S, K677A, T678R, T678A, I679L, 1679V, L680M, L680F, L680Y, D681A, D681C, D681Q, D681P, F682H, F682M, F682L, L683V, K684R, K684Q, K684T, S685H, S685P, S685R, S685G, S685N, S685A, S685Y, D686T, D686G, G687T, G687R, G687S, G687H, G687L, G687D, G687A, G687V, F688T, F688P, F688E, F688D, F688R, F688A, F688G, F688S, F688C, A689T, A689M, A689P, A689R, A689E, A689V, A689C, A689S, A689L, A689D, A689G, N690S, N690M, N690Q, N690H, N690R, N690D, N690G, N690L, N690A, N690Y, N690W, R691S, R691I, R691H, R691L, R691G, R691V, R691C, R691K, R691N, R691D, R691A, N692T, N692S, N692A, N692G, N692Q, N692H, N692Y, F693L, F693W, F693M, M694H, M694P, M694R, M694W, M694F, M694V, M694Y, M694S, M694C, Q695K, Q695S, Q695G, Q695C, Q695N, Q695E, Q695A, L696M, 1697R, 1697C, 1697A, H698A, H698G, H698Y, H698F, H698S, H698W, D699S, D699W, D699A, D700N, D700T, D700R, D700S, D700M, D700Q, D700H, D700P, D700A, D700G, D700C, D700I, D700L, D700E, D700V, S701I, S701Q, S701R, S701E, $701G, S701W, S701N, S701L, S701A, S701Y, S701C, L702R, L702V, L702S, L702C, L702F, L702P, T703S, T703G, T703D, K705N, K705R, K705V, E706A, E706G, E706V, E706S, E706C, E706L, E706I, E706R, E706Y, E706W, D707M, D707L, D707E, D707P, D707C, I708M, I708L, Q709K, Q709R, Q709M, Q709A, Q709Y, Q709T, Q709L, Q709G, Q709S, Q709C, K710N, K710R, K710S, K710P, K710L, K710A, K710V, K710I, K710D, A711S, Q712K, Q712M, Q712R, Q712V, Q712W, Q712L, Q712F, Q712S, V713K, V713N, V713P, V713A, V713S, V713T, V713Q, V713H, V713R, S714M, S714Q, S714H, S714L, S714D, S714G, S714V, S714C, S714F, S714T, S714P, S714R, S714A, S714W, G715K, G715R, G715S, G715M, G715P, G715L, G715V, G715T, G715A, Q716M, Q716H, Q716L, Q716A, Q716Y, Q716F, Q716K, Q716I, Q716S, G717K, G717R, G717H, G717P, G717A, G717S, G717C, G717N, G717T, G717I, G717L, G717V, D718K, D718N, D718R, D718S, D718M, D718H, D718L, D718T, D718Q, D718G, D718V, D718P, D718E, D718A, D718F, S719N, S719C, S719T, S719D, S719K, L720I, L720S, L720A, L720H, L720V, H721K, H721R, H721M, H721Q, H721L, H721A, H721V, H721T, H721G, H721P, H721S, E722K, E722N, E722Q, E722A, E722G, E722T, E722R, E722L, E722S, E722W, E722I, E722P, E722D, E722V, E722F, E722C, H723K, H723N, H723T, H723R, H723M, H723I, H723Q, H723A, H723S, H723C, H723L, H723G, H723E, H723V, I724V, I724L, I724M, I724A, I724C, I724F, A725K, A725N, A725T, A725R, A725M, A725Q, A725H, A725P, A725L, A725G, A725V, A725Y, A725W, A725C, A725F, N726K, N726R, N726S, N726Q, N726A, N726T, N726W, N726C, N726G, L727T, L727M, L727V, L727Y, L727W, L727G, L727C, L727A, L727S, A728Q, A728H, A728P, A728E, A728D, A728G, A728Y, A728S, A728W, A728F, A728R, A728L, A728K, G729N, G729T, G729R, G729S, G729M, G729I, G729Q, G729H, G729P, G729L, G729E, G729D, G729A, G729V, G729Y, G729W, G729C, G729F, G729K, S730K, S730N, S730T, S730M, S730H, S730P, S730A, S730G, S730Y, S730C, S730W, S730I, S730Q, S730R, S730D, P731A, P731T, P731M, P731R, P731H, P731G, P731V, P731S, A732N, A732T, A732S, A732I, A732H, A732G, A732C, A732R, A732M, A732Q, A732P, A732W, A732V, I733T, I733Q, I733A, I733V, I733Y, I733W, I733E, I733C, I733F, I733N, I733H, I733R, I733L, I733S, K734R, K734H, K734S, K734N, K734V, K734C, K734W, K735R, K735M, G736T, G736S, G736W, G736A, G736V, I737V, I737T, I737R, L738R, L738M, L738V, L738Y, L738W, L738F, L738Q, L738H, L738T, L738I, L738A, Q739K, Q739R, Q739T, Q739M, Q739I, Q739L, Q739A, Q739S, T740A, T740S, T740C, T740M, V741L, V741E, V741G, V741C, K742R, V743I, V743L, V743R, D745T, D745E, D745V, D745L, E746V, L747S, L747F, V748I, V748C, V748T, K749L, K749M, K749A, K749C, K749F, V750A, V750S, V750T, V750I, V750C, V750M, V750R, M751L, M751E, G752L, G752V, R753K, R753N, R753T, R753S, R753M, R753H, R753L, R753E, R753D, R753G, R753Y, R753C, R753F, R753P, R753Q, R753V, H754M, H754R, H754G, H754T, H754L, H754C, H754E, H754D, H754A, H754S, K755L, P756L, E757S, E757M, E757A, E757L, E757G, N758T, N758L, N758Y, N758S, N758F, N758M, N758A, N758V, I759L, I759A, V760I, V760Q, V760E, V760C, I761T, M763T, M763I, M763V, M763S, M763H, A764V, A764S, A764G, R765S, E766K, E766T, E766Y, E766W, E766R, E766M, E766G, N767K, N767T, N767M, N767P, N767E, N767A, N767G, N767F, N767R, N767H, N767L, N767V, N767S, Q768K, Q768T, Q768L, Q768V, Q768W, Q768F, Q768Y, Q768S, T769Q, T769P, T769G, T769S, T769C, T769M, T769H, T769R, T769V, T769Y, T770G, T770S, Q771I, Q771A, Q771G, Q771T, Q771P, Q771L, Q771E, Q771Y, Q771S, K772R, K772P, K772S, K772W, K772C, K772H, G773A, G773S, G773W, G773H, Q774T, Q774H, Q774L, Q774G, Q774S, Q774V, K775T, K775M, K775Q, K775H, K775L, K775E, K775D, K775A, K775V, K775Y, K775N, K775R, K775C, N776S, N776R, N776D, N776W, N776C, N776T, N776Q, N776E, S777A, S777V, S777R, R778K, R778H, R778P, R778Y, R778N, R778M, R778L, R778S, R778W, R778F, E779T, E779S, E779L, E779A, E779I, M781W, M781F, M781K, M781I, M781Q, M781L, M781D, M781G, M781V, M781Y, K782S, K782Q, K782R, K782E, K782Y, K782N, K782A, R783N, R783T, R783M, R783I, R783P, R783L, R783E, R783Q, R783H, R783A, R783G, R783V, R783Y, R783W, R783C, I784T, I784V, I784L, E785Q, E785S, E785M, E785I, E785W, E786H, E786P, E786R, E786Y, E786S, E786L, E786N, E786Q, E786D, E786A, G787S, G787L, I788L, I788V, I788T, I788A, I788C, K789N, K789H, K789G, K789V, K789C, K789Q, K789R, K789S, E790Q, E790A, E790V, E790S, E790R, E790L, L791M, G792Y, G792K, G792S, G792D, S793T, Q794H, Q794P, Q794V, Q794W, Q794T, Q794S, Q794M, Q794L, Q794A, Q794G, I795Q, I795L, K797T, K797M, K797A, K797L, K797Y, K797S, E798S, E798L, E798A, E798Y, E798T, E798V, E798W, H799T, H799L, H799V, H799W, H799K, P800R, V801M, V801I, V801P, V801C, V801S, E802P, E802G, E802V, E802L, E802N, E802S, E802R, N803S, N803G, T804H, T804P, T804Y, T804C, T804L, T804F, T804N, T804R, T804M, T804D, T804A, T804G, Q805A, Q805G, Q805Y, Q805L, L806P, L806F, Q807K, Q807T, Q807A, Q807I, Q807R, Q807L, Q807E, Q807S, N808R, N808W, N808Y, E809L, E809T, E809P, E809G, E809V, K810R, L811T, L811I, L811V, L811F, L811A, L811Y, Y812V, Y812W, Y812F, Y812M, Y812A, Y814F, Y814V, Y815N, Y815H, L816S, L816E, L816M, L816V, L816C, L816F, Q817S, N818D, N818A, N818G, N818S, G819A, R820K, R820S, R820V, R820Y, R820C, V824T, V824Q, V824L, V824D, V824C, V824E, V824G, V824Y, V824F, D825N, D825P, D825E, D825Y, Q826K, Q826M, Q826Y, Q826S, Q826N, Q826T, Q826A, E827T, E827I, E827G, E827P, E827S, E827W, L828V, D829L, 1830P, I830V, 1830S, 1830H, 1830L, N831S, N831I, N831A, N831F, N831R, N831L, N831G, N831V, N831Y, R832I, R832S, R832H, L833E, L833V, L833M, S834T, S834I, S834Q, S834R, S834V, S834N, S834L, D835N, D835H, D835S, D835C, D835L, D835T, D835A, D835G, D835V, Y836H, Y836L, V838I, V838G, I841L, V842M, V842I, V842H, V842R, Q844S, Q844A, Q844G, Q844K, Q844T, Q844R, F846I, F846M, F846Y, L847K, K848R, K848A, K848F, D849N, D849Q, I852L, 1852W, 1852N, I852M, I852A, 1852V, 1852Y, 1852S, D853S, D853V, D853Y, D853T, D853H, D853P, D853A, D853G, K855I, K855L, K855V, V856A, L857I, T858R, R859L, R859I, R859S, S860A, D861E, K862R, K862H, K862L, K862G, K862V, K862S, K862E, K862Y, G865L, G865R, G865W, G865C, S867L, S867G, S867T, S867C, D868K, D868T, D868C, D868E, D868A, N869T, N869H, N869E, N869C, N869G, N869S, V870M, V870T, V870L, P871A, P871L, S872G, E873R, E873S, E873M, E873A, E873Y, E873W, E873D, E873G, E873C, E874S, E874Q, E874H, E874P, E874L, E874D, E874A, E874G, E874V, E874Y, E874W, E874C, E874N, E874T, E874F, V875T, V875I, V875R, V875S, V875C, V876C, K877N, K877T, K877S, K877M, K877I, K877R, K877L, K877D, K877A, K877C, K877F, K877V, K877W, K878P, K878E, K878A, K878V, K878S, K878W, K878H, K878R, K878G, K878Y, M879R, M879L, M879C, M879N, M879Q, K880I, K880Q, K880L, K880A, K880G, K880T, K880R, K880V, N881T, N881S, N881M, N881Q, N881P, N881D, N881G, N881V, N881K, N881R, N881W, Y882D, Y882A, Y882S, Y882W, Y882F, Y882T, Y882R, Y882V, R884M, R884V, R884L, R884W, Q885T, Q885S, Q885R, Q885L, Q885A, Q885C, Q885V, L886Y, L886S, L886M, L887T, L887F, L887R, N888T, N888Q, N888H, N888R, N888A, N888G, N888W, N888F, N888S, N888V, A889C, A889K, K890S, K890R, K890Q, K890H, K890G, L891G, 1892V, 1892C, 1892L, T893A, T893G, Q894T, Q894H, Q894E, Q894G, Q894V, Q894L, Q894F, Q894D, Q894W, Q894C, R895K, R895E, R895S, K896R, F897W, D898W, N899R, N899S, N899W, N899C, N899T, N899A, L900D, T901M, T901S, T901C, K902T, K902M, K902I, K902A, K902V, K902Y, K902C, K902F, K902S, A903M, A903I, A903S, A903R, A903F, E904I, E904A, E904V, E904S, R905T, R905M, R905Q, R905L, R905E, R905D, R905A, R905Y, R905S, R905W, R905C, R905F, R905G, R905V, G906N, G906Q, G906C, G907S, G907H, G907A, L908I, L908V, L908F, S909D, S909L, S909N, S909T, S909R, E910H, E910V, E910F, L911M, L911H, L911E, L911D, L911V, L911Y, L911S, L911W, L911I, L911P, L911C, L911F, D912S, D912A, D912V, D912C, K913R, K913I, K913L, K913G, K913V, A914M, A914C, A914K, A914T, A914H, A914L, A914Y, G915N, G915H, G915A, G915V, G915F, G915K, G915L, G915W, F916M, 1917V, 1917L, K918R, K918W, R919Q, R919I, R919Y, Q920L, Q920A, Q920F, V922T, V922Q, V922A, V922C, V922N, V922E, V922S, E923N, E923Q, E923V, E923S, E923M, E923I, E923H, E923L, E923A, T924L, T924M, T924V, R925W, Q926S, Q926L, Q926W, Q926C, Q926F, Q926N, Q926M, 1927Q, 1927R, 1927L, T928N, T928I, T928S, T928A, T928V, T928C, K929N, K929T, K929Q, K929R, K929L, H930T, H930S, H930I, H930Q, H930E, H930A, H930C, H930F, H930M, H930L, H930V, V931L, A932T, A932S, A932V, Q933L, Q933N, Q933R, Q933M, 1934V, L935D, D936T, D936S, S937K, S937Q, S937R, S937N, S937V, S937W, R938K, R938Q, R938G, M939P, M939Y, M939L, M939F, T941Q, T941P, T941A, T941C, K942T, K942A, K942V, K942S, K942R, K942D, Y943K, Y943T, Y943H, Y943L, Y943V, Y943N, Y943R, Y943S, Y943W, D944A, E945S, E945I, E945H, E945G, E945A, E945V, E945L, N946T, N946I, N946H, N946Y, D947N, D947M, D947I, D947H, D947R, D947G, D947T, D947S, K948S, K948Q, K948R, K948L, K948D, K948W, K948N, K948V, L949K, L949Q, L949A, L949G, L949V, 1950K, 1950M, 1950L, R951M, R951E, E952Q, E952G, E952N, K954R, K954C, 1956L, T957P, T957A, T957S, T957K, T957I, T957L, L958C, K959S, S960T, S960Q, S960P, S960G, S960V, S960Y, K961T, K961L, K961A, K961N, K961G, L962Q, L962P, L962C, V963M, V963P, V963L, V963F, V963S, V963G, S964M, S964Q, S964R, S964L, S964A, S964T, D965V, D965S, D965T, D965H, D965E, F966T, R967P, R967Q, R967L, K968M, K968A, K968V, K968L, K968F, K968R, K968S, D969V, D969M, F970L, F970G, F970V, Q971R, Q971L, Q971A, Q971H, Q971G, Q971V, Q971S, F972T, F972L, F972K, F972G, F972S, Y973H, Y973E, Y973V, Y973N, Y973R, Y973A, K974F, V975S, V975I, V975H, V975R, V975G, V975L, V975M, V975A, E977Q, E977Y, E977T, E977S, E977F, 1978S, 1978E, 1978M, 1978P, 1978L, 1978G, N979H, N979A, N980T, N980S, N980I, N980D, N980C, N980M, N980L, N980A, Y981T, Y981V, Y981N, Y981M, Y981L, Y981C, H982P, H982V, H982I, H982D, H982A, H983N, A984R, A984V, H985A, H985R, H985F, D986K, L989I, L989M, L989F, N990S, N990M, N990H, A991V, V993M, V993L, G994A, T995I, T995R, T995A, T995W, A996L, A996V, A996T, A996C, 1998S, 1998L, 1998M, 1998R, K999R, K999M, K999Y, K1000I, K1000N, K1000T, K1000M, K1000R, K1000G, Y1001P, Y1001F, P1002N, K1003T, K1003H, K1003L, K1003E, K1003A, K1003R, K1003V, K1003S, K1003F, E1005T, E1005R, E1005S, E1005L, E1005G, S1006K, S1006R, S1006H, S1006G, S1006Y, S1006E, S1006A, E1007T, E1007M, E1007A, E1007G, E1007V, E1007C, E1007S, E1007Q, E1007H, E1007R, E1007W, F1008L, F1008A, F1008S, F1008M, V1009L, V1009M, V1009Q, V1009G, Y1010N, Y1010R, Y1010M, Y1010G, Y1010V, Y1010W, G1011L, D1012T, D1012S, D1012I, D1012P, D1012G, D1012A, D1012V, D1012L, Y1013N, Y1013Q, Y1013L, Y1013S, Y1013W, Y1013R, Y1013G, Y1013F, K1014T, K1014H, K1014R, K1014Y, K1014N, K1014G, V1015T, V1015I, V1015R, V1015E, V1015D, V1015G, V1015Y, V1015N, V1015Q, V1015P, V1015L, V1015S, V1015F, Y1016R, Y1016M, Y1016V, Y1016S, Y1016L, D1017S, D1017E, D1017F, V1018I, V1018P, V1018W, V1018F, R1019T, R1019M, R1019Q, R1019E, R1019A, R1019F, K1020T, K1020M, K1020E, K1020D, K1020L, K1020A, K1020S, M1021T, M1021S, M1021A, M1021C, M1021F, I1022F, A1023C, A1023L, K1024S, K1024Q, K1024A, K1024G, K1024E, K1024D, K1024C, S1025T, S1025D, E1026N, E1026I, Q1027N, Q1027I, Q1027L, Q1027A, Q1027V, Q1027T, Q1027G, Q1027C, E1028D, E1028C, E1028N, E1028Y, I1029L, I1029D, I1029M, G1030A, G1030E, G1030S, K1031P, K1031L, K1031G, K1031V, K1031Y, K1031W, K1031D, K1031S, A1032G, A1032K, A1032C, T1033Q, T1033Y, T1033S, A1034N, A1034S, A1034Q, A1034G, A1034V, A1034C, A1034L, K1035R, K1035H, K1035S, K1035F, K1035A, K1035V, Y1036M, Y1036Q, Y1036S, Y1036I, Y1036E, F1037W, F1038Y, F1038S, F1038C, S1040T, S1040I, N1041S, I1042V, I1042W, I1042C, I1042D, I1042L, M1043R, M1043S, N1044Y, F1045M, F1045A, K1047T, K1047S, K1047Q, K1047R, K1047W, K1047P, T1048S, T1048L, T1048V, E1049N, E1049A, E1049Y, E1049W, E1049Q, I1050A, I1050F, I1050L, I1050E, I1050V, T1051K, T1051Q, T1051E, T1051Y, T1051H, T1051R, T1051A, T1051W, T1051F, L1052P, L1052G, A1053T, A1053M, A1053S, A1053P, A1053R, A1053G, N1054S, N1054D, N1054G, N1054C, N1054R, N1054A, N1054V, G1055E, G1055T, E1056N, E1056L, E1056T, E1056D, E1056V, I1057N, I1057T, I1057R, I1057G, I1057V, I1057S, I1057W, R1058K, R1058V, R1058Y, R1058N, R1058S, K1059Q, K1059A, K1059V, K1059L, K1059S, K1059H, K1059P, K1059W, R1060N, R1060L, R1060E, R1060S, R1060C, R1060M, R1060H, P1061N, P1061V, P1061A, P1061G, P1061S, L1062T, L1062S, L1062I, L1062H, L1062R, L1062E, L1062D, L1062G, L1062V, I1063T, I1063H, I1063L, 11063V, 11063R, E1064A, T1065Q, T1065L, T1065V, T1065S, T1065W, T1065C, T1065M, T1065P, T1065A, N1066S, N1066C, N1066T, N1066Q, G1067S, G1067Q, G1067P, G1067L, G1067E, G1067A, G1067V, G1067W, G1067R, G1067D, E1068S, E1068D, E1068A, T1069N, T1069G, T1069Y, T1069H, T1069R, T1069D, T1069S, G1070N, G1070D, G1070P, G1070V, E1071L, E1071A, E1071V, I1072T, I1072R, I1072L, I1072A, I1072V, I1072H, V1073A, V1073L, D1075M, K1076R, G1077T, G1077S, G1077L, G1077E, G1077D, G1077R, G1077K, G1077M, G1077V, G1077C, G1077F, R1078K, R1078V, R1078T, R1078I, R1078L, R1078G, R1078S, R1078Q, D1079F, D1079T, D1079A, D1079G, F1080T, F1080M, F1080E, F1080W, F1080V, F1080H, A1081K, A1081N, A1081T, A1081L, A1081D, A1081C, A1081E, A1081G, A1081P, A1081S, A1081R, A1081H, A1081V, T1082E, T1082V, T1082I, T1082Y, T1082S, T1082L, T1082N, V1083I, V1083L, R1084K, R1084N, R1084T, R1084S, R1084Q, R1084H, R1084L, R1084E, R1084D, R1084A, R1084G, R1084M, R1084I, R1084V, R1084Y, R1084C, K1085N, K1085T, K1085S, K1085M, K1085Q, K1085H, K1085E, K1085D, K1085A, K1085G, K1085C, K1085R, K1085L, K1085Y, K1085F, V1086T, V1086E, V1086A, V1086N, V1086I, L1087F, L1087T, S1088D, S1088T, S1088P, S1088N, S1088M, S1088E, S1088A, M1089N, M1089T, M1089I, M1089E, M1089D, M1089G, M1089V, M1089S, M1089L, M1089R, M1089H, M1089C, M1089F, P1090S, P1090Q, P1090G, P1090V, P1090L, P1090Y, P1090C, P1090D, P1090M, P1090R, P1090E, P1090A, Q1091T, Q1091I, Q1091H, Q1091R, Q1091L, Q1091E, Q1091G, Q1091V, Q1091S, Q1091P, Q1091W, Q1091N, Q1091F, Q1091D, Q1091Y, Q1091C, V1092L, V1092A, V1092I, V1092Q, V1092C, N1093I, N1093R, N1093Q, N1093L, N1093A, N1093S, N1093H, I1094M, I1094L, I1094T, I1094S, I1094P, V1095K, V1095T, V1095R, V1095Q, V1095G, V1095L, K1096N, K1096T, K1096S, K1096I, K1096Q, K1096R, K1096L, K1096A, K1096V, K1096Y, K1096W, K1096C, K1096M, K1096P, K1097R, T1098K, T1098R, T1098M, T1098I, T1098H, T1098L, T1098A, T1098V, T1098C, T1098N, T1098Y, T1098S, T1098W, T1098F, E1099K, E1099R, E1099S, E1099H, E1099A, E1099G, E1099V, E1099Y, E1099N, E1099T, E1099M, E1099I, E1099Q, E1099L, E1099C, V1100K, V1100R, V1100I, V1100L, V1100D, V1100C, V1100T, V1100A, Q1101V, Q1101A, Q1101C, T1102R, T1102A, G1104S, G1104P, G1104A, G1104C, G1104T, S1106T, S1106H, S1106A, S1106Y, S1106W, S1106C, S1106F, S1106V, K1107N, K1107R, K1107Q, E1108D, E1108G, S1109K, S1109N, S1109T, S1109R, S1109M, S1109I, S1109Q, S1109L, S1109A, S1109G, S1109V, S1109C, S1109H, I1110P, L1111V, L1111S, P1112Q, P1112G, P1112D, K1113A, K1113R, K1113G, R1114N, R1114T, R1114S, R1114Q, R1114H, R1114D, R1114A, R1114G, R1114C, R1114K, R1114Y, N1115H, N1115S, N1115P, N1115F, N1115L, N1115D, N1115A, N1115W, S1116T, S1116G, S1116K, D1117G, D1117N, D1117H, D1117S, K1118N, K1118T, K1118R, K1118S, K1118Q, K1118L, K1118A, K1118G, K1118C, K1118H, K1118V, L1119G, L1119R, 11120V, 11120T, I1120L, A1121R, A1121P, A1121D, A1121Y, R1122I, K1123N, K1124R, K1124V, D1125A, D1125G, D1125C, D1125T, D1125H, D1125R, D1125N, D1125Y, D1127S, P1128F, K1129V, K1129T, K1129I, Y1131L, F1134Y, D1135M, D1135E, D1135A, D1135T, D1135S, D1135H, D1135G, D1135V, S1136K, S1136T, S1136G, T1138K, T1138N, T1138S, T1138A, T1138Y, T1138R, T1138Q, T1138G, V1139L, V1139E, V1139D, V1139G, V1139S, V1139H, A1140C, A1140T, Y1141R, S1142A, S1142V, S1142C, S1142G, V1143L, V1143A, V1143T, L1144V, L1144W, L1144A, L1144Y, L1144C, V1145N, V1145L, V1145A, V1145F, V1146Y, V1146W, V1146C, V1146K, V1146R, V1146E, V1146A, A1147H, K1148R, K1148Q, K1148L, K1148A, K1148W, K1148T, K1148C, V1149T, V1149M, V1149Q, V1149L, V1149E, E1150S, E1150H, E1150L, E1150P, E1150A, K1151N, K1151I, K1151Q, K1151H, K1151L, K1151E, K1151A, K1151G, K1151V, K1151Y, K1151S, K1151F, K1151R, K1151P, K1151C, G1152S, G1152P, G1152T, G1152R, G1152Q, G1152L, K1153N, K1153M, K1153I, K1153Q, K1153H, K1153E, K1153G, K1153V, K1153T, K1153R, K1153S, K1153L, K1153C, S1154N, S1154E, S1154D, S1154G, S1154A, K1155S, K1155P, K1155R, K1155L, K1155G, K1155V, K1155T, K1155E, K1155A, K1156T, K1156I, K1156Q, K1156P, K1156L, K1156R, K1156D, K1156V, K1156Y, K1156S, L1157T, L1157M, L1157H, L1157R, L1157A, L1157G, L1157I, K1158H, K1158E, K1158D, K1158G, K1158V, K1158Y, K1158F, K1158T, K1158S, K1158L, S1159H, S1159P, S1159L, S1159E, S1159A, S1159G, S1159F, S1159N, S1159T, S1159M, S1159Q, S1159V, V1160N, V1160E, V1160T, K1161V, K1161S, K1161T, K1161I, K1161L, K1161D, E1162G, E1162D, E1162V, E1162Y, L1163M, L1163A, L1163G, L1163T, L1163V, L1163S, L1164H, L1164V, L1164F, L1164M, L1164I, L1164E, G1165K, G1165P, G1165A, G1165S, G1165L, G1165E, G1165Y, T1167P, T1167Y, T1167Q, T1167H, T1167L, M1169L, R1171K, R1171H, R1171Q, S1172T, S1172P, S1172L, S1172G, S1172V, S1172E, S1172A, S1172Y, S1172M, S1172C, S1172F, S1173K, S1173Y, S1173L, S1173C, S1173R, S1173Q, S1173D, S1173H, S1173V, S1173W, F1174S, E1175Q, E1175L, K1176S, K1176I, K1176D, K1176A, K1176N, K1176T, K1176Q, K1176C, K1176L, K1176V, N1177T, N1177G, N1177S, N1177C, P1178R, 11179A, 11179G, I1179Y, I1179T, I1179Q, I1179E, D1180T, D1180R, D1180S, D1180P, D1180L, D1180E, D1180A, D1180G, D1180I, F1181V, F1181T, F1181M, F1181Q, F1181H, F1181L, F1181A, L1182V, L1182C, E1183M, E1183I, E1183G, E1183V, E1183S, E1183H, E1183L, E1183D, A1184N, A1184R, A1184Q, A1184V, A1184D, A1184G, A1184C, K1185N, K1185S, K1185L, K1185T, K1185E, K1185A, K1185G, K1185C, G1186P, Y1187L, Y1187F, Y1187S, Y1187P, Y1187W, K1188R, K1188A, K1188V, K1188S, K1188W, K1188F, K1188L, K1188Y, K1188C, E1189L, E1189Q, V1190M, V1190L, V1190A, V1190I, K1191T, K1191R, K1191I, K1191Q, K1191P, K1191L, K1191D, K1191A, K1191G, K1191V, K1191Y, K1191F, K1191N, K1191S, K1191E, K1191C, K1191H, K1192N, K1192H, K1192R, K1192E, K1192S, K1192L, K1192Q, K1192P, K1192A, K1192V, K1192T, D1193H, D1193Y, D1193I, D1193P, D1193R, D1193A, D1193V, D1193C, L1194S, L1194V, L1194W, L1194F, L1194Q, L1194H, L1194A, L1194E, L1194C, L1194R, L1194D, L1194G, I1195T, I1195V, I1196K, I1196T, I1196H, I1196F, I1196A, I1196M, I1196L, I1196Y, K1197L, K1197I, K1197F, K1197C, L1198T, L1198A, L1198V, L1198F, P1199Y, P1199C, P1199T, P1199R, P1199H, P1199G, P1199F, P1199A, K1200D, K1200S, Y1201G, Y1201S, S1202A, S1202V, S1202I, S1202Q, S1202H, S1202Y, L1203N, L1203F, L1203T, L1203A, L1203C, F1204I, F1204L, F1204V, E1205C, E1205K, E1205T, E1205M, E1205R, L1206S, L1206Q, L1206A, L1206Y, L1206F, L1206D, L1206G, L1206V, L1206W, E1207R, E1207C, E1207Q, N1208T, N1208P, N1208L, N1208W, N1208H, N1208G, N1208S, N1208C, G1209K, G1209T, G1209Y, G1209S, G1209C, G1209M, G1209R, G1209A, G1209V, R1210S, R1210P, R1210L, R1210E, R1210A, R1210G, R1210C, R1210M, R1210I, K1211T, K1211S, K1211M, K1211Q, K1211L, K1211A, K1211Y, K1211N, K1211W, R1212V, R1212C, M1213T, M1213I, M1213H, M1213L, M1213A, M1213V, M1213Y, M1213S, M1213W, M1213F, M1213Q, M1213P, M1213R, M1213C, L1214T, L1214M, L1214H, L1214V, L1214S, L1214C, A1215G, A1215C, A1215S, S1216T, S1216A, A1217T, A1217H, A1217P, A1217D, A1217G, A1217W, A1217F, A1217N, A1217S, A1217M, A1217C, G1218S, G1218E, G1218W, L1220M, L1220N, L1220S, L1220W, L1220C, Q1221N, Q1221S, Q1221M, Q1221I, Q1221A, Q1221V, Q1221C, Q1221T, Q1221H, Q1221L, Q1221G, K1222T, K1222R, K1222S, G1223M, G1223S, G1223N, G1223A, G1223C, N1224T, N1224D, N1224P, N1224G, E1225Q, L1226V, L1226M, L1226H, L1226P, A1227Y, A1227S, A1227G, L1228I, L1228F, L1228S, L1228V, P1229K, P1229N, P1229T, P1229S, P1229I, P1229Q, P1229D, P1229A, P1229G, P1229V, P1229C, P1229F, P1229R, P1229M, P1229H, P1229L, S1230T, S1230M, S1230Q, S1230L, S1230G, S1230V, K1231T, K1231S, K1231H, K1231P, K1231D, K1231A, K1231V, K1231W, K1231F, K1231M, K1231L, K1231E, K1231G, K1231Y, K1231C, Y1232L, Y1232N, Y1232T, Y1232R, Y1232S, Y1232D, Y1232V, V1233I, V1233Q, V1233L, V1233E, V1233W, V1233T, N1234T, N1234Q, N1234R, N1234L, N1234D, N1234A, N1234V, N1234P, N1234C, F1235L, F1235W, F1235M, F1235H, F1235A, F1235G, L1236T, L1236I, L1236V, L1236Q, Y1237K, Y1237S, Y1237G, Y1237V, Y1237R, Y1237M, Y1237Q, Y1237L, Y1237A, Y1237C, L1238Q, L1238H, L1238A, L1238V, L1238F, L1238S, L1238Y, A1239M, A1239H, A1239L, A1239W, S1240Q, S1240H, S1240L, S1240Y, S1240F, S1240N, S1240I, S1240A, S1240G, H1241N, H1241I, H1241Q, H1241L, H1241G, H1241M, H1241R, H1241D, H1241A, H1241V, H1241Y, H1241F, Y1242K, Y1242T, Y1242S, Y1242M, Y1242P, Y1242G, Y1242V, Y1242W, Y1242I, Y1242L, E1243N, E1243S, E1243M, E1243A, E1243T, E1243P, E1243V, K1244S, K1244I, K1244Q, K1244P, K1244L, K1244E, K1244D, K1244V, K1244F, K1244R, K1244M, K1244A, K1244G, L1245N, L1245Q, L1245P, L1245G, L1245V, L1245Y, L1245F, L1245S, L1245M, L1245R, L1245A, L1245W, L1245C, K1246N, K1246S, K1246R, K1246D, K1246W, K1246I, K1246L, K1246A, K1246G, K1246C, G1247S, G1247H, G1247W, G1247K, G1247T, G1247P, G1247L, G1247A, G1247Y, G1247F, S1248I, S1248R, S1248L, S1248D, S1248G, S1248Y, S1248W, S1248M, P1249N, P1249D, P1249G, P1249V, P1249R, P1249S, P1249H, P1249L, E1250R, E1250Q, E1250P, E1250G, E1250Y, E1250S, E1250F, E1250T, E1250D, D1251S, D1251W, D1251N, D1251T, D1251C, N1252K, N1252T, N1252A, N1252V, N1252S, N1252C, N1252R, N1252G, E1253N, E1253T, E1253D, E1253A, E1253G, E1253W, E1253C, E1253S, E1253R, E1253L, Q1254N, Q1254I, Q1254H, Q1254P, Q1254R, Q1254E, Q1254G, Q1254Y, Q1254W, Q1254T, Q1254D, Q1254V, Q1254C, K1255R, K1255S, K1255P, K1255G, K1255V, K1255F, K1255L, Q1256T, Q1256P, Q1256E, Q1256S, Q1256R, Q1256W, Q1256C, L1257R, L1257H, L1257P, L1257E, L1257V, L1257W, L1257S, L1257M, L1257G, L1257C, F1258K, F1258R, F1258L, F1258V, F1258S, F1258P, V1259R, V1259I, V1259L, V1259E, V1259C, V1259F, V1259A, E1260S, E1260Q, E1260R, E1260L, E1260G, E1260V, E1260Y, E1260F, E1260A, E1260C, Q1261T, Q1261D, Q1261A, Q1261V, Q1261W, Q1261L, Q1261M, Q1261H, Q1261P, Q1261G, Q1261Y, H1262N, H1262R, H1262L, H1262A, H1262W, H1262C, H1262T, H1262D, H1262V, H1262S, K1263R, K1263Q, K1263P, K1263L, K1263A, K1263N, K1263S, K1263I, K1263D, K1263V, K1263F, H1264M, H1264Q, H1264G, H1264V, H1264S, H1264I, H1264L, Y1265L, Y1265V, Y1265C, Y1265R, Y1265I, Y1265Q, Y1265H, Y1265S, L1266G, L1266C, L1266T, L1266S, L1266W, D1267N, D1267T, D1267Q, D1267P, D1267L, D1267I, D1267G, D1267V, E1268T, E1268L, E1268D, E1268G, E1268V, E1268Y, E1268S, E1268R, E1268A, I1269L, I1269V, I1269M, I1269D, I1269C, I1270T, I1270M, I1270L, I1270Q, I1270F, E1271T, E1271R, E1271H, E1271L, E1271D, E1271A, E1271W, E1271S, E1271C, Q1272T, Q1272S, Q1272A, Q1272G, Q1272W, Q1272C, Q1272I, I1273T, I1273M, I1273F, I1273L, S1274K, S1274H, S1274R, S1274A, S1274V, S1274T, S1274G, S1274L, S1274F, E1275T, E1275L, E1275Y, E1275I, E1275S, E1275W, F1276S, F1276M, F1276Y, F1276C, F1276E, F1276G, S1277T, S1277M, S1277I, S1277H, S1277E, S1277G, S1277V, S1277N, S1277F, K1278M, K1278I, K1278V, K1278S, K1278G, K1278Y, K1278L, R1279S, R1279L, R1279D, R1279G, R1279T, R1279A, R1279W, R1279C, V1280T, V1280I, V1280Q, V1280L, V1280E, V1280D, V1280A, V1280S, I1281M, I1281H, I1281R, 11281L, I1281A, 11281V, 11281Y, I1281N, I1281S, I1281C, L1282N, L1282T, L1282S, L1282R, L1282E, L1282D, L1282A, L1282G, L1282V, L1282W, L1282C, L1282Q, A1283N, A1283T, A1283G, A1283Y, A1283C, A1283S, A1283H, A1283R, A1283E, D1284G, A1285N, A1285T, A1285R, A1285L, A1285E, A1285D, A1285W, A1285F, A1285H, A1285P, A1285V, N1286T, N1286S, N1286M, N1286Q, N1286P, N1286L, N1286G, N1286V, N1286W, N1286C, N1286H, N1286E, N1286A, N1286F, L1287S, L1287Q, L1287P, L1287R, L1287E, L1287F, L1287N, L1287T, L1287I, L1287A, L1287V, L1287W, D1288N, D1288M, D1288E, D1288A, D1288G, D1288V, D1288S, D1288L, K1289R, K1289M, K1289I, K1289P, K1289E, K1289D, K1289G, K1289S, K1289C, K1289L, K1289Q, K1289V, V1290L, V1290W, V1290M, V1290H, L1291N, L1291T, L1291M, L1291Q, L1291H, L1291E, L1291A, L1291Y, L1291I, L1291S, L1291W, L1291F, S1292T, S1292M, S1292R, S1292G, S1292Y, S1292H, S1292L, S1292V, S1292C, S1292F, A1293T, A1293S, A1293P, A1293L, A1293V, A1293Y, A1293H, A1293G, Y1294T, Y1294I, Y1294R, Y1294V, Y1294W, Y1294C, Y1294S, Y1294H, Y1294L, N1295T, N1295Q, N1295P, N1295E, N1295D, N1295W, N1295F, N1295K, N1295S, N1295I, N1295H, N1295R, N1295L, N1295A, N1295G, K1296L, K1296E, K1296A, K1296Y, K1296Q, K1296R, K1296V, K1296S, K1296W, H1297R, H1297E, H1297S, H1297L, H1297F, H1297V, R1298N, R1298T, R1298Q, R1298L, R1298A, R1298G, R1298V, R1298C, R1298S, R1298F, D1299K, D1299T, D1299L, D1299A, D1299G, D1299Y, D1299S, D1299W, D1299V, D1299C, D1299F, K1300T, K1300H, K1300L, K1300D, K1300N, K1300R, K1300E, K1300V, K1300W, K1300F, P1301N, P1301D, P1301G, P1301T, P1301S, P1301V, P1301W, I1302N, I1302H, I1302W, I1302T, I1302S, I1302A, I1302V, R1303T, R1303P, R1303L, R1303E, R1303D, R1303G, R1303V, R1303K, R1303S, R1303I, R1303A, E1304N, E1304T, E1304R, E1304H, E1304Y, E1304S, E1304V, Q1305N, Q1305S, Q1305K, Q1305R, Q1305M, Q1305H, Q1305L, Q1305V, A1306T, A1306S, A1306I, A1306C, A1306N, E1307S, E1307I, E1307Q, E1307L, E1307D, E1307G, E1307V, E1307H, E1307R, E1307C, N1308R, I1309C, 11309S, 11309Q, 11309R, 11309A, 11309V, 11309Y, I1309L, 11310K, I1310M, 11310Q, I1310R, I1310V, 11310Y, 11310C, 11310T, 11310L, 11310A, 1131° F., H1311N, H1311T, H1311S, H1311P, H1311R, H1311A, H1311G, H1311K, H1311M, H1311D, H1311V, LI312M, LI312A, LI312V, LI312C, LI312S, LI312G, LI312W, LI312F, F1313T, F1313M, F1313L, F1313C, F1313A, F1313V, F1313S, F1313W, T1314N, T1314M, T1314H, T1314L, T1314G, T1314S, T1314F, T1314K, T1314Q, T1314E, T1314D, T1314A, T1314C, L1315S, L1315I, L1315H, L1315E, L1315A, L1315V, L1315Q, T1316A, T1316Q, T1316G, N1317I, N1317Q, N1317H, N1317L, N1317A, N1317Y, N1317W, N1317F, N1317T, N1317S, N1317G, N1317V, L1318K, L1318N, L1318T, L1318I, L1318Q, L1318H, L1318P, L1318E, L1318D, L1318G, L1318V, L1318Y, L1318W, L1318C, L1318M, L1318A, L1318S, G1319S, G1319N, G1319R, G1319L, A1320T, A1320R, A1320S, A1320I, A1320H, A1320G, A1320V, A1320Y, A1320C, A1320F, A1320W, P1321S, P1321H, P1321A, P1321G, P1321V, P1321C, P1321M, P1321I, P1321R, A1322N, A1322I, A1322L, A1322G, A1322V, A1322T, A1322M, A1322Q, A1322W, A1322F, A1323G, A1323S, A1323C, F1324T, F1324S, F1324I, F1324H, F1324L, F1324A, F1324G, F1324V, F1324C, F1324R, F1324M, F1324D, K1325R, Y1326M, Y1326L, Y1326V, Y1326W, Y1326F, Y1326I, Y1326Q, Y1326P, Y1326A, Y1326S, F1327L, F1327A, F1327C, F1327R, D1328T, D1328M, D1328L, D1328A, D1328S, D1328K, D1328G, D1328V, T1329K, T1329N, T1329R, T1329A, T1329G, T1329S, T1329C, T1329M, T1329I, T1329L, T1329V, T1329Y, T1329F, T1330K, T1330N, T1330P, T1330A, T1330S, T1330Q, I1331S, 11331M, I1331C, I1331L, I1331G, D1332E, D1332A, D1332V, D1332Y, D1332C, D1332S, D1332W, K1334T, K1334A, K1334G, K1334Q, K1334R, K1334S, K1334W, Y1336M, Y1336H, Y1336L, Y1336C, Y1336F, Y1336N, Y1336P, T1337N, T1337S, T1337I, T1337P, T1337A, T1337G, S1338N, S1338T, S1338M, S1338I, S1338L, S1338A, S1338G, S1338V, S1338Y, S1338W, S1338C, S1338F, S1338R, S1338Q, T1339S, T1339M, T1339I, T1339Q, T1339H, T1339R, T1339L, T1339E, T1339A, T1339G, T1339V, T1339F, T1339W, K1340N, K1340T, K1340S, K1340M, K1340I, K1340Q, K1340H, K1340P, K1340E, K1340A, K1340G, K1340V, K1340C, K1340L, K1340F, K1340R, K1340Y, E1341A, E1341I, E1341V, V1342M, V1342L, V1342S, V1342T, V1342I, V1342W, V1342C, L1343I, L1343S, L1343K, L1343E, L1343W, L1343F, D1344E, D1344A, D1344G, D1344S, D1344H, A1345S, A1345C, T1346N, T1346L, T1346K, T1346I, T1346R, T1346V, T1346Y, L1347V, L1347Y, L1347F, L1347S, I1348V, I1348C, I1348L, I1348M, I1348A, I1348K, I1348S, I1348F, H1349R, H1349A, H1349Y, H1349W, H1349V, H1349F, H1349T, H1349M, H1349S, H1349K, H1349L, Q1350R, Q1350S, Q1350W, Q1350L, Q1350K, Q1350N, Q1350T, Q1350I, S1351T, I1352K, I1352T, I1352H, I1352P, I1352R, I1352S, I1352V, I1352C, I1352L, T1353C, G1354K, G1354P, G1354V, L1355I, L1355T, L1355S, Y1356S, Y1356R, Y1356V, Y1356C, Y1356L, Y1356Q, Y1356W, E1357S, E1357T, E1357I, E1357F, T1358H, T1358R, T1358S, T1358P, R1359Q, R1359C, R1359L, R1359N, R1359V, R1359A, R1359W, R1359Y, I1360R, I1360V, I1360S, D1361N, D1361A, D1361S, D1361C, D1361G, L1362C, L1362I, L1362P, L1362V, L1362F, S1363T, S1363R, S1363M, S1363Q, S1363A, S1363G, S1363V, S1363H, S1363W, S1363L, Q1364R, Q1364M, Q1364V, Q1364C, Q1364N, Q1364S, Q1364I, Q1364H, Q1364E, Q1364G, L1365M, L1365Y, L1365T, L1365S, L1365C, G1366A, G1366K, G1366M, G1366P, G1366D, G1366S, G1367N, G1367S, G1367M, G1367P, G1367A, G1367V, G1367Y, G1367T, G1367Q, G1367D, D1368T, D1368M, D1368L, D1368S, D1368W, D1368K, D1368R, D1368H, D1368A, D1368G, D1368V;
at least one amino acid substitution selected from the group consisting of D2Q, D2C, D2N, D2R, D2M, D2V, D2Y, D2F, K3I, K3V, S6T, G8T, L9V, T13S, E24S, K26D, K26S, K26R, P28V, P28Y, P28F, S29N, K30T, K30M, K30I, K30L, K30G, K30C, K30V, K30Q, K31M, K31P, K31E, K31S, K31G, K31V, K31R, K31Q, F32T, F32M, F32L, F32C, F32A, K33S, K33P, K33D, K33A, K33G, K33V, K33W, K33N, K33H, K33R, K33L, K33E, K33C, V34R, V34C, L35E, L35A, L35N, L35S, G36A, G36S, N37T, T38G, D39L, D39W, R40N, R40T, R40S, R40L, R40F, R40V, R40E, R40A, R40G, H41T, H41R, H41L, S42Q, S42P, S42H, S42E, I43A, I43W, I43Q, I43Y, K44Q, K44G, K44C, K44D, N46L, N46C, N46K, N46M, N46S, 148M, A50C, A50T, A50V, A50S, A50N, A50L, A50Y, A50F, A50H, L51T, L51R, L51V, L521, F53L, D54K, D54T, D54R, D54S, D54N, D54L, D54E, S55P, S55E, G56A, E57K, E57Q, E57V, E57R, T58S, T58G, E60K, E60R, E60Q, E60V, E60L, E60T, A61R, A61N, A61L, T62R, L64K, L64R, K65H, K65L, K65Y, K65F, R66P, T67S, T67A, T67G, T67Q, T67C, T67L, A68K, A68T, A68R, A68I, A68Q, A68L, A68V, A68M, Y72N, Y72T, Y72I, Y72L, Y72G, Y72V, Y72C, Y72F, Y72M, T73R, K76R, N77K, N77R, N77C, R78P, C80K, C80R, C80H, C80L, C80V, C80S, C80G, C80I, S87R, E89P, A91H, A91N, K92L, K92A, K92V, S96T, S96R, S96H, S96E, H99E, H99A, H99S, H99K, E102A, E102T, E102S, E108S, E108R, E108P, E108A, E108V, H113F, H113Y, E114S, E114A, R115K, H116W, D124E, V126D, V126A, A127R, E130K, E130A, E130L, E130N, E130I, E130Q, K131Q, K131A, K131T, K131R, K131H, K140T, K140A, K140G, K140Y, K140S, K140C, K140F, K140W, K141E, V143C, S145E, T146N, D147H, K148I, K148S, K148C, L151A, L151P, L156S, L156N, A157I, L158A, A159H, L169N, L169R, L169H, L169K, N175T, N175E, N175V, D180T, D180S, D180M, D180H, D180E, D180A, D180G, D180W, D180C, V181P, D182A, D182G, K183N, K183T, K183S, K183M, K183I, K183H, K183P, K183C, K183Q, K183G, F185T, I186E, I186D, 1186Q, I186C, Q187H, L188V, V189N, V189Y, V189T, V189R, V189L, Q190E, Q190W, Q190P, T191S, Y192V, N193G, Q194L, Q194Y, L195T, L195I, L195H, L195E, L195V, L195F, F196D, F196G, F196T, F196M, N199L, N199A, P200R, I201T, I201S, I201L, N202P, A203I, A203T, A203M, A203L, A203V, A203S, G205Q, G205M, G205L, V206K, V206N, V206R, V206S, V206M, V206H, V206P, V206E, V206D, V206G, V206F, V206Q, V206Y, D207I, A208S, A208H, A208L, A208E, A208G, A208V, A208W, A208M, A208C, A210R, I211L, L212I, L212V, S213L, S213E, R215T, R215M, R215I, R215Q, R215P, R215L, R215V, R215Y, R215W, R215C, L216E, S217M, S217N, S217P, S217D, S217G, S219Q, S219L, S219A, S219V, S219F, R220T, R220Q, R220H, R220P, R220L, R220E, R220A, R220V, R220S, R220M, R220D, R220F, L222N, L222M, L222H, L222A, L222G, L222Y, L222W, L222C, L222F, L222S, E223P, N224P, N224L, L225Y, L225W, L225Q, 1226K, I226M, 1226Q, I226P, I226D, I226G, I226R, I226H, I226A, A227W, A227P, L229M, L229P, L229S, P230M, P230S, P230V, P230Y, G231K, G231D, G231M, G231A, G231F, E232H, E232I, E232P, K233E, K234I, K234Q, K234S, G236P, G236L, G236I, L237T, L237I, L237P, L237C, L237F, F238P, G239T, G239P, G239V, G239M, G239I, G239L, G239Y, G239F, N240Q, N240P, L241V, L241Y, L241W, L241F, L241T, L241S, L241I, L241A, I242L, I242Y, A243T, A243C, L244R, L244W, S245Y, S245F, L246M, L246H, L246G, L246Y, G247N, G247T, G247S, G247V, G247C, L248T, T249P, T249F, T249R, T249A, P250N, P250T, P250S, P250L, P250D, N251E, N251A, F252P, F252A, K253S, K253L, K253E, K253Y, N255V, F256Q, F256A, F256N, F256T, F256S, F256L, F256V, F256C, L258T, L258P, L258F, A259P, A259W, A259C, A259M, A259L, E260I, A262C, A262E, A262V, K263R, K263G, K263A, L264K, L264S, L264M, L264Q, L264H, L264P, Q265S, Q265L, L266T, L266C, L266V, S2671, S267P, S267L, Y271P, Y271S, Y271K, Y271Q, D272K, D272R, D272L, D272G, D272C, D272S, D272M, D272I, D272P, D272V, D273K, D273R, D273S, D273Q, D273H, D273L, D273A, D273Y, D273T, D273P, D273G, D273V, D274P, D274S, D274M, D274L, D274G, D274V, D274W, L275K, L275S, L275H, L275M, L275E, L275D, L275G, L275W, D276K, D276N, D276T, D276R, D276S, D276M, D276I, D276Q, D276H, D276P, D276L, D276G, D276W, D276C, D276F, D276A, D276V, D276Y, N277R, L278F, L279N, L279S, L279Q, L279H, L279A, L279G, L279K, L279R, L279I, L279P, L279E, L279D, L279V, L279Y, L279W, A280K, A280R, A280Q, A280H, A280L, A280Y, A280W, A280C, A280F, A280T, A280I, A280V, Q281R, Q281A, Q281Y, I282H, I282E, I282D, I282G, I282W, I282N, I282R, I282S, G283T, G283D, G283Y, G283W, G283N, G283S, G283Q, G283P, G283F, D284T, D284A, D284W, Q285S, Q285M, Q285E, Q285Y, Q285L, Q285A, Y286T, Y286E, Y286D, Y286V, Y286G, A287N, A287T, A287R, A287S, A287M, A287I, A287E, A287D, A287G, A287Y, A287W, A287F, A287K, A287Q, A287H, A287P, A287L, A287V, D288S, D288Q, D288E, D288W, L289S, L289G, L289V, L289C, F290T, F290S, F290Q, F290H, F290A, F290G, L291N, L291R, L291D, L291W, L291K, L291T, L291S, L291P, L291A, L291G, L291Y, L291C, L291F, A292R, A293S, A293I, A293L, A293D, A293G, A293V, A293T, A293H, N295K, N295R, N295C, N295Q, N295P, N295A, L296A, L296W, L296T, L296D, S297Y, S297W, S297K, S297N, S297G, D298M, D298H, D298N, A299T, A299E, A299C, L301I, L301R, L301G, L301V, L301C, L301K, L301N, S303E, D304L, D304W, R307T, R307S, R307G, V308W, T310S, T310A, T310G, T310P, T310L, T313E, K314S, K314L, K314E, K314D, K314A, K314C, I322V, 1322T, 1322C, K323N, K323T, K323M, K323I, K323Q, K323H, K323L, K323E, K323D, K323A, K323G, K323V, K323Y, K323S, K323C, D326A, E327T, H329K, H329R, Q330E, Q330S, Q330V, L332K, T333L, T333S, T333A, T333G, T333V, T333K, T333N, T333R, T333H, T333M, T333I, L334T, L334Q, L334V, L334S, L334A, L335A, A337S, A337Q, L338A, L338T, V339T, Q341L, Q341R, Q341C, Q342S, Q342E, Q342R, Q342W, L343R, L343K, L343M, L343W, L343S, P344T, P344R, P344S, P344H, P344G, E345N, E345T, E345R, E345S, E345Y, E345M, E345I, E345H, E345L, K346M, E349T, E349R, E349S, E349M, E349L, E349V, E349W, E349N, E349G, I350L, I350V, F352S, D353L, D353S, D353A, D353C, Q354R, Q354S, Q354G, Q354Y, Q354T, Q354D, S355R, S355K, S355C, S355Y, K356S, N357R, N357K, N357S, D364K, D364T, D364R, D364S, D364M, D364H, D364G, D364V, G366P, G366L, G366S, A367R, A367T, S368N, E370A, E371R, E371Q, E371L, E371K, E371S, F375H, I376V, K377A, P378V, P378F, 1379S, 1379H, E381I, E381H, E381R, E381L, K382R, D384V, G385H, L390I, V391E, V391D, V391T, V391A, K392S, K392A, L393V, N394E, N394D, N394V, R395I, R395L, R395A, R395C, L399C, T404M, T404V, F405T, F405S, F405I, F405R, F405V, S409A, S409V, S409C, H420G, R424E, R424V, R425S, R425C, E427K, D428T, F429H, K434A, D435M, N436Q, R437C, R437A, E438L, 1440L, E441R, T445S, T445W, T445R, T445E, R447T, Y450H, Y450S, Y451W, R457K, R457N, R457A, R457M, M465L, T466V, T466I, E470D, E471Q, T472K, T472R, T472S, T472H, T472P, T472A, T472N, T474R, T474H, E479D, E479W, E480S, E480A, K484L, G485K, G485L, G485A, G485V, Q489L, Q489A, S490A, S490G, E493S, M495H, T496V, N497P, N497Q, F498N, F498S, F498L, F498A, F498V, F498C, F498R, F498G, D499R, K500R, K500S, K500P, K500E, K500A, K500G, K500V, K500T, K500Q, N501R, N501H, L502V, L502A, P503R, N504E, E505T, V507S, V507L, H511R, S512R, S512L, L513K, L513I, L513V, L514T, L514V, Y515A, Y515L, Y517D, Y517G, Y517V, T519D, T519Y, T519S, T519I, T519Q, V520T, V520A, E523A, L524T, L524S, T525Y, K526L, V527T, K528R, K528I, K528V, K528W, Y529S, V530D, V530T, V530Q, T531Q, T531D, T531Y, E532G, G533A, G533R, G533E, G533D, M534S, R535L, R535D, R535A, R535G, K536I, K536D, K536V, K536F, K536N, K536E, K536Y, K536C, P537Q, P537F, A538L, A538Y, A538P, S541I, S541G, G542L, E543C, K545N, K546Q, K546C, A547M, A547H, A547P, A547V, A547F, 1548M, 1548C, V549L, V549C, D550S, L551A, L552A, T555D, T555G, T555F, T555Y, N556I, R557I, R557F, K558T, K558F, K558W, V559L, V559A, T560K, T560S, K562H, K562R, K562L, K562A, K562T, K562S, Q563V, Q563I, L564T, L564C, L564P, K565H, K565P, D567C, Y568I, Y568H, F569L, K570L, K570V, K571H, K571T, 1572C, E573R, E573H, E573Y, E573G, C574I, C574P, C574A, C574V, F575I, F575E, F575G, D576H, S577T, V578M, V578P, E579Y, E579S, E579I, 1580F, S581R, S581L, G582Q, G582L, G582V, G582R, G582Y, V583F, V583R, V583M, D585Q, R586L, R586Y, R586H, R586P, S590V, S590M, L591H, G592L, G592S, G592W, G592F, G592H, G592Y, T593G, L598S, L598W, L598A, K599I, K599H, K599L, K599A, K599Q, K599G, K599S, K602M, K602P, K602R, K602G, K602W, K602S, K602L, K602E, K602V, D603T, D603V, D603E, K604G, K604Y, D605G, F606V, L607T, N609P, N609D, N609W, E610L, E611L, E611H, E611R, E611G, E611S, N612T, N612A, E613Q, D614S, I615S, 1615L, 1615G, 1615R, L616V, D618P, D618L, 1619T, I619L, 1619M, V620A, V620T, L621H, L621W, L621F, L621S, L623M, T624S, E627Y, E627R, E627M, E627L, R629T, R629Q, R629L, R629W, R629I, R629F, I632L, E633D, E633S, E634S, E634T, E634V, L636F, K637N, K637A, K637G, T638H, T638P, T638G, T638V, Y639I, Y639A, Y639V, A640I, A640E, A640L, K646M, K646Q, K646L, K646E, K646D, K646A, K646G, K646V, K646C, K646F, K646T, K646S, K646I, K646H, K646P, V647M, M648W, M648Y, K649T, K649I, K649Q, K649H, K649P, K649L, K649E, K649D, K649A, K649G, K649V, K649Y, K649W, K649C, K649F, K649S, K649M, Q650E, Q650D, Q650A, Q650V, Q650L, K652M, K652H, K652E, K652D, K652W, K652F, K652Q, K652Y, R653T, R653S, R653M, R653I, R653L, R653E, R653D, R653A, R653G, R653V, R653C, R655L, R655Y, Y656W, T657R, T657M, T657A, G658R, G658H, G658K, G660A, L662D, L662E, R664L, R664S, R664P, K665Q, K665H, K665V, I670L, 1670H, R671L, R671S, K673H, K673Y, K673S, K673G, K673V, Q674G, Q674K, Q674N, Q674S, Q674L, S675A, G676N, G676R, G676Q, G676L, G676S, K677M, K677H, K677S, K677A, F682L, S685H, S685R, G687S, G687V, F688R, A689M, A689P, A689E, A689L, N690S, R691S, R691H, R691N, R691L, N692A, N692G, M694P, M694W, Q695G, I697C, H698S, H698W, D700T, D700S, D700Q, D700H, D700A, D700G, D700C, D700I, D700V, S701I, S701R, S701E, S701Y, L702F, T703S, K705R, E706G, E706R, I708L, K710S, K710A, A711S, Q712M, Q712W, V713P, V713T, V713R, S714L, S714T, S714M, S714R, S714A, S714F, G715R, G715P, G715L, G715S, G715A, G717P, G717A, G717T, G717I, D718K, D718R, D718M, D718T, D718Q, D718H, D718S, D718G, D718V, D718N, S719N, L720A, L720V, H721K, H721V, H721Q, H721L, H721P, E722K, E722T, E722R, E722W, E722D, E722L, H723K, H723N, H723T, H723M, H723I, H723Q, H723A, H723S, H723R, H723L, H723C, A725K, A725R, A725Q, A725H, A725W, A725V, A725C, A725L, A725N, A725P, N726R, N726A, N726K, L727V, L727G, A728Q, A728E, A728D, A728G, A728Y, A728W, A728F, A728R, A728P, A728S, A728L, G729N, G729T, G729S, G729M, G729I, G729Q, G729H, G729P, G729L, G729E, G729D, G729A, G729V, G729Y, G729W, G729C, G729F, G729R, S730N, S730P, S730G, S730C, S730K, S730R, S730H, S730D, S730A, P731T, P731M, P731V, A732N, A732S, A732M, A732T, A732Q, A732P, A732R, A732V, I733T, I733V, I733E, I733Y, I733A, I733N, K734R, K735R, G736S, G736W, G736T, G736V, I737V, L738R, L738Y, L738W, L738F, L738M, L738V, L738T, Q739R, T740A, V741L, V741G, V743L, V743I, D745T, V748T, K749L, V750S, V750I, V750C, R753N, R753M, R753H, R753D, R753G, R753S, R753P, R753C, R753T, R753L, R753Y, H754T, N758L, N758V, I759L, M763V, A764V, R765S, E766Y, E766W, E766R, N767T, N767A, N767V, N767S, Q768W, Q771A, Q771T, Q771Y, Q771S, Q774L, Q774V, K775R, K775A, N776R, N776C, N776Q, R778H, R778N, R778L, M781G, K782R, K782A, K782Y, R783T, R783I, R783P, R783L, R783M, R783Q, R783A, R783G, R783V, R783C, E785Q, E786H, K789H, K789Q, K789R, G792K, G792S, Q794T, Q794M, Q794P, Q794L, Q794V, I795Q, K797M, K797Y, K797S, E798L, E798V, H799T, H799K, V801C, V801S, E802S, T804P, T804Y, T804R, T804D, Q805A, Q805Y, L806P, Q807R, K810R, Y812F, Y812M, L816S, L816F, Q817S, N818S, N818D, R820K, R820Y, R820C, V824G, V824Y, D825P, D825N, D825Y, Q826K, E827P, I830S, N831L, N831V, N831F, R832S, S834V, D835T, D835H, D835A, D835V, Q844A, F846M, F846Y, K848R, 1852W, D853S, D853G, K855L, V856A, R859L, K862S, K862H, G865R, G865C, S867L, D868K, D868T, N869T, N869S, V870L, E873C, E874H, E874D, E874G, E874C, E874S, V875I, V875C, K877T, K877S, K877M, K877R, K877L, K877A, K877C, M879R, K880L, K880I, N881T, N881Q, N881P, N881D, N881V, N881G, Y882D, Y882A, Y882V, Y882S, Q885T, Q885S, Q885A, Q885C, L886Y, N888T, N888H, N888R, N888V, K890R, 1892V, T893G, Q894T, Q894G, F897W, N899C, N899W, T901M, K902A, A903F, R905T, R905Q, R905L, R905E, R905A, R905Y, R905S, R905C, R905V, R905W, R905F, G907S, S909D, E910F, L911M, K913G, K913V, A914M, A914T, G915H, G915A, G915F, K918R, R919Q, R919I, Q920L, V922A, V922C, E923M, E923I, E923H, E923L, E923A, E923V, T924V, Q926N, Q926M, Q926W, Q926C, 1927Q, 1927L, T928I, T928C, K929T, K929L, H930T, H930Q, H930E, H930A, H930M, H930L, H930F, A932T, A932V, Q933R, Q933L, S937Q, S937R, R938K, M939Y, M939F, T941Q, T941C, K942T, K942R, Y943H, Y943L, Y943R, Y943V, D944A, E945S, D947N, D947M, D947I, D947S, K948D, L949K, L949V, 1950L, R951M, R951E, E952N, K954R, K954C, 1956L, T957I, S960T, S960G, S960Y, K961G, L962C, V963P, V963L, V963S, V963M, S964R, S964L, S964A, D965S, K968L, K968F, K968R, D969V, D969M, F970V, Q971G, Q971S, F972K, F972G, F972S, Y973E, Y973N, Y973A, V975I, V975G, V975L, V975M, E977Q, E977S, 1978E, 1978M, 1978L, N979A, N980T, N980S, N980I, N980M, N980L, N980D, N980A, Y981L, H983N, A984R, D986K, L989M, N990S, N990M, T995R, A996T, K999R, K1003H, K1003R, K1003V, E1005L, E1005G, S1006G, E1007M, E1007T, E1007S, E1007Q, E1007R, E1007G, E1007W, E1007C, F1008L, F1008S, Y1010M, D1012S, D1012L, Y1013G, K1014R, V1015T, V1015I, V1015D, V1015G, V1015N, V1015P, V1015L, V1015F, Y1016R, Y1016M, Y1016S, V1018W, R1019E, K1020T, K1020D, K1020S, M1021S, M1021A, M1021F, A1023C, A1023L, K1024S, K1024A, K1024E, K1024D, E1026I, Q1027I, Q1027T, Q1027C, I1029L, I1029M, G1030E, K1031L, K1031D, K1031S, A1032G, A1032K, T1033S, A1034S, A1034V, A1034Q, A1034L, K1035S, K1035V, Y1036M, Y1036Q, I1042W, I1042L, T1048L, E1049N, E1049Y, I1050F, I1050L, I1050E, I1050V, T1051Q, T1051E, T1051Y, T1051R, L1052P, A1053M, A1053S, N1054A, G1055E, E1056L, I1057R, I1057V, I1057G, R1058S, K1059Q, K1059A, K1059S, R1060N, R1060C, P1061A, P1061G, P1061S, L1062I, I1063T, I1063V, T1065C, T1065A, N1066S, N1066C, G1067P, G1067L, G1067E, G1067D, G1067A, E1068S, E1068A, T1069R, T1069S, E1071L, I1072R, I1072A, G1077D, G1077L, R1078K, R1078T, R1078L, D1079A, D1079G, F1080V, F1080H, A1081G, A1081P, A1081D, A1081R, A1081H, A1081L, A1081V, T1082E, V1083L, R1084T, R1084S, R1084H, R1084Q, R1084E, R1084D, R1084V, K1085H, K1085E, K1085D, K1085A, K1085C, K1085T, K1085S, K1085G, S1088D, M1089D, M1089V, M1089E, M1089S, M1089G, M1089F, P1090Y, P1090C, P1090Q, P1090A, Q1091T, Q1091I, Q1091L, Q1091S, Q1091E, Q1091V, V1092L, V1092A, V1092C, N1093A, I1094M, I1094L, I1094P, V1095K, K1096N, K1096T, K1096I, K1096V, K1096C, K1096S, K1096R, K1096Y, K1096L, K1096A, K1096W, K1097R, T1098K, T1098R, T1098I, T1098V, T1098L, T1098S, E1099K, E1099R, E1099S, E1099H, E1099V, E1099Y, E1099A, E1099N, E1099T, E1099M, E1099Q, E1099L, E1099C, V1100K, V1100R, V1100C, V1100T, Q1101V, Q1101A, T1102R, G1104S, G1104A, G1104P, G1104C, S1106T, S1106H, S1106A, S1106Y, S1106W, S1106C, S1106F, K1107R, K1107N, S1109K, S1109T, S1109R, S1109I, S1109Q, S1109L, S1109A, S1109G, S1109V, L1111V, K1113A, R1114N, R1114T, R1114S, R1114Q, R1114H, R1114D, R1114A, R1114G, R1114C, R1114K, N1115P, N1115A, S1116T, D1117G, K1118N, K1118S, K1118Q, K1118G, K1118T, K1118H, K1118L, K1118R, A1121R, K1124V, D1125A, D1125T, D1125G, D1127S, Y1131L, D1135A, D1135T, D1135S, D1135E, D1135V, T1138N, T1138S, T1138Y, V1139E, V1139H, A1140T, S1142V, V1143L, L1144A, V1145N, A1147H, K1148A, K1148C, V1149T, V1149E, E1150S, E1150L, E1150P, K1151L, K1151G, K1151V, K1151Y, K1151F, K1151I, K1151Q, G1152S, G1152T, G1152Q, K1153I, K1153G, K1153R, K1153Q, K1153L, K1153C, S1154G, K1155S, K1155L, K1156T, K1156I, K1156P, K1156R, K1156L, L1157H, L1157R, K1158E, K1158D, K1158Y, K1158S, K1158L, S1159P, S1159L, S1159F, S1159N, S1159Q, V1160T, K1161S, K1161I, K1161L, L1163M, L1163T, L1163G, G1165P, G1165L, G1165A, T1167P, T1167H, T1167L, R1171Q, S1172E, S1172Y, S1173C, S1173L, S1173H, F1174S, K1176D, I1179T, D1180T, D1180P, D1180A, D1180I, F1181V, F1181M, F1181Q, F1181H, E1183S, E1183L, A1184N, A1184R, A1184D, A1184C, K1185C, G1186P, Y1187L, Y1187S, K1188R, K1188A, K1188L, K1188S, E1189Q, V1190L, V1190A, V1190I, K1191T, K1191R, K1191L, K1191S, K1191E, K1191G, K1191H, K1191D, K1191P, K1192E, K1192L, K1192P, K1192T, K1192Q, D1193P, L1194Q, L1194H, L1194A, L1194E, L1194S, L1194D, I1195T, I1195V, I1196L, K1197L, K1197F, K1197C, L1198A, L1198F, P1199T, P1199H, K1200S, Y1201G, S1202H, S1202Y, L1203N, F1204L, E1205T, L1206S, L1206D, L1206G, L1206W, E1207Q, N1208L, N1208H, N1208G, N1208S, N1208C, G1209K, G1209Y, G1209S, R1210S, R1210E, R1210G, R1210C, R1210I, R1210A, K1211T, K1211Q, K1211A, M1213T, M1213H, M1213S, M1213P, M1213W, M1213F, L1214S, L1214C, A1215C, A1215G, A1215S, S1216A, A1217T, A1217H, A1217P, A1217D, A1217F, A1217N, A1217S, A1217G, A1217W, A1217C, Q1221N, Q1221I, Q1221V, Q1221C, Q1221T, Q1221A, G1223C, N1224P, L1226M, A1227S, A1227G, L1228F, L1228S, P1229T, P1229A, P1229G, P1229C, P1229M, P1229H, P1229L, P1229F, S1230G, S1230T, S1230Q, S1230V, K1231P, K1231L, K1231E, K1231D, Y1232T, Y1232R, V1233L, N1234L, N1234T, N1234P, N1234C, F1235L, L1236Q, Y1237R, Y1237M, Y1237Q, Y1237C, L1238H, L1238V, L1238F, L1238Y, S1240H, S1240L, H1241G, H1241M, H1241R, H1241L, H1241A, H1241V, H1241F, Y1242K, Y1242S, Y1242P, Y1242W, Y1242T, Y1242I, Y1242L, E1243T, K1244S, L1245N, L1245S, L1245R, L1245A, L1245W, L1245C, K1246S, G1247S, G1247W, G1247F, S1248D, S1248M, P1249N, P1249R, P1249L, E1250D, N1252A, N1252G, E1253G, E1253S, E1253R, Q1254I, Q1254H, Q1254P, Q1254D, K1255P, K1255G, K1255L, K1255F, Q1256P, Q1256C, L1257E, L1257V, L1257S, L1257W, F1258L, F1258V, E1260Q, E1260R, E1260Y, E1260A, E1260C, E1260F, Q1261T, Q1261A, Q1261V, Q1261L, Q1261M, Q1261H, Q1261P, Q1261G, H1262R, H1262W, H1262T, H1262D, H1262V, H1262S, K1263Q, K1263L, K1263S, K1263I, K1263P, K1263V, K1263F, H1264L, Y1265L, Y1265C, Y1265R, Y1265I, Y1265Q, Y1265H, L1266T, D1267Q, D1267P, D1267V, E1268V, E1268R, E1268A, Q1272I, S1274H, S1274R, S1274G, F1276G, F1276Y, S1277T, S1277H, K1278S, K1278L, R1279T, V1280I, V1280L, V1280E, V1280S, I1281L, I1281S, I1281C, L1282T, L1282R, L1282A, L1282G, L1282V, L1282S, A1283T, A1283S, A1283H, A1283R, A1285L, A1285P, A1285V, N1286Q, N1286G, N1286C, N1286H, N1286L, N1286A, N1286V, N1286F, L1287S, L1287I, L1287W, L1287F, D1288A, D1288G, K1289P, K1289E, K1289G, K1289V, K1289S, K1289L, L1291Q, L1291I, L1291S, L1291F, S1292T, S1292L, S1292V, S1292Y, S1292F, A1293T, A1293H, A1293G, Y1294I, Y1294R, Y1294C, Y1294S, Y1294H, N1295T, N1295Q, N1295P, N1295E, N1295S, N1295R, N1295L, N1295G, K1296L, K1296A, K1296Q, K1296R, K1296W, H1297E, H1297F, R1298N, R1298A, R1298S, R1298F, D1299L, D1299Y, K1300T, K1300R, K1300E, K1300V, K1300F, P1301N, I1302S, I1302V, R1303D, R1303V, E1304H, E1304S, Q1305S, A1306N, A1306I, E1307S, E1307C, I1309S, I1309R, I1309Y, I1310Y, 11310V, H1311T, H1311G, H1311V, LI312F, F1313L, F1313S, T1314L, T1314G, T1314S, T1314E, T1314D, L1315A, L1315S, T1316A, T1316Q, N1317L, N1317T, N1317S, L1318N, L1318I, L1318P, L1318E, L1318V, L1318W, L1318T, L1318D, L1318A, L1318Y, G1319S, G1319N, A1320T, A1320R, A1320S, A1320V, A1320C, A1320Y, A1320F, P1321S, P1321A, P1321G, P1321M, P1321C, A1322N, A1322T, A1322M, A1322L, A1322V, A1322W, A1323S, F1324S, F1324A, F1324G, F1324C, F1324T, F1324M, F1324D, F1324V, K1325R, Y1326M, Y1326W, F1327A, D1328T, D1328L, T1329L, T1329S, T1330N, T1330Q, D1332W, K1334A, K1334T, K1334Q, K1334R, K1334S, K1334W, Y1336M, Y1336P, S1338N, S1338V, S1338Y, S1338W, S1338A, S1338F, T1339L, T1339A, T1339G, K1340N, K1340T, K1340S, K1340M, K1340I, K1340P, K1340E, K1340A, K1340V, K1340C, K1340R, K1340H, K1340G, K1340L, V1342M, V1342L, V1342I, L1343K, L1343E, D1344H, T1346L, T1346I, L1347V, I1348S, H1349R, H1349V, H1349F, H1349T, H1349S, H1349L, H1349A, H1349Y, Q1350L, Q1350R, Q1350S, I1352T, I1352R, I1352P, I1352H, G1354V, L1355T, Y1356L, Y1356Q, E1357T, E1357I, T1358H, T1358S, R1359N, R1359V, D1361S, L1362C, L1362V, S1363H, S1363V, Q1364N, L1365M, G1366D, G1367P, G1367A, D1368T, D1368M, D1368L, and D1368G;
a double amino acid substitution comprising S1106Y and an additional substitution selected from the group consisting of E60K, A68K, T474R, A725R, A728W, H99A, E108P, and E130K;
a triple amino acid substitution comprising A68K/S1106Y substitutions and an additional substitution selected from the group consisting of T474R, E60K, A725R, A728W, H99A, E108P, E130K, and T333K;
a 4-amino acid substitution comprising A68K/T474R/S1106Y substitutions and an additional subsitution selected from the group consisting of E60K, A725R, A728W, E108P, H99A, E108V, E114S, D124E, I322V, K323L, Q330E, Q330V, L332K, P344R, E345T, E345Y, E349T, Q354S, D364G, D364V, G1104P, G1104A, S1109K, S1109R, S1109A, K1113A, R1114G, D1117G, K1118S, T445S, Y451W, R457A, M465L, T466V, T472K, T472R, L51R, D54K, D54R, G56A, E57K, T58S, T58G, L64K, Y72L, K65Y, K65F, T67S, N77K, C80R, D718K, S719N, E722K, H723M, N726R, L727V, A728G, S730G, A732S, I737V, L738R, L738Y, L738W, T740A, R753G, R1084E, K1085E, K1096V, T1098K, T1098R, E1099V, G1104A, L1198A, K26S, K30L, K31E, F32T, K33D, H1349R, H1349Y, and I1352P;
a 5-amino acid substitution comprising A68K/T474R/S1106Y substitution and an additional substitution selected from the group consisting of G56A/E57K, K31E/G56A, K31E/E57K, K1085E/G56A, K1085E/E57K, and K1085E/K31E;
a 6-amino acid substitution comprising A68K/T474R/S1106Y substitution and an additional subsitution selected from the group consisting of K31E/K1085E/G56A, K31E/K1085E/E57K, K31E/G56A/E57K, and G56A/K1085E/E57K;
a 7-amino acid substitution comprising K31E/G56A/E57K/A68K/T474R/S1106Y and an additional subsitution selected from the group consisting of K1085E, G1104A, M465L, T472K, R1084E, H1349Y, R753G, E108V, E130N, H329K, Q330V, T333R, S355C, A50T, I733Y, R753S, and P1090Y;
an 8-amino acid substitution comprising K31E/G56A/E57K/A68K/T474R/S1106Y substitution and an additional subsitution selected from the group consisting of H1349Y/R753G, T472K/R753G, H329K/R753G, T333R/R753G, H329K/T472K, T333R/T472K, and H329K/T333R;
a 9-amino acid substitution comprising K31E/G56A/E57K/A68K/T474R/S1106Y substitution and an additional subsitution selected from the group consisting of H329K/T472K/R753G, T333R/T472K/R753G, H329K/T333R/R753G, and H329K/T333R/T472K; and
a 10-amino acid substitution comprising K31E, G56A, E57K, A68K, H329K, T333R, T472K, T474R, R753G, and S1106Y substitutions.

In some embodiments, the variant of Cas9 protein as disclosed herein comprises at least one amino acid substitution selected from Table 5 or Table 6. In some embodiments, the variant of Cas9 protein as disclosed herein comprises at least two amino acid substitutions selected from Table 5 or Table 6. In some embodiments, the variant of Cas9 protein as disclosed herein comprises S1106Y substitution and at least one additional substitution selected from Table 5 or Table 6. In some embodiments, the variant of Cas9 protein as disclosed herein comprises S1106Y, A68K, and T474R substitutions. In some embodiments, the variant of Cas9 protein as disclosed herein comprises S1106Y, A68K, T474R substitutions and at least one additional substitution selected from Table 5 or Table 6. In some embodiments, the variant of Cas9 protein as disclosed herein comprises S1106Y, A68K, T474R, K31E, G56A, and E57K substitutions. In some embodiments, the variant of Cas9 protein as disclosed herein comprises S1106Y, A68K, T474R, K31E, G56A, E57K, R753G, H329K, and T333R substitutions. In some embodiments, the variant of Cas9 protein as disclosed herein comprises S1106Y, A68K, T474R, H329K, and T333R substitutions.

In a third aspect, a CRISPR/Cas9 endonuclease is disclosed. The CRISPR/Cas9 endonuclease includes an isolated variant of Cas9 protein complexed with an RNA guide with a scaffold sequence shorter than 76 nucleotides to form a CRISPR/Cas9 endonuclease, the resultant CRISPR/Cas9 endonuclease cleaves a double-stranded DNA target in living cells with greater efficiency than a CRISPR/Cas9 endonuclease comprising the wild-type Cas9 protein complexed with an RNA guide with a scaffold sequence shorter than 76 nucleotides. In a fourth aspect, a method for promoting cleavage of a double-stranded DNA target in a cell by a Clustered Regularly Interspaced Short Palindromic Repeat (CRISPR) endonuclease is presented. The CRISPR endonuclease includes a CRISPR/Cas9 endonuclease having a variant of Cas9 protein and a guide RNA with a scaffold sequence shorter than 76 nucleotides. The method includes the following steps. The first step includes introducing into the cell genome editing reagents. The editing reagents includes the variant of Cas9 protein and the guide RNA with a scaffold sequence shorter than 76 nucleotides. The second step includes contacting the double-stranded DNA target with the CRISPR/Cas9 endonuclease formed from the variant of Cas9 protein complexed with a guide RNA with a scaffold sequence shorter than 76 nucleotides. The third step includes cleaving the double-stranded DNA target with the resultant CRISPR/Cas9 endonuclease. The resultant CRISPR/Cas9 endonuclease cleaves the double-stranded DNA target with greater efficiency than a CRISPR/Cas9 endonuclease comprising Cas9 protein complexed with the guide RNA with a scaffold sequence shorter than 76 nucleotides.

In a fifth aspect, a method for promoting cleavage of a double-stranded DNA target in a cell by a Clustered Regularly Interspaced Short Palindromic Repeat (CRISPR) endonuclease is disclosed. The CRISPR endonuclease includes a CRISPR/Cas9 endonuclease having a variant of Cas9 protein and a guide RNA with a scaffold sequence shorter than 76 nucleotides. The method may include, for example, the following steps. The first step includes introducing into the cell genome editing reagents comprising nucleic acids encoding an amino acid sequence of the variant of Cas9 protein and the guide RNA with a scaffold sequence shorter than 76 nucleotides. The second step includes expressing the amino acid sequence of the variant of Cas9 protein and the sgRNA with a scaffold sequence shorter than 76 nucleotides from the nucleic acids. The third step includes contacting the double-stranded DNA target with the CRISPR/Cas9 endonuclease formed from the variant of Cas9 protein complexed with the guide RNA with a scaffold sequence shorter than 76 nucleotides. The fourth step includes cleaving the double-stranded DNA target with the resultant CRISPR/Cas9 endonuclease. The resultant CRISPR/Cas9 endonuclease cleaves the double-stranded DNA target with greater efficiency than a CRISPR/Cas9 endonuclease comprising Cas9 protein complexed with the guide RNA with a scaffold sequence shorter than 76 nucleotides.

In some embodiments, methods as disclosed herein comprises introducing into the cell genome editing reagents comprising mRNA encoding an amino acid sequence of any one of the variant of Cas9 protein disclosed herein and a guide RNA with a scaffold sequence shorter than 76 nucleotides. In some embodiments, the guide RNA as disclosed herein comprises any one of the synthetic guides selected from Table 9 or Table 10. In some embodiments, the guide RNA as disclosed herein further comprises chemical modifications. It is understood in the field that CRISPR-Cas9 gRNAs require chemical modification to remain stable for potent genome editing when using DNA, mRNA, viral, and ribonucleoprotein (RNP) delivery modalities. Hendel et al. published a modification pattern that has been accepted as a standard in the field where the 5′ and 3′ termini of the fused single-guide RNA (sgRNA) are modified with phosphorothioate (PS) bonds and 2′o-methylated bases. An example 5′ sgRNA terminus of this standard modification pattern is mN*mN*mN* (first three nucleotides only) and an example of the 3′ terminus is mN*mN*mN*rN (last four nucleotides only), where N denotes any nucleotide, r-RNA base, m=2′-O-methyl base, *=phosphorothioate bond. These chemically modified sgRNAs benefit all delivery modalities, which can be important when using expressed delivery formats such as mRNA and DNA (Hendel, A., et al., Chemically modified guide RNAs enhance CRISPR-Cas genome editing in human primary cells. Nat Biotechnol, 2015. 33 (9): p. 985-989). In some embodiments, the guide RNA as disclosed herein further comprises a chemical modification pattern, wherein the 5′ terminus comprises mN*mN*mN* (first three nucleotides only) and the 3′ terminus comprises mN*mN*mN*rN (last four nucleotides only), wherein N denotes any nucleotide, r=RNA base, m=2′-O-methyl base, *-phosphorothioate bond.

In a sixth aspect, a kit for use in promoting cleavage of a DNA target site by a CRISPR/Cas9 endonuclease is disclosed. The kit includes the following components. A first component includes an isolated variant of Cas9 protein. A second component includes a guide RNA with a scaffold sequence shorter than 76 nucleotides. The CRISPR/Cas9 endonuclease formed by the components cleaves a double-stranded DNA target in living cells with greater efficiency than a CRISPR/Cas9 endonuclease comprising the wild-type Cas9 protein complexed with the guide RNA with a scaffold sequence shorter than 76 nucleotides.

The disclosure provides an isolated variant Cas9 protein, comprising an amino acid sequence selected from the group consisting of the following relative to the wild-type Cas9 amino sequence of SEQ ID NO: 133: a single amino acid substitution-comprising at least one substitution selected from Table 5 or Table 6; a double amino acid substitution comprising S1106Y substitution and an additional substitution selected from the group consisting of E60K, A68K, T474R, A725R, A728W, H99A, E108P, and E130K; a triple amino acid substitution comprising A68K/S1106Y and an additional substitution selected from the group consisting of T474R, E60K, A725R, A728W, H99A, E108P, E130K, and T333K; a 4-amino acid substitution comprising A68K/T474R/S1106Y and an additional substitution selected from the group consisting of E60K, A725R, A728W, E108P, H99A, E108V, E114S, D124E, I322V, K323L, Q330E, Q330V, L332K, P344R, E345T, E345Y, E349T, Q354S, D364G, D364V, G1104P, G1104A, S1109K, S1109R, S1109A, K1113A, R1114G, D1117G, K1118S, T445S, Y451W, R457A, M465L, T466V, T472K, T472R, L51R, D54K, D54R, G56A, E57K, T58S, T58G, L64K, Y72L, K65Y, K65F, T67S, N77K, C80R, D718K, S719N, E722K, H723M, N726R, L727V, A728G, S730G, A732S, I737V, L738R, L738Y, L738W, T740A, R753G, R1084E, K1085E, K1096V, T1098K, T1098R, E1099V, G1104A, L1198A, K26S, K30L, K31E, F32T, K33D, H1349R, H1349Y, and I1352P; a 5-amino acid substitution comprising A68K/T474R/S1106Y and an additional substitution selected from the group consisting of G56A/E57K, K31E/G56A, K31E/E57K, K1085E/G56A, K1085E/E57K, and K1085E/K31E; a 6-amino acid substitution comprising A68K/T474R/S1106Y and an additional substitution selected from the group consisting of K31E/K1085E/G56A, K31E/K1085E/E57K, K31E/G56A/E57K, and G56A/K1085E/E57K; a 7-amino acid substitution comprising K31E/G56A/E57K/A68K/T474R/S1106Y and an additional substitution selected from the group consisting of K1085E, G1104A, M465L, T472K, R1084E, H1349Y, R753G, E108V, E130N, H329K, Q330V, T333R, S355C, A50T, I733Y, R753S, and P1090Y; an 8-amino acid substitution comprising K31E/G56A/E57K/A68K/T474R/S1106Y and an additional substitution selected from the group consisting of H1349Y/R753G, T472K/R753G, H329K/R753G, T333R/R753G, H329K/T472K, T333R/T472K, and H329K/T333R; a 9-amino acid substitution comprising K31E/G56A/E57K/A68K/T474R/S1106Y and an additional substitution selected from the group consisting of H329K/T472K/R753G, T333R/T472K/R753G, H329K/T333R/R753G, and H329K/T333R/T472K; and a 10-amino acid substitution comprising K31E, G56A, E57K, A68K, H329K, T333R, T472K, T474R, R753G, and S1106Y. The disclosure provides an isolated variant Cas9 protein, wherein the isolated variant is selected from the group consisting of a Cas9 variant with the following substitutions S1106Y, A68K, T474R with SEQ ID NO: 137; a Cas9 variant with the following substitutions S1106Y, A68K, T474R, K31E, G56A, E57K with SEQ ID NO: 139; a Cas9 variant with the following substitutions S1106Y, A68K, T474R, K31E, G56A, E57K, K1085E with SEQ ID NO: 141; and a Cas9 variant with the following substitutions K31E, G56A, E57K, A68K, H329K, T333R, T474R, R753G, S1106Y with SEQ ID NO: 135, wherein the substitutions are relative to the wild-type Cas9 amino acid sequence of SEQ ID NO: 133. The disclosure provides an isolated nucleic acid encoding a variant Cas9 protein as disclosed herein. The disclosure provides an mRNA encoding a variant Cas9 protein as disclosed herein. The disclosure provides a host cell comprising a nucleic acid encoding a modified Cas9 protein as disclosed herein. The disclosure provides a host cell wherein the host cell is selected from the group consisting of bacterial cells, insect cells, plant cells, mammalian cells, an immortalized cell, a HEK293 kidney cell, a Jurkat T cell, a primary human T cell, and HSPCs, an induced pluripotent stem cell.

The disclosure provides a gene editing system, comprising: a. at least one of the variant Cas9 proteins, or a nucleic acid encoding at least one of the variant Cas9 proteins, as disclosed herein; and b. at least one of a single guide RNA (sgRNA) which has a scaffold sequence shorter than 76 nucleotides, wherein the gene editing system exhibits enhanced editing activity relative to the gene editing activity of a wild-type Cas9 having the sequence of SEQ ID NO: 133 in the presence of the sgRNA. The disclosure provides a gene editing system, wherein the sgRNA comprises a tetraloop. The disclosure provides a gene editing system, wherein the sgRNA comprises a target-specific spacer sequence, a repeat sequence, a tetraloop region, an anti-repeat region, a stem loop 1 region, linker, a stem loop 2 region, a stem loop 3 region, and a terminal region. The disclosure provides a gene editing system, wherein the sgRNA comprises a target-specific spacer sequence, a repeat sequence, a tetraloop region, an anti-repeat region, a stem loop 1 region, a linker region, and a stem loop 2 region, wherein the stem loop 3 region has been deleted. The disclosure provides a gene editing system, wherein the sgRNA comprises a 20 nt target-specific spacer sequence, a 12 nt repeat region, a 4 nt tetraloop, and a 26 nt region comprising an anti-repeat region, a stem loop 1 region, a linker, a stem loop 2 region, a stem loop 3 region, and a terminal region. The disclosure provides a gene editing system, wherein the sgRNA further comprises a poly-U terminator region. The disclosure provides a gene editing system, wherein the sgRNA has a number of deletions from the 3′ end selected from the group consisting of 24 nt, 28 nt, 33 nt, 34 nt, 36 nt, 39 nt, and 41 nt. The disclosure provides a gene editing system, wherein the sgRNA has a length selected from the group consisting of 76 nt, 72 nt, 66 nt, 67 nt, 64 nt, 61 nt, and 59 nt. The disclosure provides a gene editing system wherein the Cas9 variant has the same gene editing activity with the sgRNA as the gene editing activity of wild-type Cas9 paired with full length guides. The disclosure provides a kit comprising the gene editing system as disclosed herein and instructions for use.

The disclosure provides a composition comprising a variant Cas9 protein as disclosed herein, formulated for use in biochemical assays, industrial processes, or therapeutic applications. The disclosure provides a single guide RNA (sgRNA) which has a scaffold region shorter than 76 nucleotides. The disclosure provides a sgRNA, wherein the sgRNA comprises a tetraloop. The disclosure provides an sgRNA, wherein the sgRNA comprises a target-specific spacer sequence, a repeat sequence, a tetraloop region, an anti-repeat region, a stem loop 1 region, linker, a stem loop 2 region, a stem loop 3 region, and a terminal region. The disclosure provides an sgRNA, wherein the sgRNA comprises a target-specific spacer sequence, a repeat sequence, a tetraloop region, an anti-repeat region, a stem loop 1 region, a linker region, and a stem loop 2 region, wherein the stem loop 3 region has been deleted. The disclosure provides an sgRNA, wherein the sgRNA comprises a 20 nt target-specific spacer sequence, a 12 nt repeat region, a 4 nt tetraloop, and a 26 nt region comprising an anti-repeat region, a stem loop 1 region, a linker, a stem loop 2 region, a stem loop 3 region, and a terminal region. The disclosure provides an sgRNA, wherein the sgRNA further comprises a poly-U terminator region. The disclosure provides an sgRNA, wherein the sgRNA has a number of deletions from the 3′ end selected from the group consisting of 24 nt, 28 nt, 33 nt, 34 nt, 36 nt, 39 nt, and 41 nt. The disclosure provides an sgRNA, wherein the sgRNA has a length selected from the group consisting of 76 nt, 72 nt, 66 nt, 67 nt, 64 nt, 61 nt, and 59 nt. The disclosure provides an sgRNA, wherein a wil-type Cas9 will not edit a target nucleic acid in the presence of the sgRNA.

The disclosure provides a method of delivering a gene editing system as disclosed herein to a cell, the method comprising the steps of: (a) providing a first viral vector component encoding a sgRNA that hybridizes with a target sequence; (b) providing a second viral vector component encoding the variant Cas9 protein; wherein components (a) and (b) are located on same or different vectors of the system; and (c) transducing the cell with the viral vector(s) under conditions sufficient to express the variant Cas9 protein and the sgRNA, wherein the Cas9 and the sgRNA form a complex that binds to and edits the target sequence.

The disclosure provides a method for delivering the gene editing system as disclosed herein to a cell, comprising: (a) providing lipid nanoparticles encapsulating a sgRNA that hybridizes with a target sequence; (b) providing lipid nanoparticles encapsulating an mRNA encoding the variant Cas9 protein; wherein components (a) and (b) are located on same or different vectors of the system, (c) transducing the cell with the lipid nanoparticles under conditions sufficient to express the variant Cas9 protein and the sgRNA, wherein the variant Cas9 protein and the sgRNA are expressed and form a complex to edit a specific target sequence in the cell's genome, and wherein the Cas9 variant has the same gene editing activity with the sgRNA as the gene editing activity of wild-type Cas9 paired with full length guides.

The disclosure provides a method for delivering the gene editing system as disclosed herein to a cell, comprising: (a) providing a ribonucleoprotein (RNP) complex comprising the gene editing system; and (b) introducing the RNP complex into the cell using electroporation, wherein the gene editing system binds to and edits a target sequence within the genome of the cell, and wherein the Cas9 variant has the same gene editing activity with the sgRNA as the gene editing activity of wild-type Cas9 paired with full length guides.

The disclosure provides a method of delivering the gene editing system as disclosed herein to a cell, comprising: preparing a lipofection reagent comprising a first regulatory element operable in a eukaryotic cell operably linked to at least one nucleotide sequence encoding a sgRNA that hybridizes with a target sequence; a second regulatory element operable in a eukaryotic cell operably linked to a nucleotide sequence encoding the variant Cas9 protein; wherein components (a) and (b) are located on the same or different vectors of the system, and applying the lipofection reagent to the cell, wherein the gene editing system are expressed and form a complex to edit a specific target sequence in the cell's genome, and wherein the Cas9 variant has the same gene editing activity with the sgRNA as the gene editing activity of wild-type Cas9 paired with full length guides.

The disclosure provides a method of targeted delivery of the gene editing system as disclosed herein to a cell, comprising: (a) preparing exosomes encapsulating a first nucleotide sequence encoding a sgRNA that hybridizes with a target sequence; and (b) preparing exosomes encapsulating a second nucleotide sequence encoding the variant Cas9 protein; wherein components (a) and (b) are located on same or different vectors of the system; and (c) delivering the engineered exosomes to the cell under conditions that allow the gene editing system to edit the target sequence, and wherein the Cas9 variant has the same gene editing activity with the sgRNA as the gene editing activity of wild-type Cas9 paired with full length guides.

The disclosure provides a method wherein the cell is selected from the group consisting of bacterial cells, insect cells, plant cells, mammalian cells, an immortalized cell, a HEK293 kidney cell, a Jurkat T cell, a primary human T cell, and an induced pluripotent stem cell.

The disclosure provides a method for promoting cleavage of a nucleic acid target in a cell by a Clustered Regularly Interspaced Short Palindromic Repeat (CRISPR) endonuclease, wherein the CRISPR endonuclease comprises the gene editing system as disclosed herein, the method comprising: introducing into the cell genome editing reagents comprising the variant Cas9 protein and the sgRNA; contacting the nucleic acid target with the Cas9 endonuclease formed from the variant Cas9 protein complexed with the sgRNA; cleaving the nucleic acid target with the resultant CRISPR/Cas9 endonuclease, wherein the Cas9 variant has the same gene editing activity with the sgRNA as the gene editing activity of wild-type Cas9 paired with full length guides.

The disclosure provides a method for promoting cleavage of a nucleic acid target in a cell by a Clustered Regularly Interspaced Short Palindromic Repeat (CRISPR) endonuclease, wherein the CRISPR endonuclease comprises the gene editing system as disclosed herein, the method comprising: introducing into the cell genome editing reagents comprising nucleic acids encoding an amino acid sequence of the variant Cas9 protein and the sgRNA; expressing the amino acid sequence of the variant Cas9 protein and the sgRNA; contacting the nucleic acid target with the CRISPR/Cas9 endonuclease formed from the variant Cas9 protein complexed with the sgRNA; cleaving the nucleic acid target with the Cas9 endonuclease, wherein the Cas9 variant has the same gene editing activity with the sgRNA as the gene editing activity of wild-type Cas9 paired with full length guides.

### BRIEF DESCRIPTION OF THE DRAWINGS

FIG. 1A depicts the percent editing at VEGFA measured by T7E1 assay in HEK293 cells treated with Cas9 expression plasmids encoding wild-type Cas9 (WT) or Cas9 variants with the indicated amino acid substitution delivered alongside VEGFA full length (dots) or GD1 sgRNA expression plasmid (bars). Error bars indicated standard deviation from at least three replicates. The dashed line indicates the average editing for wild-type (WT) Cas9 expression plasmid using the GD1 guide plasmid and the line of vertical dashes indicates the average editing for WT Cas9 with the full length sgRNA guide plasmid.

FIG. 1B depicts the percent editing at VEGFA measured by T7E1 assay in HEK293 cells treated with Cas9 expression plasmids encoding wild-type Cas9 (WT) or Cas9 variants with the indicated amino acid substitution delivered alongside VEGFA full length (dots) or GD1 sgRNA expression plasmid (bars). Error bars indicated standard deviation from at least three replicates. The dashed line indicates the average editing for wild-type (WT) Cas9 expression plasmid using the GD1 guide plasmid and the vertical dashes indicate the average editing for WT Cas9 with the full length sgRNA guide plasmid.

FIG. 2 depicts the percent editing at VEGFA measured by T7E1 assay in HEK293 cells treated with Cas9 expression plasmids encoding wild-type Cas9 (WT) or Cas9 variants with the indicated amino acid substitution(s) delivered alongside VEGFA full length (dots) or GD1 sgRNA expression plasmid (bars). Error bars indicated standard deviation from at least two replicates. The dashed line indicates the average editing for wild-type (WT) Cas9 expression plasmid using the VEGFA GD1 guide plasmid and the line of vertical dashes indicates the average editing for WT Cas9 with the VEGFA full length sgRNA guide plasmid.

FIG. 3A depicts the percent editing at HPRT1 measured by T7E1 assay in HEK293 cells treated with expression plasmids encoding wild-type Cas9 (WT), Cas9 TM9 (S1106Y, A68K, T474R), or Cas9 variants with the indicated amino acid substitution(s) stacked on top of Cas9 TM9 (S1106Y, A68K, T474R) delivered alongside HPRT1 GD1 sgRNA expression plasmid. Error bars indicated standard deviation from at least two replicates. Dashed line indicates average editing with Cas9 TM9.

FIG. 3B depicts the percent editing at HPRT1 measured by T7E1 assay in HEK293 cells treated with expression plasmids encoding wild-type Cas9 (WT), Cas9 TM9 (S1106Y, A68K, T474R), or Cas9 variants with the indicated amino acid substitution(s) stacked on top of Cas9 TM9 (S1106Y, A68K, T474R), with the exception of TM65 where A68Q replaces A68K in TM9, delivered alongside HPRT1 GD1 sgRNA expression plasmid. Error bars indicated standard deviation from at least two replicates. Dashed line indicates average editing with Cas9 TM9.

FIG. 4 depicts the percent editing at HPRT1 (top panel) and VEGFA (bottom panel) measured by T7E1 assay in HEK293 cells treated with expression plasmids encoding wild-type Cas9 (WT), Cas9 TM9 (S1106Y, A68K, T474R), or Cas9 variants with the indicated amino acid substitution(s) stacked on top of Cas9 TM9 (S1106Y, A68K, T474R) delivered alongside HPRT1 or VEGFA full length (FL), GD1, or GD2 sgRNA expression plasmid. Error bars indicated standard deviation from at least two replicates. The dotted, dashed, and dot-dash lines indicated editing with TM9 using the FL, GD1, or GD2 guide expression plasmids respectively.

FIG. 5 depicts the percent editing at HPRT1 (top panel) and VEGFA (bottom panel) measured by T7E1 assay in HEK293 cells treated with expression plasmids encoding wild-type Cas9 (WT) or Cas9 variants with the indicated amino acid substitution(s) stacked on top of Cas9 TM9 (S1106Y, A68K, T474R) delivered alongside HPRT1 or VEGFA full length (FL), GD1, or GD2 sgRNA expression plasmid. Error bars indicated standard deviation from at least two repli cates.

FIG. 6A depicts the percent editing at HPRT1 measured by T7E1 assay in HEK293 cells treated with expression plasmids encoding wild-type Cas9 (WT), Cas9 TM9 (S1106Y, A68K, T474R), Cas9 TM108 (S1106Y, A68K, T474R, K31E, G56A, E57K), or Cas9 variants with the indicated amino acid substitution stacked on top of Cas9 TM108 (S1106Y, A68K, T474R, K31E, G56A, E57K) delivered alongside HPRT1 GD2 sgRNA expression plasmid. Error bars indicated standard deviation from at least two replicates. The dashed line indicates the percent editing with Cas9 TM108.

FIG. 6B depicts the percent editing at VEGFA measured by T7E1 assay in HEK293 cells treated with expression plasmids encoding wild-type Cas9 (WT), Cas9 TM9 (S1106Y, A68K, T474R), Cas9 TM108 (S1106Y, A68K, T474R, K31E, G56A, E57K), or Cas9 variants with the indicated amino acid substitution stacked on top of Cas9 TM108 (S1106Y, A68K, T474R, K31E, G56A, E57K) delivered alongside VEGFA GD2 sgRNA expression plasmid. Error bars indicated standard deviation from at least two replicates. The dashed line indicates the percent editing with Cas9 TM108.

FIG. 7 depicts the percent editing at HPRT1 (top panel) and VEGEA (bottom panel) measured by T7E1 assay in HEK293 cells treated with expression plasmids encoding wild-type Cas9 (WT), Cas9 TM9 (S1106Y, A68K, T474R), Cas9 TM108 (S1106Y, A68K, T474R, K31E, G56A, E57K), or Cas9 variants with the indicated amino acid substitution stacked on top of Cas9 TM108 (S1106Y, A68K, T474R, K31E, G56A, E57K) delivered alongside HPRT1 or VEGFA FL, GD1, or GD2 sgRNA expression plasmid. Error bars indicated standard deviation from at least two replicates. The dotted, dashed, and dot-dash lines indicated editing with TM108 using the FL, GD1, or GD2 guide expression plasmids respectively.

FIG. 8 depicts the percent editing at HPRT1 (top panel) and VEGFA (bottom panel) measured by T7E1 assay in HEK293 cells treated with expression plasmids encoding wild-type Cas9 (WT), Cas9 TM108 (S1106Y, A68K, T474R, K31E, G56A, E57K), or Cas9 variants with the indicated amino acid substitution(s) stacked on top of Cas9 TM108 (S1106Y, A68K, T474R, K31E, G56A, E57K) delivered alongside HPRT1 or VEGFA FL, GD1, or GD2 sgRNA expression plasmid. Error bars indicated standard deviation from at least two replicates. The dotted, dashed, and dot-dash lines indicated editing with TM108 using the FL, GD1, or GD2 guide expression plasmids respectively.

FIG. 9A depicts the percent editing at HPRT1 measured by T7E1 assay in HEK293 cells treated with expression plasmids encoding wild-type Cas9 (WT), Cas9 TM108 (S1106Y, A68K, T474R, K31E, G56A, E57K), or Cas9 TM110 (S1106Y, A68K, T474R, K31E, G56A, E57K, K1085E) delivered alongside HPRT1 FL, GD1, GD2, GD3, GD4, or GD5 sgRNA expression plasmid (see Table 7). Error bars indicated standard deviation from at least two replicates.

FIG. 9B depicts the percent editing at VEGFA measured by T7E1 assay in HEK293 cells treated with expression plasmids encoding wild-type Cas9 (WT), Cas9 TM108 (S1106Y, A68K, T474R, K31E, G56A, E57K), or Cas9 TM110 (S1106Y, A68K, T474R, K31E, G56A, E57K, K1085E) delivered alongside VEGFA FL, GD1, GD2, GD3, GD4, or GD5 sgRNA expression plasmid (see Table 7). Error bars indicated standard deviation from at least two replicates.

FIG. 10A depicts the percent editing at VEGFA measured by T7E1 assay in HEK293 cells treated with 2 μM Cas9 RNP formed with synthetic end-modified sgRNAs targeting VEGFA. The Cas9 RNP were formed with wild-type Cas9 (WT), Cas9 TM9 (S1106Y, A68K, T474R), Cas9 TM108 (S1106Y, A68K, T474R, K31E, G56A, E57K), or Cas9 TM136 (S1106Y, A68K, T474R, K31E, G56A, E57K, R753G, H329K, T333R) complexed with synthetic end-modified sgRNAs targeting VEGFA (Table 2). Error bars indicated standard deviation from at least two replicates.

FIG. 10B depicts the percent editing at HPRT1 measured by T7E1 assay in HEK293 cells treated with 2 μM Cas9 RNP formed with synthetic sgRNAs targeting HPRT1. The Cas9 RNP were formed with wild-type Cas9 (WT), Cas9 TM9 (S1106Y, A68K, T474R), Cas9 TM108 (S1106Y, A68K, T474R, K31E, G56A, E57K), or Cas9 TM136 (S1106Y, A68K, T474R, K31E, G56A, E57K, R753G, H329K, T333R) complexed with synthetic end-modified (mFL, mGD1, mGD2) or unmodified (uFL, uGD1,uGD2) sgRNAs targeting HPRT1 (Table 2). Error bars indicated standard deviation from at least two replicates.

FIG. 10C depicts the percent editing at HPRT1 measured by T7E1 assay in HEK293 cells treated with Cas9 RNP at 2, 0.25, or 0.0625 μM final concentration formed with synthetic end-modified full length sgRNA targeting HPRT1 (Table 2). The Cas9 RNP were formed with wild-type Cas9 (WT), Cas9 TM9 (S1106Y, A68K, T474R), Cas9 TM108 (S1106Y, A68K, T474R, K31E, G56A, E57K), or Cas9 TM136 (S1106Y, A68K, T474R, K31E, G56A, E57K, R753G, H329K, T333R). Error bars indicated standard deviation from at least two replicates.

FIG. 11 shows the features and sequence of a representative single guide RNA (SEQ ID NO: 1) with the structures of the scaffold region (repeat, tetra-loop, anti-repeat, stem loop 1, linker, stem loop 2, and stem loop 3) indicated above. The sequences of representative sgRNAs matching guide designs 1 (SEQ ID NO: 143) and 2 (SEQ ID NO: 156) made as synthetic sgRNAs and the associated features are shown below the full-length guide.

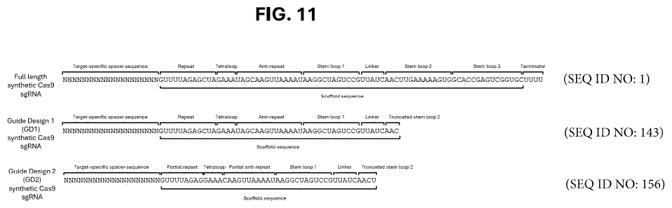

FIG. 12A depicts the percent editing at VEGFA measured by T7E1 assay in HEK293 cells treated with expression plasmid encoding Cas9 TM136 (S1106Y, A68K, T474R, K31E, G56A, E57K, R753G, H329K, T333R) delivered alongside sgRNA expression plasmids encoding various short scaffold guide RNA designs targeting VEGFA (V100, V70, V67, V66, V64, V61, V58, V70ID, V67ID, V66ID, V64ID, and V61D) where the letter corresponds to the targeted gene, the number is the 3′ position at which the guide terminates relative to a full length sgRNA (excluding the U's added during transcription termination), and ID refers to internal deletions that reduce the length by an additional 6 nucleotides (see Table 7 and FIG. 13). Error bars indicated standard deviation from at least two replicates.

FIG. 12B depicts the percent editing at HPRT1 measured by T7E1 assay in HEK293 cells treated with expression plasmid encoding Cas9 TM136 (S1106Y, A68K, T474R, K31E, G56A, E57K, R753G, H329K, T333R) delivered alongside sgRNA expression plasmids encoding various short scaffold guide RNA designs targeting HPRT1 (H100, H70, H67, H66, H64, H61, H58, H70ID, H67ID, H66ID, H64ID, and H61D) where the letter corresponds to the targeted gene, the number is the 3′ position at which the guide terminates relative to a full length sgRNA (excluding the U's added during transcription termination), and ID refers to internal deletions that reduce the length by an additional 6 nucleotides (see Table 7 and FIG. 13). Error bars indicated standard deviation from at least two replicates.

FIG. 13 shows the sequences of the short guide RNA designs tested in FIG. 12A and FIG. 12B. Italicized sequences indicate the spacer sequence and underlined U's indicate U's that are expected to be part of the plasmid expressed guide due to the poly-T termination sequence however are at positions that are not normally a U in a standard 100 nucleotide sgRNA.

### DETAILED DESCRIPTION OF THE INVENTION

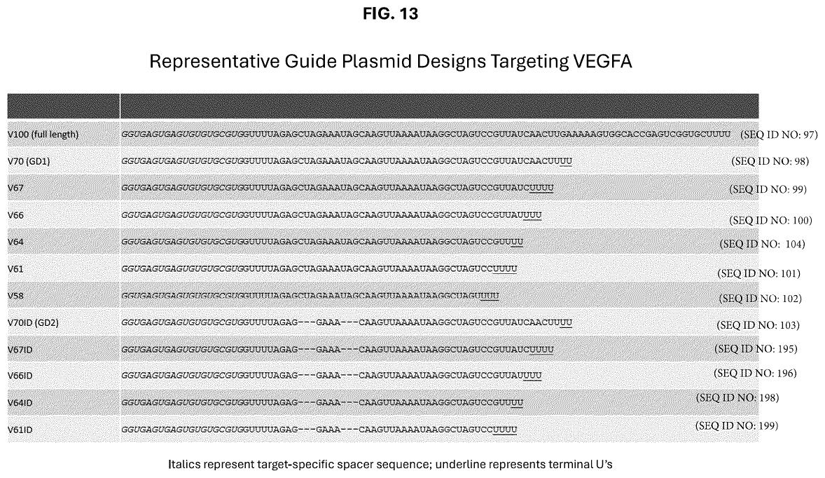

The goal of this work is to engineer a Cas9 variant capable of efficient targeted cleavage in human cells using guides with a guide RNA scaffold constant region that is less than the standard 76 nucleotides (positions 21-96 of a standard ˜100 nt sgRNA-see FIG. 13). We used a survival-based screening system in bacteria that we used previously to engineer a high-fidelity Cas9 variant, though without counter selection for off-target activity⁹. We identified guide designs that gave low but above background levels of survival on the context of the screening system. We then generated Cas9 variant expression plasmid libraries where positions in Cas9 in close proximity to the guide scaffold in the context of Cas9 ribonucleoprotein complex were mutated to every other possible amino acid. These plasmid libraries were delivered into bacteria alongside synthetic sgRNAs of varying designs to identify amino acid substitutions that resulted in a survival advantage. Mutations found to provide a survival advantage are likely to improve Cas9 activity with the shorter guide, as survival is linked to Cas9 cleavage of a toxin plasmid in the bacteria. From this screen we identified amino acid substitutions representing 1096 individual amino acid changes in Cas9 that were beneficial for survival, and likely Cas9 activity, in the context of the screen (Screen 1 in Table 5 and Table 6). A amino acid substitution was determined to be beneficial in the context of the screen if it had an enrichment higher than one in at least two replicates for either of the two short scaffold region guide designs (GD1 and GD2, see bacterial screen guide sequences in Table 2) where enrichment was calculated as: Enrichment=(number of reads for a particular amino acid substitution in the post screen sample/total number of reads for synonymous amino acid substitutions for the post-screen sample)/(number of reads for a particular amino acid substitution in the pre-screen sample/total number of reads for synonymous amino acid substitutions in the pre-screen sample). An enrichment of greater than one would mean that that fraction of reads for a amino acid substitution relative to reads corresponding to a wild-type Cas9 protein (synonymous amino acid substitution reads) increased post-selection, meaning that expression of the Cas9 variant resulted in better survival than expression of wild-type Cas9 in the context of the screen indicating that the Cas9 variant likely has better activity with short guides. An amino acid change was considered beneficial if there was at least one beneficial amino acid substitution identified that resulted in that amino acid change. To further expand the pool of candidate beneficial amino acid substitutions we conducted a second screen where instead of screening only the regions of Cas9 in close proximity to the guide scaffold region, we screened all positions of Cas9 except the start codon (positions 2-1368) using the same screening approach as before. For the second screen two technical replicate samples were sequenced for each input and post-screen sample and a amino acid substitution was considered beneficial if both biological replicate samples for either guide design had an enrichment greater than one for either set of technical replicates. A total of 7450 beneficial amino acid changes were identified. The beneficial amino acid changes are listed in Table 5. A subset of these amino acid changes were introduced into human codon-optimized Cas9 expression plasmids and tested in HEK293 cells. We found that a subset of the screen identified amino acid changes were able to improve Cas9 activity when paired with short guides in human cells.

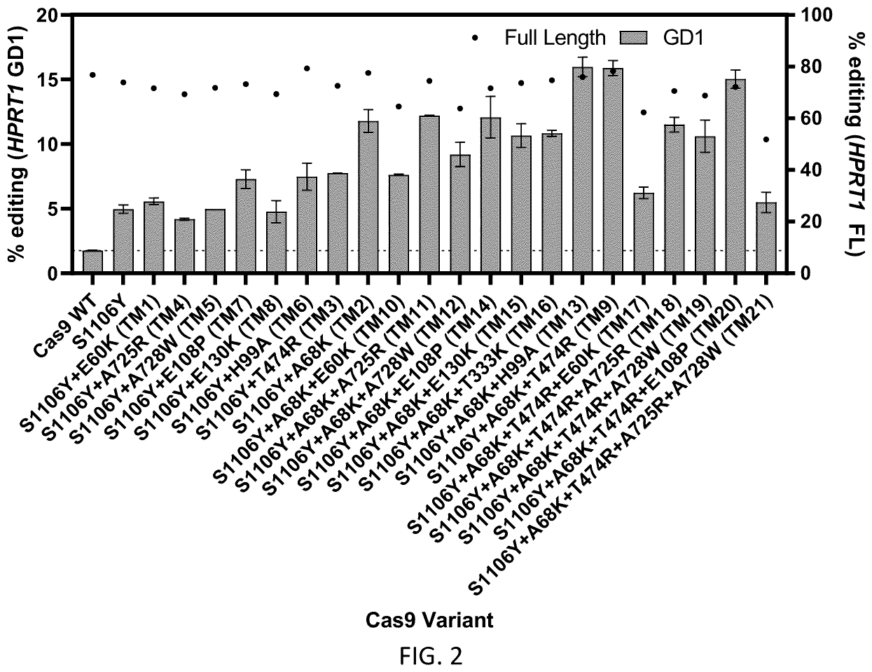

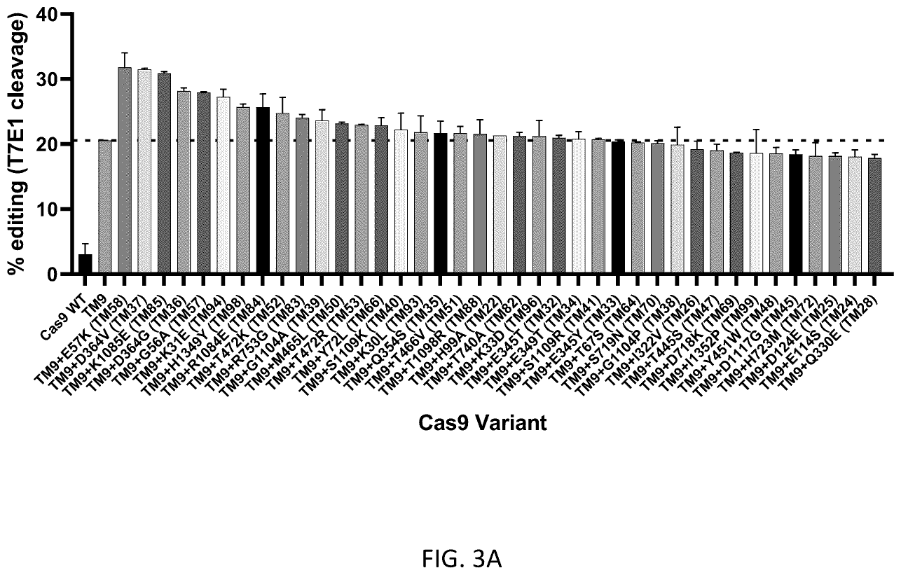

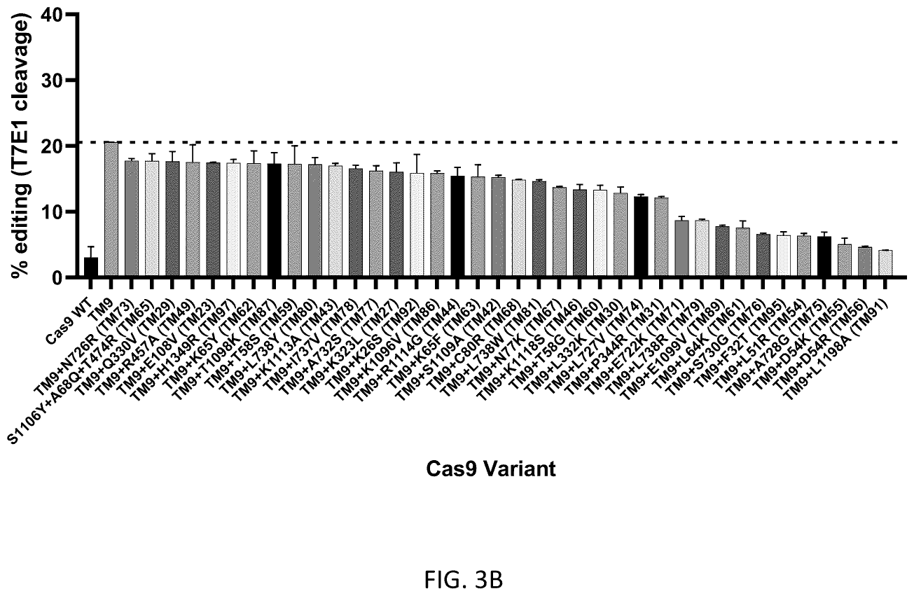

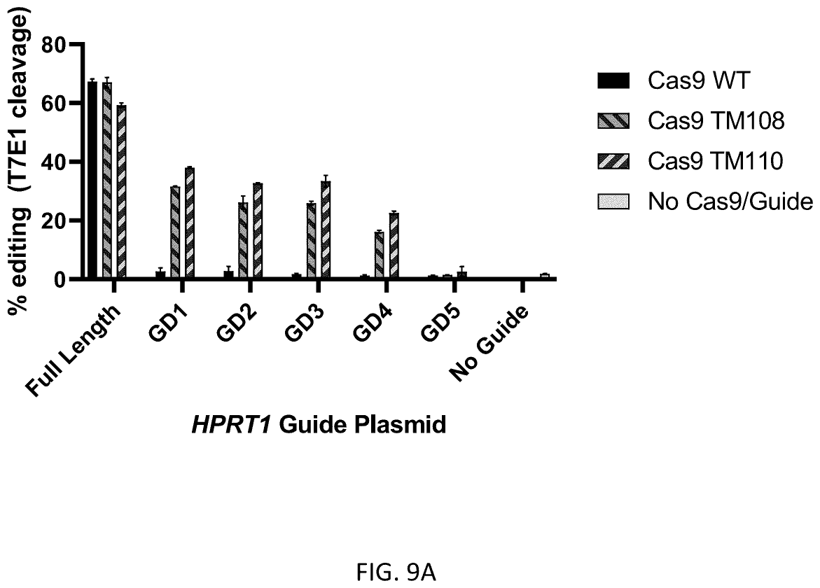

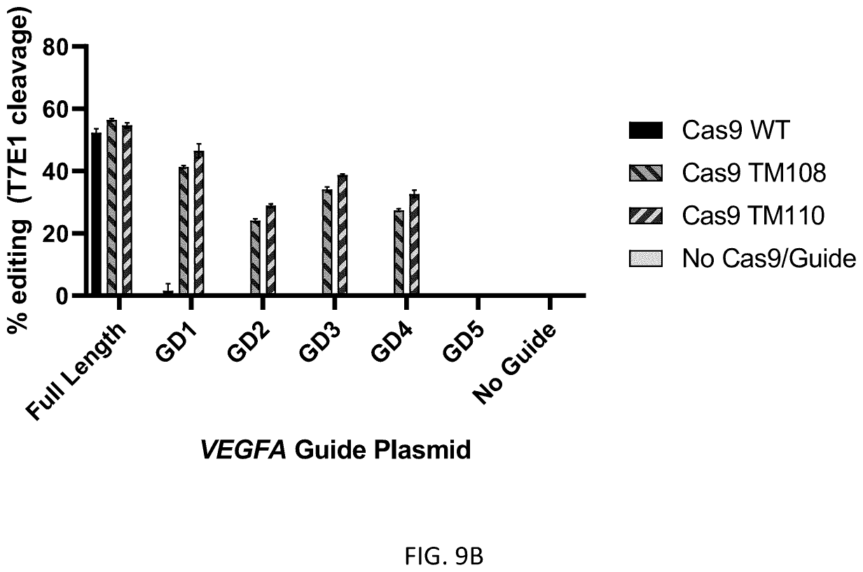

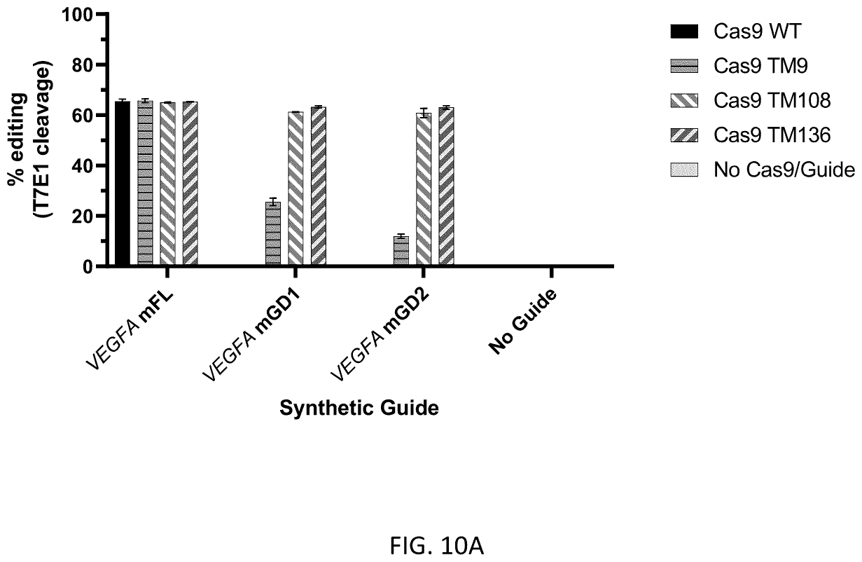

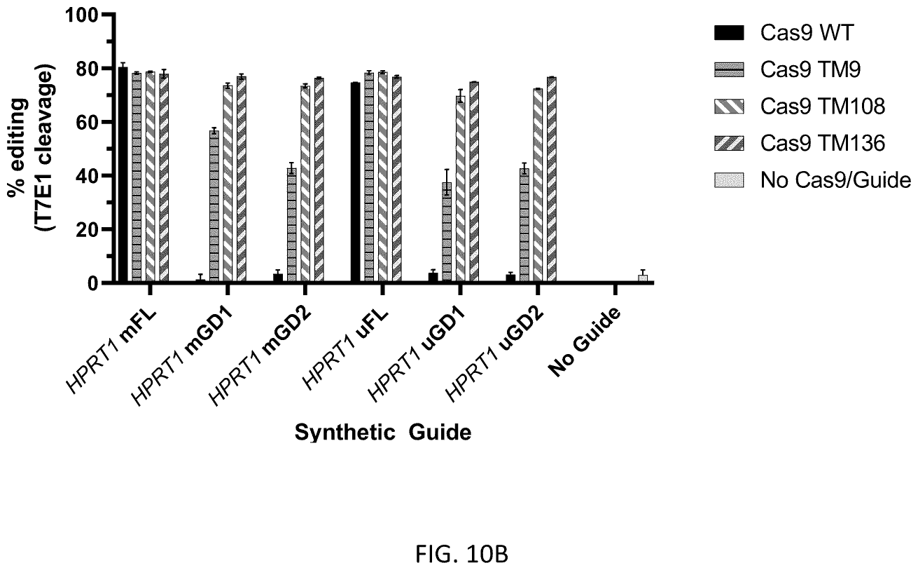

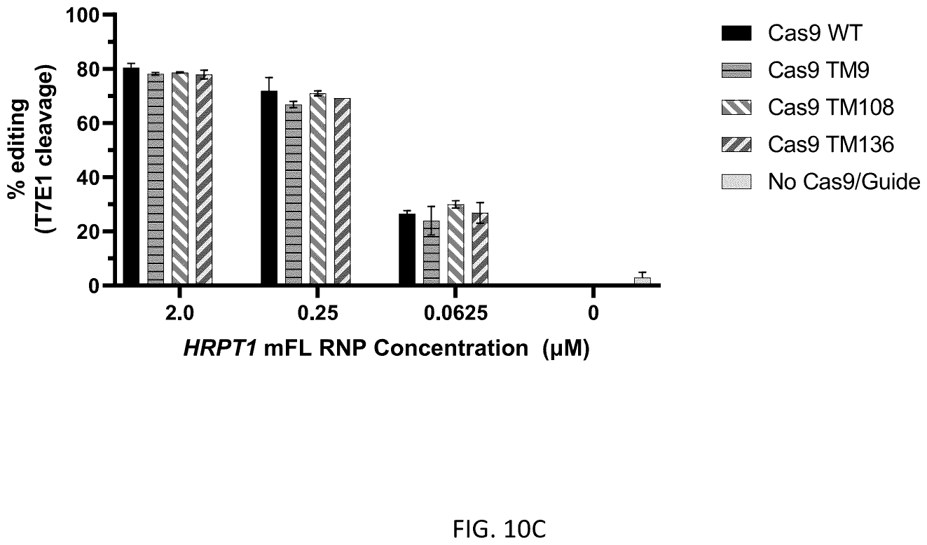

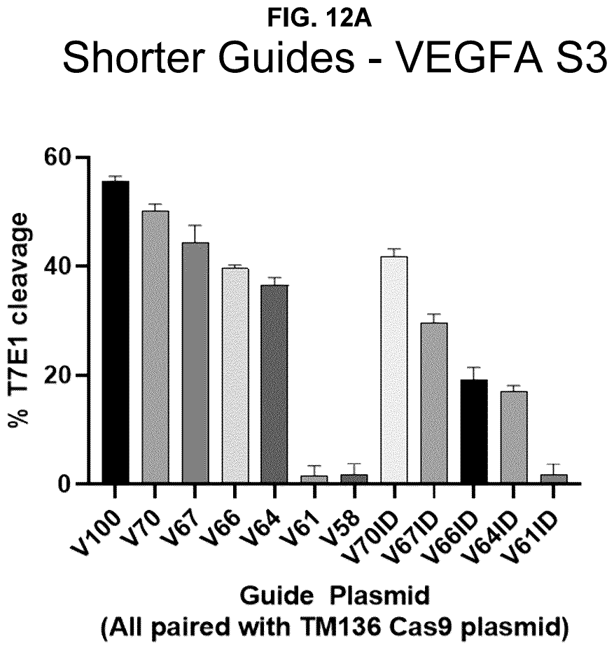

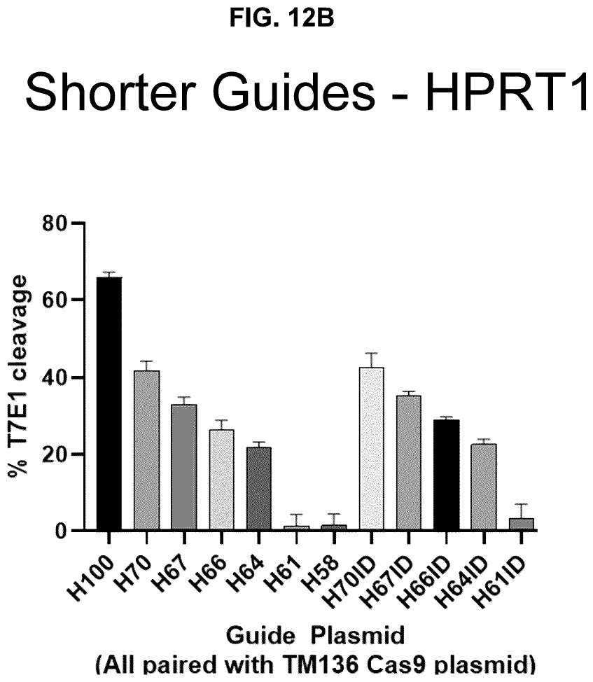

Testing stacking of the beneficial amino acid substitutions resulted in the identification of two triple amino acid substitution Cas9 variants (TM9: S1106Y+A68K+T474R and TM13: S1106Y+A68K+H99A) with the highest level of improved activity compared to Cas9 (FIG. 2). We then conducted additional stacking experiments and identified variants with further improved activity, with three being our preferred variants (TM108: S1106Y+A68K+T474R+K31E+G56A+E57K, TM110: S1106Y+A68K+T474R+K31E+G56A+E57K+K1085E, and TM136: S1106Y+A68K+T474R+K31E+G56A+E57K+R753G+H329K+T333R) (FIGS. 3-8). We found that TM9, TM108, and TM136 were also much more active with guides with a scaffold sequence shorter than 76 nucleotides when delivered as ribonucleoprotein (RNP) complex compared to Cas9 (FIG. 10). Cas9 TM108 and TM136 also demonstrated improved activity with guides with even shorter scaffold regions beyond designs GD1 and GD2 (FIG. 9, FIG. 12, FIG. 13, Table 7) suggesting that synthetic guides as short as 56 nucleotides may allow for some editing activity paired with our variants as TM136 losses activity with 55 nucleotide guides (V61ID).

We see our engineered Cas9 variants as a valuable tool to facilitate genome editing using shorter guides that can be produced more efficiently and cheaply as well as facilitating guide synthesis for prime editing by reducing the length of guide constant region. This is particularly challenging for applications such as prime editing, where guide lengths exceed 100 nucleotides^{10.}

Short sgRNAs can be produced more efficiently and cheaply. Having a Cas9 variant that that requires a shorter guide constant region will allow use of easier to synthesize guides. These Cas9 variants also have the potential to improve prime editing and/or base editing efficiency. The design of sgRNAs can have large effects on editing efficiency, a shorter constant region will change where the template portion of a sgRNA is positioned relative to the edited DNA, offering more options for guide design and potentially improving editing rates.

In some embodiments, any one of variants of Cas9 as disclosed herein can be used for applications involving use of Cas9 fused to other functional protein domains such as for base editing by adding or delivering individual components such as a deaminase domain and an uracil glycosylase inhibitor (UGI) domain into a cell. In some embodiments, the variant of Cas9 as disclosed herein comprises additional amino acid substitutions to make the variant into a Cas9 nickase or a catalytically dead Cas9 (dCas9). In some embodiments, fusion proteins comprising the variant of Cas9 as disclosed herein (e.g., dCas9, nuclease active Cas9, or Cas9 nickase) and deaminases or deaminase domains, are provided. In some embodiments, the variant of Cas9 as disclosed herein further comprises D10A, E762A, H840A, N854A, N863A, or D986A substitution. In some embodiments, the variant of Cas9 as disclosed herein further comprises N497A, R661A, Q695A, or Q926A substitution.

In some embodiments, any one of the variants of Cas9 as disclosed herein further comprises R691A substitution.

Disclosed herein are Cas9 variants that have substantially improved activity with a 50 nt (GD1) or 45 nt (GD2) scaffold region guide. This is a significant reduction in length compared to the standard 76 nucleotide scaffold region plus 4 nucleotide terminator sequence which allows synthesis of higher purity sgRNAs.

In one aspect, a method for promoting cleavage of a double-stranded DNA target in a cell in vitro, in vivo, and/or ex vivo by a Clustered Regularly Interspaced Short Palindromic Repeat (CRISPR) endonuclease is presented. The CRISPR endonuclease includes a CRISPR/Cas9 endonuclease having a variant of Cas9 protein and a guide RNA with a scaffold sequence shorter than 76 nucleotides. The method includes the following steps. The first step includes introducing into the cell genome editing reagents. The editing reagents includes the variant of Cas9 protein and the guide RNA with a scaffold sequence shorter than 76 nucleotides. The second step includes contacting the double-stranded DNA target with the CRISPR/Cas9 endonuclease formed from the variant of the Cas9 protein complexed with the guide RNA with a scaffold sequence shorter than 76 nucleotides. The third step includes cleaving the double-stranded DNA target with the resultant CRISPR/Cas9 endonuclease. The resultant CRISPR/Cas9 endonuclease cleaves the double-stranded DNA target with greater efficiency than a CRISPR/Cas9 endonuclease comprising the reference Cas9 protein complexed with the guide RNA with a scaffold sequence shorter than 76 nucleotides.

In one aspect, the isolated variant of Cas9 protein disclosed herein includes an amino acid sequence selected from the group consisting of the following relative to the wild-type Cas9 amino acid sequence of SEQ ID NO: 133:
at least one single amino acid substitution selected from the group consisting of H99A, E108P, E130K, K323A, E327T, H329R, Q330S, T333K, A337K, E345N, Q354R, Q354S, D364K, D364R, S1106Y, S1106F, T474R, E60K, K65Y, A68K, Y72V, A725R, A728W, G729M, G729I, G729W, K734R, and E1099R;
a double amino acid substitution comprising S1106Y and an additional substitution selected from the group consisting of E60K, A68K, T474R, A725R, A728W, H99A, E108P, and E130K;
a triple amino acid substitution comprising A68K/S1106Y and an additional substitution selected from the group consisting of T474R, E60K, A725R, A728W, H99A, E108P, E130K, and T333K;
a 4-amino acid substitution comprising A68K/T474R/S1106Y and an additional substitution selected from the group consisting of E60K, A725R, A728W, E108P, H99A, E108V, E114S, D124E, I322V, K323L, Q330E, Q330V, L332K, P344R, E345T, E345Y, E349T, Q354S, D364G, D364V, G1104P, G1104A, S1109K, S1109R, S1109A, K1113A, R1114G, D1117G, K1118S, T445S, Y451W, R457A, M465L, T466V, T472K, T472R, L51R, D54K, D54R, G56A, E57K, T58S, T58G, L64K, Y72L, K65Y, K65F, T67S, N77K, C80R, D718K, S719N, E722K, H723M, N726R, L727V, A728G, S730G, A732S, I737V, L738R, L738Y, L738W, T740A, R753G, R1084E, K1085E, K1096V, T1098K, T1098R, E1099V, G1104A, L1198A, K26S, K30L, K31E, F32T, K33D, H1349R, H1349Y, and I1352P;
a 5-amino acid substitution comprising A68K/T474R/S1106Y and an additional substitution selected from the group consisting of G56A/E57K, K31E/G56A, K31E/E57K, K1085E/G56A, K1085E/E57K, and K1085E/K31E;
a 6-amino acid substitution comprising A68K/T474R/S1106Y and an additional substitution selected from the group consisting of K31E/K1085E/G56A, K31E/K1085E/E57K, K31E/G56A/E57K, and G56A/K1085E/E57K;
a 7-amino acid substitution comprising K31E/G56A/E57K/A68K/T474R/S1106Y and an additional substitution selected from the group consisting of K1085E, G1104A, M465L, T472K, R1084E, H1349Y, R753G, E108V, E130N, H329K, Q330V, T333R, S355C, A50T, I733Y, R753S, and P1090Y;
an 8-amino acid substitution comprising K31E/G56A/E57K/A68K/T474R/S1106Y and an additional substitution selected from the group consisting of H1349Y/R753G, T472K/R753G, H329K/R753G, T333R/R753G, H329K/T472K, T333R/T472K, and H329K/T333R;
a 9-amino acid substitution comprising K31E/G56A/E57K/A68K/T474R/S1106Y and an additional substitution selected from the group consisting of H329K/T472K/R753G,
T333R/T472K/R753G, H329K/T333R/R753G, and H329K/T333R/T472K; and
a 10-amino acid substitution comprising K31E, G56A, E57K, A68K, H329K, T333R, T472K, T474R, R753G, and S1106Y substitutions.

In one aspect, a method for promoting cleavage of a double-stranded DNA target in a cell in vitro, in vivo, and/or ex vivo by a Clustered Regularly Interspaced Short Palindromic Repeat (CRISPR) endonuclease is disclosed. The CRISPR endonuclease includes a CRISPR/Cas9 endonuclease having a variant of Cas9 protein and an guide RNA with a scaffold sequence shorter than 76 nucleotides. The method may include, for example, the following steps. The first step includes introducing into the cell genome editing reagents comprising nucleic acids encoding an amino acid sequence of the variant of the Cas9 protein and the guide RNA with a scaffold sequence shorter than 76 nucleotides. The second step includes expressing the amino acid sequence of the variant of the Cas9 protein and the guide RNA with a scaffold sequence shorter than 76 nucleotides from the nucleic acids. The third step includes contacting the double-stranded DNA target with the CRISPR/Cas9 endonuclease formed from the variant of the Cas9 protein complexed with the guide RNA with a scaffold sequence shorter than 76 nucleotides. The fourth step includes cleaving the double-stranded DNA target with the resultant CRISPR/Cas9 endonuclease. The resultant CRISPR/Cas9 endonuclease cleaves the double-stranded DNA target with greater efficiency than a CRISPR/Cas9 endonuclease comprising the reference Cas9 protein complexed with the guide RNA with a scaffold sequence shorter than 76 nucleotides.

In one aspect, a kit for use in promoting cleavage of a DNA target site by a CRISPR/Cas9 endonuclease is disclosed. The kit includes the following components. A first component includes an isolated variant of Cas9 protein. A second component includes an sgRNA with a scaffold region shorter than 76 nucleotides. The CRISPR/Cas9 endonuclease formed by the components cleaves a double-stranded DNA target in living cells with greater efficiency than a CRISPR/Cas9 endonuclease comprising the reference Cas9 protein complexed with the guide RNA with a scaffold sequence shorter than 76 nucleotides.

Consistent with the methods disclosed herein, the variant of Cas9 protein as disclosed herein comprises at least one amino acid substitution selected from Table 5. In some embodiments, the variant of Cas9 protein as disclosed herein comprises at least two amino acid substitutions selected from Table 5 or Table 6. In some embodiments, the variant of Cas9 protein as disclosed herein comprises S1106Y substitution and at least one additional substitution selected from Table 5 or Table 6. In some embodiments, the variant of Cas9 protein as disclosed herein comprises S1106Y, A68K, T474R substitutions and at least one additional substitution selected from Table 5. In some embodiments, the variant of Cas9 protein as disclosed herein comprises S1106Y, A68K, and T474R substitutions. In some embodiments, the variant of Cas9 protein as disclosed herein comprises S1106Y, A68K, T474R, K31E, G56A, and E57K substitutions. In some embodiments, the variant of Cas9 protein as disclosed herein comprises S1106Y, A68K, T474R, K31E, G56A, E57K, R753G, H329K, and T333R substitutions.

The Cas9 variant system and sgRNA components as disclosed herein, for example, can be introduced into a cell or cells, together or separately, using various approaches in vitro, in vivo, and/or ex vivo. In certain embodiments as disclosed herein, the recipient cell is selected from an immortalized cell. In certain embodiments as disclosed herein, the immortalized cell is a HEK293 kidney cell or a Jurkat T cell. In certain embodiments as disclosed herein, the recipient cell is a primary human T cell, chimeric antigen receptor (CAR) T cells, or an induced pluripotent stem cell. Examples for introducing the Cas9 variant system and sgRNA components as disclosed herein include plasmid or viral expression vectors (which lead to endogenous expression), Cas9 mRNA with separate sgRNA transfection, or delivery of the Cas9 protein with the sgRNA as a ribonucleoprotein (RNP) complex. Effective strategies for introducing, for example, the Cas9 variant system as disclosed herein into target cells include, for example, viral vectors, such as Adeno-Associated Virus (AAV) which are widely used for CRISPR delivery because they are generally safe, induce minimal immune response, and have been approved in some gene therapy applications; lentivirus and retrovirus; and adenovirus.

Additional methods for introducing the Cas9 variant system and sgRNA components as disclosed herein, for example, into target cells in vitro, in vivo, and/or ex vivo includes, for example, Lipid Nanoparticles (LNPs) which are commonly used for delivering RNA-based therapies; Electroporation, which involves applying an electrical field to create temporary pores in the cell membrane, allowing CRISPR components (like plasmids, ribonucleoprotein complexes, or mRNA, such as sgRNA) to enter the cell. Additional methods for introducing the Cas9 variant system components as disclosed herein, for example, including a Cas9 variant, into target cells includes, for example, Ribonucleoprotein (RNP) Complexes which involves directly delivering the Cas9 protein pre-complexed with Cas9 variant system into cells, usually via electroporation or lipid-based transfection; Lipid-Based Transfection Agents (lipofection) uses lipid-based reagents to encapsulate CRISPR plasmids or RNP complexes and facilitate their uptake by cells.

Other methods for introducing the Cas9 variant system and sgRNA components as disclosed herein, for example, including a Cas9 variant, into target cells in vitro, in vivo, and/or ex vivo includes, for example, physical methods such as microinjection to directly inject CRISPR components into cells, typically used in single-cell embryos or zygotes for generating transgenic animals; nanoneedles and microfluidics which can introduce CRISPR components with minimal damage to cells; and exosome-mediated delivery, which can be engineered to carry CRISPR/Cas components and target them to specific cells.

A variety of host cells can serve as platforms for the methods and systems as disclosed herein, such as: Bacterial Cells: Escherichia coli is widely used for CRISPR applications like plasmid construction, cloning, and CRISPR screens; Yeast: Saccharomyces cerevisiae and other yeast species can be genetically modified to express CRISPR systems, especially in studies focused on gene function and genome screening in eukaryotic systems; Insect cells can serve as hosts for CRISPR expression. In particular, insect cell lines such as Sf9 (from Spodoptera frugiperda) and S2 (from Drosophila melanogaster); Mammalian Cells: Various mammalian cell lines, including HEK293, HeLa, and CHO cells, are commonly used, which are compatible with more complex CRISPR modifications, such as large gene insertions, knock-ins, or base editing, due to their complex regulatory machinery; Primary Cells and Stem Cells: Primary cells, like human or animal-derived cells, hematopoietic stem and progenitor cells (HSPCs), and induced pluripotent stem cells (iPSCs) can also be used as host cells for CRISPR systems, especially for therapeutic studies and disease modeling; Plant Cells: Plants like Arabidopsis thaliana, tobacco, and rice can serve as CRISPR host cells. Plant cells are often transformed with CRISPR machinery to study gene function, enhance traits, or improve resistance to pathogens. Various delivery systems for CRISPR components, such as plasmids, viral vectors, or ribonucleoprotein complexes, and specific promoters can be optimized for the host's transcriptional machinery.

### Examples

Example 1. Identification of amino acid substitutions that improve Cas9-activity-linked survival in a bacterial screen.

In order to identify amino acid substitutions likely to improve Cas9 activity when paired with guides with short scaffold regions, we generated saturation mutagenesis Cas9 expression plasmid libraries changing amino acids at positions in close proximity to sgRNA constant region. The proximity to the guide of positions in Cas9 was determined using the structure of Cas9 in complex with an sgRNA and DNA (See Table 1 for library designs)¹¹.

TABLE 1
Library designs

| | Cas9 Amino acid positions screened |
|---|---|
| Screen 1 Library 1 | 98-131, 322-367 |
| Screen 1 Library 2 | 150-163, 442-476, 1103-1133 |
| Screen 1 Library 3 | 50-84, 718-754, 1169-1176 |
| Screen 1 Library 4 | 20-49, 1077-1104, 1181-1200, 1348-1359 |
| Screen 2 Library 1 | 2-72 |
| Screen 2 Library 2 | 73-149 |
| Screen 2 Library 3 | 150-226 |
| Screen 2 Library 4 | 227-303 |
| Screen 2 Library 5 | 304-380 |
| Screen 2 Library 6 | 381-457 |
| Screen 2 Library 7 | 458-534 |
| Screen 2 Library 8 | 535-611 |
| Screen 2 Library 9 | 612-688 |
| Screen 2 Library 10 | 689-765 |
| Screen 2 Library 11 | 766-842 |
| Screen 2 Library 12 | 843-919 |
| Screen 2 Library 13 | 920-992 |
| Screen 2 Library 14 | 993-1069 |
| Screen 2 Library 15 | 1070-1146 |
| Screen 2 Library 16 | 1147-1223 |
| Screen 2 Library 17 | 1224-1300 |
| Screen 2 Library 18 | 1301-1368 |

These mutant Cas9 expression plasmid libraries were then delivered into E. coli cells containing a CcdB toxin expression plasmid under the control of an Arabinose inducible promoter alongside synthetic sgRNAs targeting a site in the toxin plasmid corresponding to a Cas9 target site near VEGFA in human cells. (See Table 2). The sgRNAs had shorter than standard scaffold regions of two different designs, guide design 1 (GD1) and guide design 2 (GD2) (See Table 2).

TABLE 2
Synthetic sgRNA designs

| Name | Purpose | Sequence | Spacer | Scaffold |
|---|---|---|---|---|
| VEGFA | Survival | rG*rG*rU*rGrArGrUrGrArGrUrGrUrGrUrGrCr | GGUGAGUGA | GUUUUAGAGCUA |
| sgRNA Full | screen in | GrUrGrGrUrUrUrUrArGrArGrCrUrArGrArArAr | GUGUGUGCG | GAAAUAGCAAGU |
| Length | bacteria | UrArGrCrArArGrUrUrArArArArUrArArGrGrCr | UG (SEQ ID | UAAAAUAAGGCU |
| (VEGFA FL) | | UrArGrUrCrCrGrUrUrArUrCrArArCrUrUrGrAr | NO: 14) | AGUCCGUUAUCA |
| | | ArArArArGrUrGrGrCrArCrCrGrArGrUrCrGrGr | | ACUUGAAAAAGU |
| | | U*rG*rC*rU*rU*rU*rU (SEQ ID NO: 2) | | GGCACCGAGUC |
| | | | | GGUGCUUUU |
| | | | | (SEQ ID NO: 16) |
| VEGFA | Survival | rG*rG*rU*rGrArGrUrGrArGrUrGrUrGrUrGrCr | GGUGAGUGA | GUUUUAGAGCUA |
| sgRNA | screen in | GrUrGrGrUrUrUrUrArGrArGrCrUrArGrArArAr | GUGUGUGCG | GAAAUAGCAAGU |
| Guide | bacteria | UrArGrCrArArGrUrUrArArArArUrArArGrGrCr | UG (SEQ ID | UAAAAUAAGGCU |
| Design 1 | | UrArGrUrCrCrGrUrU*rA*rU*rC*rA*rA*rC | NO: 14) | AGUCCGUUAUCA |
| (VEGFA GD1) | | (SEQ ID NO: 3) | | AC (SEQ ID NO: |
| | | | | 17) |
| VEGFA | Survival | rG*rG*rU*rGrArGrUrGrArGrUrGrUrGrUrGrCr | GGUGAGUGA | GUUUUAGAGGAA |
| sgRNA | screen in | GrUrGrGrUrUrUrUrArGrArGrGrArArArCrArAr | GUGUGUGCG | ACAAGUUAAAAU |
| Guide | bacteria | GrUrUrArArArArUrArArGrGrCrUrArGrUrCrCr | UG (SEQ ID | AAGGCUAGUCC |
| Design 2 | | GrUrUrA*rU*rC*rA*rA*rC*rU (SEQ ID NO: 4) | NO: 14) | GUUAUCAACU |
| (VEGFA GD2) | | | | (SEQ ID NO: 18) |
| VEGFA | RNP testing | mG*mG*mU*rGrArGrUrGrArGrUrGrUrGrUr | GGUGAGUGA | GUUUUAGAGCUA |
| sgRNA Full | | GrCrGrUrGrGrUrUrUrUrArGrArGrCrUrArGrA | GUGUGUGCG | GAAAUAGCAAGU |
| Length with | | rArArUrArGrCrArArGrUrUrArArArArUrArArGr | UG (SEQ ID | UAAAAUAAGGCU |
| end mods | | GrCrUrArGrUrCrCrGrUrUrArUrCrArArCrUrU | NO: 14) | AGUCCGUUAUCA |
| (VEGFA mFL) | | rGrArArArArArGrUrGrGrCrArCrCrGrArGrUrC | | ACUUGAAAAAGU |
| | | rGrGrUrGrCmU*mU*mU*rU (SEQ ID NO: 5) | | GGCACCGAGUC |
| | | | | GGUGCUUUU |
| | | | | (SEQ ID NO: 16) |
| VEGFA | RNP testing | mG*mG*mU*rGrArGrUrGrArGrUrGrUrGrUr | GGUGAGUGA | GUUUUAGAGCUA |
| sgRNA | | GrCrGrUrGrGrUrUrUrUrArGrArGrCrUrArGrA | GUGUGUGCG | GAAAUAGCAAGU |
| Guide | | rArArUrArGrCrArArGrUrUrArArArArUrArArGr | UG (SEQ ID | UAAAAUAAGGCU |
| Design 1 | | GrCrUrArGrUrCrCrGrUrUrArUmC*mA*mA*rC | NO: 14) | AGUCCGUUAUCA |
| with end | | (SEQ ID NO: 6) | | AC (SEQ ID NO: |
| mods | | | | 17) |
| (VEGFA | | | | |
| mGD1 | | | | |
| VEGFA | RNP testing | mG*mG*mU*rGrArGrUrGrArGrUrGrUrGrUr | GGUGAGUGA | GUUUUAGAGGAA |
| sgRNA | | GrCrGrUrGrGrUrUrUrUrArGrArGrGrArArArCr | GUGUGUGCG | ACAAGUUAAAAU |
| Guide | | ArArGrUrUrArArArArUrArArGrGrCrUrArGrUr | UG (SEQ ID | AAGGCUAGUCC |
| Design 2 | | CrCrGrUrUrArUrCmA*mA*mC*rU (SEQ ID | NO: 14) | GUUAUCAACU |
| with end | | NO: 7) | | (SEQ ID NO: 18) |
| mods | | | | |
| (VEGFA | | | | |
| mGD2) | | | | |
| HPRT1 | RNP testing | mA*mA*mU*rUrArUrGrGrGrGrArUrUrArCrUr | AAUUAUGGG | GUUUUAGAGCUA |
| sgRNA Full | | ArGrGrArGrUrUrUrUrArGrArGrCrUrArGrArAr | GAUUACUAG | GAAAUAGCAAGU |
| Length with | | ArUrArGrCrArArGrUrUrArArArArUrArArGrGr | GA (SEQ ID | UAAAAUAAGGCU |
| end mods | | CrUrArGrUrCrCrGrUrUrArUrCrArArCrUrUrG | NO: 15) | AGUCCGUUAUCA |
| (HPRT1 mFL) | | rArArArArArGrUrGrGrCrArCrCrGrArGrUrCrG | | ACUUGAAAAAGU |
| | | rGrUrGrCmU*mU*mU*rU (SEQ ID NO: 8) | | GGCACCGAGUC |
| | | | | GGUGCUUUU |
| | | | | (SEQ ID NO: 16) |
| HPRT1 | RNP testing | mA*mA*mU*rUrArUrGrGrGrGrArUrUrArCrUr | AAUUAUGGG | GUUUUAGAGCUA |
| sgRNA | | ArGrGrArGrUrUrUrUrArGrArGrCrUrArGrArAr | GAUUACUAG | GAAAUAGCAAGU |
| Guide | | ArUrArGrCrArArGrUrUrArArArArUrArArGrGr | GA (SEQ ID | UAAAAUAAGGCU |
| Design 1 | | CrUrArGrUrCrCrGrUrUrArUmC*mA*mA*rC | NO: 15) | AGUCCGUUAUCA |
| with end | | (SEQ ID NO: 9) | | AC (SEQ ID NO: |
| mods | | | | 17) |
| (HPRT1 | | | | |
| mGD1) | | | | |
| HPRT1 | RNP testing | mA*mA*mU*rUrArUrGrGrGrGrArUrUrArCrUr | AAUUAUGGG | GUUUUAGAGGAA |
| sgRNA | | ArGrGrArGrUrUrUrUrArGrArGrGrArArArCrAr | GAUUACUAG | ACAAGUUAAAAU |
| Guide | | ArGrUrUrArArArArUrArArGrGrCrUrArGrUrCr | GA (SEQ ID | AAGGCUAGUCC |
| Design 2 | | CrGrUrUrArUrCmA*mA*mC*rU (SEQ ID NO: | NO: 15) | GUUAUCAACU |
| with end | | 10) | | (SEQ ID NO: 18) |
| mods | | | | |
| (HPRT1 | | | | |
| mGD2) | | | | |
| HPRT1 | RNP testing | rArArUrUrArUrGrGrGrGrArUrUrArCrUrArGrG | AAUUAUGGG | GUUUUAGAGCUA |
| sgRNA Full | | rArGrUrUrUrUrArGrArGrCrUrArGrArArArUrAr | GAUUACUAG | GAAAUAGCAAGU |
| Length with | | GrCrArArGrUrUrArArArArUrArArGrGrCrUrAr | GA (SEQ ID | UAAAAUAAGGCU |
| no end mods | | GrUrCrCrGrUrUrArUrCrArArCrUrUrGrArArAr | NO: 15) | AGUCCGUUAUCA |
| (HPRT1 uFL) | | ArArGrUrGrGrCrArCrCrGrArGrUrCrGrGrUrG | | ACUUGAAAAAGU |
| | | rCrUrUrUrU (SEQ ID NO: 11) | | GGCACCGAGUC |
| | | | | GGUGCUUUU |
| | | | | (SEQ ID NO: 16) |
| HPRT1 | RNP testing | rArArUrUrArUrGrGrGrGrArUrUrArCrUrArGrG | AAUUAUGGG | GUUUUAGAGCUA |
| sgRNA | | rArGrUrUrUrUrArGrArGrCrUrArGrArArArUrAr | GAUUACUAG | GAAAUAGCAAGU |
| Guide | | GrCrArArGrUrUrArArArArUrArArGrGrCrUrAr | GA (SEQ ID | UAAAAUAAGGCU |
| Design 1 | | GrUrCrCrGrUrUrArUrCrArArC (SEQ ID NO: | NO: 15) | AGUCCGUUAUCA |
| With no end | | 12) | | AC (SEQ ID NO: |
| mods | | | | 17) |
| (HPRT1 | | | | |
| uGD1) | | | | |
| HPRT1 | RNP testing | rArArUrUrArUrGrGrGrGrArUrUrArCrUrArGrG | AAUUAUGGG | GUUUUAGAGGAA |
| sgRNA | | rArGrUrUrUrUrArGrArGrGrArArArCrArArGrUr | GAUUACUAG | ACAAGUUAAAAU |
| Guide | | UrArArArArUrArArGrGrCrUrArGrUrCrCrGrUr | GA (SEQ ID | AAGGCUAGUCC |
| Design 2 | | UrArUrCrArArCrU (SEQ ID NO: 13) | NO: 15) | GUUAUCAACU |
| With no end | | | | (SEQ ID NO: 18) |
| mods | | | | |
| (HPRT1 | | | | |
| uGD2) | | | | |

*: phosphorothioate bond, m = 2′ O-methyl RNA base

For GD1 the scaffold region is terminated at what would be position 70 in a standard 100 nt sgRNA, while for GD2 the guide is terminated at position 71, however 6 nucleotides are also deleted in the repeat and anti repeat region of the guide resulting in a 65 nucleotide guide (45 nucleotide scaffold region). If a bacterium receives a plasmid encoding a Cas9 variant that is able to cleave the toxin plasmid when paired with sgRNA of either the GD1 or GD2 designs, that improves survival chance of that bacteria on an arabinose containing plate, resulting in that Cas9 variant plasmid making up a larger portion of total plasmid isolated from the plate. The GD1 and GD2 designs were selected based on screening sequential terminal deletion guide variants as well as various repeat-anti repeat deletion variants to identify designs that gave low level but above background levels of survival under selection pressure in this screening system. Cas9 plasmid plus guide delivery was done in duplicate for each library plus guide design combination and bacteria were then plated on arabinose containing plates. Once grown, all colonies were suspended in media by scraping each plate with a cotton swab or cell scraper then the bacteria from each plate were pelleted and plasmid DNA was isolated. Plasmid DNA from the original plasmid library dilutions (input, replicate 1 and 2) and from each plate (guide design 1 or guide design 2, replicates 1 and 2) were then sequenced by NGS using 2×150 paired end sequencing on a NextSeq2000 (Illumina) targeting approximately 1-2 million reads per amplicon. (see Table 3 for NGS primers).

TABLE 3
Primer sequences

| | | Primer Sequence | |
|---|---|---|---|
| Gene | Assay | Forward | Reverse |
| VEGFA | PCR + T7E1 | CCTCAGTTCCCTGGCAACATC | CACCAAGGTTCACAGCCTGA |
| | digest | (SEQ ID NO: 19) | AAA (SEQ ID NO: 51) |
| HPRT1 | PCR + T7E1 | AAGAATGTTGTGATAAAAGGTGATG | ACACATCCATGGGACTTCTG |
| | digest | CT (SEQ ID NO: 20) | CCTC (SEQ ID NO: 52) |
| Cas9 | NGS | ACACTCTTTCCCTACACGACGCTC | GTGACTGGAGTTCAGACGTGT |
| (Bacterial | (Screen 1 | TTCCGATCTAGATGGCAAAAGTCG | GCTCTTCCGATCTGCGTAAGT |
| screen | Library 1) | ATGACTCGT (SEQ ID NO: 21) | GGTAAATGGTTGGGT (SEQ ID |
| plasmid) | | | NO: 53) |
| Cas9 | NGS | ACACTCTTTCCCTACACGACGCTC | GTGACTGGAGTTCAGACGTGT |
| (Bacterial | (Screen 1 | TTCCGATCTCGCCTCTTAGTGCCA | GCTCTTCCGATCTAGGCTTGA |
| screen | Library 1) | GCAT (SEQ ID NO: 22) | TAAATTTGTAGAATTCCTCTTG |
| plasmid) | | | G (SEQ ID NO: 54) |
| Cas9 | NGS | ACACTCTTTCCCTACACGACGCTC | GTGACTGGAGTTCAGACGTGT |
| (Bacterial | (Screen 1 | TTCCGATCTCAATATTGTTAAGAAAA | GCTCTTCCGATCTTCTACTTTC |
| screen | Library 2) | CCGAAGTGCAGAC (SEQ ID NO: 23) | GCTACAACGAGAACAGA |
| plasmid) | | | (SEQ ID NO: 55) |
| Cas9 | NGS | ACACTCTTTCCCTACACGACGCTC | GTGACTGGAGTTCAGACGTGT |
| (Bacterial | (Screen 1 | TTCCGATCTCCTGAAGGATAACCG | GCTCTTCCGATCTCTTGTCGA |
| screen | Library 2) | GGAGAAAATTGA (SEQ ID NO: 24) | CTACCTCTTCGAAGT (SEQ ID |
| plasmid) | | | NO: 56) |
| Cas9 | NGS | ACACTCTTTCCCTACACGACGCTC | GTGACTGGAGTTCAGACGTGT |
| (Bacterial | (Screen 1 | TTCCGATCTCCATTTACCACTTACG | GCTCTTCCGATCTTGGACAA |
| screen | Library 2) | CAAAAAGCTG (SEQ ID NO: 25) | GCTGAATAAACAATTTATCAAC |
| plasmid) | | | A (SEQ ID NO: 57) |
| Cas9 | NGS | ACACTCTTTCCCTACACGACGCTC | GTGACTGGAGTTCAGACGTGT |
| (Bacterial | (Screen 1 | TTCCGATCTATCGCCATTCAATCAA | GCTCTTCCGATCTGGCGGTG |
| screen | Library 3) | AAAGAACTTGA (SEQ ID NO: 26) | GAAAAACGAGTCATC (SEQ ID |
| plasmid) | | | NO: 58) |
| Cas9 | NGS | ACACTCTTTCCCTACACGACGCTC | GTGACTGGAGTTCAGACGTGT |
| (Bacterial | (Screen 1 | TTCCGATCTTTGTAGCGAAAGTAGA | GCTCTTCCGATCTAGCTTAAT |
| screen | Library 3) | AAAGGGTAAATCCAA (SEQ ID NO: | GATAAGATCTTTCTTGACCTCT |
| plasmid) | | 27) | (SEQ ID NO: 59) |
| Cas9 | NGS | ACACTCTTTCCCTACACGACGCTC | GTGACTGGAGTTCAGACGTGT |
| (Bacterial | (Screen 1 | TTCCGATCTGATGACTCCCTGACTT | GCTCTTCCGATCTTGTCCTTT |
| screen | Library 3) | TTAAAGAGGATATTCA (SEQ ID NO: | CTGGGTCGTTTGATTC (SEQ ID |
| plasmid) | | 28) | NO: 60) |
| Cas9 | NGS | ACACTCTTTCCCTACACGACGCTC | GTGACTGGAGTTCAGACGTGT |
| (Bacterial | (Screen 1 | TTCCGATCTACTGGCGAAATTGTTT | GCTCTTCCGATCTTTATCTGAA |
| screen | Library 4) | GGGAC (SEQ ID NO: 29) | TTACGTTTGGGTAAGATGCT |
| plasmid) | | | (SEQ ID NO: 61) |
| Cas9 | NGS | ACACTCTTTCCCTACACGACGCTC | GTGACTGGAGTTCAGACGTGT |
| (Bacterial | (Screen 1 | TTCCGATCTAGCGTAGCTCCTTCG | GCTCTTCCGATCTTGCAACTC |
| screen | Library 4) | AGAAGA (SEQ ID NO: 30) | GCCGGCAGA (SEQ ID NO: 62) |
| plasmid) | | | |
| Cas9 | NGS | ACACTCTTTCCCTACACGACGCTC | GTGACTGGAGTTCAGACGTGT |
| (Bacterial | (Screen 1 | TTCCGATCTATGGACAAAAAGTACT | GCTCTTCCGATCTGTTTCCCC |
| screen | Library 4) | CTATTGGCCT (SEQ ID NO: 31) | GGAGTCAAACAACA (SEQ ID |
| plasmid) | | | NO: 63) |
| Cas9 | NGS | ACACTCTTTCCCTACACGACGCTC | GTGACTGGAGTTCAGACGTGT |
| (Bacterial | (Screen 1 | TTCCGATCTCGTAAGCGCTATACC | GCTCTTCCGATCTTACGATTA |
| screen | Library 4) | AGTACGA (SEQ ID NO: 32) | CTTTCTGTTCGACTTAAGCATT |
| plasmid) | | | (SEQ ID NO: 64) |
| Cas9 | NGS | ACACTCTTTCCCTACACGACGCTC | GTGACTGGAGTTCAGACGTGT |
| (Bacterial | (Screen 2 | TTCCGATCTAGAAATAATTTTGTTTAA | GCTCTTCCGATCTTGCGATTC |
| screen | Library 1) | CTTTAATAAGGAGATATACCATG | TTACGCCGAGT (SEQ ID NO: |
| plasmid) | | (SEQ ID NO: 33) | 65) |
| Cas9 | NGS | ACACTCTTTCCCTACACGACGCTC | GTGACTGGAGTTCAGACGTGT |
| (Bacterial | (Screen 2 | TTCCGATCTAGCACGTCGCCGGTA | GCTCTTCCGATCTATCATATGT |
| screen | Library 2) | C (SEQ ID NO: 34) | GCCAGGGCTAAATAGATAAG |
| plasmid) | | | (SEQ ID NO: 66) |
| Cas9 | NGS | ACACTCTTTCCCTACACGACGCTC | GTGACTGGAGTTCAGACGTGT |
| (Bacterial | (Screen 2 | TTCCGATCTGGACAGCACTGACAA | GCTCTTCCGATCTCCAAACA |
| screen | Library 3) | AGCT (SEQ ID NO: 35) | AACCATTCTTTTTCTCGC (SEQ |
| plasmid) | | | ID NO: 67) |
| Cas9 | NGS | ACACTCTTTCCCTACACGACGCTC | GTGACTGGAGTTCAGACGTGT |
| (Bacterial | (Screen 2 | TTCCGATCTAGCAAGTCCCGTCGC | GCTCTTCCGATCTTTCGGTGT |
| screen | Library 4) | TTAG (SEQ ID NO: 36) | TAACGCGGAGAATATC (SEQ |
| plasmid) | | | ID NO: 68) |
| Cas9 | NGS | ACACTCTTTCCCTACACGACGCTC | GTGACTGGAGTTCAGACGTGT |
| (Bacterial | (Screen 2 | TTCCGATCTCGACGCCATCTTGCT | GCTCTTCCGATCTCCAATAAC |
| screen | Library 5) | TTCG (SEQ ID NO: 37) | TCTTCGGTACCATCCA (SEQ |
| plasmid) | | | ID NO: 69) |
| Cas9 | NGS | ACACTCTTTCCCTACACGACGCTC | GTGACTGGAGTTCAGACGTGT |
| (Bacterial | (Screen 2 | TTCCGATCTCAAGAGGAATTCTACA | GCTCTTCCGATCTCACGCGA |
| screen | Library 6) | AATTTATCAAGCCT (SEQ ID NO: 38) | AACGGCTATTACC (SEQ ID |
| plasmid) | | | NO: 70) |
| Cas9 | NGS | ACACTCTTTCCCTACACGACGCTC | GTGACTGGAGTTCAGACGTGT |
| (Bacterial | (Screen 2 | TTCCGATCTGTAGGCCCCTTAGCA | GCTCTTCCGATCTAAGAAAAG |
| screen | Library 7) | CGG (SEQ ID NO: 39) | CGGGCTTGCG (SEQ ID NO: |
| plasmid) | | | 71) |
| Cas9 | NGS | ACACTCTTTCCCTACACGACGCTC | GTGACTGGAGTTCAGACGTGT |
| (Bacterial | (Screen 2 | TTCCGATCTACCAAGGTTAAATACG | GCTCTTCCGATCTACGATGTC |
| screen | Library 8) | TGACGGA (SEQ ID NO: 40) | CTCGAGAATATCTTCGTT (SEQ |
| plasmid) | | | ID NO: 72) |
| Cas9 | NGS | ACACTCTTTCCCTACACGACGCTC | GTGACTGGAGTTCAGACGTGT |
| (Bacterial | (Screen 2 | TTCCGATCTATCATTAAGGATAAAG | GCTCTTCCGATCTCTGCATGA |
| screen | Library 9) | ACTTCTTAGATAATGAAGAG (SEQ ID | AGTTGCGATTAGC (SEQ ID |
| plasmid) | | NO: 41) | NO: 73) |
| Cas9 | NGS | ACACTCTTTCCCTACACGACGCTC | GTGACTGGAGTTCAGACGTGT |
| (Bacterial | (Screen 2 | TTCCGATCTCGATTCTGGATTTCTTG | GCTCTTCCGATCTGTCCTTTC |
| screen | Library 10) | AAGTCGGAC (SEQ ID NO: 42) | TGGGTCGTTTGATTCTC (SEQ |
| plasmid) | | | ID NO: 74) |
| Cas9 | NGS | ACACTCTTTCCCTACACGACGCTC | GTGACTGGAGTTCAGACGTGT |
| (Bacterial | (Screen 2 | TTCCGATCTAAAACATCGTGATTGA | GCTCTTCCGATCTTAGAATCG |
| screen | Library 11) | AATGGCG (SEQ ID NO: 43) | TCTTTGAGGAAGCTCTG (SEQ |
| plasmid) | | | ID NO: 75) |
| Cas9 | NGS | ACACTCTTTCCCTACACGACGCTC | GTGACTGGAGTTCAGACGTGT |
| (Bacterial | (Screen 2 | TTCCGATCTGCTGAGTGACTACGA | GCTCTTCCGATCTGTGATTTG |
| screen | Library 12) | CGTTGATC (SEQ ID NO: 44) | ACGAGTCTCCACTAACTG |
| plasmid) | | | (SEQ ID NO: 76) |
| Cas9 | NGS | ACACTCTTTCCCTACACGACGCTC | GTGACTGGAGTTCAGACGTGT |
| (Bacterial | (Screen 2 | TTCCGATCTGAGCGAGTTAGATAAG | GCTCTTCCGATCTTGATAAGG |
| screen | Library 13) | GCAGGA (SEQ ID NO: 45) | GCGGTCCCAAC (SEQ ID NO: |
| plasmid) | | | 77) |
| Cas9 | NGS | ACACTCTTTCCCTACACGACGCTC | GTGACTGGAGTTCAGACGTGT |
| (Bacterial | (Screen 2 | TTCCGATCTACGCGTACCTGAATG | GCTCTTCCGATCTACGCCCTT |
| screen | Library 14) | CAGTG (SEQ ID NO: 46) | TGTCCCAAACAAT (SEQ ID |
| plasmid) | | | NO: 78) |
| Cas9 | NGS | ACACTCTTTCCCTACACGACGCTC | GTGACTGGAGTTCAGACGTGT |
| (Bacterial | (Screen 2 | TTCCGATCTCGTCCGTTGATCGAAA | GCTCTTCCGATCTTTGGATTTA |
| screen | Library 15) | CAAACG (SEQ ID NO: 47) | CCCTTTTCTACTTTCGC (SEQ |
| plasmid) | | | ID NO: 79) |
| Cas9 | NGS | ACACTCTTTCCCTACACGACGCTC | GTGACTGGAGTTCAGACGTGT |
| (Bacterial | (Screen 2 | TTCCGATCTCCGTAGCCTACTCTGT | GCTCTTCCGATCTACGTACTT |
| screen | Library 16) | TCTCG (SEQ ID NO: 48) | TGATGGAAGTGCTAATTCATT |
| plasmid) | | | (SEQ ID NO: 80) |
| Cas9 | NGS | ACACTCTTTCCCTACACGACGCTC | GTGACTGGAGTTCAGACGTGT |
| (Bacterial | (Screen 2 | TTCCGATCTCCGGCGAGTTGCAGA | GCTCTTCCGATCTGGTGAATG |
| screen | Library 17) | AGG (SEQ ID NO: 49) | ATATTTTCAGCCTGCTCAC |
| plasmid) | | | (SEQ ID NO: 81) |
| Cas9 | NGS | ACACTCTTTCCCTACACGACGCTC | GTGACTGGAGTTCAGACGTGT |
| (Bacterial | (Screen 2 | TTCCGATCTCTGAGCGCTTACAATA | GCTCTTCCGATCTCAAGCTTG |
| screen | Library 18) | AGCATCG (SEQ ID NO: 50) | TCGACCTGCAG (SEQ ID NO: |
| plasmid) | | | 82) |

A first screen was conducted mutating Cas9 at codons for amino acids that are in close proximity to the guide scaffold region (Screen 1) in the context of Cas RNP. This screen involved 4 different plasmid libraries (see Table 1). Each library was delivered twice as two separate replicates for each guide design tested. Library one was delivered in one additional experiment resulting in two additional replicates however only the guide design 1 sgRNA was tested in the second experiment involving only library one. A second screen was then conducted screening all positions in Cas9 except position 1 (positions 2-1368) using the same screening method.

In order to determine if a amino acid substitution resulted in improved activity relative to wild-type Cas9, enrichment relative to synonymous amino acid substitutions was calculated. Enrichment relative to synonymous reads was calculated as Enrichment=number of reads for a particular amino acid substitution in the post screen sample/total number of reads for synonymous amino acid substitutions for the post-screen sample)/(number of reads for a particular amino acid substitution in the pre-screen sample/total number of reads for synonymous amino acid substitutions in the pre-screen sample). Only reads containing amino acid substitutions at a single codon within the read were included in the calculations. If the enrichment is greater than 1, then the amino acid substitution was overrepresented on the screen plate relative to theoretically neutral synonymous amino acid substitutions, and therefore potentially beneficial for Cas9 activity when paired with a short scaffold region guide. An amino acid change was considered beneficial if there was at least one amino acid substitution resulting in that amino acid change with an enrichment of greater than 1 for both biological replicates (separate delivery events into bacteria) for either guide design. Further, the additional two replicates for GD1 library 1 were also used in determining the final list of beneficial amino acid substitutions and amino acid substitutions that met the enrichment criteria were included in the beneficial amino acid substitution list from screen 1. A total of 1096 beneficial amino acid changes were identified from screen 1. For screen 2, two technical replicate NGS preps were done for each sample, and an amino acid change was considered beneficial if there was at least one amino acid substitution resulting in that amino acid change with an enrichment of greater than 1 for both biological replicates for either of two technical replicate sequencing samples for either guide design for screen 2. A total of 7450 beneficial amino acid changes were identified from screen 2 and are listed in Table 5.

TABLE 4
Protein expression plasmid sequences

| Plasmid | Sequence |
|---|---|
| Wild-type | TGAAGTGCTTCATGTGGCAGGAGAAAAAAGGCTGCACCGGTGCGTCAGC |
| Cas9 | AGAATATGTGATACAGGATATATTCCGCTTCCTCGCTCACTGACTCGCTAC |
| expression | GCTCGGTCGTTCGACTGCGGCGAGCGGAAATGGCTTACGAACGGGGCGG |
| plasmid | AGATTTCCTGGAAGATGCCAGGAAGATACTTAACAGGGAAGTGAGAGGG |
| (Bacterial | CCGCGGCAAAGCCGTTTTTCCATAGGCTCCGCCCCCCTGACAAGCATCACG |
| screen | AAATCTGACGCTCAAATCAGTGGTGGCGAAACCCGACAGGACTATAAAGA |
| plasmid) | TACCAGGCGTTTCCCCTGGCGGCTCCCTCGTGCGCTCTCCTGTTCCTGCCTT |
| | TCGGTTTACCGGTGTCATTCCGCTGTTATGGCCGCGTTTGTCTCATTCCACG |
| | CCTGACACTCAGTTCCGGGTAGGCAGTTCGCTCCAAGCTGGACTGTATGCA |
| | CGAACCCCCCGTTCAGTCCGACCGCTGCGCCTTATCCGGTAACTATCGTCTT |
| | GAGTCCAACCCGGAAAGACATGCAAAAGCACCACTGGCAGCAGCCACTGG |
| | TAATTGATTTAGAGGAGTTAGTCTTGAAGTCATGCGCCGGTTAAGGCTAAA |
| | CTGAAAGGACAAGTTTTGGTGACTGCGCTCCTCCAAGCCAGTTACCTCGGT |
| | TCAAAGAGTTGGTAGCTCAGAGAACCTTCGAAAAACCGCCCTGCAAGGCG |
| | GTTTTTTCGTTTTCAGAGCAAGAGATTACGCGCAGACCAAAACGATCTCAA |
| | GAAGATCATCTTATTAATCAGATAAAATATTTCTAGATTTCAGTGCAATTTA |
| | TCTCTTCAAATGTAGCACCTGAAGTCAGCCCCATACGATATAAGTTGTAATT |
| | CTCATGTTAGTCATGCCCCGCGCCCACCGGAAGGAGCTGACTGGGTTGAA |
| | GGCTCTCAAGGGCATCGGTCGAGATCCCGGTGCCTAATGAGTGAGCTAAC |
| | TTACATTAATTGCGTTGCGCTCACTGCCCGCTTTCCAGTCGGGAAACCTGTC |
| | GTGCCAGCTGCATTAATGAATCGGCCAACGCGCGGGGAGAGGCGGTTTG |
| | CGTATTGGGCGCCAGGGTGGTTTTTCTTTTCACCAGTGAGACGGGCAACA |
| | GCTGATTGCCCTTCACCGCCTGGCCCTGAGAGAGTTGCAGCAAGCGGTCC |
| | ACGCTGGTTTGCCCCAGCAGGCGAAAATCCTGTTTGATGGTGGTTAACGG |
| | CGGGATATAACATGAGCTGTCTTCGGTATCGTCGTATCCCACTACCGAGAT |
| | GTCCGCACCAACGCGCAGCCCGGACTCGGTAATGGCGCGCATTGCGCCCA |
| | GCGCCATCTGATCGTTGGCAACCAGCATCGCAGTGGGAACGATGCCCTCA |
| | TTCAGCATTTGCATGGTTTGTTGAAAACCGGACATGGCACTCCAGTCGCCT |
| | TCCCGTTCCGCTATCGGCTGAATTTGATTGCGAGTGAGATATTTATGCCAG |
| | CCAGCCAGACGCAGACGCGCCGAGACAGAACTTAATGGGCCCGCTAACA |
| | GCGCGATTTGCTGGTGACCCAATGCGACCAGATGCTCCACGCCCAGTCGC |
| | GTACCGTCTTCATGGGAGAAAATAATACTGTTGATGGGTGTCTGGTCAGA |
| | GACATCAAGAAATAACGCCGGAACATTAGTGCAGGCAGCTTCCACAGCAA |
| | TGGCATCCTGGTCATCCAGCGGATAGTTAATGATCAGCCCACTGACGCGTT |
| | GCGCGAGAAGATTGTGCACCGCCGCTTTACAGGCTTCGACGCCGCTTCGTT |
| | CTACCATCGACACCACCACGCTGGCACCCAGTTGATCGGCGCGAGATTTAA |
| | TCGCCGCGACAATTTGCGACGGCGCGTGCAGGGCCAGACTGGAGGTGGC |
| | AACGCCAATCAGCAACGACTGTTTGCCCGCCAGTTGTTGTGCCACGCGGTT |
| | GGGAATGTAATTCAGCTCCGCCATCGCCGCTTCCACTTTTTCCCGCGTTTTC |
| | GCAGAAACGTGGCTGGCCTGGTTCACCACGCGGGAAACGGTCTGATAAG |
| | AGACACCGGCATACTCTGCGACATCGTATAACGTTACTGGTTTCACATTCA |
| | CCACCCTGAATTGACTCTCTTCCGGGCGCTATCATGCCATACCGCGAAAGG |
| | TTTTGCGCCATTCGATGGTGTCCGGGATCTCGACGCTCTCCCTTATGCGACT |
| | CCTGCATTAGGAAATTAATACGACTCACTATAGGGGAATTGTGAGCGGAT |
| | AACAATTCCCCTGTAGAAATAATTTTGTTTAACTTTAATAAGGAGATATACC |
| | ATGGACAAAAAGTACTCTATTGGCCTGGATATCGGGACCAACAGCGTCGG |
| | GTGGGCTGTTATCACCGACGAGTATAAAGTACCTTCGAAAAAGTTCAAAG |
| | TGCTGGGCAACACCGATCGCCATTCAATCAAAAAGAACTTGATTGGTGCG |
| | CTGTTGTTTGACTCCGGGGAAACCGCCGAGGCGACTCGCCTTAAACGTAC |
| | AGCACGTCGCCGGTACACTCGGCGTAAGAATCGCATTTGCTATTTGCAGG |
| | AAATCTTTAGCAACGAGATGGCAAAAGTCGATGACTCGTTTTTCCACCGCC |
| | TCGAGGAAAGCTTTCTGGTGGAGGAAGACAAAAAGCATGAGCGTCACCC |
| | GATCTTCGGCAACATTGTCGATGAAGTAGCGTATCATGAAAAATACCCAAC |
| | CATTTACCACTTACGCAAAAAGCTGGTGGACAGCACTGACAAAGCTGATTT |
| | GCGCCTTATCTATTTAGCCCTGGCACATATGATTAAGTTTCGTGGTCACTTC |
| | CTGATCGAAGGAGACTTAAATCCCGACAACAGTGATGTTGATAAATTGTTT |
| | ATTCAGCTTGTCCAAACTTACAATCAACTGTTCGAGGAAAACCCGATCAAT |
| | GCCTCCGGTGTGGATGCAAAAGCCATTTTAAGTGCACGCCTTAGCAAGTCC |
| | CGTCGCTTAGAAAACCTTATCGCGCAGCTGCCCGGCGAGAAAAAGAATGG |
| | TTTGTTTGGGAACCTTATTGCCTTGAGCTTAGGCCTCACCCCGAATTTCAAA |
| | AGTAATTTCGATCTTGCAGAAGACGCCAAATTACAACTGTCGAAGGATACT |
| | TATGATGACGATCTCGATAATCTGTTAGCGCAGATTGGTGACCAATACGCC |
| | GATCTTTTTCTGGCGGCTAAAAATCTGAGCGACGCCATCTTGCTTTCGGAT |
| | ATTCTCCGCGTTAACACCGAAATCACGAAAGCGCCTCTTAGTGCCAGCATG |
| | ATTAAACGTTATGATGAACACCACCAGGACCTGACCTTACTCAAAGCGTTG |
| | GTTCGCCAGCAACTGCCAGAGAAGTACAAAGAAATCTTCTTTGATCAGTCA |
| | AAGAATGGTTATGCCGGCTATATTGACGGGGGTGCAAGCCAAGAGGAATT |
| | CTACAAATTTATCAAGCCTATTCTGGAGAAAATGGATGGTACCGAAGAGTT |
| | ATTGGTGAAGCTTAACCGTGAAGACCTCCTGCGGAAACAGCGCACATTCG |
| | ATAATGGTTCGATCCCACACCAAATCCATTTGGGGGAGTTACACGCTATTT |
| | TGCGTCGCCAGGAAGACTTTTACCCTTTCCTGAAGGATAACCGGGAGAAA |
| | ATTGAGAAGATCCTTACCTTTCGTATTCCGTATTACGTAGGCCCCTTAGCAC |
| | GGGGTAATAGCCGTTTCGCGTGGATGACACGGAAGTCGGAAGAGACGAT |
| | CACCCCGTGGAACTTCGAAGAGGTAGTCGACAAGGGCGCATCAGCGCAGT |
| | CTTTTATTGAACGTATGACGAATTTCGATAAAAACTTGCCCAATGAGAAGG |
| | TGCTTCCGAAACATTCCTTGTTATATGAATATTTTACAGTTTACAACGAGCT |
| | GACCAAGGTTAAATACGTGACGGAAGGAATGCGCAAGCCCGCTTTTCTTA |
| | GCGGTGAGCAAAAAAAGGCGATCGTCGACCTGTTATTCAAAACGAATCGT |
| | AAGGTGACTGTAAAGCAACTCAAAGAAGATTACTTCAAAAAGATTGAGTG |
| | CTTCGACAGCGTCGAAATCTCTGGGGTAGAGGATCGGTTTAACGCAAGTT |
| | TAGGTACCTACCATGACCTGCTTAAAATCATTAAGGATAAAGACTTCTTAG |
| | ATAATGAAGAGAACGAAGATATTCTCGAGGACATCGTCTTGACGTTAACCT |
| | TATTTGAGGATCGTGAAATGATTGAGGAACGCCTCAAAACTTATGCCCACC |
| | TGTTCGACGATAAGGTGATGAAGCAGCTGAAACGTCGGCGCTACACAGGA |
| | TGGGGCCGCTTGAGTCGCAAACTTATTAACGGAATCCGTGACAAGCAATC |
| | CGGCAAAACGATTCTGGATTTCTTGAAGTCGGACGGATTTGCTAATCGCAA |
| | CTTCATGCAGTTGATCCATGATGACTCCCTGACTTTTAAAGAGGATATTCAA |
| | AAGGCGCAGGTTAGTGGTCAAGGCGACAGCTTACACGAACACATCGCAAA |
| | TTTGGCTGGTTCGCCGGCCATTAAAAAGGGGATCCTCCAGACCGTGAAAG |
| | TTGTAGATGAGCTTGTTAAGGTCATGGGTCGTCATAAGCCCGAAAACATC |
| | GTGATTGAAATGGCGCGGGAGAATCAAACGACCCAGAAAGGACAAAAGA |
| | ATAGCCGTGAACGGATGAAGCGGATCGAGGAAGGCATTAAAGAGCTGGG |
| | GTCTCAAATCTTGAAGGAACACCCTGTGGAGAACACTCAGCTCCAAAATGA |
| | AAAACTTTACCTGTACTATTTGCAGAACGGACGCGATATGTACGTGGACCA |
| | AGAGTTGGATATTAATCGGCTGAGTGACTACGACGTTGATCATATCGTCCC |
| | GCAGAGCTTCCTCAAAGACGATTCTATTGACAATAAGGTACTGACGCGCTC |
| | TGATAAAAACCGTGGTAAGTCGGACAACGTGCCCTCCGAAGAGGTTGTGA |
| | AAAAGATGAAAAATTATTGGCGCCAGCTTTTAAACGCGAAGCTGATCACA |
| | CAACGTAAATTCGATAATTTGACCAAGGCTGAACGGGGTGGCCTGAGCGA |
| | GTTAGATAAGGCAGGATTTATTAAACGCCAGTTAGTGGAGACTCGTCAAA |
| | TCACCAAACATGTCGCGCAGATTTTGGACAGCCGGATGAACACCAAGTAC |
| | GATGAAAATGACAAACTGATCCGTGAGGTGAAAGTCATTACTCTGAAGTC |
| | CAAATTAGTTAGTGATTTCCGGAAGGACTTTCAATTCTACAAAGTCCGTGA |
| | AATTAATAACTATCATCACGCACATGACGCGTACCTGAATGCAGTGGTTGG |
| | GACCGCCCTTATCAAGAAATATCCTAAGCTGGAGTCGGAGTTTGTCTATGG |
| | CGACTATAAGGTATACGATGTTCGCAAAATGATTGCGAAATCTGAGCAGG |
| | AGATCGGTAAGGCAACCGCAAAATATTTCTTTTACTCAAACATTATGAATTT |
| | CTTTAAGACAGAAATCACTCTGGCCAACGGGGAGATTCGCAAACGTCCGT |
| | TGATCGAAACAAACGGCGAGACTGGCGAAATTGTTTGGGACAAAGGGCG |
| | TGATTTCGCGACGGTGCGCAAGGTACTGAGCATGCCTCAAGTCAATATTGT |
| | TAAGAAAACCGAAGTGCAGACGGGGGGTTTTCCAAGGAAAGCATCTTAC |
| | CCAAACGTAATTCAGATAAACTTATTGCACGCAAAAAGGACTGGGATCCG |
| | AAAAAGTATGGAGGCTTCGACAGTCCAACCGTAGCCTACTCTGTTCTCGTT |
| | GTAGCGAAAGTAGAAAAGGGTAAATCCAAGAAACTGAAATCTGTCAAGG |
| | AGTTGCTTGGAATCACCATTATGGAGCGTAGCTCCTTCGAGAAGAACCCG |
| | ATTGACTTTCTGGAAGCCAAAGGATATAAAGAGGTCAAGAAAGATCTTAT |
| | CATTAAGCTGCCTAAGTATTCACTCTTCGAGCTGGAAAATGGTCGTAAACG |
| | CATGCTCGCTTCTGCCGGCGAGTTGCAGAAGGGCAATGAATTAGCACTTCC |
| | ATCAAAGTACGTTAACTTCCTGTATTTGGCCAGCCATTACGAGAAACTGAA |
| | GGGGTCTCCAGAGGACAACGAACAGAAACAATTATTTGTAGAGCAGCACA |
| | AGCATTATCTTGATGAAATCATTGAGCAAATTTCCGAATTCAGTAAACGCG |
| | TAATCCTGGCCGATGCAAACCTCGACAAGGTGCTGAGCGCTTACAATAAG |
| | CATCGCGACAAACCTATCCGTGAGCAGGCTGAAAATATCATTCACCTGTTC |
| | ACATTAACGAACCTGGGCGCTCCGGCCGCTTTTAAATATTTCGACACGACA |
| | ATCGACCGTAAGCGCTATACCAGTACGAAAGAAGTGTTGGATGCGACCCT |
| | TATTCACCAGTCAATTACAGGATTATATGAGACCCGTATCGACCTTAGCCA |
| | ATTAGGTGGGGATGAGCTCGGCGCGCCTGCAGGTCGACAAGCTTGCGGC |
| | CGCATAATGCTTAAGTCGAACAGAAAGTAATCGTATCCTCAGCGGCCGCAT |
| | AATCGAAATTAATACGACTCACTATAGGGGAATTGTGAGCGGATAACAAT |
| | TCCCCATCTTAGTATATTAGTTAAGTATAAGAAGGAGATATACATATGGCA |
| | GATCTCAATTGGATATCGGCCGGCCACGCGATCGCTGACGTCGGTACCCTC |
| | GAGTCTGGTAAAGAAACCGCTGCTGCGAAATTTGAACGCCAGCACATGGA |
| | CTCGTCTACTAGCGCAGCTTAATTAACCTAGGCTGCTGCCACCGCTGAGCA |
| | ATAACTAGCATAACCCCTTGGGGCCTCTAAACGGGTCTTGAGGGGTTTTTT |
| | GCTGAAACCTCAGGCATTTGAGAAGCACACGGTCACACTGCTTCCGGTAG |
| | TCAATAAACCGGTAAACCAGCAATAGACATAAGCGGCTATTTAACGACCCT |
| | GCCCTGAACCGACGACCGGGTCGAATTTGCTTTCGAATTTCTGCCATTCAT |
| | CCGCTTATTATCACTTATTCAGGCGTAGCACCAGGCGTTTAAGGGCACCAA |
| | TAACTGCCTTAAAAAAATTACGCCCCGCCCTGCCACTCATCGCAGTACTGTT |
| | GTAATTCATTAAGCATTCTGCCGACATGGAAGCCATCACAGACGGCATGAT |
| | GAACCTGAATCGCCAGCGGCATCAGCACCTTGTCGCCTTGCGTATAATATT |
| | TGCCCATAGTGAAAACGGGGGCGAAGAAGTTGTCCATATTGGCCACGTTT |
| | AAATCAAAACTGGTGAAACTCACCCAGGGATTGGCTGAGACGAAAAACAT |
| | ATTCTCAATAAACCCTTTAGGGAAATAGGCCAGGTTTTCACCGTAACACGC |
| | CACATCTTGCGAATATATGTGTAGAAACTGCCGGAAATCGTCGTGGTATTC |
| | ACTCCAGAGCGATGAAAACGTTTCAGTTTGCTCATGGAAAACGGTGTAAC |
| | AAGGGTGAACACTATCCCATATCACCAGCTCACCGTCTTTCATTGCCATACG |
| | GAACTCCGGATGAGCATTCATCAGGCGGGCAAGAATGTGAATAAAGGCC |
| | GGATAAAACTTGTGCTTATTTTTCTTTACGGTCTTTAAAAAGGCCGTAATAT |
| | CCAGCTGAACGGTCTGGTTATAGGTACATTGAGCAACTGACTGAAATGCCT |
| | CAAAATGTTCTTTACGATGCCATTGGGATATATCAACGGTGGTATATCCAG |
| | TGATTTTTTTCTCCATTTTAGCTTCCTTAGCTCCTGAAAATCTCGATAACTCA |
| | AAAAATACGCCCGGTAGTGATCTTATTTCATTATGGTGAAAGTTGGAACCT |
| | CTTACGTGCCGATCAACGTCTCATTTTCGCCAAAAGTTGGCCCAGGGCTTC |
| | CCGGTATCAACAGGGACACCAGGATTTATTTATTCTGCGAAGTGATCTTCC |
| | GTCACAGGTATTTATTCGGCGCAAAGTGCGTCGGGTGATGCTGCCAACTTA |
| | CTGATTTAGTGTATGATGGTGTTTTTGAGGTGCTCCAGTGGCTTCTGTTTCT |
| | ATCAGCTGTCCCTCCTGTTCAGCTACTGACGGGGTGGTGCGTAACGGCAA |
| | AAGCACCGCCGGACATCAGCGCTAGCGGAGTGTATACTGGCTTACTATGT |
| | TGGCACTGATGAGGGTGTCAG (SEQ ID NO: 83) |
| Toxin | TCGCGCGTTTCGGTGATGACGGTGAAAACCTCTGACACATGCAGCTCCCG |
| expression | GAGACGGTCACAGCTTGTCTGTAAGCGGATGCCGGGAGCAGACAAGCCC |
| plasmid | GTCAGGGCGCGTCAGCGGGTGTTGGCGGGTGTCGGGGCTGGCTTAACTA |
| (Bacterial | TGCGGCATCAGAGCAGATTGTACTGAGAGTGCACCATATGCGGTGTGAAA |
| screen | TACCGCACAGATGCGTAAGGAGAAAATACCGCATCAGGCGCCATTCGCCA |
| plasmid) | TTCAGGCTGCGCAACTGTTGGGAAGGGCGATCGGTGCGGGCCTCTTCGCT |
| | ATTACGCCAGCTGGCGAAAGGGGGATGTGCTGCAAGGCGATTAAGTTGG |
| | GTAACGCCAGGGTTTTCCCAGTCACGACGTTGTAAAACGACGGCCAGTGA |
| | ATTCCCAGGCTTTACACTTTATGCTTCCGGCTCGTATAATGTGTGGAATCAC |
| | ACAGGAAACAGAATTATGTACTATTTAAAAAACACAAACTTTTGGATGTTC |
| | GGTTTATTCTTTTTCTTTTACTTTTTTATCATGGGAGCCTACTTCCCGTTTTTC |
| | CCGATTTGGCTACATGACATCAACCATATCAGCAAAAGTGATACGGGTATT |
| | ATTTTTGCCGCTATTTCTCTGTTCTCGCTATTATTCCAACCGCTGTTTGGTCT |
| | GCTTTCTGACAAACTCGGGCTGCGCAAATACCTGCTGTGGATTATTACCGG |
| | CATGTTAGTGATGTTTGCGCCGTTCTTTATTTTTATCTTCGGGCCACTGTTAC |
| | AATACAACATTTTAGTAGGATCGATTGTTGGTGGTATTTATCTAGGCTTTTG |
| | TTTTAACGCCGGTGCGCCAGCAGTAGAGGCATTTATTGAGAAAGTCAGCC |
| | GTCGCAGTAATTTCGAATTTGGTCGCGCGCGGATGTTTGGCTGTGTTGGCT |
| | GGGCGCTGTGTGCCTCGATTGTCGGCATCATGTTCACCATCAATAATCAGT |
| | TTGTTTTCTGGCTGGGCTCTGGCTGTTGCCTCATCCTCGCCGTTTTACTCTTT |
| | TTCGCCAAAACGGATGCGCCCTCTTCTGCCACGGTTGCCAATGCGGTAGGT |
| | GCCAACCATTCGGCATTTAGCCTTAAGCTGGCACTGGAACTGTTCAGACAG |
| | CCAAAACTGTGGTTTTTGTCACTGTATGTTATTGGCGTTTCCTGCACCTACG |
| | ATGTTTTTGACCAACAGTTTGCTAATTTCTTTACTTCGTTCTTTGCTACCGGT |
| | GAACAGGGTACGCGGGTATTTGGCTACGTAACGACAATGGGCGAATTACT |
| | TAACGCCTCGATTATGTTCTTTGCGCCACTGATCATTAATCGCATCGGTGG |
| | GAAAAACGCCCTGCTGCTGGCTGGCACTATTATGTCTGTACGTATTATTGG |
| | CTCATCGTTCGCCACCTCAGCGCTGGAAGTGGTTATTCTGAAAACGCTGCA |
| | TATGTTTGAAGTACCGTTCCTGCTGGTGGGCTGCTTTAAATATATTACCAGC |
| | CAGTTTGAAGTGCGTTTTTCAGCGACGATTTATCTGGTCTGTTTCTGCTTCT |
| | TTAAGCAACTGGCGATGATTTTTATGTCTGTACTGGCGGGCAATATGTATG |
| | AAAGCACCGGTTTCCAGGGCGCTTATCTGGTGCTGGGTCTGGTGGCGCTG |
| | GGCTTCACCTTAATTTCCGTGTTCACGCTTAGCGGCCCCGGCCCGCTTTCCC |
| | TGCTGCGTCGTCAGGTGAATGAAGTCGCTTAAGCAATCAATGTCGGATGC |
| | GGACCGGGAGCGCTGTGAATACAGTGCTCCCTTTTTTTATTGAGCTCGGTA |
| | CCTTATGACAACTTGACGGCTACATCATTCACTTTTTCTTCACAACCGGCAC |
| | GGAACTCGCTCGGGCTGGCCCCGGTGCATTTTTTAAATACCCGCGAGAAAT |
| | AGAGTTGATCGTCAAAACCAACATTGCGACCGACGGTGGCGATAGGCATC |
| | CGGGTGGTGCTCAAAAGCAGCTTCGCCTGGCTGATACGTTGGTCCTCGCG |
| | CCAGCTTAAGACGCTAATCCCTAACTGCTGGCGGAAAAGATGTGACAGAC |
| | GCGACGGCGACAAGCAAACATGCTGTGCGACGCTGGCGATATCAAAATTG |
| | CTGTCTGCCAGGTGATCGCTGATGTACTGACAAGCCTCGCGTACCCGATTA |
| | TCCATCGGTGGATGGAGCGACTCGTTAATCGCTTCCATGCGCCGCAGTAAC |
| | AATTGCTCAAGCAGATTTATCGCCAGCAGCTCCGAATAGCGCCCTTCCCCT |
| | TGCCCGGCGTTAATGATTTGCCCAAACAGGTCGCTGAAATGCGGCTGGTG |
| | CGCTTCATCCGGGCGAAAGAACCCCGTATTGGCAAATATTGACGGCCAGT |
| | TAAGCCATTCATGCCAGTAGGCGCGCGGACGAAAGTAAACCCACTGGTGA |
| | TACCATTCGCGAGCCTCCGGATGACGACCGTAGTGATGAATCTCTCCTGGC |
| | GGGAACAGCAAAATATCACCCGGTCGGCAAACAAATTCTCGTCCCTGATTT |
| | TTCACCACCCCCTGACCGCGAATGGTGAGATTGAGAATATAACCTTTCATT |
| | CCCAGCGGTCGGTCGATAAAAAAATCGAGATAACCGTTGGCCTCAATCGG |
| | CGTTAAACCCGCCACCAGATGGGCATTAAACGAGTATCCCGGCAGCAGGG |
| | GATCATTTTGCGCTTCAGCCATACTTTTCATACTCCCGCCATTCAGAGAAGA |
| | AACCAATTGTCCATATTGCATCAGACATTGCCGTCACTGCGTCTTTTACTGG |
| | CTCTTCTCGCTAACCAAACCGGTAACCCCGCTTATTAAAAGCATTCTGTAAC |
| | AAAGCGGGACCAAAGCCATGACAAAAACGCGTAACAAAAGTGTCTATAAT |
| | CACGGCAGAAAAGTCCACATTGATTATTTGCACGGCGTCACACTTTGCTAT |
| | GCCATAGCATTTTTATCCATAAGATTAGCGGATCCTACCTGACGCTTTTTAT |
| | CGCAACTCTCTACTGTTTCTCCATACCCGTTTTTTTGGGCTAGCGATTGAAA |
| | ACGTTGCAGTTCAAGGTTTACACCTATAAAAGAGAGAGCCGCTATCGCCTG |
| | TTTGTGGATGTACAGAGTGATATTATTGACACGCCCGGGCGACGGATGGT |
| | GATCCCCCTGGCCAGTGCACGTCTGCTGTCAGATAAAGTCTCCCGTGAACT |
| | TTACCCGGTGGTGCATATCGGGGATGAAAGCTGGCGCATGATGACCACCC |
| | AGATGGTCAGTGTGCCGGTCTCCGTCATCGGAGAAGAAGTGGCTGATCTC |
| | AGCCACCGCGAAAATGACATCAAAAACGCCATTAATCTGATGTTCTGGGG |
| | AATATAATCTAGAGGTGAGTGAGTGTGTGCGTGTGGAAGCTTGGCTGTTT |
| | TGGCGGATGAGAGAAGATTTTCAGCCTGATACAGATTAAATCAGAACGCA |
| | GAAGCGGTCTGATAAAACAGAATTTGCCTGGCGGCAGTAGCGCGGTGGTC |
| | CCACCTGACCCCATGCCGAACTCAGAAGTGAAACGCCGTAGCGCCGATGG |
| | TAGTGTGGGGTCTCCCCATGCGAGAGTAGGGAACTGCCAGGCATCAAATA |
| | AAACGAAAGGCTCAGTCGAAAGACTGGGCCTTTCGTTTTATCTGTTGTTTG |
| | TCGGTGAACGCTCTCCTGAGTAGGACAAATCCGCCGGGAGCGGATTTGAA |
| | CGTTGCGAAGCAACGGCCCGGAGGGTGGCGGGCAGGACGCCCGCCATAA |
| | ACTGCCAGGCATCAAATTAAGCAGAAGGCCATCCTGACGGATGGCCTTTTC |
| | TGCAGGCATGCAAGCTTGGCGTAATCATGGTCATAGCTGTTTCCTGTGTGA |
| | AATTGTTATCCGCTCACAATTCCACACAACATACGAGCCGGAAGCATAAAG |
| | TGTAAAGCCTGGGGTGCCTAATGAGTGAGCTAACTCACATTAATTGCGTTG |
| | CGCTCACTGCCCGCTTTCCAGTCGGGAAACCTGTCGTGCCAGCTGCATTAA |
| | TGAATCGGCCAACGCGCGGGGAGAGGCGGTTTGCGTATTGGGCGCTCTTC |
| | CGCTTCCTCGCTCACTGACTCGCTGCGCTCGGTCGTTCGGCTGCGGCGAGC |
| | GGTATCAGCTCACTCAAAGGCGGTAATACGGTTATCCACAGAATCAGGGG |
| | ATAACGCAGGAAAGAACATGTGAGCAAAAGGCCAGCAAAAGGCCAGGAA |
| | CCGTAAAAAGGCCGCGTTGCTGGCGTTTTTCCATAGGCTCCGCCCCCCTGA |
| | CGAGCATCACAAAAATCGACGCTCAAGTCAGAGGTGGCGAAACCCGACAG |
| | GACTATAAAGATACCAGGCGTTTCCCCCTGGAAGCTCCCTCGTGCGCTCTC |
| | CTGTTCCGACCCTGCCGCTTACCGGATACCTGTCCGCCTTTCTCCCTTCGGG |
| | AAGCGTGGCGCTTTCTCATAGCTCACGCTGTAGGTATCTCAGTTCGGTGTA |
| | GGTCGTTCGCTCCAAGCTGGGCTGTGTGCACGAACCCCCCGTTCAGCCCGA |
| | CCGCTGCGCCTTATCCGGTAACTATCGTCTTGAGTCCAACCCGGTAAGACA |
| | CGACTTATCGCCACTGGCAGCAGCCACTGGTAACAGGATTAGCAGAGCGA |
| | GGTATGTAGGCGGTGCTACAGAGTTCTTGAAGTGGTGGCCTAACTACGGC |
| | TACACTAGAAGAACAGTATTTGGTATCTGCGCTCTGCTGAAGCCAGTTACC |
| | TTCGGAAAAAGAGTTGGTAGCTCTTGATCCGGCAAACAAACCACCGCTGG |
| | TAGCGGTGGTTTTTTTGTTTGCAAGCAGCAGATTACGCGCAGAAAAAAAG |
| | GATCTCAAGAAGATCCTTTGATCTTTTCTACGGGGTCTGACGCTCAGTGGA |
| | ACGAAAACTCACGTTAAGGGATTTTGGTCATGAGATTATCAAAAAGGATCT |
| | TCACCTAGATCCTTTTAAATTAAAAATGAAGTTTTAAATCAATCTAAAGTAT |
| | ATATGAGTAAACTTGGTCTGACAGTTACCAATGCTTAATCAGTGAGGCACC |
| | TATCTCAGCGATCTGTCTATTTCGTTCATCCATAGTTGCCTGACTCCCCGTC |
| | GTGTAGATAACTACGATACGGGAGGGCTTACCATCTGGCCCCAGTGCTGC |
| | AATGATACCGCGAGACCCACGCTCACCGGCTCCAGATTTATCAGCAATAAA |
| | CCAGCCAGCCGGAAGGGCCGAGCGCAGAAGTGGTCCTGCAACTTTATCCG |
| | CCTCCATCCAGTCTATTAATTGTTGCCGGGAAGCTAGAGTAAGTAGTTCGC |
| | CAGTTAATAGTTTGCGCAACGTTGTTGCCATTGCTACAGGCATCGTGGTGT |
| | CACGCTCGTCGTTTGGTATGGCTTCATTCAGCTCCGGTTCCCAACGATCAA |
| | GGCGAGTTACATGATCCCCCATGTTGTGCAAAAAAGCGGTTAGCTCCTTCG |
| | GTCCTCCGATCGTTGTCAGAAGTAAGTTGGCCGCAGTGTTATCACTCATGG |
| | TTATGGCAGCACTGCATAATTCTCTTACTGTCATGCCATCCGTAAGATGCTT |
| | TTCTGTGACTGGTGAGTACTCAACCAAGTCATTCTGAGAATAGTGTATGCG |
| | GCGACCGAGTTGCTCTTGCCCGGCGTCAATACGGGATAATACCGCGCCAC |
| | ATAGCAGAACTTTAAAAGTGCTCATCATTGGAAAACGTTCTTCGGGGCGA |
| | AAACTCTCAAGGATCTTACCGCTGTTGAGATCCAGTTCGATGTAACCCACT |
| | CGTGCACCCAACTGATCTTCAGCATCTTTTACTTTCACCAGCGTTTCTGGGT |
| | GAGCAAAAACAGGAAGGCAAAATGCCGCAAAAAAGGGAATAAGGGCGA |
| | CACGGAAATGTTGAATACTCATACTCTTCCTTTTTCAATATTATTGAAGCAT |
| | TTATCAGGGTTATTGTCTCATGAGCGGATACATATTTGAATGTATTTAGAA |
| | AAATAAACAAATAGGGGTTCCGCGCACATTTCCCCGAAAAGTGCCACCTG |
| | ACGTCTAAGAAACCATTATTATCATGACATTAACCTATAAAAATAGGCGTA |
| | TCACGAGGCCCTTTCGTC (SEQ ID NO: 84) |

TABLE 5
Amino acid changes that improve Cas9-activity-based survival using short scaffold region guides
in a bacterial screen relative to the amino acid sequence of wild type Cas9 of SEQ ID NO: 133)

| Mutations above threshold of 1 | | Screen 1 | | Screen 2 |
|---|---|---|---|---|
| Cas9 Positions | Screen 1 (GD1) | (GD2) | Screen 2 (GD1) | (GD2) |
| D2T, D2Q, D2V, D2Y, D2S, D2C, D2L, | | | T, Q, V, Y, S, C, L, F | N, R, M, P, |
| D2F, D2N, D2R, D2M, D2P, D2E, D2A | | | | E, A, V, Y, C, |
| | | | | F |
| K3N, K3I, K3R, K3D, K3V, K3Y, K3S, K3F | | | N, I, R, D, V, Y, S, F | |
| K4S | | | S | |
| S6T | | | T | |
| G8P, G8T | | | P | T |
| L9V | | | | V |
| T13S, T13V | | | S, V | S |
| N14D, N14S | | | D | S |
| S15T | | | | T |
| V16I, V16L | | | I, L | |
| W18Y | | | Y | |
| A19V, A19L, A19C | | | V | L, C |
| T22E, T22D, T22V, T22Q | E, D, V | | Q | |
| D23S, D23T, D23P, D23C | | S | T, P, C | |
| E24T, E24H, E24D, E24G, E24S, E24A, | T, H, D, G | | S, H, A | T, R |
| E24R | | | | |
| Y25W, Y25F, Y25I | W, F | I | F | |
| K26N, K26H, K26R, K26E, K26D, K26V, | N, H, R, E, D, V, Y, | N, S | N, S, H, R, E, D, V, | H, E, D |
| K26Y, K26C, K26S | C | | Y | |
| V27D | | | | D |
| P28I, P28A, P28V, P28Y, P28F, P28Q, | I, A, V, Y | F | Q, A, V | H |
| P28H | | | | |
| S29R, S29L, S29A, S29K, S29Q, S29E, | R, L, A | | K, Q, L, E, A, V | K, N, I |
| S29V, S29N, S29I | | | | |
| K30T, K30M, K30I, K30L, K30A, K30G, | T, M, I, L, A, G, S, F | T, M, G, C | R, S, M, L, D, A, G, | T, S, Q, L, E, |
| K30S, K30F, K30C, K30R, K30D, K30V, | | | V, Y, W, C, F | G, V, W, C, F |
| K30Y, K30W, K30Q, K30E | | | | |
| K31N, K31R, K31M, K31Q, K31P, K31E, | N, R, M, Q, P, E, V | S, G | M, I, Q, P, L, E, D, | T, R, I, Q, E, |
| K31V, K31S, K31G, K31I, K31L, K31D, | | | A, G, V, S | V, S, W |
| K31A, K31T, K31W | | | | |
| F32T, F32M, F32L, F32A, F32Y, F32I, | T, M, L, A | T, L, Y | T, I, P, L, C | A, V |
| F32P, F32C, F32V | | | | |
| K33N, K33T, K33S, K33I, K33Q, K33H, | N, T, S, I, Q, H, P, | I, E, D, A, V, W, | N, T, S, M, P, E, A, | N, S, H, P, |
| K33P, K33R, K33L, K33E, K33D, K33A, | R, L, E, D, A, G, V, | C, F | G, V | R, L, E, D, |
| K33G, K33V, K33Y, K33C, K33F, K33W, | Y, C, F | | | G, C |
| K33M | | | | |
| V34L, V34R, V34I, V34A, V34C, V34T | L | | R, I, A, C, L | T, L |
| L35P, L35R, L35E, L35S, L35V, L35T, | P, R, E, S | V | T, E, A | K, N, S |
| L35A, L35K, L35N | | | | |
| G36A, G36S, G36N, G36Q | | A | S | N, Q |
| N37K, N37G, N37T, N37M, N37R, N37A | K, G | | T, M, R, A | |
| T38R, T38D, T38G, T38A, T38V, T38M, | R, D, G | A, V | M, D, A, G, V, S | |
| T38S | | | | |
| D39Q, D39E, D39G, D39V, D39S, D39F, | Q, E, G, V, S, F | | L, A, V | R, G, W |
| D39L, D39A, D39R, D39W | | | | |
| R40N, R40T, R40S, R40H, R40P, R40L, | N, T, S, H, P, L, E, | L, E, V | N, T, M, H, L, E, A, | V, S |
| R40E, R40G, R40V, R40W, R40C, R40F, | G, V, W, C, F | | G, V, S | |
| R40M, R40A | | | | |
| H41T, H41M, H41R, H41E, H41D, H41Y, | T, M, R, E, D, Y, C | N | I, P, C, L | T, S, M, Y, F |
| H41C, H41N, H41I, H41P, H41L, H41S, | | | | |
| H41F | | | | |
| S42M, S42H, S42R, S42E, S42A, S42L, | M, H, R, E, A, L | Q, P, E | I, H, L, E, D | H, P, E, Y |
| S42Q, S42P, S42I, S42D, S42Y | | | | |
| I43M, I43H, I43L, I43D, I43A, I43G, I43V, | M, H, L, D, A, G, V, | A, W | T, H, R, L, E, A, W, | S, Q, V, Y |
| I43Y, I43W, I43T, I43R, I43E, I43C, I43S, | Y | | C | |
| I43Q | | | | |
| K44S, K44I, K44Q, K44D, K44A, K44Y, | S, I, Q, D, A, Y | L, G, V, Y, C | M, L, E, D, A, G, Y, | P |
| K44L, K44G, K44V, K44C, K44M, K44E, | | | S, C | |
| K44P | | | | |
| K45R, K45A, K45M, K45S | R | A | M, R, S | S |
| N46S, N46H, N46P, N46L, N46E, N46G, | S, H, P, L, E, G, Y, | K, M, E | H, P, R, L, D, A, G, | I, Q, D, G, S, |
| N46Y, N46C, N46K, N46M, N46R, N46D, | C | | F | W |
| N46A, N46F, N46I, N46Q, N46W | | | | |
| L47S, L47V | | S | V | |
| I48M, I48W, I48L | M | M | W | L |
| A50I, A50L, A50V, A50C, A50T, A50S, | I, L, V, C | T, V, S | N, L, Y, F | T, S, H, L, V, |
| A50N, A50Y, A50F, A50H | | | | Y |
| L51T, L51R, L51A, L51V, L51C, L51Y, | T, R, A, V, C | R, Y | T, A, V, Y, F | R, Y, W |
| L51F, L51W | | | | |
| L52I, L52F, L52V | I | | I, F | V, F |
| F53L, F53Y | | | L, Y | |
| D54K, D54N, D54T, D54R, D54S, D54P, | K, N, T, R, S, P | K, N, A | K, R, S, H, G | N, L, E, A |
| D54A, D54H, D54G, D54L, D54E | | | | |
| S55T, S55E, S55P, S55F, S55G, S55M, | T, E | P, E, F | E, G | M, P, R |
| S55R | | | | |
| G56R, G56A, G56V, G56P, G56S | R, A | A, V | P, A, S | R, A |
| E57K, E57T, E57R, E57M, E57Q, E57L, | K, T, R, M, Q, L, V, | K, R, A | D, S | K, R, I, Q, H, |
| E57V, E57S, E57C, E57A, E57D, E57I, | S, C | | | V, S |
| E57H | | | | |
| T58S, T58G, T58R | S, G | S | S, R, G | S |
| A59L | | | L | |
| E60K, E60T, E60R, E60S, E60Q, E60H, | K, T, R, S, Q, H, A, | K, R, L, V | K, T, R, Q, D, A, G, | K, T, R, A, V, |
| E60A, E60V, E60Y, E60L, E60D, E60G | V, Y | | S | Y, L |
| A61K, A61R, A61L, A61V, A61F, A61N, | K, R | R, L, V, F | N, L | K, T, R |
| A61T | | | | |
| T62R | R | | R | |
| L64K, L64R, L64M, L64I | K, R, M | K | K, R | K, R, I |
| K65H, K65R, K65L, K65Y, K65F, K65M, | H, R, L, Y, F | H, Y, F | M, A, Y, F | H, R, Y, F |
| K65A | | | | |
| R66P | | P | | |
| T67S, T67G, T67R, T67A, T67Q, T67C, | S, G | S, R, A, G | Q, C | L |
| T67L | | | | |
| A68K, A68T, A68R, A68M, A68I, A68Q, | K, T, R, M, I, Q, L, V | K, T, R, I, Q | K, I, Q, L, V | K, T, R, M, I, |
| A68L, A68V, A68H, A68S | | | | Q, H, S |
| R70M | | M | | |
| Y72N, Y72T, Y72I, Y72L, Y72G, Y72V, | N, T, I, L, G, V, C, F | T, I, V | N, T, M, I, L, A, G, | K, T, I, L, V, |
| Y72C, Y72F, Y72M, Y72A, Y72K | | | V, C | C, F |
| T73R, T73A | R | | R, A | R |
| K76R, K76S, K76H, K76T | R | R, S | R, H | T, R |
| N77K, N77R, N77C, N77A, N77M, N77Y | K, R | K, R, C | K, A | K, R, M, Y |
| R78W, R78P | | W | P | |
| I79L | | | | L |
| C80K, C80N, C80R, C80I, C80H, C80L, | K, N, R, I, H, L, V | K, R, S, G | K, N, R, L, V | K, I, G |
| C80V, C80S, C80G | | | | |
| Q831 | | | | L |
| E84K, E84S, E84G, E84F, E84D | K, S, G | F | S | D |
| I85M, I85L | | | M, L | |
| S87T, S87M, S87G, S87V, S87K, S87R, | | | T, M, G, V | K, R, H, E, |
| S87H, S87E, S87A, S87W | | | | A, W |
| N88I, N88E, N88V, N88S, N88Q, N88A, | | | I, E, V, S | I, Q, E, A, F |
| N88F | | | | |
| E89P, E89T, E89S, E89A | | | P | T, S, A |
| M90L | | | L | |
| A91K, A91T, A91H, A91E, A91S, A91C, | | | K, T, H, E, S, C, L | N, V |
| A91L, A91N, A91V | | | | |
| K92N, K92S, K92I, K92L, K92V, K92T, | | | N, S, I, L, V | T, M, P, A, G, |
| K92M, K92P, K92A, K92G | | | | V |
| V93T, V93E, V93M | | | T, E | M, E |
| D95T, D95M, D95P, D95G, D95V, D95C, | | | T, M, P, G, V, C, L | K, I, Q, R, G, |
| D95L, D95K, D95I, D95Q, D95R | | | | C, L |
| S96T, S96H, S96A, S96K, S96N, S96R, | | | T, H, A | K, N, T, R, |
| S96L, S96E, S96V, S96Y | | | | H, L, E, V, Y |
| F98W | | | W | |
| H99K, H99R, H99E, H99A, H99Y, H99C, | K, R, E, A, Y, C, F, | Q, E, A, S, W, | N, T, Q, R, L, G, V | K, S, E, D, A, |
| H99F, H99M, H99D, H99Q, H99S, H99W, | M, D | L | | Y, C |
| H99L, H99N, H99T, H99G, H99V | | | | |
| L101R | | | | R |
| E102A, E102G, E102N, E102T, E102Q, | | A, G | N | T, Q, R, A, S |
| E102R, E102S | | | | |
| F105S | | S | | |
| V107I, V107T | | | | T |
| E108I, E108P, E108A, E108S, E108C, | I, P, A, S, C, T, M | R, H, P, A, V | P | N, H, P, A, V, |
| E108T, E108M, E108R, E108H, E108V, | | | | S |
| E108N | | | | |
| K112T, K112P, K112A | T | P | A | |
| H113Y, H113C, H113F | Y, C | F | | Y |
| E114T, E114S, E114Q, E114N, E114A, | T, S, Q | | N, A | T, S, P, A |
| E114P | | | | |
| R115K | K | | | |
| H116W | | | | W |
| I118F, I118M, I118S | F | | | M, S |
| F119Y | Y | | Y | |
| G120S, G120P | | S, P | | |
| I122V, I122L, I122R | V | L | R, L | L |
| V123T, V123H, V123S, V123L, V123N, | T, H, S | L | N, M, D, G | |
| V123M, V123D, V123G | | | | |
| D124K, D124E, D124M, D124R, D124S, | K, E, M, R, S | K, A | M | E |
| D124A | | | | |
| E125D | | | D | |
| V126I, V126D, V126A | I | D, A | | |
| A127K, A127S, A127N, A127R, A127L | K, S | N, R, L | R | K, N |
| Y128H | | | H | |
| H129L | | | | L |
| E130K, E130R, E130I, E130Q, E130A, | K, R, I, Q, A, M, D, | N, R, G | N, T, L, D, G | N, R, I, Q, H, |
| E130M, E130D, E130L, E130N, E130G, | L | | | D |
| E130T, E130H | | | | |
| K131R, K131M, K131Q, K131L, K131E, | R, M, Q, L, E, A | T, R | N, T, D, L | N, Q, H, R, |
| K131A, K131T, K131N, K131D, K131H, | | | | S |
| K131S | | | | |
| Y132S | | | S | |
| I135S | | | S | |
| Y136F | | | F | |
| K140N, K140T, K140Q, K140H, K140L, | | | N, T, Q, H, L, A, G, | N, T, A, Y, W, |
| K140A, K140G, K140Y, K140S, K140C, | | | Y, S, C, F | C, F |
| K140F, K140W | | | | |
| K141R, K141M, K141Q, K141H, K141E, | | | R, M, Q, H, E, A, V, | Q, D |
| K141A, K141V, K141W, K141F, K141D | | | W, F | |
| L142Q | | | Q | |
| V143A, V143T, V143C, V143L | | | A | T, C, L |
| D144T, D144I | | | T, I | |
| S145G, S145E, S145A, S145C | | | G | E, A, G, C |
| T146R, T146P, T146A, T146Y, T146S, | | | R, P, A, Y, S | N, I, G, S, L |
| T146N, T146I, T146G, T146L | | | | |
| D147K, D147N, D147H, D147A, D147F, | | | K, N, H, A, F | H, P, R |
| D147P, D147R | | | | |
| K148R, K148I, K148A, K148S, K148C | | | R | I, A, S, C |
| A149G, A149L, A149E, A149S | | | G | L, E, S |
| L151A, L151C, L151P | | A, C | P | |
| I154V, I154L | V | | L | V |
| L156S, L156N, L156C, L156M, L156H, | S | N, C | M, H | V, S |
| L156V | | | | |
| A157I | | I | | |
| L158V, L158S, L158A, L158T, L158C | V, S | A | T, C | A, V |
| A159S, A159H | S | H | S | H |
| M161S, M161L, M161C | | | S, L | C |
| I162L | | | | L |
| F164Y | | | Y | |
| F168L | | | L | |
| L169N, L169R, L169H, L169F, L169K, | | | N, R, H, F | K, N, R, H, |
| L169G | | | | G |
| D173E, D173P | | | E | P, E |
| N175T, N175M, N175E, N175A, N175V, | | | T, M, E, A, V, C | R, S |
| N175C, N175R, N175S | | | | |
| P176N, P176S, P176D, P176A, P176C | | | N, S, D, A | C |
| D177Y, D177M, D177E, D177C | | | Y | M, E, C |
| N178R | | | | R |
| D180T, D180S, D180M, D180H, D180L, | | | T, S, M, H, L, E, A, | S, H, E, G |
| D180E, D180A, D180G, D180Y, D180W, | | | G, Y, W, C, F | |
| D180C, D180F | | | | |
| V181N, V181P, V181E, V181D, V181S, | | | N, P, E, D, S | N, R, P, L, A |
| V181R, V181L, V181A | | | | |
| D182H, D182A, D182G, D182E, D182W | | | H, A, G | E, A, G, W |
| K183N, K183T, K183S, K183M, K183I, | | | N, T, S, M, I, Q, H, | N, M, I, Q, |
| K183Q, K183H, K183P, K183C, K183F, | | | P, C, F | H, R, L, E, A, |
| K183R, K183L, K183E, K183A, K183G, | | | | G, V, C |
| K183V | | | | |
| L184I | | | | I |
| F185T, F185Q, F185L, F185E, F185A, | | | T, Q, L, E, A | S, I, A, G, V, |
| F185S, F185I, F185G, F185V, F185Y | | | | Y |
| I186K, I186E, I186D, I186S, I186W, I186T, | | | K, E, D, S, W | T, Q, P, G, C, |
| I186Q, I186P, I186G, I186C, I186F | | | | F |
| Q187P, Q187D, Q187Y, Q187W, Q187T, | | | P, D, Y, W | T, R, S, H, P, |
| Q187R, Q187S, Q187H, Q187C | | | | C |
| L188I, L188Q, L188P, L188E, L188V, | | | I, Q, P, E, V, Y, S, F | D, G, V |
| L188Y, L188S, L188F, L188D, L188G | | | | |
| V189N, V189Y, V189W, V189C, V189T, | | | N, Y, W, C | T, R, L, F |
| V189R, V189L, V189F | | | | |
| Q190T, Q190E, Q190A, Q190V, Q190W, | | | T, E, A, V, W, C | P, R, G, C |
| Q190C, Q190P, Q190R, Q190G | | | | |
| T191S, T191P, T191L, T191E, T191D, | | | S, P, L, E, D, A, V | K, P, G |
| T191A, T191V, T191K, T191G | | | | |
| Y192H, Y192V, Y192F, Y192S, Y192L, | | | H, V, F | S, L, W |
| Y192W | | | | |
| N193Q, N193A, N193G, N193W, N193L, | | | Q, A, G, W | L, V |
| N193V | | | | |
| Q194I, Q194H, Q194L, Q194E, Q194G, | | | I, H, L, E, G, Y, S, | T, I, L, D, V, |
| Q194Y, Q194S, Q194W, Q194C, Q194T, | | | W, C | W, C |
| Q194D, Q194V | | | | |
| L195T, L195M, L195I, L195Q, L195H, | | | T, M, I, Q, H, E, D, | T, H, D, G, C |
| L195E, L195D, L195A, L195G, L195V, | | | A, G, V, S, W, F | |
| L195S, L195W, L195F, L195C | | | | |
| F196M, F196L, F196E, F196D, F196G, | | | M, L, E, D, G, V, W | T, S, M, A, |
| F196V, F196W, F196T, F196S, F196A, | | | | G, V, C |
| F196C | | | | |
| E197D, E197V, E197S, E197P | | | D, V | S, P, D |
| E198T, E198D, E198C, E198R, E198H, | | | T, D, C | R, H, P, G, Y, |
| E198P, E198G, E198Y, E198S, E198L | | | | S, L |
| N199K, N199R, N199E, N199A, N199G, | | | K, R, E, A, G | S, I, Q, P, L, |
| N199S, N199I, N199Q, N199P, N199L, | | | | D, A, C |
| N199D, N199C | | | | |
| P200K, P200T, P200V, P200F, P200N, | | | K, T, V, F | N, R, G, C, L |
| P200R, P200G, P200C, P200L | | | | |
| I201T, I201S, I201M, I201L, I201E, I201G, | | | T, S, M, L, E, G, V, | K, S, R, E, D, |
| I201V, I201Y, I201C, I201F, I201K, I201R, | | | Y, C, F | V, Y, W |
| I201D, I201W | | | | |
| N202K, N202R, N202S, N202P, N202D, | | | K, R, S, P, D, A, Y, F | K, R, M, I, L, |
| N202A, N202Y, N202F, N202M, N202I, | | | | G, V |
| N202L, N202G, N202V | | | | |
| A203I, A203Q, A203L, A203E, A203W, | | | I, Q, L, E, W, F | T, M, I, H, R, |
| A203F, A203T, A203M, A203H, A203R, | | | | L, V, S, W |
| A203V, A203S | | | | |
| S204N, S204M, S204R, S204L, S204A, | | | N, M, R, L, A, V, C | T, P, G, W, F |
| S204V, S204C, S204T, S204P, S204G, | | | | |
| S204W, S204F | | | | |
| G205T, G205I, G205Q, G205E, G205V, | | | T, I, Q, E, V, W | N, M, I, Q, |
| G205W, G205N, G205M, G205R, G205L, | | | | R, L, E, D, Y, |
| G205D, G205Y, G205S | | | | S |
| V206K, V206N, V206R, V206S, V206M, | | | K, N, R, S, M, H, P, | K, R, S, M, |
| V206H, V206P, V206L, V206E, V206D, | | | L, E, D, A, G, Y, W, | Q, H, P, E, |
| V206A, V206G, V206Y, V206W, V206F, | | | F | D, G, Y, W, F |
| V206Q | | | | |
| D207T, D207R, D207P, D207L, D207A, | | | T, R, P, L, A, G, V, Y, | I, L, W |
| D207G, D207V, D207Y, D207S, D207I, | | | S | |
| D207W | | | | |
| A208T, A208S, A208H, A208P, A208L, | | | T, S, H, P, L, E, D, | T, M, I, E, D, |
| A208E, A208D, A208G, A208V, A208Y, | | | G, V, Y, W, C, F | G, V, W, C |
| A208W, A208C, A208F, A208M, A208I | | | | |
| K209N, K209G | | | N, G | |
| A210N, A210T, A210M, A210P, A210G, | | | N, T, M, P, G, S | K, R, Q, P, |
| A210S, A210K, A210R, A210Q, A210V | | | | G, V |
| I211S, I211M, I211H, I211P, I211L, I211E, | | | S, M, H, P, L, E, A, | T, H, R, L, D, |
| I211A, 1211G, 1211V, 1211F, I211T, 1211R, | | | G, V, F | A, G, F |
| I211D | | | | |
| L212I, L212V, L212F | | | I, V, F | F |
| S213N, S213T, S213M, S213L, S213E, | | | N, T, M, L, E, C | R, I, Q, L, E, |
| S213C, S213R, S213I, S213Q, S213A, | | | | A, V |
| S213V | | | | |
| A214S, A214D, A214W, A214G | | | S, D, W | G |
| R215T, R215S, R215M, R215I, R215Q, | | | T, S, M, I, Q, P, L, E, | N, M, Q, L, |
| R215P, R215L, R215E, R215A, R215V, | | | A, V, Y, W, C, F | Y, C |
| R215Y, R215W, R215C, R215F, R215N | | | | |
| L216E, L216D, L216Y, L216H | | | E, D, Y | H, E, D |
| S217R, S217M, S217Q, S217E, S217D, | | | R, M, Q, E, D, A, C | N, M, I, Q, P, |
| S217A, S217C, S217N, S217I, S217P, | | | | D, G, V, Y |
| S217G, S217V, S217Y | | | | |
| S219T, S219I, S219Q, S219H, S219L, | | | T, I, Q, H, L, E, D, | P, L, E, V, F |
| S219E, S219D, S219A, S219G, S219W, | | | A, G, W, C, F | |
| S219C, S219F, S219P, S219V | | | | |
| R220K, R220T, R220S, R220M, R220Q, | | | K, T, S, M, Q, H, P, | S, M, I, Q, |
| R220H, R220P, R220L, R220E, R220D, | | | L, E, D, A, G, V, C, | H, P, E, D, |
| R220A, R220G, R220V, R220C, R220F, | | | F | G, V, W, F |
| R220I, R220W | | | | |
| R221E, R221G, R221Y | | | E, G | G, Y |
| L222N, L222T, L222S, L222M, L222I, | | | N, T, S, M, I, Q, H, | N, S, M, Q, |
| L222Q, L222H, L222E, L222A, L222G, | | | E, A, G, Y, W, C, F | H, A, G, Y, |
| L222Y, L222W, L222C, L222F | | | | W, C, F |
| E223S, E223P, E223D | | | S | P, D |
| N224T, N224P, N224E, N224D, N224A, | | | T, P, E, D, A, V, S, | T, P, E |
| N224V, N224S, N224W, N224L | | | W, L | |
| L225K, L225S, L225M, L225Q, L225G, | | | K, S, M, Q, G, V, Y, | S, Q, A, Y, C |
| L225V, L225Y, L225W, L225A, L225C | | | W | |
| I226K, I226N, I226T, I226S, I226M, | | | K, N, T, S, M, Q, P, | K, R, M, Q, |
| I226Q, I226P, I226L, I226D, I226G, I226V, | | | L, D, G, V | H, E, D, A, |
| I226R, I226H, I226E, I226A, I226W, I226F | | | | G, W, F |
| A227T, A227S, A227L, A227D, A227W, | | | T, S, L, D, W, C, F | H, P, G, Y, C |
| A227C, A227F, A227H, A227P, A227G, | | | | |
| A227Y | | | | |
| Q228N, Q228T, Q228P, Q228E, Q228G, | | | N, T, P, E, G, V, S, | L, A, G, V, Y |
| Q228V, Q228S, Q228W, Q228L, Q228A, | | | W | |
| Q228Y | | | | |
| L229K, L229M, L229H, L229P, L229D, | | | K, M, H, P, D, Y, W, | T, R, S, A, F |
| L229Y, L229W, L229F, L229T, L229R, | | | F | |
| L229S, L229A | | | | |
| P230M, P230H, P230G, P230S, P230L, | | | M, H, G, S | H, L, V, Y, W |
| P230V, P230Y, P230W | | | | |
| G231K, G231N, G231T, G231R, G231M, | | | K, N, T, R, M, I, Q, | M, A, L, F |
| G231I, G231Q, G231E, G231D, G231V, | | | E, D, V, S | |
| G231S, G231A, G231L, G231F | | | | |
| E232H, E232R, E232L, E232V, E232N, | | | H, R, L, V | N, M, I, P, R, |
| E232M, E232I, E232P, E232D, E232A, | | | | D, A, G, V, |
| E232G, E232W, E232C | | | | W, C |
| K233T, K233S, K233Q, K233R, K233L, | | | T, S, Q, R, L, E, G, | N, P, E, A, C |
| K233E, K233G, K233V, K233N, K233P, | | | V | |
| K233A, K233C | | | | |
| K234M, K234I, K234Q, K234L, K234D, | | | M, I, Q, L, D, A, G, | S, Q, H, R, A |
| K234A, K234G, K234Y, K234W, K234S, | | | Y, W | |
| K234H, K234R | | | | |
| N235M, N235P, N235E, N235V, N235Y, | | | M, P, E, V, Y, S, F | T, I, L, A, W |
| N235S, N235F, N235T, N235I, N235L, | | | | |
| N235A, N235W | | | | |
| G236T, G236I, G236H, G236P, G236L, | | | T, I, H, P, L, A, V | S, I, Q, H, P, |
| G236A, G236V, G236S, G236Q, G236E, | | | | L, E, D, V |
| G236D | | | | |
| L237N, L237T, L237S, L237H, L237W, | | | N, T, S, H, W, F | R, M, I, H, P, |
| L237F, L237R, L237M, L237I, L237P, | | | | G, Y, W, C, F |
| L237G, L237Y, L237C | | | | |
| F238K, F238N, F238S, F238Q, F238H, | | | K, N, S, Q, H, P, L, | R, S, M, L, |
| F238P, F238L, F238G, F238R, F238M, | | | G | E, A, V, C |
| F238E, F238A, F238V, F238C | | | | |
| G239K, G239T, G239R, G239I, G239Q, | | | K, T, R, I, Q, P, L, A, | K, T, M, I, Q, |
| G239P, G239L, G239A, G239V, G239Y, | | | V, Y, W, F | H, P, L, E, D, |
| G239W, G239F, G239M, G239H, G239E, | | | | A, V, Y, W, |
| G239D, G239C | | | | C, F |
| N240L, N240G, N240S, N240Q, N240P, | | | L, G | S, Q, P, V, |
| N240V, N240W, N240F | | | | W, F |
| L241T, L241I, L241Q, L241V, L241Y, | | | T, I, Q, V, Y, W, F | T, S, I, Q, H, |
| L241W, L241F, L241S, L241H, L241A, | | | | A, G, V, Y, W |
| L241G | | | | |
| I242K, I242N, I242T, I242S, I242Q, | | | K, N, T, S, Q, H, L, | K, S, M, Q, |
| I242H, I242L, I242E, I242A, I242G, I242V, | | | E, A, G, V, Y, W, C, | L, G, V, Y, F |
| I242Y, I242W, I242C, I242F, I242M | | | F | |
| A243N, A243T, A243I, A243H, A243D, | | | N, T, I, H, D, V, C | N, T, I, Q, R, |
| A243V, A243C, A243Q, A243R, A243L, | | | | L, G, V, Y, S, |
| A243G, A243Y, A243S | | | | C |
| L244M, L244S, L244W, L244R, L244Q, | | | M, S, W | R, M, Q, D, |
| L244D, L244Y, L244C | | | | Y, W, C |
| S245M, S245I, S245L, S245W, S245Y, | | | M, I, L, W | Y, W, F |
| S245F | | | | |
| L246N, L246S, L246M, L246H, L246G, | | | N, S, M, H, G, Y, F | T, S, H, G, Y, |
| L246Y, L246F, L246T, L246C | | | | C, F |
| G247N, G247T, G247S, G247Q, G247L, | | | N, T, S, Q, L, W, C | T, S, M, R, V, |
| G247W, G247C, G247M, G247R, G247V | | | | C |
| L248N, L248T, L248H, L248A, L248G, | | | N, T, H, A, G, V, F | K, T, M, Q, |
| L248V, L248F, L248K, L248M, L248Q, | | | | E, C |
| L248E, L248C | | | | |
| T249N, T249S, T249Q, T249P, T249A, | | | N, S, Q, P, A, W, F | R, A, V, F |
| T249W, T249F, T249R, T249V | | | | |
| P250N, P250S, P250M, P250H, P250G, | | | N, S, M, H, G | N, T, S, Q, L, |
| P250T, P250Q, P250L, P250D, P250A, | | | | D, A, W, C |
| P250W, P250C | | | | |
| N251T, N251I, N251H, N251R, N251E, | | | T, I, H, R, E | L, E, A, G, V, |
| N251L, N251A, N251G, N251V, N251S | | | | S |
| F252T, F252A, F252V, F252Q, F252P, | | | T, A, V | Q, P, L, A |
| F252L | | | | |
| K253N, K253T, K253R, K253S, K253M, | | | N, T, R, S, M, I, L, | N, S, Q, L, |
| K253I, K253L, K253G, K253Q, K253E, | | | G | E, G, V, Y, |
| K253V, K253Y, K253W, K253C, K253F | | | | W, C, F |
| S254T, S254A, S254G, S254F, S254P, | | | T, A, G, F | P, V |
| S254V | | | | |
| N255T, N255S, N255P, N255E, N255G, | | | T, S, P, E, G, V, Y, C | I, P, D, A, G, |
| N255V, N255Y, N255C, N255I, N255D, | | | | V |
| N255A | | | | |
| F256N, F256Q, F256H, F256L, F256E, | | | N, Q, H, L, E, A, G, | N, T, S, R, L, |
| F256A, F256G, F256V, F256Y, F256T, | | | V, Y | A, G, V, C |
| F256S, F256R, F256C | | | | |
| D257S, D257Q, D257L, D257V | | | S | Q, L, V |
| L258R, L258S, L258M, L258G, L258Y, | | | R, S, M, G, Y, F | T, P, E, D, F |
| L258F, L258T, L258P, L258E, L258D | | | | |
| A259N, A259M, A259I, A259Q, A259P, | | | N, M, I, Q, P, G, V, | M, P, L, E, |
| A259G, A259V, A259S, A259W, A259C, | | | S, W, C, F | W, C |
| A259F, A259L, A259E | | | | |
| E260D, E260A, E260G, E260C, E260I, | | | D, A, G, C | I, L, D, V |
| E260L, E260V | | | | |
| D261E, D261R, D261L, D261A | | | E | R, L, A |
| A262N, A262R, A262L, A262D, A262C, | | | N, R, L, D, C | M, E, G, V |
| A262M, A262E, A262G, A262V | | | | |
| K263R, K263E, K263G, K263A, K263S | | | R, E, G | A, S |
| L264K, L264Y, L264C, L264S, L264M, | | | K, Y, C | S, M, Q, H, |
| L264Q, L264H, L264P, L264G, L264V, | | | | P, G, V, W, F |
| L264W, L264F | | | | |
| Q265T, Q265M, Q265W, Q265S, Q265L | | | T, M, W | S, L |
| L266T, L266V, L266C, L266F, L266S, | | | T, V, C, F | S, I, E, V, Y, |
| L266I, L266E, L266Y | | | | C |
| S267I, S267P, S267L, S267A, S267V, | | | I, P, L, A, V | K, T, I, L, A, |
| S267K, S267T, S267Y | | | | Y |
| K268N, K268T, K268E, K268P, K268R, | | | N, T, E | P, R, L |
| K268L | | | | |
| D269L, D269V | | | L, V | |
| T270R, T270S, T270Q, T270E, T270A, | | | R, S, Q, E | S, A, G, V, C |
| T270G, T270V, T270C | | | | |
| Y271K, Y271N, Y271P, Y271L, Y271S, | | | K, N, P, L, S | K, N, R, Q, F |
| Y271R, Y271Q, Y271F | | | | |
| D272K, D272R, D272H, D272L, D272A, | | | K, R, H, L, A, G, Y, | K, T, R, S, M, |
| D272G, D272Y, D272W, D272C, D272F, | | | W, C, F | I, Q, P, L, A, |
| D272T, D272S, D272M, D272I, D272Q, | | | | G, V, C |
| D272P, D272V | | | | |
| D273K, D273N, D273T, D273R, D273S, | | | K, N, T, R, S, Q, H, | K, T, R, S, P, |
| D273Q, D273H, D273P, D273L, D273A, | | | P, L, A, V, Y, C | E, A, G, V, W |
| D273V, D273Y, D273C, D273E, D273G, | | | | |
| D273W | | | | |
| D274Q, D274P, D274L, D274E, D274A, | | | Q, P, L, E, A, G, V, | N, T, S, M, I, |
| D274G, D274V, D274Y, D274N, D274T, | | | Y | Q, P, L, G, V, |
| D274S, D274M, D274I, D274W | | | | W |
| L275K, L275S, L275H, L275A, L275Y, | | | K, S, H, A, Y, F | K, N, T, M, I, |
| L275F, L275N, L275T, L275M, L275I, | | | | H, E, D, G, |
| L275E, L275D, L275G, L275W, L275C | | | | Y, W, C |
| D276K, D276N, D276T, D276R, D276S, | | | K, N, T, R, S, M, I, | K, N, T, R, S, |
| D276M, D276I, D276Q, D276H, D276P, | | | Q, H, P, L, A, G, V, | M, I, Q, H, P, |
| D276L, D276A, D276G, D276V, D276Y, | | | Y, W, C, F | L, E, A, G, V, |
| D276W, D276C, D276F, D276E | | | | Y, W, C, F |
| N277K, N277I, N277E, N277Y, N277R, | | | K, I, E, Y | K, R, M, I, Q, |
| N277M, N277Q, N277H, N277G, N277F | | | | H, G, F |
| L278Q, L278A, L278F, L278W | | | Q, A, F | W |
| L279N, L279S, L279I, L279Q, L279H, | | | N, S, I, Q, H, E, D, | K, N, T, R, S, |
| L279E, L279D, L279A, L279G, L279Y, | | | | M, I, Q, H, P, |
| L279F, L279K, L279T, L279R, L279M, | | | A, G, Y, F | E, D, A, G, |
| L279P, L279V, L279W | | | | V, Y, W, F |
| A280K, A280N, A280R, A280S, A280I, | | | K, N, R, S, I, Q, H, | K, N, T, R, S, |
| A280Q, A280H, A280P, A280L, A280D, | | | | M, I, Q, H, P, |
| A280G, A280V, A280Y, A280W, A280C, | | | P, L, D, G, V, Y, W, | L, G, V, Y, W, |
| A280F, A280T, A280M | | | C, F | C, F |
| Q281T, Q281P, Q281L, Q281E, Q281V, | | | T, P, L, E, V, S | M, P, R, A, |
| Q281S, Q281M, Q281R, Q281A, Q281G, | | | | G, Y, F |
| Q281Y, Q281F | | | | |
| I282T, I282H, I282E, I282D, I282A, | | | T, H, E, D, A, G, V, | N, Q, H, R, |
| I282G, I282V, I282W, I282F, I282N, | | | W, F | E, D, A, S, W |
| I282Q, I282R, I282S | | | | |
| G283T, G283H, G283D, G283Y, G283W, | | | T, H, D, Y, W, C, L, | N, T, S, M, I, |
| G283C, G283L, G283F, G283N, G283S, | | | F | Q, P, D, V, Y, |
| G283M, G283I, G283Q, G283P, G283V | | | | W, C, L, F |
| D284S, D284H, D284T, D284A, D284Y, | | | S, H | T, A, Y, W |
| D284W | | | | |
| Q285T, Q285S, Q285M, Q285L, Q285E, | | | T, S, M, L, E, D, Y, | N, M, H, P, |
| Q285D, Q285Y, Q285W, Q285C, Q285N, | | | W, C | R, L, D, A, Y, |
| Q285H, Q285P, Q285R, Q285A, Q285F | | | | W, F |
| Y286N, Y286T, Y286Q, Y286E, Y286D, | | | N, T, Q, E, D, V, C | R, H, E, D, |
| Y286V, Y286C, Y286R, Y286H, Y286A, | | | | A, G, S |
| Y286G, Y286S | | | | |
| A287N, A287T, A287R, A287S, A287M, | | | N, T, R, S, M, I, Q, | K, N, R, S, |
| A287I, A287Q, A287H, A287P, A287E, | | | H, P, E, D, G, V, Y, | M, I, Q, H, P, |
| A287D, A287G, A287V, A287Y, A287W, | | | W, F | L, E, D, G, V, |
| A287F, A287K, A287L | | | | W, F |
| D288M, D288W, D288S, D288Q, D288P, | | | M, W | S, M, Q, P, L, |
| D288L, D288E, D288A, D288G, D288V, | | | | E, A, G, V, Y, |
| D288Y, D288F | | | | W, F |
| L289N, L289S, L289M, L289Q, L289H, | | | N, S, M, Q, H, P, D, | T, S, M, D, |
| L289P, L289D, L289G, L289V, L289Y, | | | G, V, Y, C | A, V, W, C |
| L289C, L289T, L289A, L289W | | | | |
| F290N, F290T, F290Y, F290W, F290S, | | | N, T, Y, W | T, S, I, Q, H, |
| F290I, F290Q, F290H, F290P, F290E, | | | | P, E, A, G, Y, |
| F290A, F290G, F290C | | | | C |
| L291K, L291N, L291R, L291H, L291D, | | | K, N, R, H, D, A, Y, | K, N, T, R, S, |
| L291A, L291Y, L291W, L291F, L291T, | | | W, F | M, I, Q, H, P, |
| L291S, L291M, L291I, L291Q, L291P, | | | | E, D, A, G, |
| L291E, L291G, L291V, L291C | | | | V, Y, W, C, F |
| A292N, A292R, A292P, A292S, A292E | | | N, R, P, S | E |
| A293K, A293N, A293S, A293I, A293L, | | | K, N, S, I, L, E, D, | T, H, L, D, V, |
| A293E, A293D, A293G, A293V, A293C, | | | G, V, C | C, F |
| A293T, A293H, A293F | | | | |
| K294R, K294E, K294W, K294T, K294G, | | | R, E, W | T, G, Y, S, F |
| K294Y, K294S, K294F | | | | |
| N295K, N295T, N295R, N295I, N295Q, | | | K, T, R, I, Q, P, E, V, | T, R, S, Q, P, |
| N295P, N295E, N295V, N295Y, N295W, | | | Y, W, C, F | A, G, V |
| N295C, N295F, N295S, N295A, N295G | | | | |
| L296A, L296V, L296Y, L296S, L296W, | | | A, V, Y, S, W, C, F | T, D, A, W |
| L296C, L296F, L296T, L296D | | | | |
| S297N, S297M, S297G, S297Y, S297W, | | | N, M, G, Y, W | K, N, G, Y, L |
| S297K, S297L | | | | |
| D298M, D298H, D298A, D298Y, D298N, | | | M, H, A, Y | N, R, S, Q, |
| D298R, D298S, D298Q, D298G | | | | G |
| A299T, A299R, A299E, A299V, A299C, | | | T, R, E, V, C | S, C |
| A299S | | | | |
| I300R, I300Q, I300A, I300H, I300V | | | R, Q, A | H, V |
| L301K, L301T, L301S, L301I, L301R, | | | K, T, S, I, R, A, G, V, | K, N, T, S, Q, |
| L301A, L301G, L301V, L301C, L301N, | | | C | R, V |
| L301Q | | | | |
| S303M, S303H, S303E, S303A, S303W, | | | M, H, E, A, W, L, F | K, R, E, G |
| S303L, S303F, S303K, S303R, S303G | | | | |
| D304N, D304M, D304I, D304Q, D304L, | | | N, M, I, Q, L, V, W | L, W, C |
| D304V, D304W, D304C | | | | |
| R307N, R307T, R307S, R307A, R307V, | | | N, T, S, A, V, C | Q, G |
| R307C, R307Q, R307G | | | | |
| V308Q, V308D, V308G, V308W, V308L | | | Q, D, G, W, L | |
| N309K, N309R, N309E, N309A, N309G, | | | K, R, E, A, G, S | P, D, G |
| N309S, N309P, N309D | | | | |
| T310K, T310S, T310P, T310A, T310G, | | | K, S, P, A, G, W | P, L |
| T310W, T310L | | | | |
| E311D, E311K, E311G | | | D | K, D, G |
| I312M, I312E, I312D, I312V, I312C, | | | M, E, D, V, C | N, T, L, E, A, |
| I312N, I312T, I312L, I312A, I312G, I312S | | | | G, S, C |
| T313S, T313E, T313D, T313C | | | S, E, D, C | S, E |
| K314N, K314T, K314S, K314H, K314P, | | | N, T, S, H, P, L, E, | C |
| K314L, K314E, K314D, K314A, K314G, | | | D, A, G, V, Y | |
| K314V, K314Y, K314C | | | | |
| A319S, A319Y, A319N, A319T | | | S, Y | N, T |
| S320A, S320G | | | A, G | |
| M321Y, M321F | | | Y, F | |
| I322T, I322M, I322V, I322S, I322C | T, M, V, S, C | V | T, S, M, V, C | V |
| K323N, K323T, K323R, K323S, K323M, | N, T, R, S, M, I, Q, | M, I, Q, L, A, V | N, T, S, M, I, Q, H, | N, R, M, I, |
| K323I, K323Q, K323H, K323L, K323E, | H, L, E, D, A, G, V, | | L, E, D, A, G, V, Y, | Q, L, D, V, C |
| K323D, K323A, K323G, K323V, K323C, | C, F, Y | | C, F | |
| K323F, K323Y | | | | |
| Y325V, Y325H | | V | H | |
| D326E, D326N, D326Q, D326A | E | | N, Q, E | A |
| E327T, E327S, E327I | T, S, I | | | |
| H329A, H329G, H329M, H329S, H329K, | A, G, M, S | K, R | G, C | R, S |
| H329R, H329C | | | | |
| Q330N, Q330T, Q330R, Q330L, Q330E, | N, T, R, L, E, D, G, | V | G, S, C, F | R, L, D, A, |
| Q330D, Q330G, Q330V, Q330C, Q330S, | V, C, S | | | C, F |
| Q330F, Q330A | | | | |
| D331E | E | | E | |
| L332K, L332R, L332N, L332C | K, R, N | K | C | |
| T333R, T333L, T333S, T333C, T333I, | R, L, S, C, I, A, G, | K, N, R, Q, H | N, S, L, A | K, R, M, I, L, |
| T333A, T333G, T333V, T333W, T333K, | V, W | | | G, V, C |
| T333N, T333Q, T333H, T333M | | | | |
| L334T, L334S, L334M, L334I, L334Q, | T, S, M, I, Q, H, R, | M | R, S, E, D, G, V, C | K, T, I, E, A, |
| L334H, L334R, L334E, L334A, L334G, | E, A, G, V, W | | | V, W |
| L334V, L334W, L334D, L334C, L334K | | | | |
| L335V, L335C, L335W, L335A, L335M, | V, C, W | A | | M, I |
| L335I | | | | |
| A337K, A337T, A337R, A337S, A337M, | K, T, R, S, M, L, Y, | S, Q, G, V | N, R, H, G, S, L | R, W |
| A337L, A337Y, A337C, A337Q, A337G, | C | | | |
| A337V, A337N, A337H, A337W | | | | |
| L338N, L338R, L338S, L338V, L338Y, | N, R, S, V, Y, C | A | T, R, V, S, C, F | Q, A |
| L338C, L338A, L338T, L338F, L338Q | | | | |
| V339T, V339I, V339C | T, I, C | | C | I |
| R340K | K | | | |
| Q341T, Q341R, Q341M, Q341L, Q341D, | T, R, M, L, D, A, G, | K, R, C | T, G, V, S, C | R, H, L, A, V, |
| Q341A, Q341G, Q341S, Q341K, Q341C, | S | | | F |
| Q341V, Q341H, Q341F | | | | |
| Q342K, Q342S, Q342E, Q342W, Q342C, | K, S, E, W, C, F, N, | R, L, W | M, G, S, W | K, R |
| Q342F, Q342N, Q342A, Q342G, Q342R, | A, G | | | |
| Q342L, Q342M | | | | |
| L343R, L343A, L343V, L343K, L343M, | R, A, V, K, M | D, V, W | K, T, R, M, A, G, V, | S |
| L343D, L343W, L343T, L343G, L343S | | | W | |
| P344N, P344T, P344R, P344G, P344S, | N, T, R, G, S | R, G | R | K, H, G |
| P344K, P344H | | | | |
| E345K, E345N, E345T, E345R, E345S, | K, N, T, R, S, M, Q, | S, H, Y | N, T, R, M, I, H, P, | N, T, S, A, V |
| E345M, E345Q, E345H, E345L, E345A, | H, L, A, G, V, Y, C, | | L, G, V, Y, C, F | |
| E345G, E345V, E345Y, E345C, E345F, | F | | | |
| E345I, E345P | | | | |
| K346R, K346N, K346C, K346M, K346Q, | R, N, C | M, Q, E, V, S | M, R, D | Q |
| K346E, K346V, K346S, K346D | | | | |
| Y347K, Y347R | K, R | | K, R | |
| K348R, K348I, K348A | | | | R, I, A |
| E349K, E349N, E349T, E349R, E349S, | K, N, T, R, S, M, I, | M, L, G, C | N, R, M, I, H, L, D, | N, T, I, D, G, |
| E349M, E349I, E349Q, E349H, E349P, | Q, H, P, L, D, A, G, | | G, V, S, C | V, S |
| E349L, E349D, E349A, E349G, E349V, | V, Y, W | | | |
| E349Y, E349W, E349C | | | | |
| I350T, I350L, I350V, I350M | T, L, V | | T, M, V | L, V |
| F352Y, F352A, F352C, F352T, F352S, | Y, A, C | T, S | R, L, A, V, Y, S, C | I, S |
| F352R, F352L, F352V, F352I | | | | |
| D353M, D353L, D353E, D353V, D353S, | M, L, E, V, S, C | | N, T, M, Q, H, E, A, | Q |
| D353C, D353N, D353T, D353Q, D353H, | | | V, C | |
| D353A | | | | |
| Q354R, Q354S, Q354A, Q354G, Q354Y, | R, S, A, G, Y | | T, R, H, P, L, E, D, | K, L, A, V, S |
| Q354T, Q354H, Q354P, Q354L, Q354E, | | | G | |
| Q354D, Q354K, Q354V | | | | |
| S355N, S355M, S355I, S355H, S355R, | N, M, I, H, R, A, K, | T, I, C | K, R, L, D, G, V | K, T, R, Y |
| S355A, S355K, S355G, S355T, S355C, | G | | | |
| S355L, S355D, S355V, S355Y | | | | |
| K356S, K356R, K356F, K356L, K356D | S | R, F | R, L | D |
| N357R, N357K, N357S, N357E, N357I, | R, K, S | E | K, R, I, Y, W | Y |
| N357Y, N357W | | | | |
| Y359N, Y359F | | | N, F | |
| A360G | G | | G | |
| Y362F, Y362N | F | | N, F | |
| I363V, I363S, I363P | V | | V | S, P |
| D364K, D364T, D364R, D364S, D364M, | K, T, R, S, M, H, L, | | K, N, T, R, S, M, H, | Q |
| D364H, D364L, D364A, D364G, D364V, | A, G, V, C, F | | L, A, G, V, W, C | |
| D364C, D364F, D364N, D364W, D364Q | | | | |
| G365S, G365A | | S | | A |
| G366S, G366H, G366P, G366Y, G366L, | S, H, P, Y, L, F | | K, T, L, A, Y, S, F | H, P |
| G366F, G366K, G366T, G366A | | | | |
| A367K, A367T, A367S, A367R, A367C | K, T, S | R | T, R, C | K |
| S368N, S368T, S368R, S368L, S368H | | | N, T, R, L | N, H |
| E370S, E370H, E370D, E370V, E370K, | | | S, H, D, V | K, M, P, A, |
| E370M, E370P, E370A, E370G, E370W | | | | G, W |
| E371K, E371N, E371R, E371M, E371Q, | | | K, N, R, M, Q, L, D | K, N, S, Q, |
| E371L, E371D, E371S, E371A, E371G | | | | L, A, G |
| F372L | | | | L |
| Y373Q | | | Q | |
| K374N | | | N | |
| F375T, F375S, F375H, F375L, F375V, | | | T, S, H, L, V, Y | A, W |
| F375Y, F375A, F375W | | | | |
| I376V | | | V | |
| K377N, K377M, K377Q, K377H, K377R, | | | N, M, Q, H, R, A, S | S |
| K377A, K377S | | | | |
| P378K, P378T, P378H, P378R, P378V, | | | K, T | H, R, V, S, L, |
| P378S, P378L, P378F | | | | F |
| I379K, I379N, I379T, I379Q, I379Y, I379S, | | | K, N, T, Q, Y, S, W, | H, P, R, F |
| I379W, I379C, I379H, I379P, I379R, I379F | | | C | |
| L380V | | | V | |
| E381S, E381I, E381Q, E381H, E381R, | | | S, I, Q, H, R, L, D, | N, S, H |
| E381L, E381D, E381G, E381V, E381N | | | G, V | |
| K382Q, K382R, K382L, K382P | | | Q, R, L | P |
| M383H, M383L | | | H, L | |
| D384I, D384P, D384G, D384V, D384Y, | | | I, P, G, V, Y, S | N, Q, R, L, |
| D384S, D384N, D384Q, D384R, D384L, | | | | A, G, V, C |
| D384A, D384C | | | | |
| G385Q, G385R, G385L, G385E, G385A, | | | Q, R, L, E, A, S, C | K, H, D, S |
| G385S, G385C, G385K, G385H, G385D | | | | |
| T386N, T386A, T386R, T386V | | | N, A | N, R, A, V |
| E387K, E387T, E387D, E387C, E387R, | | | K, T, D, C | R, D, V, C |
| E387V | | | | |
| E388D, E388P, E388G | | | D | P, G |
| L389V, L389F | | | V, F | F |
| L390T, L390R, L390Q, L390E, L390V, | | | T, R, Q, E, V, S | M, I, E, S |
| L390S, L390M, L390I | | | | |
| V391N, V391T, V391S, V391I, V391H, | | | N, T, S, I, H, E, D, F | K, T, S, P, L, |
| V391E, V391D, V391F, V391K, V391P, | | | | E, D, A |
| V391L, V391A | | | | |
| K392S, K392A, K392R | | | S, A | R |
| L393I, L393V, L393C | | | I, V, C | |
| N394M, N394Q, N394L, N394E, N394D, | | | M, Q, L, E, D, G, W | H, D, A, G, |
| N394G, N394W, N394H, N394A, N394V, | | | | V, Y |
| N394Y | | | | |
| R395I, R395L, R395A, R395C, R395M | | | I, L, A, C | M, C |
| L399M, L399C | | | M, C | M, C |
| T404M, T404V, T404S | | | M, V, S | |
| F405T, F405I, F405L, F405W, F405S, | | | T, I, L, W | T, S, I, Q, R, |
| F405Q, F405R, F405A, F405V | | | | A, V |
| D406H, D406E, D406Q, D406A | | | H, E | Q, E, A |
| N407Y | | | | Y |
| S409A, S409V, S409C, S409F | | | A, V, C | A, V, C, F |
| L416R, L416E, L416A, L416Y, L416W, | | | R, E, A, Y, W, F | M |
| L416F, L416M | | | | |
| G417Q, G417E, G417K, G417T | | | Q, E | K, T, Q, E |
| L419M | | | M | |
| H420Q, H420A, H420S, H420G, H420L | | | Q, A | S, G, L |
| A421P, A421E, A421D, A421G, A421V, | | | P, E, D, G, V, S | N, E |
| A421S, A421N | | | | |
| I422M, I422V, I422T, I422L | | | M, V | T, L |
| L423M, L423I | | | M, I | |
| R424M, R424Q, R424E, R424V, R424S, | | | M, Q, E, V, S | K, I, L, E, A, |
| R424K, R424I, R424L, R424A, R424C | | | | S, C |
| R425K, R425S, R425A, R425C | | | K | S, A, C |
| E427T, E427K, E427R, E427S | | | T | K, R, S |
| D428M, D428Q, D428R, D428L, D428E, | | | M, Q, R, L, E, G, V, | T, I, C |
| D428G, D428V, D428S, D428W, D428C, | | | S, W, C | |
| D428T, D428I | | | | |
| F429H, F429S, F429W, F429C, F429Y | | | H, S, W, C | H, Y |
| Y430W, Y430F | | | | W, F |
| P431Q, P431G, P431V, P431K, P431D | | | Q, G, V | K, D |
| F432V | | | V | |
| L433R | | | R | |
| K434N, K434R, K434S, K434E, K434D, | | | N, R, S, E, D, A | A, F |
| K434A, K434F | | | | |
| D435T, D435S, D435A, D435V, D435Y, | | | T, S, A, V, Y, C | M, Q, W |
| D435C, D435M, D435Q, D435W | | | | |
| N436Q, N436H, N436E, N436G, N436T, | | | Q, H, E, G | T, E, A |
| N436A | | | | |
| R437M, R437L, R437V, R437C, R437K, | | | M, L, V, C | K, N, A |
| R437N, R437A | | | | |
| E438K, E438Q, E438L, E438V, E438S, | | | K, Q, L, V, S | G, V, W |
| E438G, E438W | | | | |
| I440F, I440L, I440V | | | F | L, V |
| E441D, E441V, E441R | | | D, V | R |
| K442L, K442T, K442Q | L | | T | Q |
| T445S, T445H, T445E, T445A, T445Y, | S, H, E, A, Y, W | R, E | N, S, E, A, G, Y, W | S |
| T445W, T445R, T445N, T445G | | | | |
| F446C | | | C | |
| R447T, R447I, R447V | T | | T, I, V | I |
| Y450H, Y450C, Y450S | H, C | S | H, C | H |
| Y451W | W | W | W | |
| R457K, R457N, R457M, R457A, R457S | K, N, M, A | K, M | K, N, M, S | K |
| N459H, N459T | | | H | T |
| F462W | | | | W |
| A463S | | | S | |
| M465L, M465V | L | L | L, V | L |
| T466I, T466V, T466K, T466H | I, V | I | K | H |
| K468E, K468L | | E | L | |
| S469T, S469R, S469M, S469K, S469L | T, R | M | K | R, L |
| E470N, E470D, E470A, E470T, E470M, | N, D, A | | N, T, M, D | N, V |
| E470V | | | | |
| E471Q | | | | Q |
| T472K, T472R, T472S, T472H, T472P, | K, R, S, H, P, A | K, R, S, H | K, P, A | K, N, R, S, H |
| T472A, T472N | | | | |
| T474R, T474Y, T474H | R, Y | R | | R, H |
| P475N | | N | | |
| E479N, E479W, E479D | | | N, W | D, W |
| E480S, E480F, E480A | | | S, F | A |
| K484L, K484V, K484M | | | L, V | M |
| G485K, G485P, G485R, G485L, G485E, | | | K, P, R, L, E, D, A, | H, A, V, W |
| G485D, G485A, G485W, G485H, G485V | | | W | |
| S487C | | | | C |
| A488L, A488V, A488S | | | L, V | S, V |
| Q489N, Q489T, Q489R, Q489L, Q489D, | | | N, T, R, L, D, A | |
| Q489A | | | | |
| S490N, S490T, S490A, S490G, S490L | | | N, T, A, G | T, L |
| F491Y, F491S | | | Y, S | Y |
| I492V, I492Y | | | | V, Y |
| E493S, E493D, E493Y, E493F | | | S, D, Y, F | |
| R494T, R494V, R494L | | | T, V | L |
| M495H, M495F | | | H, F | F |
| T496Q, T496G, T496N, T496V, T496S | | | Q, G | N, V, S |
| N497Q, N497P, N497R, N497M, N497I, | | | Q, P | R, M, I, Q, P, |
| N497W | | | | W |
| F498N, F498T, F498S, F498H, F498L, | | | N, T, S, H, L, D, A, | N, R, S, E, G |
| F498D, F498A, F498V, F498C, F498R, | | | V, C | |
| F498E, F498G | | | | |
| D499R | | | | R |
| K500R, K500S, K500P, K500E, K500A, | | | R, S, P, E, A, G, V, | T, Q, H, E, G |
| K500G, K500V, K500W, K500C, K500F, | | | W, C, F | |
| K500T, K500Q, K500H | | | | |
| N501T, N501R, N501H, N501L, N501E, | | | T, R, H, L, E | S, C |
| N501S, N501C | | | | |
| L502V, L502A | | | V | A |
| P503R, P503V | | | | R, V |
| N504K, N504P, N504E, N504A, N504Y, | | | K, P, E, A, Y, L | K, T, Q, E, C |
| N504L, N504T, N504Q, N504C | | | | |
| E505Q, E505D, E505V, E505T | | | Q, D, V | T |
| K506T, K506M, K506A, K506V | | | T, M | A, V |
| V507S, V507L, V507I, V507E, V507A | | | S, L | S, I, L, E, A |
| L508H | | | H | |
| P509S, P509A | | | S | A |
| K510R, K510T | | | R | T |
| H511K, H511R, H511S, H511Q | | | K, R, S | R, Q |
| S512R, S512L | | | | R, L |
| L513K, L513T, L513P, L513M, L513I, | | | K, T, P | M, I, V, W |
| L513V, L513W | | | | |
| L514H, L514T, L514V, L514S | | | H | T, V, S |
| Y515N, Y515L, Y515A, Y515F, Y515K, | | | N, L, A, F | K, N, Q, R, |
| Y515Q, Y515R, Y515V | | | | L, V |
| E516S, E516D, E516G, E516L | | | S, D, G | L |
| Y517N, Y517L, Y517D, Y517G, Y517S, | | | N, L, D, G, S, F | E, V, S |
| Y517F, Y517E, Y517V | | | | |
| F518Y | | | Y | Y |
| T519D, T519Y, T519S, T519I, T519Q, | | | D, Y, S | I, Q, P |
| T519P | | | | |
| V520T, V520G, V520A | | | T, G | A, G |
| Y521L, Y521V | | | L | V |
| E523S, E523V, E523C, E523A | | | S, V, C | A |
| L524T, L524I, L524S, L524F | | | T | I, S, F |
| T525N, T525A, T525V, T525Y, T525S, | | | N, A, V | Y, S, L |
| T525L | | | | |
| K526H, K526P, K526R, K526S, K526L, | | | H, P, R, S | L, V, S, F |
| K526V, K526F | | | | |
| V527T, V527I, V527F, V527S, V527M, | | | T, I, F | T, S, M, A |
| V527A | | | | |
| K528R, K528I, K528Q, K528H, K528A, | | | R, I, Q, H, A, G, V, | I, P, S, W |
| K528G, K528V, K528W, K528P, K528S | | | W | |
| Y529V, Y529S, Y529R, Y529W | | | V, S | R, W |
| V530I, V530H, V530L, V530E, V530D, | | | I, H, L, E, D, G, W | T, S, M, Q, |
| V530G, V530W, V530T, V530S, V530M, | | | | A, Y, F |
| V530Q, V530A, V530Y, V530F | | | | |
| T531R, T531Q, T531L, T531E, T531D, | | | R, Q, L, E, D, A, V, | N, S, F |
| T531A, T531V, T531Y, T531N, T531S, | | | Y | |
| T531F | | | | |
| E532M, E532G, E532W, E532K, E532I, | | | M, G, W | K, I, Q, D, G, |
| E532Q, E532D, E532S | | | | S |
| G533T, G533Q, G533L, G533A, G533N, | | | T, Q, L, A | N, R, Q, E, D |
| G533R, G533E, G533D | | | | |
| M534R, M534S, M534L, M534N, M534T, | | | R, S, L | N, T, S, I, P, |
| M534I, M534P, M534G, M534V | | | | G, V |
| R535T, R535I, R535L, R535D, R535V, | | | T, I, L, D, V, S, C, F | I, D, A, G, V, |
| R535S, R535C, R535F, R535A, R535G, | | | | W |
| R535W | | | | |
| K536T, K536I, K536H, K536P, K536L, | | | T, I, H, P, L, E, D, A, | N, R, M, I, |
| K536E, K536D, K536A, K536G, K536V, | | | G, V, Y, S, W, C, F | H, L, E, D, |
| K536Y, K536S, K536W, K536C, K536F, | | | | A, V, Y, S, C, |
| K536N, K536R, K536M | | | | F |
| P537I, P537Q, P537H, P537D, P537A, | | | I, Q, H, D, A, V, C, | Q, E, D, A, |
| P537V, P537C, P537F, P537E | | | F | V, C, F |
| A538N, A538Q, A538L, A538Y, A538W, | | | N, Q, L, Y, W | T, M, H, P, |
| A538T, A538M, A538H, A538P, A538D, | | | | D, G, Y |
| A538G | | | | |
| F539R, F539S, F539P, F539L, F539D, | | | R, S, P, L, D, V, W | K, T, S, C |
| F539V, F539W, F539K, F539T, F539C | | | | |
| L540T, L540I, L540V | | | T, I, V | I |
| S541I, S541P, S541R, S541V, S541Y, | | | I, P, R, V, Y | R, D, A, G, |
| S541D, S541A, S541G, S541C | | | | V, C |
| G542N, G542Q, G542L, G542S, G542C, | | | N, Q, L, S, C | K, R, M, Q, |
| G542K, G542R, G542M, G542A | | | | L, A |
| E543S, E543P, E543L, E543G, E543V, | | | S, P, L, G, V, C | N, T, D, G, F |
| E543C, E543N, E543T, E543D, E543F | | | | |
| Q544T, Q544R, Q544S, Q544L, Q544A, | | | T, R, S, L, A, G | N, R, V |
| Q544G, Q544N, Q544V | | | | |
| K545N, K545R | | | N | R |
| K546R, K546E, K546A, K546V, K546T, | | | R, E, A, V | T, I, Q, L, G, |
| K546I, K546Q, K546L, K546G, K546Y, | | | | Y, S, C |
| K546S, K546C | | | | |
| A547N, A547M, A547D, A547G, A547H, | | | N, M, D, G | H, P, V, L, F |
| A547P, A547V, A547L, A547F | | | | |
| I548T, I548M, I548V, I548L, I548C | | | T, M, V | L, C |
| V549L, V549E, V549D, V549S, V549C | | | L, E, D, S, C | S, C |
| D550S, D550M, D550G, D550V, D550C, | | | S, M, G, V, C | R |
| D550R | | | | |
| L551K, L551E, L551D, L551N, L551S, | | | K, E, D | N, S, H, E, |
| L551H, L551A, L551Y, L551F | | | | A, Y, F |
| L552H, L552A, L552S | | | H, A | A, S |
| F553L, F553K, F553Y | | | L | K, Y |
| K554R, K554Y, K554T | | | R, Y | T, R |
| T555R, T555L, T555E, T555D, T555G, | | | R, L, E, D, G, Y, S, | N, I, P, L, Y, |
| T555Y, T555S, T555C, T555F, T555N, | | | C, F | C |
| T555I, T555P | | | | |
| N556T, N556Q, N556S, N556I, N556L, | | | T, Q | S, I, L, D, A |
| N556D, N556A | | | | |
| R557T, R557I, R557L, R557A, R557V, | | | T, I, L, A, V, W, C | I, A, Y, F |
| R557W, R557C, R557Y, R557F | | | | |
| K558N, K558T, K558R, K558L, K558Y, | | | N, T, R, L, Y, C, F | S, R, V, W, F |
| K558C, K558F, K558S, K558V, K558W | | | | |
| V559N, V559L, V559A, V559P, V559G, | | | N, L, A | P, G, W |
| V559W | | | | |
| T560I, T560Q, T560L, T560G, T560V, | | | I, Q, L, G, V, F | K, N, S, L, W |
| T560F, T560K, T560N, T560S, T560W | | | | |
| V561P, V561H, V561D, V561A | | | P | H, D, A |
| K562N, K562Q, K562H, K562P, K562R, | | | N, Q, H, P, R, L, A | T, S, R |
| K562L, K562A, K562T, K562S | | | | |
| Q563I, Q563L, Q563V, Q563W | | | I, L, V, W | I |
| L564T, L564I, L564V, L564C, L564P, | | | T, I, V, C | P, G |
| L564G | | | | |
| K565H, K565D, K565A, K565Y, K565C, | | | H, D, A, Y, C | M, Q, P, R, |
| K565M, K565Q, K565P, K565R, K565L, | | | | L, G, F |
| K565G, K565F | | | | |
| E566D, E566Y, E566L, E566A | | | D, Y, L | D, A |
| D567M, D567V, D567S, D567C, D567H, | | | M, V, S, C | H, E, A |
| D567E, D567A | | | | |
| Y568I, Y568L, Y568H | | | I, L | H |
| F569M, F569W, F569L | | | | M, W, L |
| K570M, K570A, K570G, K570S, K570W, | | | M, A, G, S, W, F | L, V, C, F |
| K570F, K570L, K570V, K570C | | | | |
| K571H, K571P, K571G, K571L, K571T, | | | H, P, G, L | T, M, R, D, |
| K571M, K571R, K571D, K571S, K571C | | | | S, C |
| I572T, I572A, I572S, I572C, I572F, I572L | | | T, A, S, C, F | L |
| E573R, E573S, E573Q, E573H, E573D, | | | R, S, Q, H, D, G, Y, | H, G, W |
| E573G, E573Y, E573L, E573F, E573W | | | L, F | |
| C574K, C574I, C574P, C574R, C574S, | | | K, I, P, R, S | A, G, V |
| C574A, C574G, C574V | | | | |
| F575K, F575T, F575I, F575D, F575G, | | | K, T, I, D, G, S | M, I, H, L, E, |
| F575S, F575M, F575H, F575L, F575E, | | | | D, A, G |
| F575A | | | | |
| D576N, D576I, D576G, D576L, D576H, | | | N, I, G, L | H, R, E, V, C |
| D576R, D576E, D576V, D576C | | | | |
| S577T, S577R, S577P, S577L, S577M, | | | T, R, P, L | M, V |
| S577V | | | | |
| V578S, V578M, V578D, V578I, V578P, | | | S, M, D | S, M, I, P, L |
| V578L | | | | |
| E579T, E579R, E579D, E579A, E579Y, | | | T, R, D, A, Y, S | I, Y, L |
| E579S, E579I, E579L | | | | |
| I580T, I580M, I580L, I580V, I580F | | | T, M | L, V, F |
| S581T, S581M, S581Q, S581K, S581R, | | | T, M, Q | K, R, L, G, V, |
| S581L, S581G, S581V, S581Y, S581C | | | | Y, C |
| G582N, G582T, G582S, G582Q, G582L, | | | N, T, S, Q, L, V, Y, F | R, Q, P, A, Y, |
| G582V, G582Y, G582F, G582R, G582P, | | | | W |
| G582A, G582W | | | | |
| V583R, V583M, V583P, V583W, V583F, | | | R, M, P, W, F | R, S, M, Y |
| V583S, V583Y | | | | |
| E584T, E584W, E584M, E584L, E584D, | | | T, W | M, L, D, G |
| E584G | | | | |
| D585Q, D585N, D585T, D585L | | | Q | N, T, L |
| R586S, R586H, R586L, R586G, R586Y, | | | S, H, L, G, Y, C | S, H, P, G, F |
| R586C, R586P, R586F | | | | |
| F587M, F587Y, F587L | | | M, Y, L | |
| N588K, N588T, N588R, N588M, N588E, | | | K, T, R, M, E, G | K, S, H, C |
| N588G, N588S, N588H, N588C | | | | |
| A589K, A589T, A589Y, A589C, A589N, | | | K, T, Y, C | N, R, F |
| A589R, A589F | | | | |
| S590R, S590H, S590A, S590V, S590C, | | | R, H, A, V, C, F | M, P, L, A, |
| S590F, S590M, S590P, S590L | | | | C, F |
| L591C, L591H | | | C | H |
| G592M, G592L, G592V, G592Y, G592S, | | | M, L, V, Y, S, W, F | T, M, H, L, V, |
| G592W, G592F, G592T, G592H | | | | Y, S, W |
| T593G, T593S, T593C | | | G | S, C |
| Y594M, Y594F | | | M | F |
| H595R, H595G, H595F, H595S | | | R, G, F | S |
| L597V, L597Q | | | V | Q |
| L598S, L598H, L598R, L598V, L598Y, | | | S, H, R, V, Y, W | H, A, G, V |
| L598W, L598A, L598G | | | | |
| K599T, K599I, K599Q, K599H, K599L, | | | T, I, Q, H, L, A, V, Y, | T, Q, R, A, |
| K599A, K599V, K599Y, K599W, K599R, | | | W | G, V, Y, S |
| K599G, K599S | | | | |
| I600E, I600F, I600P, I600G | | | E, F | P, G |
| I601S, I601F, I601T, I601M, I601G, I601V, | | | S, F | T, M, G, V, C |
| I601C | | | | |
| K602N, K602M, K602I, K602Q, K602H, | | | N, M, I, Q, H, P, R, | S, M, P, L, E, |
| K602P, K602R, K602L, K602E, K602A, | | | L, E, A, G, W, F | G, V |
| K602G, K602W, K602F, K602S, K602V | | | | |
| D603T, D603L, D603V, D603Y, D603H, | | | T, L, V, Y | H, E, Y |
| D603E | | | | |
| K604R, K604G, K604Y, K604E | | | R, G, Y | E |
| D605T, D605E, D605W, D605G | | | T, E, W | E, G |
| F606K, F606R, F606V, F606L | | | K, R, V, L | |
| L607F, L607T, L607I, L607Y, L607W | | | F | T, I, Y, W, F |
| D608S, D608Q, D608V, D608H | | | S, Q, V | H |
| N609S, N609M, N609P, N609R, N609L, | | | S, M, P, R, L, E, G, | K, H, R, D, |
| N609E, N609G, N609Y, N609K, N609H, | | | Y | V, W |
| N609D, N609V, N609W | | | | |
| E610N, E610R, E610I, E610L, E610K, | | | N, R, I, L | K, D, G |
| E610D, E610G | | | | |
| E611Q, E611L, E611A, E611C, E611T, | | | Q, L, A, C | T, H, R, G, V, |
| E611H, E611R, E611G, E611V, E611Y, | | | | Y, S, F |
| E611S, E611F | | | | |
| N612T, N612V, N612L, N612A, N612W, | | | T, V | T, L, A, W, C |
| N612C | | | | |
| E613K, E613M, E613Q, E613R, E613L, | | | K, M, Q, R, L, S | A, V |
| E613S, E613A, E613V | | | | |
| D614N, D614S, D614E, D614P, D614V, | | | N, S, E | P, E, V, Y, L |
| D614Y, D614L | | | | |
| I615R, I615S, I615M, I615H, I615P, | | | R, S, M, H, P, L, D, | K, T, R, S, P, |
| I615L, I615D, I615G, I615K, I615T, I615C, | | | G | C, F |
| I615F | | | | |
| L616V, L616F, L616C | | | V, F | C, F |
| E617T | | | | T |
| D618P, D618L | | | | P, L |
| I619T, I619M, I619L, I619V, I619F, I619A, | | | T, M, L, V, F | M, A, W |
| I619W | | | | |
| V620A, V620T, V620L, V620G | | | A | T, L, G |
| L621N, L621H, L621E, L621Y, L621W, | | | N, H, E, Y, W, F | N, T, M, H, |
| L621F, L621T, L621M, L621V, L621S | | | | V, S, W, F |
| L623N, L623M, L623V, L623T | | | N, M, V | T, M |
| T624S, T624C | | | | S, C |
| L625R | | | R | R |
| E627M, E627I, E627Q, E627H, E627P, | | | M, I, Q, H, P, L, V, | R, M, I, Q, |
| E627L, E627V, E627Y, E627C, E627R | | | Y, C | H, L, Y, C |
| R629N, R629T, R629M, R629Q, R629H, | | | N, T, M, Q, H, L, A, | I, Q, Y, F |
| R629L, R629A, R629Y, R629W, R629C, | | | Y, W, C, F | |
| R629F, R6291 | | | | |
| I632L, I632V | | | L, V | |
| E633H, E633D, E633S, E633G | | | H, D, S | D, G, S |
| E634M, E634Q, E634S, E634T, E634L, | | | M, Q, S | T, M, L, G, V |
| E634G, E634V | | | | |
| L636T, L636M, L636Y, L636S, L636F | | | T, M, Y, S, F | F |
| K637N, K637T, K637S, K637Q, K637D, | | | N, T, S, Q, D, G, W | N, R, S, Q, |
| K637G, K637W, K637R, K637L, K637A, | | | | L, A, G, V |
| K637V | | | | |
| T638K, T638N, T638S, T638Q, T638H, | | | K, N, S, Q, H, E, D, | N, S, P, L, G, |
| T638E, T638D, T638A, T638G, T638C, | | | A, G, C | V, C |
| T638P, T638L, T638V | | | | |
| Y639I, Y639L, Y639A, Y639V, Y639S, | | | I, L, A, V, S | K, T, P, C |
| Y639K, Y639T, Y639P, Y639C | | | | |
| A640M, A640I, A640G, A640V, A640L, | | | M, I, G, V, L | S, R, E, G, L |
| A640S, A640R, A640E | | | | |
| H641K, H641S, H641I, H641R, H641L, | | | K, S, I, R, L, E, A, C | N, Q, V |
| H641E, H641A, H641C, H641N, H641Q, | | | | |
| H641V | | | | |
| L642T, L642R, L642S, L642Q, L642A, | | | T, R, S | T, Q, A, V, Y |
| L642V, L642Y | | | | |
| F643I, F643L, F643G, F643W | | | I | L, G, W |
| D644T, D644P, D644R, D644L | | | T, P, R | L |
| D645W | | | W | |
| K646N, K646T, K646S, K646M, K646Q, | | | N, T, S, M, Q, H, P, | T, S, M, I, Q, |
| K646H, K646P, K646R, K646L, K646E, | | | R, L, E, D, A, G, V, | H, P, L, E, D, |
| K646D, K646A, K646G, K646V, K646Y, | | | Y, W, C, F | A, G, V, Y, |
| K646W, K646C, K646F, K646I | | | | W, C, F |
| V647L, V647D, V647G, V647S, V647T, | | | L, D, G, S | T, M, H |
| V647M, V647H | | | | |
| M648T, M648S, M648D, M648Y, M648W, | | | T, S, D, Y, W | T, Y, W, L, F |
| M648L, M648F | | | | |
| K649T, K649S, K649M, K649I, K649Q, | | | T, S, M, I, Q, H, P, | N, T, R, S, |
| K649H, K649P, K649L, K649E, K649D, | | | L, E, D, A, G, V, Y, | M, I, Q, H, P, |
| K649A, K649G, K649V, K649Y, K649W, | | | W, C, F | L, E, D, A, |
| K649C, K649F, K649N, K649R | | | | G, V, Y, W, |
| | | | | C, F |
| Q650T, Q650I, Q650E, Q650D, Q650V, | | | T, I, E, D, V, Y, F | N, T, M, I, P, |
| Q650Y, Q650F, Q650N, Q650M, Q650P, | | | | E, D, A, V, C, |
| Q650A, Q650C, Q650L | | | | L |
| L651I, L651V | | | I, V | |
| K652T, K652M, K652I, K652Q, K652H, | | | T, M, I, Q, H, P, E, | N, M, Q, H, |
| K652P, K652E, K652D, K652G, K652Y, | | | D, G, Y, W, F | R, L, E, V, Y, |
| K652W, K652F, K652N, K652R, K652L, | | | | S, W, F |
| K652V, K652S | | | | |
| R653T, R653S, R653M, R653I, R653H, | | | T, S, M, I, H, P, L, E, | N, T, S, I, H, |
| R653P, R653L, R653E, R653D, R653A, | | | D, A, G, V, Y, C, F | L, E, D, A, |
| R653G, R653V, R653Y, R653C, R653F, | | | | G, W, C, F |
| R653N, R653W | | | | |
| R654K, R654T, R654M, R654I, R654A, | | | K | T, M, I, A, V |
| R654V | | | | |
| R655T, R655M, R655I, R655L, R655A, | | | T, M, I, L, A, G, Y, | K, T, M, V, Y, |
| R655G, R655Y, R655W, R655K, R655V, | | | W | S, F |
| R655S, R655F | | | | |
| Y656W, Y656L | | | W | L, W |
| T657K, T657R, T657M, T657L, T657A, | | | K, R, M, L, A, W | R, E, G, W |
| T657W, T657E, T657G | | | | |
| G658K, G658T, G658R, G658H, G658N, | | | K, T, R, H | K, N, T, R, S, |
| G658S, G658Q, G658A | | | | Q, A |
| W659C | | | C | |
| G660S, G660Q, G660C, G660A | | | S, Q, C | A |
| R661G, R661Y, R661S, R661L | | | G | Y, S, L |
| L662K, L662N, L662T, L662M, L662D, | | | K, N, T, M, D, G | I, R, E, A, V |
| L662G, L662I, L662R, L662E, L662A, | | | | |
| L662V | | | | |
| R664Q, R664L, R664A, R664Y, R664C, | | | Q, L, A, Y, C | S, P, A, G, V |
| R664S, R664P, R664G, R664V | | | | |
| K665T, K665S, K665M, K665Q, K665P, | | | T, S, M, Q, P, E, Y | T, Q, H, A, V, |
| K665E, K665Y, K665H, K665A, K665V, | | | | Y, C, L, F |
| K665C, K665L, K665F | | | | |
| L666T, L6661 | | | T | I |
| I667L, I667V, I667C | | | L, V | C |
| N668T, N668M, N668L, N668D, N668C | | | T, M, L | D, C |
| G669S | | | | S |
| I670L, I670M, I670H, I670V | | | L | M, H, L, V |
| R671K, R671T, R671M, R671E, R671V, | | | K, T, M, E, V, C, L, F | H, P, V, S, C |
| R671C, R671L, R671F, R671H, R671P, | | | | |
| R671S | | | | |
| K673S, K673H, K673A, K673Y, K673C, | | | S, H, A, Y, C | T, S, M, R, L, |
| K673T, K673M, K673R, K673L, K673G, | | | | A, G, V |
| K673V | | | | |
| Q674T, Q674S, Q674E, Q674A, Q674G, | | | T, S, E, A, G, Y | K, N, S, R, L, |
| Q674Y, Q674K, Q674N, Q674R, Q674L | | | | E, G |
| S675Q, S675E, S675T, S675R, S675A | | | Q, E | T, R, A |
| G676K, G676N, G676R, G676Q, G676L, | | | K, N, R, Q, L, Y, W | E, S, F |
| G676Y, G676W, G676E, G676S, G676F | | | | |
| K677R, K677M, K677Q, K677H, K677Y, | | | R, M, Q, H, Y, F | T, S, A |
| K677F, K677T, K677S, K677A | | | | |
| T678R, T678A | | | R | A |
| I679L, I679V | | | L, V | |
| L680M, L680F, L680Y | | | M, F | Y |
| D681A, D681C, D681Q, D681P | | | A, C | Q, P, C |
| F682H, F682M, F682L | | | H | M, L |
| L683V | | | V | |
| K684R, K684Q, K684T | | | R, Q | T |
| S685H, S685P, S685R, S685G, S685N, | | | H, P, R, G | N, H, R, A, Y |
| S685A, S685Y | | | | |
| D686T, D686G | | | T | G |
| G687T, G687R, G687S, G687H, G687L, | | | T, R, S, H, L, D | A, V |
| G687D, G687A, G687V | | | | |
| F688T, F688P, F688E, F688D, F688R, | | | T, P, E, D | R, A, G, S, C |
| F688A, F688G, F688S, F688C | | | | |
| A689T, A689M, A689P, A689R, A689E, | | | T, M, P, R, E, V, C | S, L, E, D, G |
| A689V, A689C, A689S, A689L, A689D, | | | | |
| A689G | | | | |
| N690S, N690M, N690Q, N690H, N690R, | | | S, M, Q, H, R, D, | M, H, A, Y, |
| N690D, N690G, N690L, N690A, N690Y, | | | G, L | W |
| N690W | | | | |
| R691S, R691I, R691H, R691L, R691G, | | | S, I, H, L, G, V, C | K, N, L, D, A |
| R691V, R691C, R691K, R691N, R691D, | | | | |
| R691A | | | | |
| N692T, N692S, N692A, N692G, N692Q, | | | T, S, A, G | Q, H, G, Y |
| N692H, N692Y | | | | |
| F693L, F693W, F693M | | | L, W | M |
| M694H, M694P, M694R, M694W, M694F, | | | H, P, R, W, F | V, Y, S, W, C |
| M694V, M694Y, M694S, M694C | | | | |
| Q695K, Q695S, Q695G, Q695C, Q695N, | | | K, S, G, C | K, N, E, A, G |
| Q695E, Q695A | | | | |
| L696M | | | | M |
| I697R, I697C, I697A | | | R, C | A |
| H698A, H698G, H698Y, H698F, H698S, | | | A, G, Y, F | S, W |
| H698W | | | | |
| D699S, D699W, D699A | | | S, W | S, A |
| D700N, D700T, D700R, D700S, D700M, | | | N, T, R, S, M, Q, H, | T, I, L, E, G, |
| D700Q, D700H, D700P, D700A, D700G, | | | P, A, G, C | V |
| D700C, D700I, D700L, D700E, D700V | | | | |
| S701I, S701Q, S701R, S701E, S701G, | | | I, Q, R, E, G, W | N, R, L, A, Y, |
| S701W, S701N, S701L, S701A, S701Y, | | | | C |
| S701C | | | | |
| L702R, L702V, L702S, L702C, L702F, | | | R, V, S, C, F | P |
| L702P | | | | |
| T703S, T703G, T703D | | | S, G | D |
| K705N, K705R, K705V | | | | N, R, V |
| E706A, E706G, E706V, E706S, E706C, | | | A, G, V, S, C, L | I, R, A, Y, W |
| E706L, E706I, E706R, E706Y, E706W | | | | |
| D707M, D707L, D707E, D707P, D707C | | | M, L, E | M, P, C |
| I708M, I708L | | | M, L | L |
| Q709K, Q709R, Q709M, Q709A, Q709Y, | | | K, R, M, A, Y | T, L, A, G, S, |
| Q709T, Q709L, Q709G, Q709S, Q709C | | | | C |
| K710N, K710R, K710S, K710P, K710L, | | | N, R, S, P, L, A, V | S, I, D, A |
| K710A, K710V, K710I, K710D | | | | |
| A711S | | | | S |
| Q712K, Q712M, Q712R, Q712V, Q712W, | | | K, M, R, V, W, L, F | M, S, L |
| Q712L, Q712F, Q712S | | | | |
| V713K, V713N, V713P, V713A, V713S, | | | K, N, P, A, S | T, Q, H, R |
| V713T, V713Q, V713H, V713R | | | | |
| S714M, S714Q, S714H, S714L, S714D, | | | M, Q, H, L, D, G, V, | T, M, P, R, L, |
| S714G, S714V, S714C, S714F, S714T, | | | C, F | D, A, W, F |
| S714P, S714R, S714A, S714W | | | | |
| G715K, G715R, G715S, G715M, G715P, | | | K, R, S, M, P, L, V | T, R, S, M, A, |
| G715L, G715V, G715T, G715A | | | | V |
| Q716M, Q716H, Q716L, Q716A, Q716Y, | | | M, H, L, A, Y, F | K, M, I, S |
| Q716F, Q716K, Q716I, Q716S | | | | |
| G717K, G717R, G717H, G717P, G717A, | | | K, R, H, P, A, S, C | N, T, I, P, L, |
| G717S, G717C, G717N, G717T, G717I, | | | | A, V, C |
| G717L, G717V | | | | |
| D718K, D718N, D718R, D718S, D718M, | K, N, R, S, M, H, L | T, Q, H, G, V | K, T, R, S, Q, P, L, | K, N, R, Q, |
| D718H, D718L, D718T, D718Q, D718G, | | | G, V | L, E, A, G, F |
| D718V, D718P, D718E, D718A, D718F | | | | |
| S719N, S719C, S719T, S719D, S719K | N, C | N, C | N, T, D, C | K, D |
| L720I, L720S, L720A, L720H, L720V | I, S | A | A | H, V |
| H721K, H721R, H721M, H721Q, H721L, | K, R, M, Q, L, A, V | Q, L, V | K, T, R, Q, L, A, G, | R, P, L, V, S |
| H721A, H721V, H721T, H721G, H721P, | | | V | |
| H721S | | | | |
| E722K, E722N, E722Q, E722A, E722G, | K, N, Q, A, G | K, T, R, L, S, W | I, P, D, A, G, V, F | K, N, T, R, L, |
| E722T, E722R, E722L, E722S, E722W, | | | | D, V, C, F |
| E722I, E722P, E722D, E722V, E722F, | | | | |
| E722C | | | | |
| H723K, H723N, H723T, H723R, H723M, | K, N, T, R, M, I, Q, | N, T, R, M, I, | N, S, M, I, Q, L, E, | N, T, R, M, |
| H723I, H723Q, H723A, H723S, H723C, | A, S, C | Q, L, G | A, G, V, C | Q, E |
| H723L, H723G, H723E, H723V | | | | |
| I724V, I724L, I724M, I724A, I724C, I724F | V, L | | M, L, A, V, C | V, F |
| A725K, A725N, A725T, A725R, A725M, | K, N, T, R, M, Q, H, | K, R, Q, V, C | K, N, R, Q, H, L, V, | K, N, T, R, |
| A725Q, A725H, A725P, A725L, A725G, | P, L, G, V, Y, W, C, | | Y, W, F | M, H, P, G, |
| A725V, A725Y, A725W, A725C, A725F | F | | | W |
| N726K, N726R, N726S, N726Q, N726A, | K, R, S, Q | R, A | K, T, R, W, C | K, R, G |
| N726T, N726W, N726C, N726G | | | | |
| L727T, L727M, L727V, L727Y, L727W, | T, M, V, Y, W | T, G, C | A, G, V, Y, S | V, Y, W |
| L727G, L727C, L727A, L727S | | | | |
| A728Q, A728H, A728P, A728E, A728D, | Q, H, P, E, D, G, Y, | R, P, L, D, G, | Q, P, L, G, Y, W, F | K, R, Q, P, E, |
| A728G, A728Y, A728S, A728W, A728F, | S, W, F | S, W, F | | D, G, S, W, F |
| A728R, A728L, A728K | | | | |
| G729N, G729T, G729R, G729S, G729M, | N, T, R, S, M, I, Q, | N, T, R, S, M, I, | K, N, T, R, S, M, I, | N, T, R, S, |
| G729I, G729Q, G729H, G729P, G729L, | H, P, L, E, D, A, V, | Q, H, L, E, D, | Q, H, L, E, D, A, V, | M, I, Q, H, P, |
| G729E, G729D, G729A, G729V, G729Y, | Y, W, C, F | A, V, Y, W, C, F | Y, W, C, F | L, E, D, A, V, |
| G729W, G729C, G729F, G729K | | | | Y, W, C, F |
| S730K, S730N, S730T, S730M, S730H, | K, N, T, M, H, P, A, | N, P, G, W, C | K, N, T, M, I, Q, G, | K, N, R, I, H, |
| S730P, S730A, S730G, S730Y, S730C, | G, Y, C | | C | P, D, A, G, C |
| S730W, S730I, S730Q, S730R, S730D | | | | |
| P731A, P731T, P731M, P731R, P731H, | A | T, M | R, H, A, G, V | A, S |
| P731G, P731V, P731S | | | | |
| A732N, A732T, A732S, A732I, A732H, | N, T, S, I, H, G, C | R, S, M | N, T, S, Q, P, G, W, | N, T, R, G, V |
| A732G, A732C, A732R, A732M, A732Q, | | | C | |
| A732P, A732W, A732V | | | | |
| I733T, I733Q, I733A, I733V, I733Y, I733W, | T, Q, A, V, Y, W | E, Y | T, Q, A, V, C, F | N, H, R, L, |
| I733E, I733C, I733F, I733N, I733H, | | | | V, S |
| I733R, I733L, I733S | | | | |
| K734R, K734H, K734S, K734N, K734V, | R, H, S | N | R, V, C | R, W |
| K734C, K734W | | | | |
| K735R, K735M | R | | R, M | R |
| G736T, G736S, G736W, G736A, G736V | T, S | W | T, A, V | S, A |
| I737V, I737T, I737R | V | V | V | T, R, V |
| L738R, L738M, L738V, L738Y, L738W, | R, M, V, Y, W, F | M, V, Y, W, F | R, M, Q, H, V, W, F | T, R, M, I, A, |
| L738F, L738Q, L738H, L738T, L738I, | | | | Y, W, F |
| L738A | | | | |
| Q739K, Q739R, Q739T, Q739M, Q739I, | K, R | | T, M, I, L, A, S | R |
| Q739L, Q739A, Q739S | | | | |
| T740A, T740S, T740C, T740M | A, S, C | M, C | | A, S |
| V741L, V741E, V741G, V741C | L | | L, E, G, C | E, G |
| K742R | R | | R | |
| V743I, V743L, V743R | I, L | I | I, L | I, R |
| D745T, D745E, D745V, D745L | | T, E | V | L |
| E746V | | | V | |
| L747S, L747F | | | | S, F |
| V748I, V748C, V748T | I | C | I | T |
| K749L, K749M, K749A, K749C, K749F | L | M | | L, A, C, F |
| V750A, V750S, V750T, V750I, V750C, | A | S | T, I, A, C | S, M, R, A |
| V750M, V750R | | | | |
| M751L, M751E | | L | L, E | |
| G752L, G752V | | | | L, V |
| R753K, R753N, R753T, R753S, R753M, | K, N, T, S, M, H, L, | S, P, L, G, C | K, N, S, Q, H, G, C, | N, T, S, M, P, |
| R753H, R753L, R753E, R753D, R753G, | E, D, G, Y, C, F | | F | L, V, Y |
| R753Y, R753C, R753F, R753P, R753Q, | | | | |
| R753V | | | | |
| H754M, H754R, H754G, H754T, H754L, | M, R, G | T, R, L, C | R, M, E, D, A, G, S | M, L, C |
| H754C, H754E, H754D, H754A, H754S | | | | |
| K755L | | | L | L |
| P756L | | | | L |
| E757S, E757M, E757A, E757L, E757G | | | S, M, A | L, G |
| N758T, N758L, N758Y, N758S, N758F, | | | T, L, Y, S, F | M, A, V, Y |
| N758M, N758A, N758V | | | | |
| I759L, I759A | | | L | A |
| V760I, V760Q, V760E, V760C | | | I, Q | E, C |
| I761T | | | | T |
| M763T, M763I, M763V, M763S, M763H | | | T, I, V, S | H, V |
| A764V, A764S, A764G | | | V | S, G |
| R765S | | | S | S |
| E766K, E766T, E766Y, E766W, E766R, | | | K, T, Y, W | T, R, M, G, |
| E766M, E766G | | | | W |
| N767K, N767T, N767M, N767P, N767E, | | | K, T, M, P, E, A, G, | R, H, L, V, S, |
| N767A, N767G, N767F, N767R, N767H, | | | F | F |
| N767L, N767V, N767S | | | | |
| Q768K, Q768T, Q768L, Q768V, Q768W, | | | K, T, L, V, W, F | Y, S, W, F |
| Q768F, Q768Y, Q768S | | | | |
| T769Q, T769P, T769G, T769S, T769C, | | | Q, P, G, S, C | M, H, R, G, |
| T769M, T769H, T769R, T769V, T769Y | | | | V, Y |
| T770G, T770S | | | G, S | S |
| Q771I, Q771A, Q771G, Q771T, Q771P, | | | I, A, G | T, P, L, E, Y, |
| Q771L, Q771E, Q771Y, Q771S | | | | S |
| K772R, K772P, K772S, K772W, K772C, | | | R, P, S, W, C | H, P |
| K772H | | | | |
| G773A, G773S, G773W, G773H | | | A, S, W | H |
| Q774T, Q774H, Q774L, Q774G, Q774S, | | | T, H, L, G, S | V |
| Q774V | | | | |
| K775T, K775M, K775Q, K775H, K775L, | | | T, M, Q, H, L, E, D, | N, R, M, H, |
| K775E, K775D, K775A, K775V, K775Y, | | | A, V, Y | A, C |
| K775N, K775R, K775C | | | | |
| N776S, N776R, N776D, N776W, N776C, | | | S, R, D, W, C | T, S, Q, E, W |
| N776T, N776Q, N776E | | | | |
| S777A, S777V, S777R | | | A, V | R |
| R778K, R778H, R778P, R778Y, R778N, | | | K, H, P, Y | N, M, L, S, |
| R778M, R778L, R778S, R778W, R778F | | | | W, F |
| E779T, E779S, E779L, E779A, E779I | | | T, S, L, A | I |
| M781W, M781F, M781K, M781I, M781Q, | | | W, F | K, I, Q, L, D, |
| M781L, M781D, M781G, M781V, M781Y | | | | G, V, Y, W |
| K782S, K782Q, K782R, K782E, K782Y, | | | S, Q, R, E, Y | N, A, Y |
| K782N, K782A | | | | |
| R783N, R783T, R783M, R783I, R783P, | | | N, T, M, I, P, L, E | M, I, Q, H, |
| R783L, R783E, R783Q, R783H, R783A, | | | | A, G, V, Y, |
| R783G, R783V, R783Y, R783W, R783C | | | | W, C |
| I784T, I784V, I784L | | | T, V | L |
| E785Q, E785S, E785M, E785I, E785W | | | Q | S, M, I, W |
| E786H, E786P, E786R, E786Y, E786S, | | | H, P, R, Y, S, L | N, Q, H, D, |
| E786L, E786N, E786Q, E786D, E786A | | | | A, L |
| G787S, G787L | | | S | L |
| I788L, I788V, I788T, I788A, I788C | | | L, V | T, A, V, C |
| K789N, K789H, K789G, K789V, K789C, | | | N, H, G, V, C | Q, H, R, S |
| K789Q, K789R, K789S | | | | |
| E790Q, E790A, E790V, E790S, E790R, | | | Q, A, V, S | R, L |
| E790L | | | | |
| L791M | | | M | |
| G792Y, G792K, G792S, G792D | | | Y | K, S, D |
| S793T | | | T | |
| Q794H, Q794P, Q794V, Q794W, Q794T, | | | H, P, V, W | T, S, M, P, L, |
| Q794S, Q794M, Q794L, Q794A, Q794G | | | | A, G, V |
| I795Q, I795L | | | | Q, L |
| K797T, K797M, K797A, K797L, K797Y, | | | T, M, A | L, A, Y, S |
| K797S | | | | |
| E798S, E798L, E798A, E798Y, E798T, | | | S, L, A, Y | T, L, V, W |
| E798V, E798W | | | | |
| H799T, H799L, H799V, H799W, H799K | | | T, L, V, W | K |
| P800R | | | | R |
| V801M, V801I, V801P, V801C, V801S | | | M, I, P, C | S |
| E802P, E802G, E802V, E802L, E802N, | | | P, G, V, L | N, S, R |
| E802S, E802R | | | | |
| N803S, N803G | | | | S, G |
| T804H, T804P, T804Y, T804C, T804L, | | | H, P, Y, C, L, F | N, R, M, D, |
| T804F, T804N, T804R, T804M, T804D, | | | | A, G |
| T804A, T804G | | | | |
| Q805A, Q805G, Q805Y, Q805L | | | A, G, Y, L | Y |
| L806P, L806F | | | | P, F |
| Q807K, Q807T, Q807A, Q807I, Q807R, | | | K, T, A | I, R, L, E, S |
| Q807L, Q807E, Q807S | | | | |
| N808R, N808W, N808Y | | | R, W | Y |
| E809L, E809T, E809P, E809G, E809V | | | L | T, P, L, G, V |
| K810R | | | | R |
| L811T, L811I, L811V, L811F, L811A, | | | T, I, V, F | A, V, Y |
| L811Y | | | | |
| Y812V, Y812W, Y812F, Y812M, Y812A | | | V, W, F | M, A, F |
| Y814F, Y814V | | | F | V |
| Y815N, Y815H | | | | N, H |
| L816S, L816E, L816M, L816V, L816C, | | | S, E | M, V, C, F |
| L816F | | | | |
| Q817S | | | | S |
| N818D, N818A, N818G, N818S | | | D, A, G, S | D |
| G819A | | | A | |
| R820K, R820S, R820V, R820Y, R820C | | | K, S | V, Y, C |
| V824T, V824Q, V824L, V824D, V824C, | | | T, Q, L, D, C | L, E, G, Y, F |
| V824E, V824G, V824Y, V824F | | | | |
| D825N, D825P, D825E, D825Y | | | N, P | N, E, Y |
| Q826K, Q826M, Q826Y, Q826S, Q826N, | | | K, M, Y, S | N, T, A |
| Q826T, Q826A | | | | |
| E827T, E827I, E827G, E827P, E827S, | | | T, I, G | P, S, W |
| E827W | | | | |
| L828V | | | | V |
| D829L | | | L | |
| I830P, I830V, I830S, I830H, I830L | | | P, V, S | H, L, S |
| N831S, N831I, N831A, N831F, N831R, | | | S, I, A, F | R, I, L, G, V, |
| N831L, N831G, N831V, N831Y | | | | Y, F |
| R832I, R832S, R832H | | | I, S | H |
| L833E, L833V, L833M | | | E, V | M |
| S834T, S834I, S834Q, S834R, S834V, | | | T, I, Q, R, V | N, Q, L |
| S834N, S834L | | | | |
| D835N, D835H, D835S, D835C, D835L, | | | N, H, S, C, L | T, H, A, G, V, |
| D835T, D835A, D835G, D835V | | | | L |
| Y836H, Y836L | | | H, L | |
| V838I, V838G | | | I, G | I, G |
| I841L | | | | L |
| V842M, V842I, V842H, V842R | | | M, I, H | R |
| Q844S, Q844A, Q844G, Q844K, Q844T, | | | S, A, G | K, T, R |
| Q844R | | | | |
| F846I, F846M, F846Y | | | I | M, Y |
| L847K | | | K | |
| K848R, K848A, K848F | | | R | A, F |
| D849N, D849Q | | | N, Q | |
| I852L, I852W, I852N, I852M, I852A, | | | L, W | N, M, L, A, |
| I852V, I852Y, I852S | | | | V, Y, S, W |
| D853S, D853V, D853Y, D853T, D853H, | | | S, V, Y | T, S, H, P, A, |
| D853P, D853A, D853G | | | | G |
| K855I, K855L, K855V | | | I, L, V | |
| V856A | | | A | |
| L857I | | | I | |
| T858R | | | | R |
| R859L, R859I, R859S | | | L | I, S |
| S860A | | | A | |
| D861E | | | | E |
| K862R, K862H, K862L, K862G, K862V, | | | R, H, L, G, V, S | H, E, Y |
| K862S, K862E, K862Y | | | | |
| G865L, G865R, G865W, G865C | | | L | R, W, C |
| S867L, S867G, S867T, S867C | | | L, G | T, G, C |
| D868K, D868T, D868C, D868E, D868A | | | K, T, C | E, A |
| N869T, N869H, N869E, N869C, N869G, | | | T, H, E, C | T, G, S |
| N869S | | | | |
| V870M, V870T, V870L | | | M | T, L |
| P871A, P871L | | | A | L |
| S872G | | | G | |
| E873R, E873S, E873M, E873A, E873Y, | | | R, S, M, A, Y, W | D, G, Y, C |
| E873W, E873D, E873G, E873C | | | | |
| E874S, E874Q, E874H, E874P, E874L, | | | S, Q, H, P, L, D, A, | N, T, S, D, V, |
| E874D, E874A, E874G, E874V, E874Y, | | | G, V, Y, W, C | Y, W, C, F |
| E874W, E874C, E874N, E874T, E874F | | | | |
| V875T, V875I, V875R, V875S, V875C | | | T | I, R, S, C |
| V876C | | | | C |
| K877N, K877T, K877S, K877M, K877I, | | | N, T, S, M, I, R, L, | D, V, W, C |
| K877R, K877L, K877D, K877A, K877C, | | | D, A, C, F | |
| K877F, K877V, K877W | | | | |
| K878P, K878E, K878A, K878V, K878S, | | | P, E, A, V, S, W | H, R, E, G, |
| K878W, K878H, K878R, K878G, K878Y | | | | Y, W |
| M879R, M879L, M879C, M879N, M879Q | | | R, L, C | N, Q, C |
| K880I, K880Q, K880L, K880A, K880G, | | | I, Q, L, A, G | T, R, I, Q, V |
| K880T, K880R, K880V | | | | |
| N881T, N881S, N881M, N881Q, N881P, | | | T, S, M, Q, P, D, G, | K, R, G, W |
| N881D, N881G, N881V, N881K, N881R, | | | V | |
| N881W | | | | |
| Y882D, Y882A, Y882S, Y882W, Y882F, | | | D, A, S, W, F | T, R, V, S |
| Y882T, Y882R, Y882V | | | | |
| R884M, R884V, R884L, R884W | | | M, V, L | W |
| Q885T, Q885S, Q885R, Q885L, Q885A, | | | T, S, R, L, A, C | V, C |
| Q885C, Q885V | | | | |
| L886Y, L886S, L886M | | | Y, S | M |
| L887T, L887F, L887R | | | T, F | R |
| N888T, N888Q, N888H, N888R, N888A, | | | T, Q, H, R, A, G, W, | S, R, A, V |
| N888G, N888W, N888F, N888S, N888V | | | F | |
| A889C, A889K | | | C | K |
| K890S, K890R, K890Q, K890H, K890G | | | S | R, Q, H, G |
| L891G | | | | G |
| I892V, I892C, I892L | | | V | V, C, L |
| T893A, T893G | | | A, G | G |
| Q894T, Q894H, Q894E, Q894G, Q894V, | | | T, H, E, G, V, L, F | D, G, W, C |
| Q894L, Q894F, Q894D, Q894W, Q894C | | | | |
| R895K, R895E, R895S | | | K, E | S |
| K896R | | | R | |
| F897W | | | | W |
| D898W | | | W | |
| N899R, N899S, N899W, N899C, N899T, | | | R, S, W, C | T, A, W, C |
| N899A | | | | |
| L900D | | | D | |
| T901M, T901S, T901C | | | | M, S, C |
| K902T, K902M, K902I, K902A, K902V, | | | T, M, I, A, V, Y, C, F | S, V |
| K902Y, K902C, K902F, K902S | | | | |
| A903M, A903I, A903S, A903R, A903F | | | M, I, S | R, F |
| E904I, E904A, E904V, E904S | | | I | A, V, S |
| R905T, R905M, R905Q, R905L, R905E, | | | T, M, Q, L, E, D, A, | T, M, Q, E, |
| R905D, R905A, R905Y, R905S, R905W, | | | Y, S, W, C, F | G, V, Y, W, F |
| R905C, R905F, R905G, R905V | | | | |
| G906N, G906Q, G906C | | | N, Q, C | |
| G907S, G907H, G907A | | | S | S, H, A |
| L908I, L908V, L908F | | | I, V | I, F |
| S909D, S909L, S909N, S909T, S909R | | | D, L | N, T, R |
| E910H, E910V, E910F | | | | H, V, F |
| L911M, L911H, L911E, L911D, L911V, | | | M, H, E, D, V, Y, S, | M, I, P, V, Y, |
| L911Y, L911S, L911W, L911I, L911P, | | | W | W, C, F |
| L911C, L911F | | | | |
| D912S, D912A, D912V, D912C | | | S | A, V, C |
| K913R, K913I, K913L, K913G, K913V | | | R, I, L | G, V |
| A914M, A914C, A914K, A914T, A914H, | | | M, C | K, T, M, H, L, |
| A914L, A914Y | | | | Y, C |
| G915N, G915H, G915A, G915V, G915F, | | | N, H, A, V, F | K, L, W, F |
| G915K, G915L, G915W | | | | |
| F916M | | | | M |
| I917V, I917L | | | V | L |
| K918R, K918W | | | | R, W |
| R919Q, R919I, R919Y | | | Q | I, Y |
| Q920L, Q920A, Q920F | | | | L, A, F |
| V922T, V922Q, V922A, V922C, V922N, | | | T, Q, A, C | N, E, S |
| V922E, V922S | | | | |
| E923N, E923Q, E923V, E923S, E923M, | | | N, Q, V | S, M, I, H, L, |
| E923I, E923H, E923L, E923A | | | | A, V |
| T924L, T924M, T924V | | | L | M, I |
| R925W | | | W | |
| Q926S, Q926L, Q926W, Q926C, Q926F, | | | S, L, W, C, F | N, M, W, C, |
| Q926N, Q926M | | | | F |
| I927Q, I927R, I927L | | | | Q, R, L |
| T928N, T928I, T928S, T928A, T928V, | | | N, I, S | A, V, C |
| T928C | | | | |
| K929N, K929T, K929Q, K929R, K929L | | | N, T, Q, R | Q, L |
| H930T, H930S, H930I, H930Q, H930E, | | | T, S, I, Q, E, A, C, F | T, M, Q, L, E, |
| H930A, H930C, H930F, H930M, H930L, | | | | V, F |
| H930V | | | | |
| V931L | | | L | |
| A932T, A932S, A932V | | | | T, S, V |
| Q933L, Q933N, Q933R, Q933M | | | L | N, R, M, L |
| I934V | | | V | |
| L935D | | | | D |
| D936T, D936S | | | | T, S |
| S937K, S937Q, S937R, S937N, S937V, | | | K, Q, R | N, V, W |
| S937W | | | | |
| R938K, R938Q, R938G | | | K | Q, G |
| M939P, M939Y, M939L, M939F | | | | P, Y, L, F |
| T941Q, T941P, T941A, T941C | | | Q, P, A | C |
| K942T, K942A, K942V, K942S, K942R, | | | T, A, V, S | T, R, D |
| K942D | | | | |
| Y943K, Y943T, Y943H, Y943L, Y943V, | | | K, T, H, L, V | N, R, V, S, W |
| Y943N, Y943R, Y943S, Y943W | | | | |
| D944A | | | | A |
| E945S, E945I, E945H, E945G, E945A, | | | S, I, H, G | A, V, L |
| E945V, E945L | | | | |
| N946T, N946I, N946H, N946Y | | | T, I, H | T, I, Y |
| D947N, D947M, D947I, D947H, D947R, | | | N, M, I, H, R, G | T, S |
| D947G, D947T, D947S | | | | |
| K948S, K948Q, K948R, K948L, K948D, | | | S, Q, R, L, D, W | N, S, V |
| K948W, K948N, K948V | | | | |
| L949K, L949Q, L949A, L949G, L949V | | | K, Q | A, G, V |
| I950K, I950M, I950L | | | K, M | L |
| R951M, R951E | | | M | E |
| E952Q, E952G, E952N | | | Q, G | N |
| K954R, K954C | | | | R, C |
| I956L | | | L | |
| T957P, T957A, T957S, T957K, T957I, | | | P, A, S | K, I, L, S |
| T957L | | | | |
| L958C | | | | C |
| K959S | | | | S |
| S960T, S960Q, S960P, S960G, S960V, | | | T, Q, P | G, V, Y |
| S960Y | | | | |
| K961T, K961L, K961A, K961N, K961G | | | T, L, A | N, G |
| L962Q, L962P, L962C | | | Q | P, C |
| V963M, V963P, V963L, V963F, V963S, | | | M, P, L, F | S, M, G, F |
| V963G | | | | |
| S964M, S964Q, S964R, S964L, S964A, | | | M, Q, R, L, A | T |
| S964T | | | | |
| D965V, D965S, D965T, D965H, D965E | | | V, S | T, H, E, V |
| F966T | | | | T |
| R967P, R967Q, R967L | | | P | Q, P, L |
| K968M, K968A, K968V, K968L, K968F, | | | M, A, V, L, F | R, S |
| K968R, K968S | | | | |
| D969V, D969M | | | V | M |
| F970L, F970G, F970V | | | L | L, G, V |
| Q971R, Q971L, Q971A, Q971H, Q971G, | | | R, L, A | H, G, V, S |
| Q971V, Q971S | | | | |
| F972T, F972L, F972K, F972G, F972S | | | T, L | K, G, S |
| Y973H, Y973E, Y973V, Y973N, Y973R, | | | H, E, V | N, R, A |
| Y973A | | | | |
| K974F | | | | F |
| V975S, V975I, V975H, V975R, V975G, | | | S, I, H, R, G, L | M, A, G, L |
| V975L, V975M, V975A | | | | |
| E977Q, E977Y, E977T, E977S, E977F | | | Q, Y | T, S, F |
| I978S, I978E, I978M, I978P, I978L, I978G | | | S, E | M, P, L, G |
| N979H, N979A | | | H | A |
| N980T, N980S, N980I, N980D, N980C, | | | T, S, I, D, C | T, M, L, D, A |
| N980M, N980L, N980A | | | | |
| Y981T, Y981V, Y981N, Y981M, Y981L, | | | T, V | N, M, L, C |
| Y981C | | | | |
| H982P, H982V, H982I, H982D, H982A | | | P, V | I, P, D, A |
| H983N | | | | N |
| A984R, A984V | | | | R, V |
| H985A, H985R, H985F | | | A | R, F |
| D986K | | | | K |
| L989I, L989M, L989F | | | I | M, F |
| N990S, N990M, N990H | | | S | M, H |
| A991V | | | V | |
| V993M, V993L | | | M, L | |
| G994A | | | | A |
| T995I, T995R, T995A, T995W | | | I, R | A, W |
| A996L, A996V, A996T, A996C | | | L, V | T, C |
| I998S, I998L, I998M, I998R | | | S, L | M, R |
| K999R, K999M, K999Y | | | R | M, Y |
| K1000I, K1000N, K1000T, K1000M, | | | I | N, T, M, R, G |
| K1000R, K1000G | | | | |
| Y1001P, Y1001F | | | | P, F |
| P1002N | | | | N |
| K1003T, K1003H, K1003L, K1003E, | | | T, H, L, E, A | R, V, S, F |
| K1003A, K1003R, K1003V, K1003S, | | | | |
| K1003F | | | | |
| E1005T, E1005R, E1005S, E1005L, | | | T, R | S, L, G |
| E1005G | | | | |
| S1006K, S1006R, S1006H, S1006G, | | | K, R, H, G, Y | E, A, G, Y |
| S1006Y, S1006E, S1006A | | | | |
| E1007T, E1007M, E1007A, E1007G, | | | T, M, A, G, V, C | T, S, Q, H, R, |
| E1007V, E1007C, E1007S, E1007Q, | | | | G, W, C |
| E1007H, E1007R, E1007W | | | | |
| F1008L, F1008A, F1008S, F1008M | | | L, A | S, M, A |
| V1009L, V1009M, V1009Q, V1009G | | | L | M, Q, G |
| Y1010N, Y1010R, Y1010M, Y1010G, | | | N, R, M, G | V, W |
| Y1010V, Y1010W | | | | |
| G1011L | | | | L |
| D1012T, D1012S, D1012I, D1012P, | | | T, S, I, P, G | A, G, V, L |
| D1012G, D1012A, D1012V, D1012L | | | | |
| Y1013N, Y1013Q, Y1013L, Y1013S, | | | N, Q, L, S, W | N, R, L, G, F |
| Y1013W, Y1013R, Y1013G, Y1013F | | | | |
| K1014T, K1014H, K1014R, K1014Y, | | | T, H, R, Y | N, G |
| K1014N, K1014G | | | | |
| V1015T, V1015I, V1015R, V1015E, | | | T, I, R, E, D, G, Y | N, I, Q, P, L, |
| V1015D, V1015G, V1015Y, V1015N, | | | | G, S, F |
| V1015Q, V1015P, V1015L, V1015S, | | | | |
| V1015F | | | | |
| Y1016R, Y1016M, Y1016V, Y1016S, | | | R, M, V | S, L |
| Y1016L | | | | |
| D1017S, D1017E, D1017F | | | S | E, F |
| V1018I, V1018P, V1018W, V1018F | | | I | P, W, F |
| R1019T, R1019M, R1019Q, R1019E, | | | T, M, Q, E, A, F | |
| R1019A, R1019F | | | | |
| K1020T, K1020M, K1020E, K1020D, | | | T, M, E, D, L | D, A, S |
| K1020L, K1020A, K1020S | | | | |
| M1021T, M1021S, M1021A, M1021C, | | | T | S, A, C, F |
| M1021F | | | | |
| I1022F | | | F | |
| A1023C, A1023L | | | C | L |
| K1024S, K1024Q, K1024A, K1024G, | | | S, Q, A, G | E, D, A, C |
| K1024E, K1024D, K1024C | | | | |
| S1025T, S1025D | | | | T, D |
| E1026N, E10261 | | | N | N, I |
| Q1027N, Q1027I, Q1027L, Q1027A, | | | N, I, L, A, V | T, G, C |
| Q1027V, Q1027T, Q1027G, Q1027C | | | | |
| E1028D, E1028C, E1028N, E1028Y | | | D, C | N, Y |
| I1029L, I1029D, I1029M | | | L, D | M |
| G1030A, G1030E, G1030S | | | A | E, S |
| K1031P, K1031L, K1031G, K1031V, | | | P, L, G, V, Y, W | P, D, V, S |
| K1031Y, K1031W, K1031D, K1031S | | | | |
| A1032G, A1032K, A1032C | | | G | K, C |
| T1033Q, T1033Y, T1033S | | | Q, Y, S | |
| A1034N, A1034S, A1034Q, A1034G, | | | N, S, Q, G, V, C | Q, L |
| A1034V, A1034C, A1034L | | | | |
| K1035R, K1035H, K1035S, K1035F, | | | R, H, S, F | A, V, S |
| K1035A, K1035V | | | | |
| Y1036M, Y1036Q, Y1036S, Y1036I, | | | M, Q, S | M, I, Q, E |
| Y1036E | | | | |
| F1037W | | | W | |
| F1038Y, F1038S, F1038C | | | Y, S, C | |
| S1040T, S1040I | | | T | I |
| N1041S | | | S | |
| I1042V, I1042W, I1042C, I1042D, I1042L | | | V, W, C | D, W, L |
| M1043R, M1043S | | | R | S |
| N1044Y | | | Y | |
| F1045M, F1045A | | | M, A | |
| K1047T, K1047S, K1047Q, K1047R, | | | T, S, Q, R, W | P, R |
| K1047W, K1047P | | | | |
| T1048S, T1048L, T1048V | | | S, L, V | |
| E1049N, E1049A, E1049Y, E1049W, | | | N, A, Y, W | Q, Y |
| E1049Q | | | | |
| I1050A, I1050F, I1050L, I1050E, I1050V | | | A, F | L, E, V |
| T1051K, T1051Q, T1051E, T1051Y, | | | K, Q, E, Y | H, R, A, W, |
| T1051H, T1051R, T1051A, T1051W, | | | | F |
| T1051F | | | | |
| L1052P, L1052G | | | P | G |
| A1053T, A1053M, A1053S, A1053P, | | | T, M | T, S, P, R, G |
| A1053R, A1053G | | | | |
| N1054S, N1054D, N1054G, N1054C, | | | S, D, G, C | R, A, G, V |
| N1054R, N1054A, N1054V | | | | |
| G1055E, G1055T | | | E | T |
| E1056N, E1056L, E1056T, E1056D, | | | N, L | T, D, V |
| E1056V | | | | |
| I1057N, I1057T, I1057R, I1057G, I1057V, | | | N, T, R, G, V, S | G, W |
| I1057S, I1057W | | | | |
| R1058K, R1058V, R1058Y, R1058N, | | | K, V, Y | N, Y, S |
| R1058S | | | | |
| K1059Q, K1059A, K1059V, K1059L, | | | Q, A, V, L | S, H, P, A, W |
| K1059S, K1059H, K1059P, K1059W | | | | |
| R1060N, R1060L, R1060E, R1060S, | | | N, L, E, S, C | M, H, L |
| R1060C, R1060M, R1060H | | | | |
| P1061N, P1061V, P1061A, P1061G, | | | N, V | A, G, S |
| P1061S | | | | |
| L1062T, L1062S, L1062I, L1062H, | | | T, S, I, H, R, E, D, G | D, V |
| L1062R, L1062E, L1062D, L1062G, | | | | |
| L1062V | | | | |
| I1063T, I1063H, I1063L, I1063V, I1063R | | | T, H, L, V | T, R |
| E1064A | | | | A |
| T1065Q, T1065L, T1065V, T1065S, | | | Q, L, V, S, W, C | M, P, A, S |
| T1065W, T1065C, T1065M, T1065P, | | | | |
| T1065A | | | | |
| N1066S, N1066C, N1066T, N1066Q | | | S, C | T, Q |
| G1067S, G1067Q, G1067P, G1067L, | | | S, Q, P, L, E, A, V, | R, P, L, D, A |
| G1067E, G1067A, G1067V, G1067W, | | | W | |
| G1067R, G1067D | | | | |
| E1068S, E1068D, E1068A | | | | S, D, A |
| T1069N, T1069G, T1069Y, T1069H, | | | N, G, Y | H, R, D, S |
| T1069R, T1069D, T1069S | | | | |
| G1070N, G1070D, G1070P, G1070V | | | N, D | N, P, V |
| E1071L, E1071A, E1071V | | | L, A, V | |
| I1072T, I1072R, I1072L, I1072A, I1072V, | | | T, R, L, A, V | H |
| I1072H | | | | |
| V1073A, V1073L | | | | A, L |
| D1075M | | | | M |
| K1076R | | | R | |
| G1077T, G1077S, G1077L, G1077E, | T, S, L, E, D | R | K, D | M, L, V, C, F |
| G1077D, G1077R, G1077K, G1077M, | | | | |
| G1077V, G1077C, G1077F | | | | |
| R1078K, R1078V, R1078T, R1078I, | K, V | K, T, I, L | G | S, Q |
| R1078L, R1078G, R1078S, R1078Q | | | | |
| D1079F, D1079T, D1079A, D1079G | F | | T, A | G |
| F1080T, F1080M, F1080E, F1080W, | T, M, E, W | V | T, H, V | |
| F1080V, F1080H | | | | |
| A1081K, A1081N, A1081T, A1081L, | K, N, T, L, D, C | L, E, D, G | T, P, D, S, C | R, H, L, E, |
| A1081D, A1081C, A1081E, A1081G, | | | | G, V |
| A1081P, A1081S, A1081R, A1081H, | | | | |
| A1081V | | | | |
| T1082E, T1082V, T1082I, T1082Y, | | E, V | I, Y, S, L | N |
| T1082S, T1082L, T1082N | | | | |
| V1083I, V1083L | I | L | | |
| R1084K, R1084N, R1084T, R1084S, | K, N, T, S, Q, H, L, | T, S, M, I, Q, E, | T, M, L, E, D, G, V, | Q, H, G, V |
| R1084Q, R1084H, R1084L, R1084E, | E, D, A, G | V | Y, S, C | |
| R1084I, R1084V, R1084Y, R1084C | | | | |
| R1084D, R1084A, R1084G, R1084M, | | | | |
| R1084I, R1084V, R1084Y, R1084C | | | | |
| K1085N, K1085T, K1085S, K1085M, | N, T, S, M, Q, H, E, | N, R, H, A, G, | T, R, S, Q, E, G, C, | T, E, Y, F |
| K1085Q, K1085H, K1085E, K1085D, | D, A, G, C | C | L | |
| K1085A, K1085G, K1085C, K1085R, | | | | |
| K1085L, K1085Y, K1085F | | | | |
| V1086T, V1086E, V1086A, V1086N, | T | E | A | N, T, I |
| V1086I | | | | |
| L1087F, L1087T | | | F | T |
| S1088D, S1088T, S1088P, S1088N, | D | T, P | N, M, E | D, A |
| S1088M, S1088E, S1088A | | | | |
| M1089N, M1089T, M1089I, M1089E, | N, T, I, E, D, G, V, S | T, G, V, S, L | N, T, R, H, L, E, D, | G, F |
| M1089D, M1089G, M1089V, M1089S, | | | S, C | |
| M1089L, M1089R, M1089H, M1089C, | | | | |
| M1089F | | | | |
| P1090S, P1090Q, P1090G, P1090V, | S, Q, G, V, L | Y, C | D, G | S, M, Q, R, |
| P1090L, P1090Y, P1090C, P1090D, | | | | E, A, V, C |
| P1090M, P1090R, P1090E, P1090A | | | | |
| Q1091T, Q1091I, Q1091H, Q1091R, | T, I, H, R, L, E, G, V | S, P, L, E, V, W | N, T, S, L, E, F | E, D, G, V, Y, |
| Q1091L, Q1091E, Q1091G, Q1091V, | | | | C |
| Q1091S, Q1091P, Q1091W, Q1091N, | | | | |
| Q1091F, Q1091D, Q1091Y, Q1091C | | | | |
| V1092L, V1092A, V1092I, V1092Q, | | L, A | I, Q, C, L | L |
| V1092C | | | | |
| N1093I, N1093R, N1093Q, N1093L, | | I, R | I, Q, L, A, S | H |
| N1093A, N1093S, N1093H | | | | |
| I1094M, I1094L, I1094T, I1094S, I1094P | M, L | | T, S, M, P, L | M, L |
| V1095K, V1095T, V1095R, V1095Q, | K, T, R | K, Q, G, L | | K, L |
| V1095G, V1095L | | | | |
| K1096N, K1096T, K1096S, K1096I, | N, T, S, I, Q, R, L, | N, S, R, V, Y, C | N, R, I, Q, L, A, V, | N, T, R, M, I, |
| K1096Q, K1096R, K1096L, K1096A, | A, V, Y, W, C | | C | P, L, V, Y, S, |
| K1096V, K1096Y, K1096W, K1096C, | | | | W, C |
| K1096M, K1096P | | | | |
| K1097R | | R | | |
| T1098K, T1098R, T1098M, T1098I, | K, R, M, I, H, L, A, | K, R, L, V | K, N, R, L, Y, S, W | K, R, M, V, F |
| T1098H, T1098L, T1098A, T1098V, | V, C | | | |
| T1098C, T1098N, T1098Y, T1098S, | | | | |
| T1098W, T1098F | | | | |
| E1099K, E1099R, E1099S, E1099H, | K, R, S, H, A, G, V, | A | N, T, R, S, M, I, Q, | K, R, S, M, |
| E1099A, E1099G, E1099V, E1099Y, | Y | | H, L, V, C | H, L, A, C |
| E1099N, E1099T, E1099M, E1099I, | | | | |
| E1099Q, E1099L, E1099C | | | | |
| V1100K, V1100R, V1100I, V1100L, | K, R, I, L | K, R, D, C | K, T, A | T |
| V1100D, V1100C, V1100T, V1100A | | | | |
| Q1101V, Q1101A, Q1101C | V | | A, C | |
| T1102R, T1102A | R | A | | |
| G1104S, G1104P, G1104A, G1104C, | S, P, A, C | P, A | T, A, S, C | P, A, C |
| G1104T | | | | |
| S1106T, S1106H, S1106A, S1106Y, | T, H, A, Y, W, C, F | T, H, Y, F | T, H, A, Y, W, C, F | T, H, A, V, Y, |
| S1106W, S1106C, S1106F, S1106V | | | | W, C, F |
| K1107N, K1107R, K1107Q | N, R | R | N | Q |
| E1108D, E1108G | D, G | G | G | G |
| S1109K, S1109N, S1109T, S1109R, | K, N, T, R, M, I, Q, | K, T, R, L, A, G, | I, Q, H, G, V, C | R, M, L, A, V |
| S1109M, S1109I, S1109Q, S1109L, | L, A, G, V, C | V | | |
| S1109A, S1109G, S1109V, S1109C, | | | | |
| S1109H | | | | |
| I1110P | | | P | |
| L1111V, L1111S | | V | V, S | |
| P1112Q, P1112G, P1112D | | | Q, G | Q, D |
| K1113A, K1113R, K1113G | A | A | R, A | G |
| R1114N, R1114T, R1114S, R1114Q, | N, T, S, Q, H, D, A, | K, G | T, S, A, Y | N, T, S, H, |
| R1114H, R1114D, R1114A, R1114G, | G, C | | | D, A, G, C |
| R1114C, R1114K, R1114Y | | | | |
| N1115H, N1115S, N1115P, N1115F, | H | S | S, H, P, F | L, D, A, W, F |
| N1115L, N1115D, N1115A, N1115W | | | | |
| S1116T, S1116G, S1116K | T, G | | G | K, T |
| D1117G, D1117N, D1117H, D1117S | G | G | N | H, G, S |
| K1118N, K1118T, K1118R, K1118S, | N, T, R, S, Q, L, A, | T, S | T, H, L, V | T, R, S, Q |
| K1118Q, K1118L, K1118A, K1118G, | G, C | | | |
| K1118C, K1118H, K1118V | | | | |
| L1119G, L1119R | | G | | R |
| 11120V, 11120T, 11120L | V | | T, L, V | V |
| A1121R, A1121P, A1121D, A1121Y | R | | P, D, Y | |
| R1122I | | | | I |
| K1123N | | | | N |
| K1124R, K1124V | R | V | | R |
| D1125A, D1125G, D1125C, D1125T, | A, G, C | | T, H, R, C | N, A, G, Y, C |
| D1125H, D1125R, D1125N, D1125Y | | | | |
| D1127S | | S | | |
| P1128F | | | | F |
| K1129V, K1129T, K1129I | | V | T, V | I |
| Y1131L | | L | | |
| F1134Y | | | Y | |
| D1135M, D1135E, D1135A, D1135T, | | | M, E, A | T, S, H, E, A, |
| D1135S, D1135H, D1135G, D1135V | | | | G, V |
| S1136K, S1136T, S1136G | | | K, T, G | |
| T1138K, T1138N, T1138S, T1138A, | | | K, N, S, A, Y | R, Q, G |
| T1138Y, T1138R, T1138Q, T1138G | | | | |
| V1139L, V1139E, V1139D, V1139G, | | | L, E, D, G, S | H, E, G |
| V1139S, V1139H | | | | |
| A1140C, A1140T | | | C | T |
| Y1141R | | | R | |
| S1142A, S1142V, S1142C, S1142G | | | A, V, C | G, C |
| V1143L, V1143A, V1143T | | | L, A | T |
| L1144V, L1144W, L1144A, L1144Y, | | | V, W | A, Y, C |
| L1144C | | | | |
| V1145N, V1145L, V1145A, V1145F | | | N, L, A, F | |
| V1146Y, V1146W, V1146C, V1146K, | | | Y, W, C | K, R, E, A, Y |
| V1146R, V1146E, V1146A | | | | |
| A1147H | | | H | |
| K1148R, K1148Q, K1148L, K1148A, | | | R, Q, L, A, W | T, A, C |
| K1148W, K1148T, K1148C | | | | |
| V1149T, V1149M, V1149Q, V1149L, | | | T, M, Q, L, E | |
| V1149E | | | | |
| E1150S, E1150H, E1150L, E1150P, | | | S, H, L | P, A |
| E1150A | | | | |
| K1151N, K1151I, K1151Q, K1151H, | | | N, I, Q, H, L, E, A, | R, I, Q, P, L, |
| K1151L, K1151E, K1151A, K1151G, | | | G, V, Y, S, F | E, C |
| K1151V, K1151Y, K1151S, K1151F, | | | | |
| K1151R, K1151P, K1151C | | | | |
| G1152S, G1152P, G1152T, G1152R, | | | S, P | T, R, Q, L |
| G1152Q, G1152L | | | | |
| K1153N, K1153M, K1153I, K1153Q, | | | N, M, I, Q, H, E, G, | T, R, S, M, I, |
| K1153H, K1153E, K1153G, K1153V, | | | V | Q, L, V, C |
| K1153T, K1153R, K1153S, K1153L, | | | | |
| K1153C | | | | |
| S1154N, S1154E, S1154D, S1154G, | | | N, E, D, G | A |
| S1154A | | | | |
| K1155S, K1155P, K1155R, K1155L, | | | S, P, R, L, G, V | T, E, A, G |
| K1155G, K1155V, K1155T, K1155E, | | | | |
| K1155A | | | | |
| K1156T, K1156I, K1156Q, K1156P, | | | T, I, Q, P, L | R, I, L, D, V, |
| K1156L, K1156R, K1156D, K1156V, | | | | Y, S |
| K1156Y, K1156S | | | | |
| L1157T, L1157M, L1157H, L1157R, | | | T, M, H, R, A, G | I |
| L1157A, L1157G, L1157I | | | | |
| K1158H, K1158E, K1158D, K1158G, | | | H, E, D, G, V, Y, F | T, D, S, L |
| K1158V, K1158Y, K1158F, K1158T, | | | | |
| K1158S, K1158L | | | | |
| S1159H, S1159P, S1159L, S1159E, | | | H, P, L, E, A, G, F | N, T, M, Q, V |
| S1159A, S1159G, S1159F, S1159N, | | | | |
| S1159T, S1159M, S1159Q, S1159V | | | | |
| V1160N, V1160E, V1160T | | | N, E | T |
| K1161V, K1161S, K1161T, K1161I, | | | V, S | T, I, L, D |
| K1161L, K1161D | | | | |
| E1162G, E1162D, E1162V, E1162Y | | | G | D, V, Y |
| L1163M, L1163A, L1163G, L1163T, | | | M, A, G | T, A, G, V, S |
| L1163V, L1163S | | | | |
| L1164H, L1164V, L1164F, L1164M, | | | H, V, F | M, I, E |
| L1164I, L1164E | | | | |
| G1165K, G1165P, G1165A, G1165S, | | | K, P, A, S | P, L, E, A, Y |
| G1165L, G1165E, G1165Y | | | | |
| T1167P, T1167Y, T1167Q, T1167H, | | | P, Y | Q, H, L |
| T1167L | | | | |
| M1169L | | | L | |
| R1171K, R1171H, R1171Q | K | | H | Q |
| S1172T, S1172P, S1172L, S1172G, | T, P, L, G, V | E, A, Y | M, C, L | T, A, F |
| S1172V, S1172E, S1172A, S1172Y, | | | | |
| S1172M, S1172C, S1172F | | | | |
| S1173K, S1173Y, S1173L, S1173C, | K, Y | L, C | R, Q, L, D | H, V, W |
| S1173R, S1173Q, S1173D, S1173H, | | | | |
| S1173V, S1173W | | | | |
| F1174S | | | | S |
| E1175Q, E1175L | | | Q | L |
| K1176S, K1176I, K1176D, K1176A, | S, I, D, A | | N, T, I, Q, S, C | L, D, V |
| K1176N, K1176T, K1176Q, K1176C, | | | | |
| K1176L, K1176V | | | | |
| N1177T, N1177G, N1177S, N1177C | | | T | G, S, C |
| P1178R | | | R | |
| I1179A, I1179G, I1179Y, I1179T, I1179Q, | | | A, G, Y | T, Q, E, Y |
| I1179E | | | | |
| D1180T, D1180R, D1180S, D1180P, | | | T, R, S, P, L, E, A, G | I, P, A |
| D1180L, D1180E, D1180A, D1180G, | | | | |
| D1180I | | | | |
| F1181V, F1181T, F1181M, F1181Q, | | | V | T, M, Q, H, |
| F1181H, F1181L, F1181A | | | | L, A |
| L1182V, L1182C | | | V | C |
| E1183M, E1183I, E1183G, E1183V, | | | M, I, G, V, S | H, L, D |
| E1183S, E1183H, E1183L, E1183D | | | | |
| A1184N, A1184R, A1184Q, A1184V, | | | N, R, Q, V | D, G, C |
| A1184D, A1184G, A1184C | | | | |
| K1185N, K1185S, K1185L, K1185T, | | | N, S, L | T, E, A, G, C |
| K1185E, K1185A, K1185G, K1185C | | | | |
| G1186P | | | | P |
| Y1187L, Y1187F, Y1187S, Y1187P, | | | L, F | S, P, W, F |
| Y1187W | | | | |
| K1188R, K1188A, K1188V, K1188S, | | | R, A, V, S, W, F | L, Y, S, C |
| K1188W, K1188F, K1188L, K1188Y, | | | | |
| K1188C | | | | |
| E1189L, E1189Q | | | L | Q |
| V1190M, V1190L, V1190A, V1190I | | | M, L, A | I |
| K1191T, K1191R, K1191I, K1191Q, | T, R, I, Q, P, L, D, A, | N, S, Q, E, G, | T, H, D, G | N, P, R, E, Y |
| K1191P, K1191L, K1191D, K1191A, | G, V, Y, F | C, F | | |
| K1191G, K1191V, K1191Y, K1191F, | | | | |
| K1191N, K1191S, K1191E, K1191C, | | | | |
| K1191H | | | | |
| K1192N, K1192H, K1192R, K1192E, | N, H, R | E, S, L | Q, P, A, V | T, Q, L |
| K1192S, K1192L, K1192Q, K1192P, | | | | |
| K1192A, K1192V, K1192T | | | | |
| D1193H, D1193Y, D1193I, D1193P, | | H, Y | I, P, R, A, V | A, C |
| D1193R, D1193A, D1193V, D1193C | | | | |
| L1194S, L1194V, L1194W, L1194F, | S, V, W, F | Q, H, A | E, S, C, F | R, D, G |
| L1194Q, L1194H, L1194A, L1194E, | | | | |
| L1194C, L1194R, L1194D, L1194G | | | | |
| I1195T, I1195V | | | T | V |
| I1196K, I1196T, I1196H, I1196F, I1196A, | K, T, H, F | | A | M, L, Y |
| I1196M, I1196L, I1196Y | | | | |
| K1197L, K1197I, K1197F, K1197C | L | | I, F | C |
| L1198T, L1198A, L1198V, L1198F | T, A | A | V | V, F |
| P1199Y, P1199C, P1199T, P1199R, | Y, C | T, R, H | T, R, G, Y, F | A, G |
| P1199H, P1199G, P1199F, P1199A | | | | |
| K1200D, K1200S | | | D | S |
| Y1201G, Y1201S | | | | G, S |
| S1202A, S1202V, S1202I, S1202Q, | | | A, V | I, Q, H, Y |
| S1202H, S1202Y | | | | |
| L1203N, L1203F, L1203T, L1203A, | | | N, F | T, A, C |
| L1203C | | | | |
| F1204I, F1204L, F1204V | | | I, L, V | |
| E1205C, E1205K, E1205T, E1205M, | | | C | K, T, M, R |
| E1205R | | | | |
| L1206S, L1206Q, L1206A, L1206Y, | | | S, Q, A, Y, F | S, D, G, V, |
| L1206F, L1206D, L1206G, L1206V, | | | | W |
| L1206W | | | | |
| E1207R, E1207C, E1207Q | | | R, C | Q |
| N1208T, N1208P, N1208L, N1208W, | | | T, P, L, W | H, G, S, C |
| N1208H, N1208G, N1208S, N1208C | | | | |
| G1209K, G1209T, G1209Y, G1209S, | | | K, T, Y, S, C | K, M, R, A, V |
| G1209C, G1209M, G1209R, G1209A, | | | | |
| G1209V | | | | |
| R1210S, R1210P, R1210L, R1210E, | | | S, P, L, E, A, G, C | M, I, L, A, C |
| R1210A, R1210G, R1210C, R1210M, | | | | |
| R1210I | | | | |
| K1211T, K1211S, K1211M, K1211Q, | | | T, S, M, Q, L, A, Y | N, W |
| K1211L, K1211A, K1211Y, K1211N, | | | | |
| K1211W | | | | |
| R1212V, R1212C | | | V | C |
| M1213T, M1213I, M1213H, M1213L, | | | T, I, H, L, A, V, Y, S, | Q, P, R, A, Y, |
| M1213A, M1213V, M1213Y, M1213S, | | | W, F | W, C, F |
| M1213W, M1213F, M1213Q, M1213P, | | | | |
| M1213R, M1213C | | | | |
| L1214T, L1214M, L1214H, L1214V, | | | T, M, H | M, V, S, C |
| L1214S, L1214C | | | | |
| A1215G, A1215C, A1215S | | | G, C | G, S |
| S1216T, S1216A | | | T | A |
| A1217T, A1217H, A1217P, A1217D, | | | T, H, P, D, G, W, F | N, S, M, G, |
| A1217G, A1217W, A1217F, A1217N, | | | | W, C, F |
| A1217S, A1217M, A1217C | | | | |
| G1218S, G1218E, G1218W | | | S, E, W | |
| L1220M, L1220N, L1220S, L1220W, | | | M | N, S, W, C |
| L1220C | | | | |
| Q1221N, Q1221S, Q1221M, Q1221I, | | | N, S, M, I, A, V, C | N, T, S, M, |
| Q1221A, Q1221V, Q1221C, Q1221T, | | | | H, L, A, G |
| Q1221H, Q1221L, Q1221G | | | | |
| K1222T, K1222R, K1222S | | | T, R | S |
| G1223M, G1223S, G1223N, G1223A, | | | M, S | N, A, C |
| G1223C | | | | |
| N1224T, N1224D, N1224P, N1224G | | | T, D | P, G |
| E1225Q | | | Q | |
| L1226V, L1226M, L1226H, L1226P | | | V | M, H, P, V |
| A1227Y, A1227S, A1227G | | | Y | S, G |
| L1228I, L1228F, L1228S, L1228V | | | I, F | S, V |
| P1229K, P1229N, P1229T, P1229S, | | | K, N, T, S, I, Q, D, | R, M, H, L, F |
| P1229I, P1229Q, P1229D, P1229A, | | | A, G, V, C, F | |
| P1229G, P1229V, P1229C, P1229F, | | | | |
| P1229R, P1229M, P1229H, P1229L | | | | |
| S1230T, S1230M, S1230Q, S1230L, | | | T, M, Q, L, G | T, Q, V |
| S1230G, S1230V | | | | |
| K1231T, K1231S, K1231H, K1231P, | | | T, S, H, P, D, A, V, | T, S, M, H, L, |
| K1231D, K1231A, K1231V, K1231W, | | | W, F | E, D, G, Y, C |
| K1231F, K1231M, K1231L, K1231E, | | | | |
| K1231G, K1231Y, K1231C | | | | |
| Y1232L, Y1232N, Y1232T, Y1232R, | | | L | N, T, R, S, D, |
| Y1232S, Y1232D, Y1232V | | | | V |
| V1233I, V1233Q, V1233L, V1233E, | | | I, Q, L, E, W | T, Q |
| V1233W, V1233T | | | | |
| N1234T, N1234Q, N1234R, N1234L, | | | T, Q, R, L, D, A, V | T, Q, P, C |
| N1234D, N1234A, N1234V, N1234P, | | | | |
| N1234C | | | | |
| F1235L, F1235W, F1235M, F1235H, | | | L, W | M, H, L, A, |
| F1235A, F1235G | | | | G |
| L1236T, L1236I, L1236V, L1236Q | | | T, I, V | Q |
| Y1237K, Y1237S, Y1237G, Y1237V, | | | K, S, G, V | R, M, Q, L, |
| Y1237R, Y1237M, Y1237Q, Y1237L, | | | | A, G, C |
| Y1237A, Y1237C | | | | |
| L1238Q, L1238H, L1238A, L1238V, | | | Q, H, A, V, F | S, V, Y |
| L1238F, L1238S, L1238Y | | | | |
| A1239M, A1239H, A1239L, A1239W | | | M, H, L, W | |
| S1240Q, S1240H, S1240L, S1240Y, | | | Q, H, L, Y, F | N, I, A, G |
| S1240F, S1240N, S1240I, S1240A, | | | | |
| S1240G | | | | |
| H1241N, H1241I, H1241Q, H1241L, | | | N, I, Q, L, G | M, R, L, D, |
| H1241G, H1241M, H1241R, H1241D, | | | | A, V, Y, F |
| H1241A, H1241V, H1241Y, H1241F | | | | |
| Y1242K, Y1242T, Y1242S, Y1242M, | | | K, T, S, M, P, G, V, | T, I, L |
| Y1242P, Y1242G, Y1242V, Y1242W, | | | W | |
| Y1242I, Y1242L | | | | |
| E1243N, E1243S, E1243M, E1243A, | | | N, S, M, A | T, P, V |
| E1243T, E1243P, E1243V | | | | |
| K1244S, K1244I, K1244Q, K1244P, | | | S, I, Q, P, L, E, D, V, | R, S, M, A, G |
| K1244L, K1244E, K1244D, K1244V, | | | F | |
| K1244F, K1244R, K1244M, K1244A, | | | | |
| K1244G | | | | |
| L1245N, L1245Q, L1245P, L1245G, | | | N, Q, P, G, V, Y, F | N, S, M, P, |
| L1245V, L1245Y, L1245F, L1245S, | | | | R, A, W, C |
| L1245M, L1245R, L1245A, L1245W, | | | | |
| L1245C | | | | |
| K1246N, K1246S, K1246R, K1246D, | | | N, S, R, D, W | N, S, I, L, A, |
| K1246W, K1246I, K1246L, K1246A, | | | | G, C |
| K1246G, K1246C | | | | |
| G1247S, G1247H, G1247W, G1247K, | | | S, H, W | K, T, P, L, A, |
| G1247T, G1247P, G1247L, G1247A, | | | | Y, F |
| G1247Y, G1247F | | | | |
| S1248I, S1248R, S1248L, S1248D, | | | I, R, L, D, G, Y, W | M, R |
| S1248G, S1248Y, S1248W, S1248M | | | | |
| P1249N, P1249D, P1249G, P1249V, | | | N, D, G, V | N, R, S, H, L |
| P1249R, P1249S, P1249H, P1249L | | | | |
| E1250R, E1250Q, E1250P, E1250G, | | | R, Q, P, G, Y, S, F | T, D, G |
| E1250Y, E1250S, E1250F, E1250T, | | | | |
| E1250D | | | | |
| D1251S, D1251W, D1251N, D1251T, | | | S, W | N, T, C |
| D1251C | | | | |
| N1252K, N1252T, N1252A, N1252V, | | | K, T, A, V, S, C | R, G |
| N1252S, N1252C, N1252R, N1252G | | | | |
| E1253N, E1253T, E1253D, E1253A, | | | N, T, D, A, G, W, C | S, R, L |
| E1253G, E1253W, E1253C, E1253S, | | | | |
| E1253R, E1253L | | | | |
| Q1254N, Q1254I, Q1254H, Q1254P, | | | N, I, H, P, R, E, G, | T, P, D, V, C |
| Q1254R, Q1254E, Q1254G, Q1254Y, | | | Y, W | |
| Q1254W, Q1254T, Q1254D, Q1254V, | | | | |
| Q1254C | | | | |
| K1255R, K1255S, K1255P, K1255G, | | | R, S, P, G, V, F | L, F |
| K1255V, K1255F, K1255L | | | | |
| Q1256T, Q1256P, Q1256E, Q1256S, | | | T, P, E, S | R, W, C |
| Q1256R, Q1256W, Q1256C | | | | |
| L1257R, L1257H, L1257P, L1257E, | | | R, H, P, E, V, W | S, M, G, V, |
| L1257V, L1257W, L1257S, L1257M, | | | | W, C |
| L1257G, L1257C | | | | |
| F1258K, F1258R, F1258L, F1258V, | | | K, R, L, V, S | P, V |
| F1258S, F1258P | | | | |
| V1259R, V1259I, V1259L, V1259E, | | | R, I, L, E, C, F | A, F |
| V1259C, V1259F, V1259A | | | | |
| E1260S, E1260Q, E1260R, E1260L, | | | S, Q, R, L, G, V, Y, F | A, G, C, F |
| E1260G, E1260V, E1260Y, E1260F, | | | | |
| E1260A, E1260C | | | | |
| Q1261T, Q1261D, Q1261A, Q1261V, | | | T, D, A, V, W, L | M, H, P, G, Y |
| Q1261W, Q1261L, Q1261M, Q1261H, | | | | |
| Q1261P, Q1261G, Q1261Y | | | | |
| H1262N, H1262R, H1262L, H1262A, | | | N, R, L, A, W, C | T, D, V, S |
| H1262W, H1262C, H1262T, H1262D, | | | | |
| H1262V, H1262S | | | | |
| K1263R, K1263Q, K1263P, K1263L, | | | R, Q, P, L, A | N, S, I, P, D, |
| K1263A, K1263N, K1263S, K1263I, | | | | V, F |
| K1263D, K1263V, K1263F | | | | |
| H1264M, H1264Q, H1264G, H1264V, | | | M, Q, G, V | S, I, L |
| H1264S, H1264I, H1264L | | | | |
| Y1265L,Y1265V,Y1265C,Y1265R, | | | L, V, C | R, I, Q, H, S, |
| Y1265I, Y1265Q, Y1265H, Y1265S | | | | C |
| L1266G, L1266C, L1266T, L1266S, | | | G, C | T, S, W |
| L1266W | | | | |
| D1267N, D1267T, D1267Q, D1267P, | | | N, T, Q, P, L | I, G, V |
| D1267L, D1267, D1267G, D1267V | | | | |
| E1268T, E1268L, E1268D, E1268G, | | | T, L, D, G, V, Y, S | T, R, A, Y, S |
| E1268V, E1268Y, E1268S, E1268R, | | | | |
| E1268A | | | | |
| I1269L, I1269V, I1269M, I1269D, I1269C | | | L, V | M, D, V, C |
| I1270T, I1270M, I1270L, I1270Q, I1270F | | | T, M, L | Q, F |
| E1271T, E1271R, E1271H, E1271L, | | | T, R, H, L, D, A, W | S, W, C |
| E1271D, E1271A, E1271W, E1271S, | | | | |
| E1271C | | | | |
| Q1272T, Q1272S, Q1272A, Q1272G, | | | T, S, A, G, W, C | I |
| Q1272W, Q1272C, Q1272I | | | | |
| I1273T, I1273M, I1273F, I1273L | | | T, M, F | L, F |
| S1274K, S1274H, S1274R, S1274A, | | | K, H, R, A, V | T, R, G, L, F |
| S1274V, S1274T, S1274G, S1274L, | | | | |
| S1274F | | | | |
| E1275T, E1275L, E1275Y, E1275I, | | | T, L, Y | I, S, W |
| E1275S, E1275W | | | | |
| F1276S, F1276M, F1276Y, F1276C, | | | S, M, Y, C | E, G, Y |
| F1276E, F1276G | | | | |
| S1277T, S1277M, S1277I, S1277H, | | | T, M, I, H, E, G, V | N, H, F |
| S1277E, S1277G, S1277V, S1277N, | | | | |
| S1277F | | | | |
| K1278M, K1278I, K1278V, K1278S, | | | M, I, V, S | G, Y, S, L |
| K1278G, K1278Y, K1278L | | | | |
| R1279S, R1279L, R1279D, R1279G, | | | S, L, D, G | T, A, W, C |
| R1279T, R1279A, R1279W, R1279C | | | | |
| V1280T, V1280I, V1280Q, V1280L, | | | T, I, Q, L, E, D, A | S |
| V1280E, V1280D, V1280A, V1280S | | | | |
| I1281M, I1281H, I1281R, I1281L, I1281A, | | | M, H, R, L, A, V, Y | N, S, M, H, |
| I1281V, I1281Y, I1281N, I1281S, I1281C | | | | C |
| L1282N, L1282T, L1282S, L1282R, | | | N, T, S, R, E, D, A, | S, Q, R, V, C |
| L1282E, L1282D, L1282A, L1282G, | | | G, V, W, C | |
| L1282V, L1282W, L1282C, L1282Q | | | | |
| A1283N, A1283T, A1283G, A1283Y, | | | N, T, G, Y, C | T, S, H, R, E |
| A1283C, A1283S, A1283H, A1283R, | | | | |
| A1283E | | | | |
| D1284G | | | G | |
| A1285N, A1285T, A1285R, A1285L, | | | N, T, R, L, E, D, W, | H, P, V |
| A1285E, A1285D, A1285W, A1285F, | | | F | |
| A1285H, A1285P, A1285V | | | | |
| N1286T, N1286S, N1286M, N1286Q, | | | T, S, M, Q, P, L, G, | H, L, E, A, |
| N1286P, N1286L, N1286G, N1286V, | | | V, W, C | G, V, F |
| N1286W, N1286C, N1286H, N1286E, | | | | |
| N1286A, N1286F | | | | |
| L1287S, L1287Q, L1287P, L1287R, | | | S, Q, P, R, E, F | N, T, S, I, A, |
| L1287E, L1287F, L1287N, L1287T, | | | | V, W, F |
| L1287I, L1287A, L1287V, L1287W | | | | |
| D1288N, D1288M, D1288E, D1288A, | | | N, M, E, A | A, G, V, S, L |
| D1288G, D1288V, D1288S, D1288L | | | | |
| K1289R, K1289M, K1289I, K1289P, | | | R, M, I, P, E, D, G, | Q, V, S, C, L |
| K1289E, K1289D, K1289G, K1289S, | | | S, C, L | |
| K1289C, K1289L, K1289Q, K1289V | | | | |
| V1290L, V1290W, V1290M, V1290H | | | L, W | M, H |
| L1291N, L1291T, L1291M, L1291Q, | | | N, T, M, Q, H, E, A, | I, S, W, F |
| L1291H, L1291E, L1291A, L1291Y, | | | Y | |
| L1291I, L1291S, L1291W, L1291F | | | | |
| S1292T, S1292M, S1292R, S1292G, | | | T, M, R, G, Y | T, H, L, V, Y, |
| S1292Y, S1292H, S1292L, S1292V, | | | | C, F |
| S1292C, S1292F | | | | |
| A1293T, A1293S, A1293P, A1293L, | | | T, S, P, L, V, Y | H, G |
| A1293V, A1293Y, A1293H, A1293G | | | | |
| Y1294T, Y1294I, Y1294R, Y1294V, | | | T, I, R, V, W, C | S, H, L, V |
| Y1294W, Y1294C, Y1294S, Y1294H, | | | | |
| Y1294L | | | | |
| N1295T, N1295Q, N1295P, N1295E, | | | T, Q, P, E, D, W, F | K, S, I, H, R, |
| N1295D, N1295W, N1295F, N1295K, | | | | L, A, G |
| N1295S, N1295I, N1295H, N1295R, | | | | |
| N1295L, N1295A, N1295G | | | | |
| K1296L, K1296E, K1296A, K1296Y, | | | L, E, A, Y | Q, R, V, S, W |
| K1296Q, K1296R, K1296V, K1296S, | | | | |
| K1296W | | | | |
| H1297R, H1297E, H1297S, H1297L, | | | R, E, S, L, F | V |
| H1297F, H1297V | | | | |
| R1298N, R1298T, R1298Q, R1298L, | | | N, T, Q, L, A, G, V, | N, S, V, C, F |
| R1298A, R1298G, R1298V, R1298C, | | | C | |
| R1298S, R1298F | | | | |
| D1299K, D1299T, D1299L, D1299A, | | | K, T, L, A, G, Y, S, | V, C, F |
| D1299G, D1299Y, D1299S, D1299W, | | | W | |
| D1299V, D1299C, D1299F | | | | |
| K1300T, K1300H, K1300L, K1300D, | | | T, H, L, D | N, R, E, V, |
| K1300N, K1300R, K1300E, K1300V, | | | | W, F |
| K1300W, K1300F | | | | |
| P1301N, P1301D, P1301G, P1301T, | | | N, D, G | N, T, S, V, W |
| P1301S, P1301V, P1301W | | | | |
| I1302N, I1302H, I1302W, I1302T, I1302S, | | | N, H, W | T, S, A, V |
| I1302A, I1302V | | | | |
| R1303T, R1303P, R1303L, R1303E, | | | T, P, L, E, D, G, V | K, S, I, A, V |
| R1303D, R1303G, R1303V, R1303K, | | | | |
| R1303S, R1303I, R1303A | | | | |
| E1304N, E1304T, E1304R, E1304H, | | | N, T, R, H, Y, S | V |
| E1304Y, E1304S, E1304V | | | | |
| Q1305N, Q1305S, Q1305K, Q1305R, | | | N, S | K, N, R, M, |
| Q1305M, Q1305H, Q1305L, Q1305V | | | | H, L, V |
| A1306T, A1306S, A1306I, A1306C, | | | T, S, I, C | N, I |
| A1306N | | | | |
| E1307S, E1307I, E1307Q, E1307L, | | | S, I, Q, L, D, G, V | H, R, C |
| E1307D, E1307G, E1307V, E1307H, | | | | |
| E1307R, E1307C | | | | |
| N1308R | | | R | |
| I1309C, I1309S, I1309Q, I1309R, I1309A, | | | C | S, Q, R, A, V, |
| I1309V, I1309Y, I1309L | | | | Y, L |
| I1310K, I1310M, I1310Q, I1310R, I1310V, | | | K, M, Q, R, V, Y, C | T, L, A, V, Y, |
| I1310Y, I1310C, I1310T, I1310L, I1310A, | | | | C, F |
| I1310F | | | | |
| H1311N, H1311T, H1311S, H1311P, | | | N, T, S, P, R, A, G | K, M, D, G, |
| H1311R, H1311A, H1311G, H1311K, | | | | V |
| H1311M, H1311D, H1311V | | | | |
| L1312M, L1312A, L1312V, L1312C, | | | M, A, V, C | S, G, W, F |
| L1312S, L1312G, L1312W, L1312F | | | | |
| F1313T, F1313M, F1313L, F1313C, | | | T, M, L, C | M, A, V, S, |
| F1313A, F1313V, F1313S, F1313W | | | | W, C |
| T1314N, T1314M, T1314H, T1314L, | | | N, M, H, L, G, S, F | K, Q, L, E, D, |
| T1314G, T1314S, T1314F, T1314K, | | | | A, G, S, C |
| T1314Q, T1314E, T1314D, T1314A, | | | | |
| T1314C | | | | |
| L1315S, L1315I, L1315H, L1315E, | | | S, I, H, E, A, V | S, Q |
| L1315A, L1315V, L1315Q | | | | |
| T1316A, T1316Q, T1316G | | | A | Q, G |
| N1317I, N1317Q, N1317H, N1317L, | | | I, Q, H, L, A, Y, W, | T, S, I, A, G, |
| N1317A, N1317Y, N1317W, N1317F, | | | F | V |
| N1317T, N1317S, N1317G, N1317V | | | | |
| L1318K, L1318N, L1318T, L1318I, | | | K, N, T, I, Q, H, P, | N, T, M, Q, |
| L1318Q, L1318H, L1318P, L1318E, | | | E, D, G, V, Y, W, C | H, E, D, A, |
| L1318D, L1318G, L1318V, L1318Y, | | | | V, Y, S, W |
| L1318W, L1318C, L1318M, L1318A, | | | | |
| L1318S | | | | |
| G1319S, G1319N, G1319R, G1319L | | | S | N, R, L |
| A1320T, A1320R, A1320S, A1320I, | | | T, R, S, I, H, G, V, Y, | S, V, Y, W, F |
| A1320H, A1320G, A1320V, A1320Y, | | | C, F | |
| A1320C, A1320F, A1320W | | | | |
| P1321S, P1321H, P1321A, P1321G, | | | S, H, A, G, V, C | M, I, R, A, C |
| P1321V, P1321C, P1321M, P1321I, | | | | |
| P1321R | | | | |
| A1322N, A1322I, A1322L, A1322G, | | | N, I, L, G, V | N, T, M, Q, |
| A1322V, A1322T, A1322M, A1322Q, | | | | L, G, V, W, F |
| A1322W, A1322F | | | | |
| A1323G, A1323S, A1323C | | | G | S, C |
| F1324T, F1324S, F1324I, F1324H, | | | T, S, I, H, L, A, G, V, | T, R, M, D, |
| F1324L, F1324A, F1324G, F1324V, | | | C | A, V, C |
| F1324C, F1324R, F1324M, F1324D | | | | |
| K1325R | | | R | R |
| Y1326M, Y1326L, Y1326V, Y1326W, | | | M, L, V, W, F | M, I, Q, P, A, |
| Y1326F, Y1326I, Y1326Q, Y1326P, | | | | S, F |
| Y1326A, Y1326S | | | | |
| F1327L, F1327A, F1327C, F1327R | | | L, A, C | R, L, A |
| D1328T, D1328M, D1328L, D1328A, | | | T, M, L, A, S | K, L, G, V |
| D1328S, D1328K, D1328G, D1328V | | | | |
| T1329K, T1329N, T1329R, T1329A, | | | K, N, R, A, G, S, C | M, I, L, V, Y, |
| T1329G, T1329S, T1329C, T1329M, | | | | S, F |
| T1329I, T1329L, T1329V, T1329Y, T1329F | | | | |
| T1330K, T1330N, T1330P, T1330A, | | | K, N, P, A, S | N, Q |
| T1330S, T1330Q | | | | |
| I1331S, I1331M, I1331C, I1331L, I1331G | | | S, M, C | L, G |
| D1332E, D1332A, D1332V, D1332Y, | | | E, A, V, Y, C | S, W |
| D1332C, D1332S, D1332W | | | | |
| K1334T, K1334A, K1334G, K1334Q, | | | T, A, G | T, Q, R, A, S, |
| K1334R, K1334S, K1334W | | | | W |
| Y1336M, Y1336H, Y1336L, Y1336C, | | | M, H, L, C, F | N, P |
| Y1336F, Y1336N, Y1336P | | | | |
| T1337N, T1337S, T1337I, T1337P, | | | N, S, I, P, A | I, G |
| T1337A, T1337G | | | | |
| S1338N, S1338T, S1338M, S1338I, | | | N, T, M, I, L, A, G, | R, M, Q, A, |
| S1338L, S1338A, S1338G, S1338V, | | | V, Y, W, C, F | G, V, Y, W, |
| S1338Y, S1338W, S1338C, S1338F, | | | | C, F |
| S1338R, S1338Q | | | | |
| T1339S, T1339M, T1339I, T1339Q, | | | S, M, I, Q, H, R, L, | M, L, W |
| T1339H, T1339R, T1339L, T1339E, | | | E, A, G, V, F | |
| T1339A, T1339G, T1339V, T1339F, | | | | |
| T1339W | | | | |
| K1340N, K1340T, K1340S, K1340M, | | | N, T, S, M, I, Q, H, | N, R, M, H, |
| K1340I, K1340Q, K1340H, K1340P, | | | P, E, A, G, V, C, L, F | G, V, Y, C, L |
| K1340E, K1340A, K1340G, K1340V, | | | | |
| K1340C, K1340L, K1340F, K1340R, | | | | |
| K1340Y | | | | |
| E1341A, E1341I, E1341V | | | A | I, V |
| V1342M, V1342L, V1342S, V1342T, | | | M, L, S | T, I, L, W, C |
| V1342I, V1342W, V1342C | | | | |
| L1343I, L1343S, L1343K, L1343E, | | | I, S | K, E, W, F |
| L1343W, L1343F | | | | |
| D1344E, D1344A, D1344G, D1344S, | | | E, A, G, S | H |
| D1344H | | | | |
| A1345S, A1345C | | | S, C | |
| T1346N, T1346L, T1346K, T1346I, | | | N, L | K, I, R, V, Y |
| T1346R, T1346V, T1346Y | | | | |
| L1347V, L1347Y, L1347F, L1347S | | | V, Y, F | S |
| I1348V, I1348C, I1348L, I1348M, I1348A, | V, C | L | M, L, A, V | K, S, V, F |
| I1348K, I1348S, I1348F | | | | |
| H1349R, H1349A, H1349Y, H1349W, | R, A, Y, W | V, F | T, M, S | K, R, L, A, Y, |
| H1349V, H1349F, H1349T, H1349M, | | | | F |
| H1349S, H1349K, H1349L | | | | |
| Q1350R, Q1350S, Q1350W, Q1350L, | R | R, S, W, L | K, R, L | N, T, R, I, L, |
| Q1350K, Q1350N, Q1350T, Q1350I | | | | S, W |
| S1351T | | | T | |
| I1352K, I1352T, I1352H, I1352P, I1352R, | K, T, H, P, R, S | P, R | R, V, C | K, S, H, P, L, |
| I1352S, I1352V, I1352C, I1352L | | | | C |
| T1353C | | C | | C |
| G1354K, G1354P, G1354V | | | | K, P, V |
| L1355I, L1355T, L1355S | I | | I | T, S |
| Y1356S, Y1356R, Y1356V,Y1356C, | S | | S, R, V, C, L | S, Q, V, W |
| Y1356L,Y1356Q,Y1356W | | | | |
| E1357S, E1357T, E1357I, E1357F | S | T | T, S | I, F |
| T1358H, T1358R, T1358S, T1358P | H | | R, S | P |
| R1359Q, R1359C, R1359L, R1359N, | Q, C, L | N, V | Q, A, V, W, L | Y, L |
| R1359V, R1359A, R1359W, R1359Y | | | | |
| I1360R, I1360V, I1360S | | | | R, V, S |
| D1361N, D1361A, D1361S, D1361C, | | | N, A, S, C | A, G, S |
| D1361G | | | | |
| L1362C, L1362I, L1362P, L1362V, | | | C | I, P, V, F |
| L1362F | | | | |
| S1363T, S1363R, S1363M, S1363Q, | | | T, R, M, Q, A, G, V | H, V, W, L |
| S1363A, S1363G, S1363V, S1363H, | | | | |
| S1363W, S1363L | | | | |
| Q1364R, Q1364M, Q1364V, Q1364C, | | | R, M, V, C | N, R, S, I, H, |
| Q1364N, Q1364S, Q1364I, Q1364H, | | | | E, G |
| Q1364E, Q1364G | | | | |
| L1365M, L1365Y, L1365T, L1365S, | | | M, Y | T, M, S, C |
| L1365C | | | | |
| G1366A, G1366K, G1366M, G1366P, | | | A | K, M, P, D, S |
| G1366D, G1366S | | | | |
| G1367N, G1367S, G1367M, G1367P, | | | N, S, M, P, A, V, Y | T, S, Q, D, A |
| G1367A, G1367V, G1367Y, G1367T, | | | | |
| G1367Q, G1367D | | | | |
| D1368T, D1368M, D1368L, D1368S, | | | T, M, L, S, W | K, R, H, A, |
| D1368W, D1368K, D1368R, D1368H, | | | | G, V |
| D1368A, D1368G, D1368V | | | | |

TABLE 6
Amino acid changes that improve Cas9-activity-based survival with an enrichment
score of at least 1.5 using short scaffold regions guides in a bacterial screen
relative to the amino acid sequence of wild type Cas9 of SEQ ID NO: 133)

| Mutations above threshold of 1.5 | Screen 1 | Screen 1 | Screen 2 | Screen 2 |
|---|---|---|---|---|
| Cas9 Position | (GD1) | (GD2) | (GD1) | (GD2) |
| D2Q, D2C, D2N, D2R, D2M, D2V, D2Y, | | | Q, C | N, R, M, V, |
| D2F | | | | Y, C, F |
| K3I, K3V | | | I, V | |
| S6T | | | T | |
| G8T | | | | T |
| L9V | | | | V |
| T13S | | | | S |
| E24S | | | S | |
| K26D, K26S, K26R | D | S | R | D |
| P28V, P28Y, P28F | V, Y | F | | |
| S29N | | | | N |
| K30T, K30M, K30I, K30L, K30G, K30C, | T, M, I, L, G | T, M, G, C | G, V, C | Q, V |
| K30V, K30Q | | | | |
| K31M, K31P, K31E, K31S, K31G, K31V, | M, P, E | S, G | M, P, E, V, | R, Q, E, V, S |
| K31R, K31Q | | | S | |
| F32T, F32M, F32L, F32C, F32A | T, M, L | T, L | T, L, C | A |
| K33S, K33P, K33D, K33A, K33G, K33V, | S, P, D, A, | D, A, V, W | N, S, G | N, S, H, P, |
| K33W, K33N, K33H, K33R, K33L, K33E, | G, V | | | R, L, E, D, |
| K33C | | | | G, C |
| V34R, V34C | | | R, C | |
| L35E, L35A, L35N, L35S | E | | E, A | N, S |
| G36A, G36S | | A | S | |
| N37T | | | T | |
| T38G | | | G | |
| D39L, D39W | | | L | W |
| R40N, R40T, R40S, R40L, R40F, R40V, | N, T, S, L, F | L, V | E, A, G, V | S |
| R40E, R40A, R40G | | | | |
| H41T, H41R, H41L | T, R | | L | |
| S42Q, S42P, S42H, S42E | | Q, P | | H, P, E |
| I43A, I43W, I43Q, I43Y | | A, W | | Q, Y |
| K44Q, K44G, K44C, K44D | Q | G, C | D | |
| N46L, N46C, N46K, N46M, N46S | L, C | K, M | | S |
| I48M | | M | | |
| A50C, A50T, A50V, A50S, A50N, A50L, | C | T, V, S | N, L, Y, F | H, V |
| A50Y, A50F, A50H | | | | |
| L51T, L51R, L51V | T, R, V | R | | R |
| L52I | I | | | |
| F53L | | | L | |
| D54K, D54T, D54R, D54S, D54N, D54L, | K, T, R, S | K, N | R, S | L, E |
| D54E | | | | |
| S55P, S55E | | P, E | | P |
| G56A | A | A | A | A |
| E57K, E57Q, E57V, E57R | K, Q, V | K, R | | R |
| T58S, T58G | S, G | S | S, G | |
| E60K, E60R, E60Q, E60V, E60L, E60T | K, R, Q, V | K, R, L, V | K, T | K, R, V |
| A61R, A61N, A61L | R | R | N, L | |
| T62R | R | | R | |
| L64K, L64R | K | K | | K, R |
| K65H, K65L, K65Y, K65F | H, L, Y, F | H, Y, F | Y, F | H, Y, F |
| R66P | | P | | |
| T67S, T67A, T67G, T67Q, T67C, T67L | | S, A, G | Q, C | L |
| A68K, A68T, A68R, A68I, A68Q, A68L, | K, T, R, I, | K, T, R, Q | K, Q, L, V | K, T, R, M, I, |
| A68V, A68M | Q, L | | | Q |
| Y72N, Y72T, Y72I, Y72L, Y72G, Y72V, | N, T, I, L, | I, V | N, T, M, I, | T, I, V, C |
| Y72C, Y72F, Y72M | G, V, C, F | | L, V, C | |
| T73R | R | | | R |
| K76R | | R | | |
| N77K, N77R, N77C | K | K, R, C | K | K |
| R78P | | | P | |
| C80K, C80R, C80H, C80L, C80V, C80S, | K, R, H, L, | K, R, S, G | R, L, V | K, I |
| C80G, C80I | V | | | |
| S87R | | | | R |
| E89P | | | P | |
| A91H, A91N | | | H | N |
| K92L, K92A, K92V | | | L | A, V |
| S96T, S96R, S96H, S96E | | | | T, R, H, E |
| H99E, H99A, H99S, H99K | | E, A, S | | K, A |
| E102A, E102T, E102S | | A | | T, A, S |
| E108S, E108R, E108P, E108A, E108V | S | R, P, A, V | | P, V |
| H113F, H113Y | | F | | Y |
| E114S, E114A | S | | | S, A |
| R115K | K | | | |
| H116W | | | | W |
| D124E | E | | | |
| V126D, V126A | | D, A | | |
| A127R | | R | R | |
| E130K, E130A, E130L, E130N, E130I, | K, A, L | N | | I, Q |
| E130Q | | | | |
| K131Q, K131A, K131T, K131R, K131H | Q, A | T, R | | Q, H |
| K140T, K140A, K140G, K140Y, K140S, | | | T, A, G, Y, | T, Y, W, C, F |
| K140C, K140F, K140W | | | S, C, F | |
| K141E | | | E | |
| V143C | | | | C |
| S145E | | | | E |
| T146N | | | | N |
| D147H | | | H | |
| K148I, K148S, K148C | | | | I, S, C |
| L151A, L151P | | A | P | |
| L156S, L156N | S | N | | |
| A157I | | I | | |
| L158A | | A | | A |
| A159H | | H | | H |
| L169N, L169R, L169H, L169K | | | N, R, H | K, N, R, H |
| N175T, N175E, N175V | | | T, E, V | |
| D180T, D180S, D180M, D180H, D180E, | | | T, S, M, H, | H, E, G |
| D180A, D180G, D180W, D180C | | | E, A, G, | |
| | | | W, C | |
| V181P | | | P | |
| D182A, D182G | | | A, G | G |
| K183N, K183T, K183S, K183M, K183I, | | | N, T, S, M, | N, M, I, Q, |
| K183H, K183P, K183C, K183Q, K183G | | | I, H, P, C | H, G, C |
| F185T | | | T | |
| I186E, I186D, I186Q, I186C | | | E, D | Q, C |
| Q187H | | | | H |
| L188V | | | V | |
| V189N, V189Y, V189T, V189R, V189L | | | N, Y | T, R, L |
| Q190E, Q190W, Q190P | | | E, W | P |
| T191S | | | S | |
| Y192V | | | V | |
| N193G | | | G | |
| Q194L, Q194Y | | | L, Y | |
| L195T, L195I, L195H, L195E, L195V, | | | T, I, H, E, | T, H |
| L195F | | | V, F | |
| F196D, F196G, F196T, F196M | | | D, G | T, M, G |
| N199L, N199A | | | | L, A |
| P200R | | | | R |
| I201T, I201S, I201L | | | T, S, L | S |
| N202P | | | P | |
| A203I, A203T, A203M, A203L, A203V, | | | I | T, M, L, V, S |
| A203S | | | | |
| G205Q, G205M, G205L | | | Q | M, L |
| V206K, V206N, V206R, V206S, V206M, | | | K, N, R, S, | Q, D, G, Y |
| V206H, V206P, V206E, V206D, V206G, | | | M, H, P, E, | |
| V206F, V206Q, V206Y | | | D, G, F | |
| D207I | | | | I |
| A208S, A208H, A208L, A208E, A208G, | | | S, H, L, E, | M, E, G, V, |
| A208V, A208W, A208M, A208C | | | G, V, W | C |
| A210R | | | | R |
| I211L | | | | L |
| L212I, L212V | | | I, V | |
| S213L, S213E | | | L, E | L |
| R215T, R215M, R215I, R215Q, R215P, | | | T, M, I, Q, | L, Y |
| R215L, R215V, R215Y, R215W, R215C | | | P, L, V, Y, | |
| | | | W, C | |
| L216E | | | E | |
| S217M, S217N, S217P, S217D, S217G | | | M | N, M, P, D, |
| | | | | G |
| S219Q, S219L, S219A, S219V, S219F | | | Q, L, A | V, F |
| R220T, R220Q, R220H, R220P, R220L, | | | T, Q, H, P, | S, M, P, E, |
| R220E, R220A, R220V, R220S, R220M, | | | L, E, A, V | D, V, F |
| R220D, R220F | | | | |
| L222N, L222M, L222H, L222A, L222G, | | | N, M, H, | N, S, H, A, |
| L222Y, L222W, L222C, L222F, L222S | | | A, G, Y, | G, Y, W, C, |
| | | | W, C, F | F |
| E223P | | | | P |
| N224P, N224L | | | P, L | P |
| L225Y, L225W, L225Q | | | Y, W | Q, Y |
| I226K, I226M, I226Q, I226P, I226D, | | | K, M, Q, P, | R, Q, H, A |
| I226G, I226R, I226H, I226A | | | D, G | |
| A227W, A227P | | | W | P |
| L229M, L229P, L229S | | | M, P | S |
| P230M, P230S, P230V, P230Y | | | M, S | V, Y |
| G231K, G231D, G231M, G231A, G231F | | | K, D | M, A, F |
| E232H, E232I, E232P | | | H | I, P |
| K233E | | | | E |
| K234I, K234Q, K234S | | | I, Q | S |
| G236P, G236L, G236I | | | P, L | I, P |
| L237T, L237I, L237P, L237C, L237F | | | T | I, P, C, F |
| F238P | | | P | |
| G239T, G239P, G239V, G239M, G239I, | | | T, P, V | T, M, I, L, V, |
| G239L, G239Y, G239F | | | | Y, F |
| N240Q, N240P | | | | Q, P |
| L241V, L241Y, L241W, L241F, L241T, | | | V, Y, W, F | T, S, I, A, Y |
| L241S, L241I, L241A | | | | |
| I242L, I242Y | | | L, Y | Y |
| A243T, A243C | | | | T, C |
| L244R, L244W | | | | R, W |
| S245Y, S245F | | | | Y, F |
| L246M, L246H, L246G, L246Y | | | M, H, G | H, Y |
| G247N, G247T, G247S, G247V, G247C | | | N, T, S | S, V, C |
| L248T | | | | T |
| T249P, T249F, T249R, T249A | | | P, F | R, A |
| P250N, P250T, P250S, P250L, P250D | | | N | N, T, S, L, D |
| N251E, N251A | | | E | A |
| F252P, F252A | | | | P, A |
| K253S, K253L, K253E, K253Y | | | S, L | L, E, Y |
| N255V | | | V | |
| F256Q, F256A, F256N, F256T, F256S, | | | Q, A | N, T, S, L, A, |
| F256L, F256V, F256C | | | | V, C |
| L258T, L258P, L258F | | | | T, P, F |
| A259P, A259W, A259C, A259M, A259L | | | P, W, C | M, L, W |
| E260I | | | | I |
| A262C, A262E, A262V | | | C | E, V |
| K263R, K263G, K263A | | | R, G | A |
| L264K, L264S, L264M, L264Q, L264H, | | | K | S, M, Q, H, |
| L264P | | | | P |
| Q265S, Q265L | | | | S, L |
| L266T, L266C, L266V | | | T, C | V |
| S267I, S267P, S267L | | | I, P | I, L |
| Y271P, Y271S, Y271K, Y271Q | | | P, S | K, Q |
| D272K, D272R, D272L, D272G, D272C, | | | K, R, L, G, | K, R, S, M, I, |
| D272S, D272M, D272I, D272P, D272V | | | C | P, L, V |
| D273K, D273R, D273S, D273Q, | | | K, R, S, Q, | K, T, R, S, P, |
| D273H, D273L, D273A, D273Y, D273T, | | | H, L, A, Y | A, G, V |
| D273P, D273G, D273V | | | | |
| D274P, D274S, D274M, D274L, D274G, | | | P | S, M, P, L, |
| D274V, D274W | | | | G, V, W |
| L275K, L275S, L275H, L275M, L275E, | | | K, S, H | K, M, H, E, |
| L275D, L275G, L275W | | | | D, G, W |
| D276K, D276N, D276T, D276R, D276S, D276M, | | | K, N, T, R, | K, N, T, R, |
| D276I, D276Q, D276H, D276P, D276L, D276G, | | | S, M, I, Q, | M, I, Q, H, |
| D276W, D276C, D276F, D276A, D276V, D276Y | | | H, P, L, G, | P, L, A, G, V, |
| | | | W, C, F | Y, C, F |
| N277R | | | | R |
| L278F | | | F | |
| L279N, L279S, L279Q, L279H, L279A, | | | N, S, Q, | K, N, R, S, I, |
| L279G, L279K, L279R, L279I, L279P, | | | H, A, G | Q, H, P, E, |
| L279E, L279D, L279V, L279Y, L279W | | | | D, A, G, V, |
| | | | | Y, W |
| A280K, A280R, A280Q, A280H, A280L, | | | K, R, Q, | K, T, R, I, Q, |
| A280Y, A280W, A280C, A280F, A280T, | | | H, L, Y, W, | H, L, V, Y, |
| A280I, A280V | | | C, F | W, F |
| Q281R, Q281A, Q281Y | | | | R, A, Y |
| I282H, I282E, I282D, I282G, I282W, | | | H, E, D, | N, H, R, S |
| I282N, I282R, I282S | | | G, W | |
| G283T, G283D, G283Y, G283W, | | | T, D, Y, W | N, S, Q, P, |
| G283N, G283S, G283Q, G283P, G283F | | | | D, Y, W, F |
| D284T, D284A, D284W | | | | T, A, W |
| Q285S, Q285M, Q285E, Q285Y, Q285L, | | | S, M, E, Y | L, A |
| Q285A | | | | |
| Y286T, Y286E, Y286D, Y286V, Y286G | | | T, E, D, V | D, G |
| A287N, A287T, A287R, A287S, A287M, A287I, A287E, | | | N, T, R, S, | K, N, R, S, I, |
| A287D, A287G, A287Y, A287W, A287F, A287K, | | | M, I, E, D, | Q, H, P, L, |
| A287Q, A287H, A287P, A287L, A287V | | | G, Y, W, F | D, G, V, W, |
| | | | | F |
| D288S, D288Q, D288E, D288W | | | | S, Q, E, W |
| L289S, L289G, L289V, L289C | | | S, G, V | V, C |
| F290T, F290S, F290Q, F290H, F290A, | | | | T, S, Q, H, |
| F290G | | | | A, G |
| L291N, L291R, L291D, L291W, L291K, | | | N, R, D, | K, N, T, R, |
| L291T, L291S, L291P, L291A, L291G, | | | W | S, P, A, G, Y, |
| L291Y, L291C, L291F | | | | W, C, F |
| A292R | | | R | |
| A293S, A293I, A293L, A293D, A293G, | | | S, I, L, D, | T, H, L, D, V |
| A293V, A293T, A293H | | | G, V | |
| N295K, N295R, N295C, N295Q, N295P, | | | K, R, C | Q, P, A |
| N295A | | | | |
| L296A, L296W, L296T, L296D | | | A, W | T, D, A |
| S297Y, S297W, S297K, S297N, S297G | | | Y, W | K, N, G |
| D298M, D298H, D298N | | | M, H | N |
| A299T, A299E, A299C | | | T, E, C | C |
| L301I, L301R, L301G, L301V, L301C, | | | I, R, G, V, | K, N, R |
| L301K, L301N | | | C | |
| S303E | | | E | |
| D304L, D304W | | | L, W | W |
| R307T, R307S, R307G | | | T, S | G |
| V308W | | | W | |
| T310S, T310A, T310G, T310P, T310L | | | S, A, G | P, L |
| T313E | | | E | |
| K314S, K314L, K314E, K314D, K314A, | | | S, L, E, D, | C |
| K314C | | | A | |
| I322V, I322T, I322C | V | V | T, V, C | |
| K323N, K323T, K323M, K323I, K323Q, | N, T, M, I, | M, I, Q, L, | S, M, I, Q, | M, L |
| K323H, K323L, K323E, K323D, K323A, | Q, H, L, E, | A | H, L, E, D, | |
| K323G, K323V, K323Y, K323S, K323C | D, A, G, V, | | A, V, C | |
| | Y | | | |
| D326A | | | | A |
| E327T | T | | | |
| H329K, H329R | | K, R | | |
| Q330E, Q330S, Q330V | E, S | V | | |
| L332K | | K | | |
| T333L, T333S, T333A, T333G, T333V, | L, S, A, G, | K, N, R, H | N | R, M, I |
| T333K, T333N, T333R, T333H, T333M, | V | | | |
| T333I | | | | |
| L334T, L334Q, L334V, L334S, L334A | T, Q, V | | S | T, A |
| L335A | | A | | |
| A337S, A337Q | S | S, Q | | |
| L338A, L338T | | A | T | |
| V339T | T | | | |
| Q341L, Q341R, Q341C | L | R, C | | |
| Q342S, Q342E, Q342R, Q342W | S, E | R, W | | |
| L343R, L343K, L343M, L343W, L343S | R, K, M | W | | S |
| P344T, P344R, P344S, P344H, P344G | T, R, S | R | | H, G |
| E345N, E345T, E345R, E345S, E345Y, | N, T, R, S | S, Y | N, R, M, I, | |
| E345M, E345I, E345H, E345L | | | H, L | |
| K346M | | M | | |
| E349T, E349R, E349S, E349M, E349L, | T, R, S, M, | M | R, M, L | N, G |
| E349V, E349W, E349N, E349G | L, V, W | | | |
| I1350L, I350V | L | | V | |
| F352S | | S | | |
| D353L, D353S, D353A, D353C | L, S | | A, C | |
| Q354R, Q354S, Q354G, Q354Y, Q354T, | R, S, G, Y | | T, R, D, G | S |
| Q354D | | | | |
| S355R, S355K, S355C, S355Y | R, K | C | K, R | R, Y |
| K356S | S | | | |
| N357R, N357K, N357S | R, K, S | | R | |
| D364K, D364T, D364R, D364S, D364M, | K, T, R, S, | | R, H, G | |
| D364H, D364G, D364V | M, H, G, V | | | |
| G366P, G366L, G366S | P, L | | L, S | |
| A367R, A367T | | R | T | |
| S368N | | | N | |
| E370A | | | | A |
| E371R, E371Q, E371L, E371K, E371S | | | R, Q, L | K, S |
| F375H | | | H | |
| I376V | | | V | |
| K377A | | | A | |
| P378V, P378F | | | | V, F |
| I379S, I379H | | | S | H |
| E381I, E381H, E381R, E381L | | | I, H, R, L | |
| K382R | | | R | |
| D384V | | | | V |
| G385H | | | | H |
| L390I | | | | I |
| V391E, V391D, V391T, V391A | | | E, D | T, E, A |
| K392S, K392A | | | S, A | |
| L393V | | | V | |
| N394E, N394D, N394V | | | E, D | V |
| R395I, R395L, R395A, R395C | | | I, L, A, C | |
| L399C | | | | C |
| T404M, T404V | | | M, V | |
| F405T, F405S, F405I, F405R, F405V | | | T | T, S, I, R, V |
| S409A, S409V, S409C | | | A, V, C | A, V, C |
| H420G | | | | G |
| R424E, R424V | | | E, V | |
| R425S, R425C | | | | S, C |
| E427K | | | | K |
| D428T | | | | T |
| F429H | | | H | |
| K434A | | | A | |
| D435M | | | | M |
| N436Q | | | Q | |
| R437C, R437A | | | C | A |
| E438L | | | L | |
| I440L | | | | L |
| E441R | | | | R |
| T445S, T445W, T445R, T445E | S, W | R | S, E | |
| R447T | T | | | |
| Y450H, Y450S | H | S | H | H |
| Y451W | W | W | | |
| R457K, R457N, R457A, R457M | K, N, A | M | | |
| M465L | L | L | L | L |
| T466V, T466I | V | I | | |
| E470D | D | | D | |
| E471Q | | | | Q |
| T472K, T472R, T472S, T472H, T472P, | K, R, S | K, R, S, H | P, A | K, N, R |
| T472A, T472N | | | | |
| T474R, T474H | R | R | | R, H |
| E479D, E479W | | | | D, W |
| E480S, E480A | | | S | A |
| K484L | | | L | |
| G485K, G485L, G485A, G485V | | | K, L, A | V |
| Q489L, Q489A | | | L, A | |
| S490A, S490G | | | A, G | |
| E493S | | | S | |
| M495H | | | H | |
| T496V | | | | V |
| N497P, N497Q | | | P | Q |
| F498N, F498S, F498L, F498A, F498V, | | | N, S, L, A, | N, R, G |
| F498C, F498R, F498G | | | V, C | |
| D499R | | | | R |
| K500R, K500S, K500P, K500E, K500A, | | | R, S, P, E, | T, Q |
| K500G, K500V, K500T, K500Q | | | A, G, V | |
| N501R, N501H | | | R, H | |
| L502V, L502A | | | V | A |
| P503R | | | | R |
| N504E | | | E | |
| E505T | | | | T |
| V507S, V507L | | | | S, L |
| H511R | | | | R |
| S512R, S512L | | | | R, L |
| L513K, L513I, L513V | | | K | I, V |
| L514T, L514V | | | | T, V |
| Y515A, Y515L | | | A | L |
| Y517D, Y517G, Y517V | | | D, G | V |
| T519D, T519Y, T519S, T519I, T519Q | | | D, Y, S | I, Q |
| V520T, V520A | | | T | A |
| E523A | | | | A |
| L524T, L524S | | | T | S |
| T525Y | | | | Y |
| K526L | | | | L |
| V527T | | | T | |
| K528R, K528I, K528V, K528W | | | R, I, V, W | I |
| Y529S | | | S | |
| V530D, V530T, V530Q | | | D | T, Q |
| T531Q, T531D, T531Y | | | Q, D, Y | |
| E532G | | | G | |
| G533A, G533R, G533E, G533D | | | A | R, E, D |
| M534S | | | S | |
| R535L, R535D, R535A, R535G | | | L | D, A, G |
| K536I, K536D, K536V, K536F, K536N, | | | I, D, V, F | N, E, Y, C |
| K536E, K536Y, K536C | | | | |
| P537Q, P537F | | | | Q, F |
| A538L, A538Y, A538P | | | L, Y | P |
| S541I, S541G | | | I | G |
| G542L | | | L | |
| E543C | | | C | |
| K545N | | | N | |
| K546Q, K546C | | | | Q, C |
| A547M, A547H, A547P, A547V, A547F | | | M | H, P, V, F |
| I548M, I548C | | | M | C |
| V549L, V549C | | | L, C | |
| D550S | | | S | |
| L551A | | | | A |
| L552A | | | | A |
| T555D, T555G, T555F, T555Y | | | D, G, F | Y |
| N556I | | | | I |
| R557I, R557F | | | I | F |
| K558T, K558F, K558W | | | T, F | W |
| V559L, V559A | | | L, A | |
| T560K, T560S | | | | K, S |
| K562H, K562R, K562L, K562A, K562T, | | | H, R, L, A | T, S |
| K562S | | | | |
| Q563V, Q563I | | | V | I |
| L564T, L564C, L564P | | | T, C | P |
| K565H, K565P | | | H | P |
| D567C | | | C | |
| Y568I, Y568H | | | I | H |
| F569L | | | | L |
| K570L, K570V | | | | L, V |
| K571H, K571T | | | H | T |
| I572C | | | C | |
| E573R, E573H, E573Y, E573G | | | R, H, Y | G |
| C574I, C574P, C574A, C574V | | | I, P | A, V |
| F575I, F575E, F575G | | | I | I, E, G |
| D576H | | | | H |
| S577T | | | T | |
| V578M, V578P | | | M | P |
| E579Y, E579S, E579I | | | Y, S | I, Y |
| I580F | | | | F |
| S581R, S581L | | | | R, L |
| G582Q, G582L, G582V, G582R, G582Y | | | Q, L, V | R, Q, Y |
| V583F, V583R, V583M | | | F | R, M |
| D585Q | | | Q | |
| R586L, R586Y, R586H, R586P | | | L, Y | H, P |
| S590V, S590M | | | V | M |
| L591H | | | | H |
| G592L, G592S, G592W, G592F, | | | L, S, W, F | H, Y |
| G592H, G592Y | | | | |
| T593G | | | G | |
| L598S, L598W, L598A | | | S, W | A |
| K599I, K599H, K599L, K599A, K599Q, | | | I, H, L, A | Q, A, G, S |
| K599G, K599S | | | | |
| K602M, K602P, K602R, K602G, K602W, | | | M, P, R, | S, L, E, V |
| K602S, K602L, K602E, K602V | | | G, W | |
| D603T, D603V, D603E | | | T, V | E |
| K604G, K604Y | | | G, Y | |
| D605G | | | | G |
| F606V | | | V | |
| L607T | | | | T |
| N609P, N609D, N609W | | | P | D, W |
| E610L | | | L | |
| E611L, E611H, E611R, E611G, E611S | | | L | H, R, G, S |
| N612T, N612A | | | | T, A |
| E613Q | | | Q | |
| D614S | | | S | |
| I615S, I615L, I615G, I615R | | | S, L, G | R, S |
| L616V | | | V | |
| D618P, D618L | | | | P, L |
| I619T, I619L, I619M | | | T, L | M |
| V620A, V620T | | | A | T |
| L621H, L621W, L621F, L621S | | | H, W, F | S, W, F |
| L623M | | | | M |
| T624S | | | | S |
| E627Y, E627R, E627M, E627L | | | Y | R, M, L |
| R629T, R629Q, R629L, R629W, R629I, | | | T, Q, L, W | I, F |
| R629F | | | | |
| I632L | | | L | |
| E633D, E633S | | | D, S | D |
| E634S, E634T, E634V | | | S | T, V |
| L636F | | | F | |
| K637N, K637A, K637G | | | | N, A, G |
| T638H, T638P, T638G, T638V | | | H | P, G, V |
| Y639I, Y639A, Y639V | | | I, A, V | |
| A640I, A640E, A640L | | | I | E, L |
| K646M, K646Q, K646L, K646E, K646D, | | | M, Q, L, | T, S, I, Q, H, |
| K646A, K646G, K646V, K646C, K646F, | | | E, D, A, | P, L, E, D, A |
| K646T, K646S, K646I, K646H, K646P | | | G, V, C, F | |
| V647M | | | | M |
| M648W, M648Y | | | W | Y |
| K649T, K649I, K649Q, K649H, K649P, K649L, K649E, | | | T, I, Q, H, | S, M, Q, H, |
| K649D, K649A, K649G, K649V, K649Y, K649W, | | | P, L, E, D, | P, E, G, V, |
| K649C, K649F, K649S, K649M | | | A, G, V, Y, | W, C, F |
| | | | W, C, F | |
| Q650E, Q650D, Q650A, Q650V, Q650L | | | E | E, D, A, V, L |
| K652M, K652H, K652E, K652D, K652W, | | | M, H, E, | Q, H, Y, W |
| K652F, K652Q, K652Y | | | D, W, F | |
| R653T, R653S, R653M, R653I, R653L, | | | T, S, M, I, | L, G |
| R653E, R653D, R653A, R653G, R653V, | | | L, E, D, A, | |
| R653C | | | G, V, C | |
| R655L, R655Y | | | L, Y | |
| Y656W | | | W | W |
| T657R, T657M, T657A | | | R, M, A | R |
| G658R, G658H, G658K | | | R, H | K |
| G660A | | | | A |
| L662D, L662E | | | D | E |
| R664L, R664S, R664P | | | L | S, P |
| K665Q, K665H, K665V | | | | Q, H, V |
| I670L, I670H | | | L | H, L |
| R671L, R671S | | | L | S |
| K673H, K673Y, K673S, K673G, K673V | | | H, Y | S, G, V |
| Q674G, Q674K, Q674N, Q674S, Q674L | | | G | K, N, S, L |
| S675A | | | | A |
| G676N, G676R, G676Q, G676L, G676S | | | N, R, Q, L | S |
| K677M, K677H, K677S, K677A | | | M, H | S, A |
| F682L | | | | L |
| S685H, S685R | | | H, R | R |
| G687S, G687V | | | S | V |
| F688R | | | | R |
| A689M, A689P, A689E, A689L | | | M, P, E | L |
| N690S | | | S | |
| R691S, R691H, R691N, R691L | | | S, H | N, L |
| N692A, N692G | | | A, G | |
| M694P, M694W | | | P, W | |
| Q695G | | | G | G |
| I697C | | | C | |
| H698S, H698W | | | | S, W |
| D700T, D700S, D700Q, D700H, D700A, | | | T, S, Q, H, | I, G, V |
| D700G, D700C, D700I, D700V | | | A, G, C | |
| S701I, S701R, S701E, S701Y | | | I, R, E | R, Y |
| L702F | | | F | |
| T703S | | | S | |
| K705R | | | | R |
| E706G, E706R | | | G | R |
| I708L | | | | L |
| K710S, K710A | | | | S, A |
| A711S | | | | S |
| Q712M, Q712W | | | M, W | |
| V713P, V713T, V713R | | | P | T, R |
| S714L, S714T, S714M, S714R, S714A, | | | L | T, M, R, A, F |
| S714F | | | | |
| G715R, G715P, G715L, G715S, G715A | | | R, P, L | S, A |
| G717P, G717A, G717T, G717I | | | P, A | T, I |
| D718K, D718R, D718M, D718T, D718Q, | K, R, M | T, Q, H | K, R, S, G, | K, N, R, G |
| D718H, D718S, D718G, D718V, D718N | | | V | |
| S719N | N | N | | |
| L720A, L720V | | A | | V |
| H721K, H721V, H721Q, H721L, H721P | K, V | Q, L, V | V | P, V |
| E722K, E722T, E722R, E722W, E722D, | K | K, T, R, W | D | T, R, L |
| E722L | | | | |
| H723K, H723N, H723T, H723M, H723I, | K, N, T, M, | R, M, Q, L | M, A, C | N, R |
| H723Q, H723A, H723S, H723R, H723L, | I, Q, A, S | | | |
| H723C | | | | |
| A725K, A725R, A725Q, A725H, A725W, | K, R, Q, H, | K, R, Q, V, | R, L | K, N, R, H, |
| A725V, A725C, A725L, A725N, A725P | W | C | | P |
| N726R, N726A, N726K | R | R, A | K | R |
| L727V, L727G | V | G | | V |
| A728Q, A728E, A728D, A728G, A728Y, | Q, E, D, G, | R, P, D, G, | Q, L, G, Y, | R, P, E, D, |
| A728W, A728F, A728R, A728P, A728S, | Y, W, F | S, W, F | W | G, S, W, F |
| A728L | | | | |
| G729N, G729T, G729S, G729M, G729I, | N, T, S, M, | N, T, R, S, | N, R, M, I, | N, T, R, S, |
| G729Q, G729H, G729P, G729L, G729E, | I, Q, H, P, | M, I, Q, H, | Q, H, L, E, | M, I, Q, H, |
| G729D, G729A, G729V, G729Y, | L, E, D, A, | L, E, D, A, | A, V, Y, W, | P, L, E, D, A, |
| G729W, G729C, G729F, G729R | V, Y, W, C, | V, Y, W, C, | C | V, Y, W, C, F |
| | F | F | | |
| S730N, S730P, S730G, S730C, S730K, | N, P, G, C | P, G, C | N, G, C | K, N, R, H, |
| S730R, S730H, S730D, S730A | | | | P, D, A, G, |
| | | | | C |
| P731T, P731M, P731V | | T, M | V | |
| A732N, A732S, A732M, A732T, A732Q, | N, S | S, M | N, T, S, Q, | T, R, V |
| A732P, A732R, A732V | | | P | |
| I733T, I733V, I733E, I733Y, I733A, | T, V | E, Y | A | N, V |
| I733N | | | | |
| K734R | R | | R | R |
| K735R | R | | R | |
| G736S, G736W, G736T, G736V | S | W | T, V | |
| I737V | V | V | V | |
| L738R, L738Y, L738W, L738F, L738M, | R, Y, W, F | M, V, Y, W, | R, V, W | T, R, W, F |
| L738V, L738T | | F | | |
| Q739R | R | | | |
| T740A | A | | | |
| V741L, V741G | L | | G | G |
| V743L, V743I | L | I | | |
| D745T | | T | | |
| V748T | | | | T |
| K749L | | | | L |
| V750S, V750I, V750C | | S | I, C | |
| R753N, R753M, R753H, R753D, | N, M, H, D, | S, P, G, C | N, S, G | N, T, S, P, L, |
| R753G, R753S, R753P, R753C, R753T, | G | | | Y |
| R753L, R753Y | | | | |
| H754T | | T | | |
| N758L, N758V | | | L | V |
| I759L | | | L | |
| M763V | | | | V |
| A764V | | | V | |
| R765S | | | S | S |
| E766Y, E766W, E766R | | | Y, W | R |
| N767T, N767A, N767V, N767S | | | T, A | V, S |
| Q768W | | | W | |
| Q771A, Q771T, Q771Y, Q771S | | | A | T, Y, S |
| Q774L, Q774V | | | L | V |
| K775R, K775A | | | | R, A |
| N776R, N776C, N776Q | | | R, C | Q |
| R778H, R778N, R778L | | | H | N, L |
| M781G | | | | G |
| K782R, K782A, K782Y | | | R | A, Y |
| R783T, R783I, R783P, R783L, R783M, | | | T, I, P, L | M, I, Q, A, |
| R783Q, R783A, R783G, R783V, R783C | | | | G, V, C |
| E785Q | | | Q | |
| E786H | | | | H |
| K789H, K789Q, K789R | | | H | Q, R |
| G792K, G792S | | | | K, S |
| Q794T, Q794M, Q794P, Q794L, Q794V | | | | T, M, P, L, V |
| I795Q | | | | Q |
| K797M, K797Y, K797S | | | M | Y, S |
| E798L, E798V | | | | L, V |
| H799T, H799K | | | T | K |
| V801C, V801S | | | C | S |
| E802S | | | | S |
| T804P, T804Y, T804R, T804D | | | P, Y | R, D |
| Q805A, Q805Y | | | A, Y | |
| L806P | | | | P |
| Q807R | | | | R |
| K810R | | | | R |
| Y812F, Y812M | | | F | M |
| L816S, L816F | | | S | F |
| Q817S | | | | S |
| N818S, N818D | | | S | D |
| R820K, R820Y, R820C | | | K | Y, C |
| V824G, V824Y | | | | G, Y |
| D825P, D825N, D825Y | | | P | N, Y |
| Q826K | | | K | |
| E827P | | | | P |
| I830S | | | | S |
| N831L, N831V, N831F | | | | L, V, F |
| R832S | | | S | |
| S834V | | | V | |
| D835T, D835H, D835A, D835V | | | | T, H, A, V |
| Q844A | | | A | |
| F846M, F846Y | | | | M, Y |
| K848R | | | R | |
| I852W | | | W | W |
| D853S, D853G | | | | S, G |
| K855L | | | L | |
| V856A | | | A | |
| R859L | | | L | |
| K862S, K862H | | | S | H |
| G865R, G865C | | | | R, C |
| S867L | | | L | |
| D868K, D868T | | | K, T | |
| N869T, N869S | | | | T, S |
| V870L | | | | L |
| E873C | | | | C |
| E874H, E874D, E874G, E874C, E874S | | | H, D, G, C | S |
| V875I, V875C | | | | I, C |
| K877T, K877S, K877M, K877R, K877L, | | | T, S, M, R, | C |
| K877A, K877C | | | L, A, C | |
| M879R | | | R | |
| K880L, K880I | | | L | |
| N881T, N881Q, N881P, N881D, N881V, | | | T, Q, P, D, | G |
| N881G | | | V | |
| Y882D, Y882A, Y882V, Y882S | | | D, A | V, S |
| Q885T, Q885S, Q885A, Q885C | | | T, S, A | C |
| L886Y | | | Y | |
| N888T, N888H, N888R, N888V | | | T, H, R | R, V |
| K890R | | | | R |
| I892V | | | V | |
| T893G | | | G | |
| Q894T, Q894G | | | T | G |
| F897W | | | | W |
| N899C, N899W | | | C | W |
| T901M | | | | M |
| K902A | | | A | |
| A903F | | | | F |
| R905T, R905Q, R905L, R905E, R905A, | | | T, Q, L, E, | T, Q, E, V, Y, |
| R905Y, R905S, R905C, R905V, R905W, | | | A, Y, S, C | W, F |
| R905F | | | | |
| G907S | | | S | |
| S909D | | | D | |
| E910F | | | | F |
| L911M | | | | M |
| K913G, K913V | | | | G, V |
| A914M, A914T | | | M | T |
| G915H, G915A, G915F | | | H, A | F |
| K918R | | | | R |
| R919Q, R919I | | | Q | I |
| Q920L | | | | L |
| V922A, V922C | | | A, C | |
| E923M, E923I, E923H, E923L, E923A, | | | | M, I, H, L, |
| E923V | | | | A, V |
| T924V | | | | V |
| Q926N, Q926M, Q926W, Q926C | | | | N, M, W, C |
| I927Q, I927L | | | | Q, L |
| T928I, T928C | | | I | C |
| K929T, K929L | | | T | L |
| H930T, H930Q, H930E, H930A, | | | T, Q, E, A | T, M, Q, L, |
| H930M, H930L, H930F | | | | E, F |
| A932T, A932V | | | | T, V |
| Q933R, Q933L | | | | R, L |
| S937Q, S937R | | | Q, R | |
| R938K | | | K | |
| M939Y, M939F | | | | Y, F |
| T941Q, T941C | | | Q | C |
| K942T, K942R | | | T | T, R |
| Y943H, Y943L, Y943R, Y943V | | | H, L | R, V |
| D944A | | | | A |
| E945S | | | S | |
| D947N, D947M, D947I, D947S | | | N, M, I | S |
| K948D | | | D | |
| L949K, L949V | | | K | V |
| I950L | | | | L |
| R951M, R951E | | | M | E |
| E952N | | | | N |
| K954R, K954C | | | | R, C |
| I956L | | | L | I |
| T957I | | | | |
| S960T, S960G, S960Y | | | T | G, Y |
| K961G | | | | G |
| L962C | | | | C |
| V963P, V963L, V963S, V963M | | | P, L | S, M |
| S964R, S964L, S964A | | | R, L, A | |
| D965S | | | S | |
| K968L, K968F, K968R | | | L, F | R |
| D969V, D969M | | | V | M |
| F970V | | | | V |
| Q971G, Q971S | | | | G, S |
| F972K, F972G, F972S | | | | K, G, S |
| Y973E, Y973N, Y973A | | | E | N, A |
| V975I, V975G, V975L, V975M | | | I, G, L | M |
| E977Q, E977S | | | Q | S |
| I978E, I978M, I978L | | | E | M, L |
| N979A | | | | A |
| N980T, N980S, N980I, N980M, N980L, | | | T, S, I | M, L, D, A |
| N980D, N980A | | | | |
| Y981L | | | | L |
| H983N | | | | N |
| A984R | | | | R |
| D986K | | | | K |
| L989M | | | | M |
| N990S, N990M | | | S | M |
| T995R | | | R | |
| A996T | | | | T |
| K999R | | | R | |
| K1003H, K1003R, K1003V | | | H | R, V |
| E1005L, E1005G | | | | L, G |
| S1006G | | | | G |
| E1007M, E1007T, E1007S, E1007Q, | | | M | T, S, Q, R, |
| E1007R, E1007G, E1007W, E1007C | | | | G, W, C |
| F1008L, F1008S | | | L | S |
| Y1010M | | | M | |
| D1012S, D1012L | | | S | L |
| Y1013G | | | | G |
| K1014R | | | R | |
| V1015T, V1015I, V1015D, V1015G, | | | T, I, D, G | N, I, P, L, G, |
| V1015N, V1015P, V1015L, V1015F | | | | F |
| Y1016R, Y1016M, Y1016S | | | R, M | S |
| V1018W | | | | W |
| R1019E | | | E | |
| K1020T, K1020D, K1020S | | | T, D | S |
| M1021S, M1021A, M1021F | | | | S, A, F |
| A1023C, A1023L | | | C | L |
| K1024S, K1024A, K1024E, K1024D | | | S, A | E, D |
| E1026I | | | | I |
| Q1027I, Q1027T, Q1027C | | | I | T, C |
| I1029L, I1029M | | | L | M |
| G1030E | | | | E |
| K1031L, K1031D, K1031S | | | L | D, S |
| A1032G, A1032K | | | G | K |
| T1033S | | | S | |
| A1034S, A1034V, A1034Q, A1034L | | | S, V | Q, L |
| K1035S, K1035V | | | S | V |
| Y1036M, Y1036Q | | | M | Q |
| I1042W, I1042L | | | | W, L |
| T1048L | | | L | |
| E1049N, E1049Y | | | N | Y |
| I1050F, I1050L, I1050E, I1050V | | | F | L, E, V |
| T1051Q, T1051E, T1051Y, T1051R | | | Q, E, Y | R |
| L1052P | | | P | |
| A1053M, A1053S | | | M | S |
| N1054A | | | | A |
| G1055E | | | E | |
| E1056L | | | L | |
| I1057R, I1057V, I1057G | | | R, V | G |
| R1058S | | | | S |
| K1059Q, K1059A, K1059S | | | Q, A | S |
| R1060N, R1060C | | | N, C | |
| P1061A, P1061G, P1061S | | | | A, G, S |
| L1062I | | | I | |
| I1063T, I1063V | | | T, V | T |
| T1065C, T1065A | | | C | A |
| N1066S, N1066C | | | S, C | |
| G1067P, G1067L, G1067E, G1067D, | | | P, L, E | L, D, A |
| G1067A | | | | |
| E1068S, E1068A | | | | S, A |
| T1069R, T1069S | | | | R, S |
| E1071L | | | L | |
| I1072R, I1072A | | | R, A | |
| G1077D, G1077L | | | D | L |
| R1078K, R1078T, R1078L | | K, T, L | | |
| D1079A, D1079G | | | A | G |
| F1080V, F1080H | | V | H | |
| A1081G, A1081P, A1081D, A1081R, | | G | P, D | R, H, L, V |
| A1081H, A1081L, A1081V | | | | |
| T1082E | | E | | |
| V1083L | | L | | |
| R1084T, R1084S, R1084H, R1084Q, | T, S, H | Q, E | D, S | V |
| R1084E, R1084D, R1084V | | | | |
| K1085H, K1085E, K1085D, K1085A, | H, E, D | H, A, C | T, S, G | |
| K1085C, K1085T, K1085S, K1085G | | | | |
| S1088D | D | | | |
| M1089D, M1089V, M1089E, M1089S, | D, V | V | E, S | G, F |
| M1089G, M1089F | | | | |
| P1090Y, P1090C, P1090Q, P1090A | | Y, C | | Q, A, C |
| Q1091T, Q1091I, Q1091L, Q1091S, | T, I, L | S, L, E, V | T, S | V |
| Q1091E, Q1091V | | | | |
| V1092L, V1092A, V1092C | | L, A | C | |
| N1093A | | | A | |
| I1094M, I1094L, I1094P | M, L | | P, L | |
| V1095K | | K | | K |
| K1096N, K1096T, K1096I, K1096V, | N, T, I, V, C | N, S, R, V, | N, R, L, A, | T, R, I, V, S, |
| K1096C, K1096S, K1096R, K1096Y, | | Y, C | C | W, C |
| K1096L, K1096A, K1096W | | | | |
| K1097R | | R | | |
| T1098K, T1098R, T1098I, T1098V, | K, R, I | K, R, V | K, R, L, S | K, V |
| T1098L, T1098S | | | | |
| E1099K, E1099R, E1099S, E1099H, | K, R, S, H, | A | N, T, R, S, | K, R, S |
| E1099V, E1099Y, E1099A, E1099N, | V, Y | | M, Q, H, | |
| E1099T, E1099M, E1099Q, E1099L, | | | L, V, C | |
| E1099C | | | | |
| V1100K, V1100R, V1100C, V1100T | | K, R, C | K, T | T |
| Q1101V, Q1101A | V | | A | |
| T1102R | R | | | |
| G1104S, G1104A, G1104P, G1104C | S, A | P, A | A | A, C |
| S1106T, S1106H, S1106A, S1106Y, | T, H, A, Y, | T, H, Y, F | T, H, A, Y, | T, H, Y, W, |
| S1106W, S1106C, S1106F | W, C, F | | W, C, F | C, F |
| K1107R, K1107N | | R | N | |
| S1109K, S1109T, S1109R, S1109, | K, T, R, I, | K, T, R, L, | I, Q, G | R, L, A, V |
| S1109Q, S1109L, S1109A, S1109G, | Q, L, A, G, | A, V | | |
| S1109V | V | | | |
| L1111V | | | V | |
| K1113A | A | A | A | |
| R1114N, R1114T, R1114S, R1114Q, | N, T, S, Q, | K, G | A | N, T, S |
| R1114H, R1114D, R1114A, R1114G, | H, D, A, G, | | | |
| R1114C, R1114K | C | | | |
| N1115P, N1115A | | | P | A |
| S1116T | T | | | |
| D1117G | G | | | G |
| K1118N, K1118S, K1118Q, K1118G, | N, S, Q, G | T, S | H, L | R, S, Q |
| K1118T, K1118H, K1118L, K1118R | | | | |
| A1121R | R | | | |
| K1124V | | V | | |
| D1125A, D1125T, D1125G | A | | T | G |
| D1127S | | S | | |
| Y1131L | | L | | |
| D1135A, D1135T, D1135S, D1135E, | | | A | T, S, E, A, V |
| D1135V | | | | |
| T1138N, T1138S, T1138Y | | | N, S, Y | |
| V1139E, V1139H | | | E | H, E |
| A1140T | | | | T |
| S1142V | | | V | |
| V1143L | | | L | |
| L1144A | | | | A |
| V1145N | | | N | |
| A1147H | | | H | |
| K1148A, K1148C | | | A | C |
| V1149T, V1149E | | | T, E | |
| E1150S, E1150L, E1150P | | | S, L | P |
| K1151L, K1151G, K1151V, K1151Y, | | | L, G, V, Y, | I, Q, L |
| K1151F, K1151I, K1151Q | | | F | |
| G1152S, G1152T, G1152Q | | | S | T, Q |
| K1153I, K1153G, K1153R, K1153Q, | | | I, G | R, Q, L, C |
| K1153L, K1153C | | | | |
| S1154G | | | G | |
| K1155S, K1155L | | | S, L | |
| K1156T, K1156I, K1156P, K1156R, | | | T, I, P | R, L |
| K1156L | | | | |
| L1157H, L1157R | | | H, R | |
| K1158E, K1158D, K1158Y, K1158S, | | | E, D, Y | D, S, L |
| K1158L | | | | |
| S1159P, S1159L, S1159F, S1159N, | | | P, L, F | N, Q |
| S1159Q | | | | |
| V1160T | | | | T |
| K1161S, K1161I, K1161L | | | S | I, L |
| L1163M, L1163T, L1163G | | | M | T, G |
| G1165P, G1165L, G1165A | | | P | L, A |
| T1167P, T1167H, T1167L | | | P | H, L |
| R1171Q | | | | Q |
| S1172E, S1172Y | | E, Y | | |
| S1173C, S1173L, S1173H | | C | L | H |
| F1174S | | | | S |
| K1176D | | | | D |
| I1179T | | | | T |
| D1180T, D1180P, D1180A, D1180I | | | T, P, A | I, P, A |
| F1181V, F1181M, F1181Q, F1181H | | | V | M, Q, H |
| E1183S, E1183L | | | S | L |
| A1184N, A1184R, A1184D, A1184C | | | N, R | D, C |
| K1185C | | | | C |
| G1186P | | | | P |
| Y1187L,Y1187S | | | L | S |
| K1188R, K1188A, K1188L, K1188S | | | R, A | L, S |
| E1189Q | | | | Q |
| V1190L, V1190A, V1190I | | | L, A | I |
| K1191T, K1191R, K1191L, K1191S, | T, R, L | S, E, G | T, H, D | P |
| K1191E, K1191G, K1191H, K1191D, | | | | |
| K1191P | | | | |
| K1192E, K1192L, K1192P, K1192T, | | E, L | P | T, Q, L |
| K1192Q | | | | |
| D1193P | | | P | |
| L1194Q, L1194H, L1194A, L1194E, | | Q, H, A | E, S | D |
| L1194S, L1194D | | | | |
| I1195T, I1195V | | | T | V |
| I1196L | | | | L |
| K1197L, K1197F, K1197C | L | | F | C |
| L1198A, L1198F | A | A | | F |
| P1199T, P1199H | | T, H | | |
| K1200S | | | | S |
| Y1201G | | | | G |
| S1202H, S1202Y | | | | H, Y |
| L1203N | | | N | |
| F1204L | | | L | |
| E1205T | | | | T |
| L1206S, L1206D, L1206G, L1206W | | | S | D, G, W |
| E1207Q | | | | Q |
| N1208L, N1208H, N1208G, N1208S, | | | L | H, G, S, C |
| N1208C | | | | |
| G1209K, G1209Y, G1209S | | | K, Y, S | |
| R1210S, R1210E, R1210G, R1210C, | | | S, E, G, C | I, A, C |
| R1210I, R1210A | | | | |
| K1211T, K1211Q, K1211A | | | T, Q, A | |
| M1213T, M1213H, M1213S, M1213P, | | | T, H, S | P, W, F |
| M1213W, M1213F | | | | |
| L1214S, L1214C | | | | S, C |
| A1215C, A1215G, A1215S | | | C | G, S |
| S1216A | | | | A |
| A1217T, A1217H, A1217P, A1217D, | | | T, H, P, D, | N, S, G, W, |
| A1217F, A1217N, A1217S, A1217G, | | | F | C, F |
| A1217W, A1217C | | | | |
| Q1221N, Q1221I, Q1221V, Q1221C, | | | N, I, V, C | T, A |
| Q1221T, Q1221A | | | | |
| G1223C | | | | C |
| N1224P | | | | P |
| L1226M | | | | M |
| A1227S, A1227G | | | | S, G |
| L1228F, L1228S | | | F | S |
| P1229T, P1229A, P1229G, P1229C, | | | T, A, G, C | M, H, L, F |
| P1229M, P1229H, P1229L, P1229F | | | | |
| S1230G, S1230T, S1230Q, S1230V | | | G | T, Q, V |
| K1231P, K1231L, K1231E, K1231D | | | P | L, E, D |
| Y1232T, Y1232R | | | | T, R |
| V1233L | | | L | |
| N1234L, N1234T, N1234P, N1234C | | | L | T, P, C |
| F1235L | | | | L |
| L1236Q | | | | Q |
| Y1237R, Y1237M, Y1237Q, Y1237C | | | | R, M, Q, C |
| L1238H, L1238V, L1238F, L1238Y | | | H, V, F | V, Y |
| S1240H, S1240L | | | H, L | |
| H1241G, H1241M, H1241R, H1241L, | | | G | M, R, L, A, |
| H1241A, H1241V, H1241F | | | | V, F |
| Y1242K, Y1242S, Y1242P, Y1242W, | | | K, S, P, W | T, I, L |
| Y1242T, Y1242I, Y1242L | | | | |
| E1243T | | | | T |
| K1244S | | | S | |
| L1245N, L1245S, L1245R, L1245A, | | | | N, S, R, A, |
| L1245W, L1245C | | | | W, C |
| K1246S | | | | S |
| G1247S, G1247W, G1247F | | | S, W | F |
| S1248D, S1248M | | | D | M |
| P1249N, P1249R, P1249L | | | | N, R, L |
| E1250D | | | | D |
| N1252A, N1252G | | | A | G |
| E1253G, E1253S, E1253R | | | G | S, R |
| Q1254I, Q1254H, Q1254P, Q1254D | | | I, H | P, D |
| K1255P, K1255G, K1255L, K1255F | | | P, G | L, F |
| Q1256P, Q1256C | | | P | C |
| L1257E, L1257V, L1257S, L1257W | | | E, V | S, V, W |
| F1258L, F1258V | | | L | V |
| E1260Q, E1260R, E1260Y, E1260A, | | | Q, R, Y | A, C, F |
| E1260C, E1260F | | | | |
| Q1261T, Q1261A, Q1261V, Q1261L, | | | T, A, V, L | M, H, P, G |
| Q1261M, Q1261H, Q1261P, Q1261G | | | | |
| H1262R, H1262W, H1262T, H1262D, | | | R, W | T, D, V, S |
| H1262V, H1262S | | | | |
| K1263Q, K1263L, K1263S, K1263I, | | | Q, L | S, I, P, V, F |
| K1263P, K1263V, K1263F | | | | |
| H1264L | | | | L |
| Y1265L, Y1265C, Y1265R, Y1265I, | | | L, C | R, I, Q, H |
| Y1265Q, Y1265H | | | | |
| L1266T | | | | T |
| D1267Q, D1267P, D1267V | | | Q, P | V |
| E1268V, E1268R, E1268A | | | V | R, A |
| Q1272I | | | | I |
| S1274H, S1274R, S1274G | | | H, R | G |
| F1276G, F1276Y | | | | G, Y |
| S1277T, S1277H | | | T, H | H |
| K1278S, K1278L | | | S | L |
| R1279T | | | | T |
| V1280I, V1280L, V1280E, V1280S | | | I, L, E | S |
| I1281L, I1281S, I1281C | | | L | S, C |
| L1282T, L1282R, L1282A, L1282G, | | | T, R, A, G, | S |
| L1282V, L1282S | | | V | |
| A1283T, A1283S, A1283H, A1283R | | | T | S, H, R |
| A1285L, A1285P, A1285V | | | L | P, V |
| N1286Q, N1286G, N1286C, N1286H, | | | Q, G, C | H, L, A, G, |
| N1286L, N1286A, N1286V, N1286F | | | | V, F |
| L1287S, L1287I, L1287W, L1287F | | | | S, I, W, F |
| D1288A, D1288G | | | A | G |
| K1289P, K1289E, K1289G, K1289V, | | | P, E, G | V, S, L |
| K1289S, K1289L | | | | |
| L1291Q, L1291I, L1291S, L1291F | | | Q | I, S, F |
| S1292T, S1292L, S1292V, S1292Y, | | | T | T, L, V, Y, F |
| S1292F | | | | |
| A1293T, A1293H, A1293G | | | T | H, G |
| Y1294I, Y1294R, Y1294C, Y1294S, | | | I, R, C | S, H |
| Y1294H | | | | |
| N1295T, N1295Q, N1295P, N1295E, | | | T, Q, P, E | S, R, L, G |
| N1295S, N1295R, N1295L, N1295G | | | | |
| K1296L, K1296A, K1296Q, K1296R, | | | L, A | Q, R, W |
| K1296W | | | | |
| H1297E, H1297F | | | E, F | |
| R1298N, R1298A, R1298S, R1298F | | | N, A | N, S, F |
| D1299L, D1299Y | | | L, Y | |
| K1300T, K1300R, K1300E, K1300V, | | | T | R, E, V, F |
| K1300F | | | | |
| P1301N | | | | N |
| I1302S, I1302V | | | | S, V |
| R1303D, R1303V | | | D | V |
| E1304H, E1304S | | | H, S | |
| Q1305S | | | S | |
| A1306N, A1306I | | | | N, I |
| E1307S, E1307C | | | S | C |
| I1309S, I1309R, I1309Y | | | | S, R, Y |
| I1310Y, I1310V | | | Y | V |
| H1311T, H1311G, H1311V | | | T | G, V |
| L1312F | | | | F |
| F1313L, F1313S | | | L | S |
| T1314L, T1314G, T1314S, T1314E, | | | L, G, S | E, D, S |
| T1314D | | | | |
| L1315A, L1315S | | | A | S |
| T1316A, T1316Q | | | A | Q |
| N1317L, N1317T, N1317S | | | L | T, S |
| L1318N, L1318I, L1318P, L1318E, | | | N, I, P, E, | T, E, D, A, Y |
| L1318V, L1318W, L1318T, L1318D, | | | V, W | |
| L1318A, L1318Y | | | | |
| G1319S, G1319N | | | S | N |
| A1320T, A1320R, A1320S, A1320V, | | | T, R, S, V, | S, V, Y, F |
| A1320C, A1320Y, A1320F | | | C | |
| P1321S, P1321A, P1321G, P1321M, | | | S, A, G | M, C |
| P1321C | | | | |
| A1322N, A1322T, A1322M, A1322L, | | | N | N, T, M, L, |
| A1322V, A1322W | | | | V, W |
| A1323S | | | | S |
| F1324S, F1324A, F1324G, F1324C, | | | S, A, G, C | T, M, D, A, V |
| F1324T, F1324M, F1324D, F1324V | | | | |
| K1325R | | | | R |
| Y1326M, Y1326W | | | M, W | |
| F1327A | | | | A |
| D1328T, D1328L | | | T, L | |
| T1329L, T1329S | | | | L, S |
| T1330N, T1330Q | | | N | N, Q |
| D1332W | | | | W |
| K1334A, K1334T, K1334Q, K1334R, | | | A | T, Q, R, A, |
| K1334S, K1334W | | | | S, W |
| Y1336M, Y1336P | | | M | P |
| S1338N, S1338V, S1338Y, S1338W, | | | N, V, Y, W | A, F |
| S1338A, S1338F | | | | |
| T1339L, T1339A, T1339G | | | L, A, G | L |
| K1340N, K1340T, K1340S, K1340M, | | | N, T, S, M, | R, M, H, G, |
| K1340I, K1340P, K1340E, K1340A, | | | I, P, E, A, | C, L |
| K1340V, K1340C, K1340R, K1340H, | | | V, C | |
| K1340G, K1340L | | | | |
| V1342M, V1342L, V1342I | | | M, L | I, L |
| L1343K, L1343E | | | | K, E |
| D1344H | | | | H |
| T1346L, T1346I | | | L | I |
| L1347V | | | V | |
| I1348S | | | | S |
| H1349R, H1349V, H1349F, H1349T, | R | V, F | T, S | R, L, A, Y |
| H1349S, H1349L, H1349A, H1349Y | | | | |
| Q1350L, Q1350R, Q1350S | | L | | R, S |
| I1352T, I1352R, I1352P, I1352H | T, R | P, R | R | H, P |
| G1354V | | | | V |
| L1355T | | | | T |
| Y1356L,Y1356Q | | | L | Q |
| E1357T, E1357I | | T | T | I |
| T1358H, T1358S | H | | S | |
| R1359N, R1359V | | N, V | | |
| D1361S | | | | S |
| L1362C, L1362V | | | C | V |
| S1363H, S1363V | | | | H, V |
| Q1364N | | | | N |
| L1365M | | | | M |
| G1366D | | | | D |
| G1367P, G1367A | | | P, A | |
| D1368T, D1368M, D1368L, D1368G | | | T, M, L | G |

Table 5 indicates the amino acid changes that were identified as beneficial from either screen as described previously with at least one corresponding amino acid substitution with an enrichment relative to synonymous amino acid substitutions of at least 1 in the post-screen sample relative to the pre-screen (input) sample for at least two replicates for either guide design. Each row corresponds to a position in Cas9 at which at least one beneficial amino acid substitution was identified, with column 1 indicating the Cas9 amino acid substitution(s) (original amino acid in wild-type Cas9 at that position, Cas9 position, new amino acid identified as beneficial at that position) with the Cas9 position of the row contained within each amino acid substitution identifier, column 2 indicating amino acid substitutions that were identified as beneficial from Screen 1 using synthetic guides of guide design 1 (see Table 2 VEGFA GD1), column 3 indicating amino acid substitutions that were identified as beneficial from Screen 1 using synthetic guides of guide design 2 (see Table 2 VEGFA GD2), column 4 indicating amino acid substitutions that were identified as beneficial from Screen 2 using synthetic guides of guide design 1 (see Table 2 VEGFA GD1), and column 5 indicating amino acid substitutions that were identified as beneficial from Screen 2 using synthetic guides of guide design 2 (see Table 2 VEGFA GD2). For columns 2-5, only the single letter abbreviation of the new beneficial amino acid identified from each screen at the Cas9 position of the corresponding row is shown.

Table 6 indicates the amino acid changes that were identified as beneficial from either screen and had at least one corresponding amino acid substitution with an enrichment relative to synonymous amino acid substitution reads of at least 1.5 in the post-screen sample relative to the pre-screen (input) sample for at least two replicates using either guide design, i.e. amino acid changes with an enrichment of at least 1.5. Each row corresponds to a position in Cas9 at which at least one amino acid substitution with an enrichment greater than 1.5 was identified, with column 1 indicating the Cas9 amino acid substitution(s) (original amino acid in wild-type Cas9 at that position, Cas9 position, new amino acid identified as beneficial at that position) with the Cas9 position of the row contained within each amino acid substitution identifier, column 2 indicating amino acid substitutions with an enrichment of at least 1.5 from Screen 1 using synthetic guides of guide design 1 (see Table 2 VEGFA GD1), column 3 indicating amino acid substitutions with an enrichment of at least 1.5 from Screen 1 using synthetic guides of guide design 2 (see Table 2 VEGFA GD2), column 4 indicating amino acid substitutions with an enrichment of at least 1.5 from Screen 2 using synthetic guides of guide design 1 (see Table 2 VEGFA GD1), and column 5 indicating amino acid substitutions with an enrichment of at least 1.5 from Screen 2 using synthetic guides of guide design 2 (see Table 2 VEGFA GD2). For columns 2-5, only the single letter abbreviation of the new beneficial amino acid identified from each screen at the Cas9 position of the corresponding row is shown.

### Example 2. A subset of tested screen identified amino acid substitutions improve

Cas9 activity with a short guide in human cells.

In order to test if the amino acid substitutions identified from the screen in bacteria improve Cas9 activity with short scaffold region guides in human cells, we generated human codon-optimized Cas9 expression plasmids to express Cas9 variants containing 28 screen-identified amino acid substitutions. Expression plasmids to express sgRNAs corresponding to the full length, GD1, and GD2 designs were ordered from IDT in a pUCIDT plasmid (see Table 7).

TABLE 7
Guide plasmid sequences

| Guide | | Expression construct (U6 |
|---|---|---|
| name | Guide sequence | promoter + guide + terminator) |
| HPRT1 | AAUUAUGGGGAUUA | Acgcgtgagggcctatttcccatgattccttcatatttgcatatacgata |
| sgRNA full | CUAGGAguuuuagagcua | caaggctgttagagagataattggaattaatttgactgtaaacacaaag |
| length (FL) | gaaauagcaaguuaaaauaagg | atattagtacaaaatacgtgacgtagaaagtaataatttcttgggtagttt |
| | cuaguccguuaucaacuugaaa | gcagttttaaaattatgttttaaaatggactatcatatgcttaccgtaactt |
| | aaguggcaccgagucggugcu | gaaagtatttcgatttcttggctttatatatcttgtggaaaggacgaaaca |
| | uuu (SEQ ID NO: 85) | ccAATTATGGGGATTACTAGGAgttttagagctaga |
| | | aatagcaagttaaaataaggctagtccgttatcaacttgaaaaagtgg |
| | | caccgagtcggtgcttttttt (SEQ ID NO: 109) |
| HPRT1 | AAUUAUGGGGAUUA | Acgcgtgagggcctatttcccatgattccttcatatttgcatatacgata |
| sgRNA | CUAGGAguuuuagagcua | caaggctgttagagagataattggaattaatttgactgtaaacacaaag |
| (GD1/H70) | gaaauagcaaguuaaaauaagg | atattagtacaaaatacgtgacgtagaaagtaataatttcttgggtagttt |
| | cuaguccguuaucaacuuuu | gcagttttaaaattatgttttaaaatggactatcatatgcttaccgtaactt |
| | (SEQ ID NO: 86) | gaaagtatttcgatttcttggctttatatatcttgtggaaaggacgaaaca |
| | | ccAATTATGGGGATTACTAGGAgttttagagctaga |
| | | aatagcaagttaaaataaggctagtccgttatcaacttttttt (SEQ ID |
| | | NO: 110) |
| HPRT1 | AAUUAUGGGGAUUA | Acgcgtgagggcctatttcccatgattccttcatatttgcatatacgata |
| sgRNA | CUAGGAguuuuagaggaa | caaggctgttagagagataattggaattaatttgactgtaaacacaaag |
| (GD2/H70 | acaaguuaaaauaaggcuaguc | atattagtacaaaatacgtgacgtagaaagtaataatttcttgggtagttt |
| ID) | cguuaucaacuuuu (SEQ ID | gcagttttaaaattatgttttaaaatggactatcatatgcttaccgtaactt |
| | NO: 87) | gaaagtatttcgatttcttggctttatatatcttgtggaaaggacgaaaca |
| | | CCAATTATGGGGATTACTAGGAgttttagaggaaa |
| | | caagttaaaataaggctagtccgttatcaacttttttt (SEQ ID NO: |
| | | 111) |
| HPRT1 | AAUUAUGGGGAUUA | Acgcgtgagggcctatttcccatgattccttcatatttgcatatacgata |
| sgRNA | CUAGGAguuuuagagcua | caaggctgttagagagataattggaattaatttgactgtaaacacaaag |
| (GD3/H67) | gaaauagcaaguuaaaauaagg | atattagtacaaaatacgtgacgtagaaagtaataatttcttgggtagttt |
| | (SEQ ID NO: 88) | gcagttttaaaattatgttttaaaatggactatcatatgcttaccgtaactt |
| | | gaaagtatttcgatttcttggctttatatatcttgtggaaaggacgaaaca |
| | | ccAATTATGGGGATTACTAGGAgttttagagctaga |
| | | aatagcaagttaaaataaggctagtccgttatcttttttt (SEQ ID |
| | | NO: 112) |
| HPRT1 | AAUUAUGGGGAUUA | Acgcgtgagggcctatttcccatgattccttcatatttgcatatacgata |
| sgRNA | CUAGGAguuuuagagcua | caaggctgttagagagataattggaattaatttgactgtaaacacaaag |
| (GD4/H64) | gaaauagcaaguuaaaauaagg | atattagtacaaaatacgtgacgtagaaagtaataatttcttgggtagttt |
| | cuaguccguuuu (SEQ ID | gcagttttaaaattatgttttaaaatggactatcatatgcttaccgtaactt |
| | NO: 89) | gaaagtatttcgatttcttggctttatatatcttgtggaaaggacgaaaca |
| | | CCAATTATGGGGATTACTAGGAgttttagagctaga |
| | | aatagcaagttaaaataaggctagtccgttttttt (SEQ ID NO: |
| | | 113) |
| HPRT1 | AAUUAUGGGGAUUA | Acgcgtgagggcctatttcccatgattccttcatatttgcatatacgata |
| sgRNA | CUAGGAguuuuagagcua | caaggctgttagagagataattggaattaatttgactgtaaacacaaag |
| (GD5/H61) | gaaauagcaaguuaaaauaagg | atattagtacaaaatacgtgacgtagaaagtaataatttcttgggtagttt |
| | cuaguccuuuu (SEQ ID | gcagttttaaaattatgttttaaaatggactatcatatgcttaccgtaactt |
| | NO: 90) | gaaagtatttcgatttcttggctttatatatcttgtggaaaggacgaaaca |
| | | CCAATTATGGGGATTACTAGGAgttttagagctaga |
| | | aatagcaagttaaaataaggctagtccttttttt (SEQ ID NO: |
| | | 114) |
| HPRT1 | AAUUAUGGGGAUUACUA | Acgcgtgagggcctatttcccatgattccttcatatttgcatatacgat |
| sgRNA | GGAguuuuagagcuagaaau | acaaggctgttagagagataattggaattaatttgactgtaaacaca |
| (H58) | agcaaguuaaaauaaggcuag | aagatattagtacaaaatacgtgacgtagaaagtaataatttcttgg |
| | uuuu (SEQ ID NO: 91) | gtagtttgcagttttaaaattatgttttaaaatggactatcatatgctta |
| | | ccgtaacttgaaagtatttcgatttcttggctttatatatcttgtggaaa |
| | | ggacgaaacaccAATTATGGGGATTACTAGGAgttttagag |
| | | ctagaaatagcaagttaaaataaggctagttttttt (SEQ ID NO: |
| | | 115) |
| HPRT1 | AAUUAUGGGGAUUACUA | Acgcgtgagggcctatttcccatgattccttcatatttgcatatacgat |
| sgRNA | GGAguuuuagagcuagaaau | acaaggctgttagagagataattggaattaatttgactgtaaacaca |
| (H66) | agcaaguuaaaauaaggcuag | aagatattagtacaaaatacgtgacgtagaaagtaataatttcttgg |
| | uccguuauuuu (SEQ ID | gtagtttgcagttttaaaattatgttttaaaatggactatcatatgctta |
| | NO: 92) | ccgtaacttgaaagtatttcgatttcttggctttatatatcttgtggaaa |
| | | ggacgaaacaccAATTATGGGGATTACTAGGAgttttagag |
| | | ctagaaatagcaagttaaaataaggctagtccgttattttttt (SEQ |
| | | ID NO: 116) |
| HPRT1 | AAUUAUGGGGAUUACUA | Acgcgtgagggcctatttcccatgattccttcatatttgcatatacgat |
| sgRNA | GGAguuuuagaggaaacaag | acaaggctgttagagagataattggaattaatttgactgtaaacaca |
| (H67ID) | uuaaaauaaggcuaguccguu | aagatattagtacaaaatacgtgacgtagaaagtaataatttcttgg |
| | aucuuuu (SEQ ID NO: | gtagtttgcagttttaaaattatgttttaaaatggactatcatatgctta |
| | 93) | ccgtaacttgaaagtatttcgatttcttggctttatatatcttgtggaaa |
| | | ggacgaaacaccAATTATGGGGATTACTAGGAgttttagag |
| | | gaaacaagttaaaataaggctagtccgttatcttttttt (SEQ ID |
| | | NO: 117) |
| HPRT1 | AAUUAUGGGGAUUACUA | Acgcgtgagggcctatttcccatgattccttcatatttgcatatacgat |
| sgRNA | GGAguuuuagaggaaacaag | acaaggctgttagagagataattggaattaatttgactgtaaacaca |
| (H66ID) | uuaaaauaaggcuaguccguu | aagatattagtacaaaatacgtgacgtagaaagtaataatttcttgg |
| | auuuu (SEQ ID NO: 94) | gtagtttgcagttttaaaattatgttttaaaatggactatcatatgctta |
| | | ccgtaacttgaaagtatttcgatttcttggctttatatatcttgtggaaa |
| | | ggacgaaacaccAATTATGGGGATTACTAGGAgttttagag |
| | | gaaacaagttaaaataaggctagtccgttattttttt (SEQ ID |
| | | NO: 118) |
| HPRT1 | AAUUAUGGGGAUUACUA | Acgcgtgagggcctatttcccatgattccttcatatttgcatatacgat |
| sgRNA | GGAguuuuagaggaaacaag | acaaggctgttagagagataattggaattaatttgactgtaaacaca |
| (H64ID) | uuaaaauaaggcuaguccguu | aagatattagtacaaaatacgtgacgtagaaagtaataatttcttgg |
| | uu (SEQ ID NO: 95) | gtagtttgcagttttaaaattatgttttaaaatggactatcatatgctta |
| | | ccgtaacttgaaagtatttcgatttcttggctttatatatcttgtggaaa |
| | | ggacgaaacaccAATTATGGGGATTACTAGGAgttttagag |
| | | gaaacaagttaaaataaggctagtccgttttttt (SEQ ID NO: |
| | | 119) |
| HPRT1 | AAUUAUGGGGAUUACUA | Acgcgtgagggcctatttcccatgattccttcatatttgcatatacgat |
| sgRNA | GGAguuuuagaggaaacaag | acaaggctgttagagagataattggaattaatttgactgtaaacaca |
| (H61ID) | uuaaaauaaggcuaguccuuu | aagatattagtacaaaatacgtgacgtagaaagtaataatttcttgg |
| | u (SEQ ID NO: 96) | gtagtttgcagttttaaaattatgttttaaaatggactatcatatgctta |
| | | ccgtaacttgaaagtatttcgatttcttggctttatatatcttgtggaaa |
| | | ggacgaaacaccAATTATGGGGATTACTAGGAgttttagag |
| | | gaaacaagttaaaataaggctagtccttttttt (SEQ ID NO: |
| | | 120) |
| VEGFA | GGUGAGUGAGUGUG | Acgcgtgagggcctatttcccatgattccttcatatttgcatatacgata |
| sgRNA full | UGCGUGguuuuagagcua | caaggctgttagagagataattggaattaatttgactgtaaacacaaag |
| length (FL) | gaaauagcaaguuaaaauaagg | atattagtacaaaatacgtgacgtagaaagtaataatttcttgggtagttt |
| | cuaguccguuaucaacuugaaa | gcagttttaaaattatgttttaaaatggactatcatatgcttaccgtaactt |
| | aaguggcaccgagucggugcu | gaaagtatttcgatttcttggctttatatatcttgtggaaaggacgaaaca |
| | uuu (SEQ ID NO: 97) | ccGGTGAGTGAGTGTGTGCGTGgttttagagctaga |
| | | aatagcaagttaaaataaggctagtccgttatcaacttgaaaaagtgg |
| | | caccgagtcggtgcttttttt (SEQ ID NO: 121) |
| VEGFA | GGUGAGUGAGUGUG | Acgcgtgagggcctatttcccatgattccttcatatttgcatatacgata |
| sgRNA | UGCGUGguuuuagagcua | caaggctgttagagagataattggaattaatttgactgtaaacacaaag |
| (GD1/V70) | gaaauagcaaguuaaaauaagg | atattagtacaaaatacgtgacgtagaaagtaataatttcttgggtagttt |
| | cuaguccguuaucaacuuuu | gcagttttaaaattatgttttaaaatggactatcatatgcttaccgtaactt |
| | (SEQ ID NO: 98) | gaaagtatttcgatttcttggctttatatatcttgtggaaaggacgaaaca |
| | | ccGGTGAGTGAGTGTGTGCGTGgttttagagctaga |
| | | aatagcaagttaaaataaggctagtccgttatcaacttttttt (SEQ ID |
| | | NO: 122) |
| VEGFA | GGUGAGUGAGUGUG | Acgcgtgagggcctatttcccatgattccttcatatttgcatatacgata |
| sgRNA | UGCGUGguuuuagaggaa | caaggctgttagagagataattggaattaatttgactgtaaacacaaag |
| (GD2/V70ID) | acaaguuaaaauaaggcuaguc | atattagtacaaaatacgtgacgtagaaagtaataatttcttgggtagttt |
| | cguuaucaacuuuu (SEQ ID | gcagttttaaaattatgttttaaaatggactatcatatgcttaccgtaactt |
| | NO: 99) | gaaagtatttcgatttcttggctttatatatcttgtggaaaggacgaaaca |
| | | ccGGTGAGTGAGTGTGTGCGTGgttttagaggaaa |
| | | caagttaaaataaggctagtccgttatcaacttttttt (SEQ ID NO: |
| | | 123) |
| VEGFA | GGUGAGUGAGUGUG | Acgcgtgagggcctatttcccatgattccttcatatttgcatatacgata |
| sgRNA | UGCGUGguuuuagagcua | caaggctgttagagagataattggaattaatttgactgtaaacacaaag |
| (GD3/V67) | gaaauagcaaguuaaaauaagg | atattagtacaaaatacgtgacgtagaaagtaataatttcttgggtagttt |
| | cuaguccguuaucuuuu(SEQ | gcagttttaaaattatgttttaaaatggactatcatatgcttaccgtaactt |
| | ID NO: 100) | gaaagtatttcgatttcttggctttatatatcttgtggaaaggacgaaaca |
| | | ccGGTGAGTGAGTGTGTGCGTGgttttagagctaga |
| | | aatagcaagttaaaataaggctagtccgttatcttttttt (SEQ ID |
| | | NO: 124) |
| VEGFA | GGUGAGUGAGUGUG | Acgcgtgagggcctatttcccatgattccttcatatttgcatatacgata |
| sgRNA | UGCGUGguuuuagagcua | caaggctgttagagagataattggaattaatttgactgtaaacacaaag |
| (GD4/V64) | gaaauagcaaguuaaaauaagg | atattagtacaaaatacgtgacgtagaaagtaataatttcttgggtagttt |
| | cuaguccguuuu (SEQ ID | gcagttttaaaattatgttttaaaatggactatcatatgcttaccgtaactt |
| | NO: 101) | gaaagtatttcgatttcttggctttatatatcttgtggaaaggacgaaaca |
| | | ccGGTGAGTGAGTGTGTGCGTGgttttagagctaga |
| | | aatagcaagttaaaataaggctagtccgttttttt (SEQ ID NO: |
| | | 125) |
| VEGFA | GGUGAGUGAGUGUG | Acgcgtgagggcctatttcccatgattccttcatatttgcatatacgata |
| sgRNA | UGCGUGguuuuagagcua | caaggctgttagagagataattggaattaatttgactgtaaacacaaag |
| (GD5/V61) | gaaauagcaaguuaaaauaagg | atattagtacaaaatacgtgacgtagaaagtaataatttcttgggtagttt |
| | cuaguccuuuu (SEQ ID | gcagttttaaaattatgttttaaaatggactatcatatgcttaccgtaactt |
| | NO: 102) | gaaagtatttcgatttcttggctttatatatcttgtggaaaggacgaaaca |
| | | ccGGTGAGTGAGTGTGTGCGTGgttttagagctaga |
| | | aatagcaagttaaaataaggctagtccttttttt (SEQ ID NO: |
| | | 126) |
| VEGFA | GGUGAGUGAGUGUGUG | Acgcgtgagggcctatttcccatgattccttcatatttgcatatacgat |
| sgRNA | CGUGguuuuagagcuagaaa | acaaggctgttagagagataattggaattaatttgactgtaaacaca |
| (H58) | uagcaaguuaaaauaaggcua | aagatattagtacaaaatacgtgacgtagaaagtaataatttcttgg |
| | guuuu (SEQ ID NO: 103) | gtagtttgcagttttaaaattatgttttaaaatggactatcatatgctta |
| | | ccgtaacttgaaagtatttcgatttcttggctttatatatcttgtggaaa |
| | | ggacgaaacaccGGTGAGTGAGTGTGTGCGTGgttttaga |
| | | gctagaaatagcaagttaaaataaggctagttttttt (SEQ ID |
| | | NO: 127) |
| VEGFA | GGUGAGUGAGUGUGUG | Acgcgtgagggcctatttcccatgattccttcatatttgcatatacgat |
| sgRNA | CGUGguuuuagagcuagaaa | acaaggctgttagagagataattggaattaatttgactgtaaacaca |
| (H66) | uagcaaguuaaaauaaggcua | aagatattagtacaaaatacgtgacgtagaaagtaataatttcttgg |
| | guccguuauuuu (SEQ ID | gtagtttgcagttttaaaattatgttttaaaatggactatcatatgctta |
| | NO: 104) | ccgtaacttgaaagtatttcgatttcttggctttatatatcttgtggaaa |
| | | ggacgaaacaccGGTGAGTGAGTGTGTGCGTGgttttaga |
| | | gctagaaatagcaagttaaaataaggctagtccgttattttttt |
| | | (SEQ ID NO: 128) |
| VEGFA | GGUGAGUGAGUGUGUG | Acgcgtgagggcctatttcccatgattccttcatatttgcatatacgat |
| sgRNA | CGUGguuuuagaggaaacaa | acaaggctgttagagagataattggaattaatttgactgtaaacaca |
| (H67ID) | guuaaaauaaggcuaguccgu | aagatattagtacaaaatacgtgacgtagaaagtaataatttcttgg |
| | uaucuuuu (SEQ ID NO: | gtagtttgcagttttaaaattatgttttaaaatggactatcatatgctta |
| | 105) | ccgtaacttgaaagtatttcgatttcttggctttatatatcttgtggaaa |
| | | ggacgaaacaccGGTGAGTGAGTGTGTGCGTGgttttaga |
| | | ggaaacaagttaaaataaggctagtccgttatcttttttt (SEQ ID |
| | | NO: 129) |
| VEGFA | GGUGAGUGAGUGUGUG | Acgcgtgagggcctatttcccatgattccttcatatttgcatatacgat |
| sgRNA | CGUGguuuuagaggaaacaa | acaaggctgttagagagataattggaattaatttgactgtaaacaca |
| (H66ID) | guuaaaauaaggcuaguccgu | aagatattagtacaaaatacgtgacgtagaaagtaataatttcttgg |
| | uauuuu (SEQ ID NO: | gtagtttgcagttttaaaattatgttttaaaatggactatcatatgctta |
| | 106) | ccgtaacttgaaagtatttcgatttcttggctttatatatcttgtggaaa |
| | | ggacgaaacaccGGTGAGTGAGTGTGTGCGTGgttttaga |
| | | ggaaacaagttaaaataaggctagtccgttattttttt (SEQ ID |
| | | NO: 130) |
| VEGFA | GGUGAGUGAGUGUGUG | Acgcgtgagggcctatttcccatgattccttcatatttgcatatacgat |
| sgRNA | CGUGguuuuagaggaaacaa | acaaggctgttagagagataattggaattaatttgactgtaaacaca |
| (H64ID) | guuaaaauaaggcuaguccgu | aagatattagtacaaaatacgtgacgtagaaagtaataatttcttgg |
| | uuu (SEQ ID NO: 107) | gtagtttgcagttttaaaattatgttttaaaatggactatcatatgctta |
| | | ccgtaacttgaaagtatttcgatttcttggctttatatatcttgtggaaa |
| | | ggacgaaacaccGGTGAGTGAGTGTGTGCGTGgttttaga |
| | | ggaaacaagttaaaataaggctagtccgttttttt (SEQ ID NO: |
| | | 131) |
| VEGFA | GGUGAGUGAGUGUGUG | Acgcgtgagggcctatttcccatgattccttcatatttgcatatacgat |
| sgRNA | CGUGguuuuagaggaaacaa | acaaggctgttagagagataattggaattaatttgactgtaaacaca |
| (H61ID) | guuaaaauaaggcuaguccuu | aagatattagtacaaaatacgtgacgtagaaagtaataatttcttgg |
| | uu (SEQ ID NO: 108) | gtagtttgcagttttaaaattatgttttaaaatggactatcatatgctta |
| | | ccgtaacttgaaagtatttcgatttcttggctttatatatcttgtggaaa |
| | | ggacgaaacaccGGTGAGTGAGTGTGTGCGTGgttttaga |
| | | ggaaacaagttaaaataaggctagtccttttttt (SEQ ID NO: |
| | | 132) |

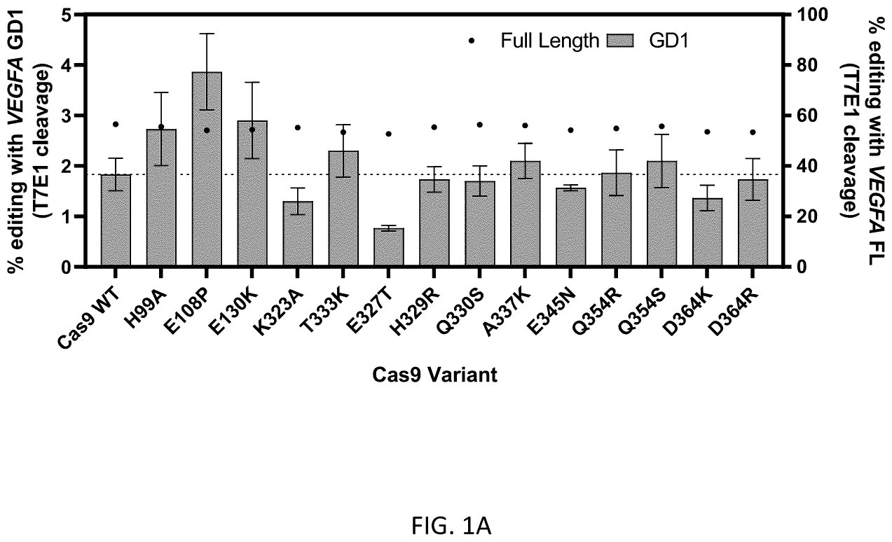

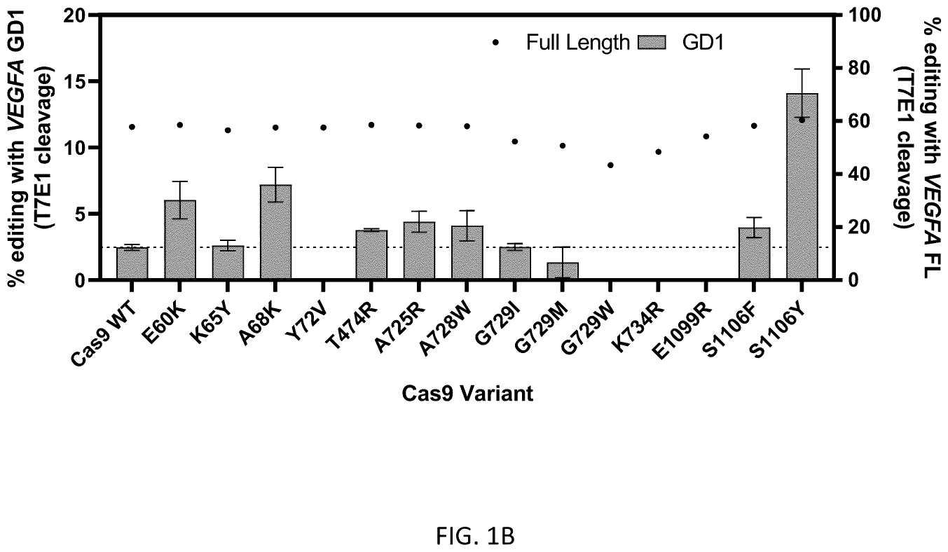

The transcription termination signal used with the U6 promoter results in the addition of on average around four U's to the end of the guide, as positions 71 and 72 are both normally U's in the context of the full length guide, terminating at position 70 or 71 would result in the same guide being expressed, therefore both GD1 and GD2 coding sequences terminated at position 70 of the guide followed by 7 T's to terminate transcription, with plasmid expressed GD2 only being different from GD1 in that it contains the internal deletions. Cas9 variant expression plasmids were delivered alongside VEGFA GD1 sgRNA expression plasmid into HEK293 cells. After 72 hours, genomic DNA was isolated and editing was measured by T7E1 assay (see Table 3 for T7E1 assay primer sequences). Of the amino acid substitutions tested, eight (E60K, A68K, T474R, A725R, A728W, S1106F, S1106Y, E108P) provided a substantial boost in editing while two more (H99A, E130K) provided a marginal improvement (see FIG. 1). Notably S1106Y provided the largest boost in editing activity, and this amino acid substitutions was the most highly enriched amino acid substitution in screen 1 in bacteria. These results demonstrate that many of the amino acid substitutions identified in the screen are able to improve Cas9 activity when paired with a short scaffold region guide in human cells.

Example 3. Screen-identified amino acid substitutions can be stacked together to further improve editing activity.

An ideal short guide compatible Cas9 variant would be able to achieve editing levels when paired with a short scaffold region guide equal to or greater than that of Cas9 paired with full-length sgRNA. While the Cas9 S1106Y variant is much more active with a short guide than wild-type Cas9, it is still much less active with a short guide than a full-length guide. We therefore tested if stacking multiple amino acid substitutions together can further improve activity when using short scaffold region guides. Multiple stacked amino acid substitution variants were tested as described in example 2 however the GD1 HPRT1 sgRNA expression plasmid was used instead of VEGFA in order to reduce the chance of identifying variants with improved activity at only VEGFA. The results of this testing are shown in FIG. 2.

Some of the amino acid substitutions that were individually beneficial at VEGFA provided a substantial boost in editing activity at HPRT1 when paired with S1106Y, notably H99A, A68K, and T474R. From the initial stacking, S1106Y+A68K+T474R (TM9) and S1106Y+A68K+H99A (TM13) were identified as particularly beneficial for improving Cas9 activity with short guides. We then selected 77 amino acid substitutions to combine individually with the TM9 amino acid substitutions to then test in HEK293 cells. We tested these four amino acid substitution variants with the HPRT1 GD1 sgRNA expression plasmid, and the results are shown in FIG. 3. Various amino acid substitutions (E57K, D364V, K1085E, D364G, G56A, K31E, H1349Y, R1084E, T472K, R753G, G1104A, M465L, T472R, Y72L, S1109K, K30L, Q354S, T466V, T1098R, H99A, T740A, K33D, E345T) had average editing from two replicates above that of the TM9, and all variants tested had higher average editing than Cas9.

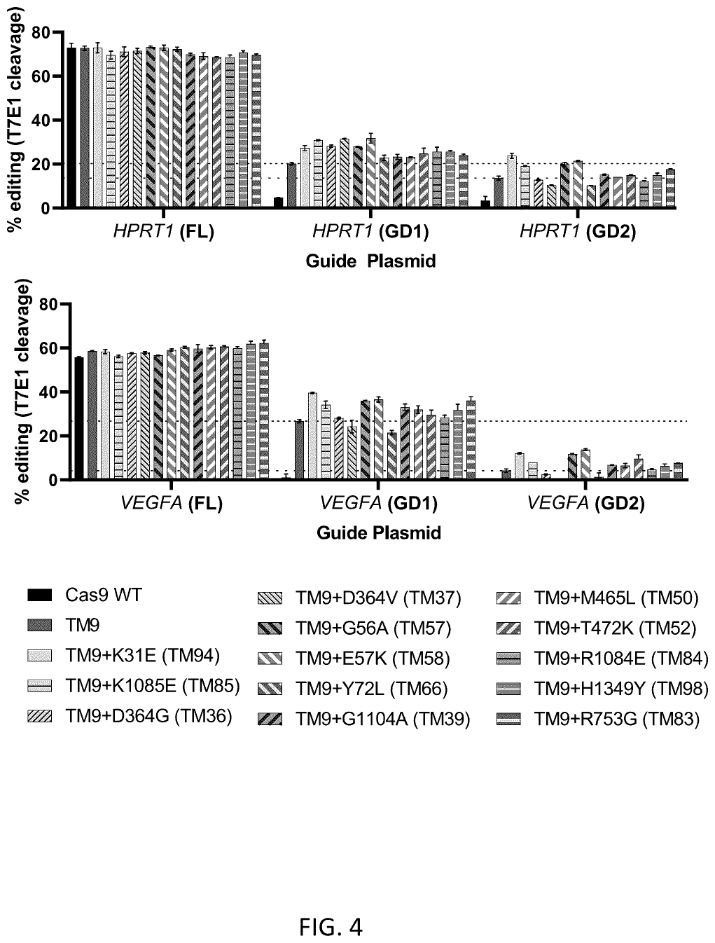

We tested the top 13 variants with full length, GD1, and GD2 sgRNA guide plasmids targeting HPRT1 or VEGFA, as shown in FIG. 4. From this we found that K31E, K1085E, G56A, E57K, G1104A, M465L, T472K, and R753G provided a consistent benefit across the two sites with the different guide designs.

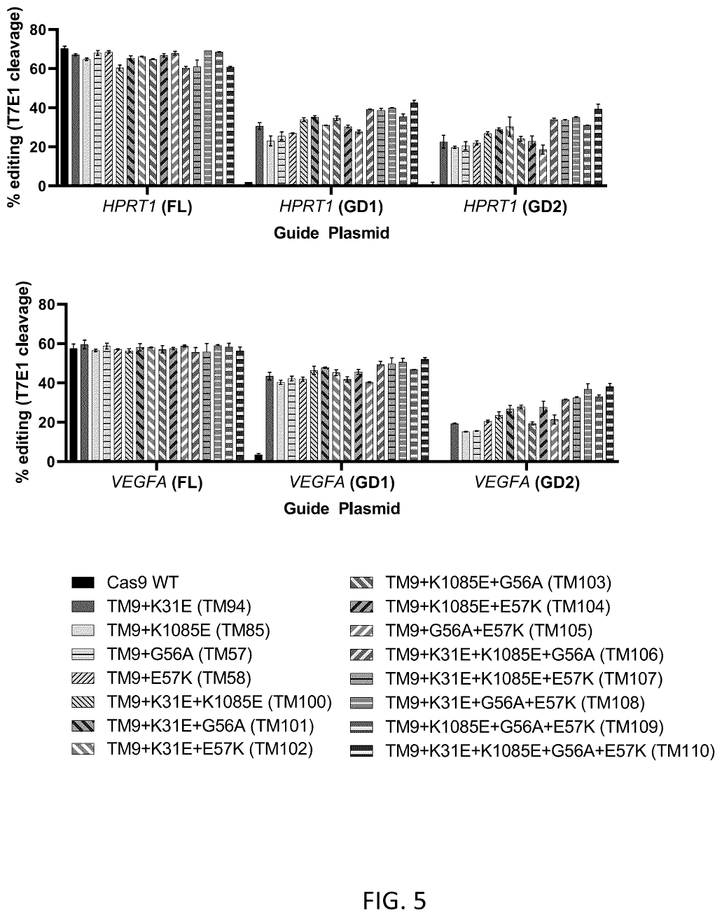

We then tested all combinations of the top 4 most beneficial amino acid substitutions (K31E, K1085E, G56A, E57K); the data are shown in FIG. 5. We found that while the combination of all four amino acid substitutions on top of TM9 (TM110) provided the largest benefit for the shorter guides, any variant with both the K31E and K1085E amino acid substitutions (TM100, TM106, TM107, and TM110) had reduced activity with the HPRT1 full length guide. We therefore decided to do further stacking with second best variant, Cas9 TM108 (S1106Y, A68K, T474R, K31E, G56A, E57K). Mutations that showed some benefit with TM9 in FIG. 4 but were not included in the stacking that generated TM108 were stacked on top of TM108 (R753G, T472K, G1104A, M465L, H1349Y, R1084E).

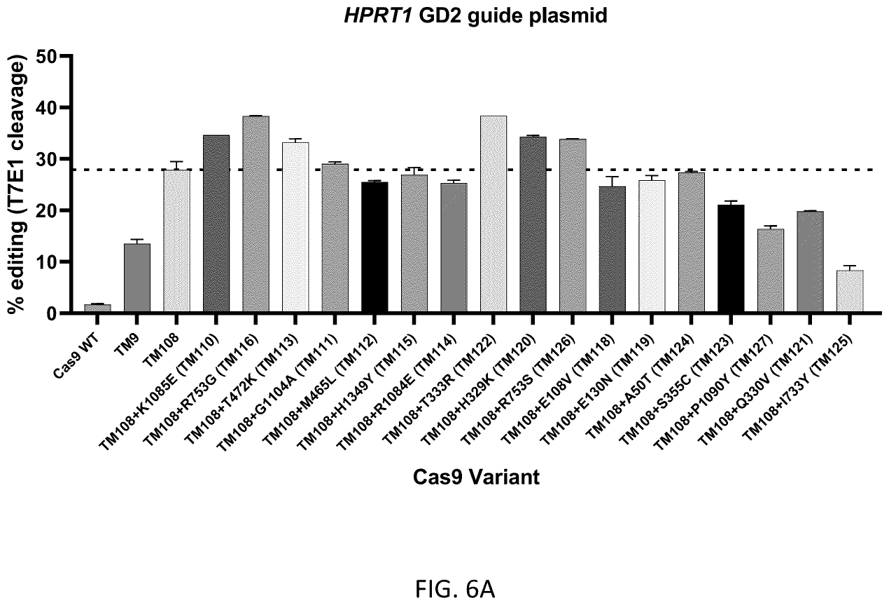

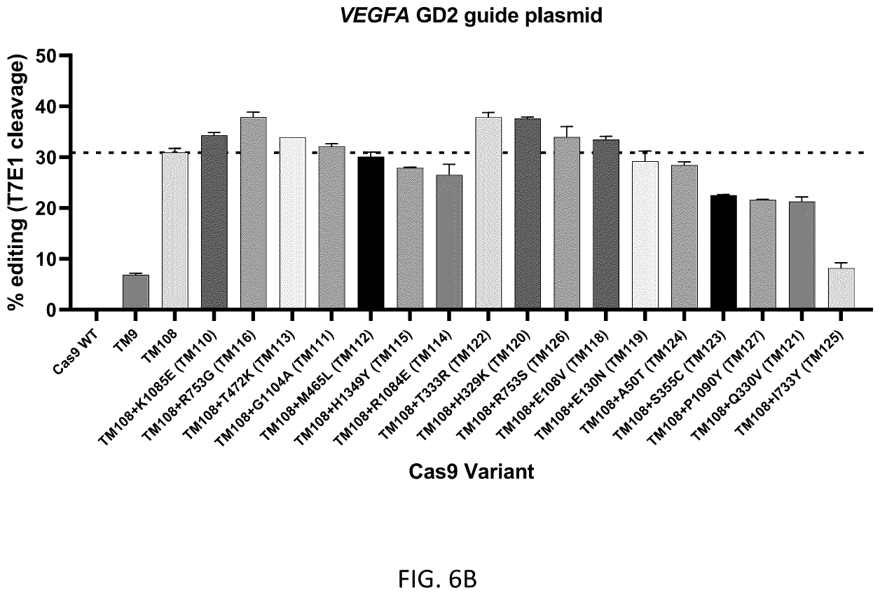

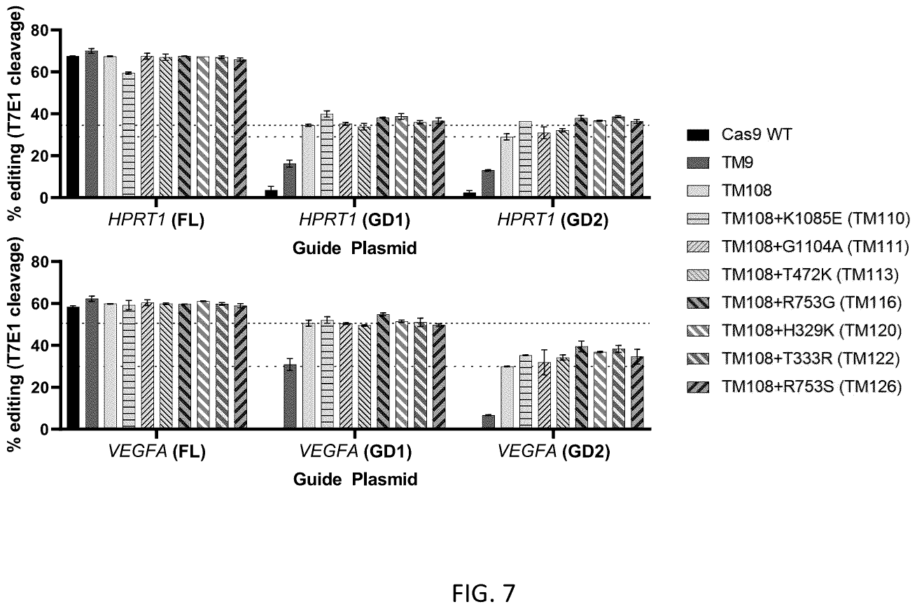

Additional amino acid substitutions that were not yet tested in HEK293 cells that were beneficially specifically with the GD2 guide in the bacteria screen (T333R, H329K, R753S, E108V, E130N, A50T, S355C, P1090Y, Q330V, and I733Y) were also selected for stacking with TM108 amino acid substitutions. The TM108 plus single amino acid substitution variants (7 amino acid substitutions relative to wild-type Cas9), were tested with both HPRT1 and VEGFA GD2 sgRNA plasmids. The results are shown in FIG. 6. From this testing, R753G, T472K, G1104A, T333R, H329K, and R753S were found to provide a consistent benefit added to TM108. These variants were then further tested with the full length and GD1 sgRNA plasmids, results shown in FIG. 7, and while they individually provided only a minimal additional benefit with the GD1 guide, none of the amino acid substitutions hurt activity with the full length guide with the exception of the previously identified K1085E+K31E combination (TM110).

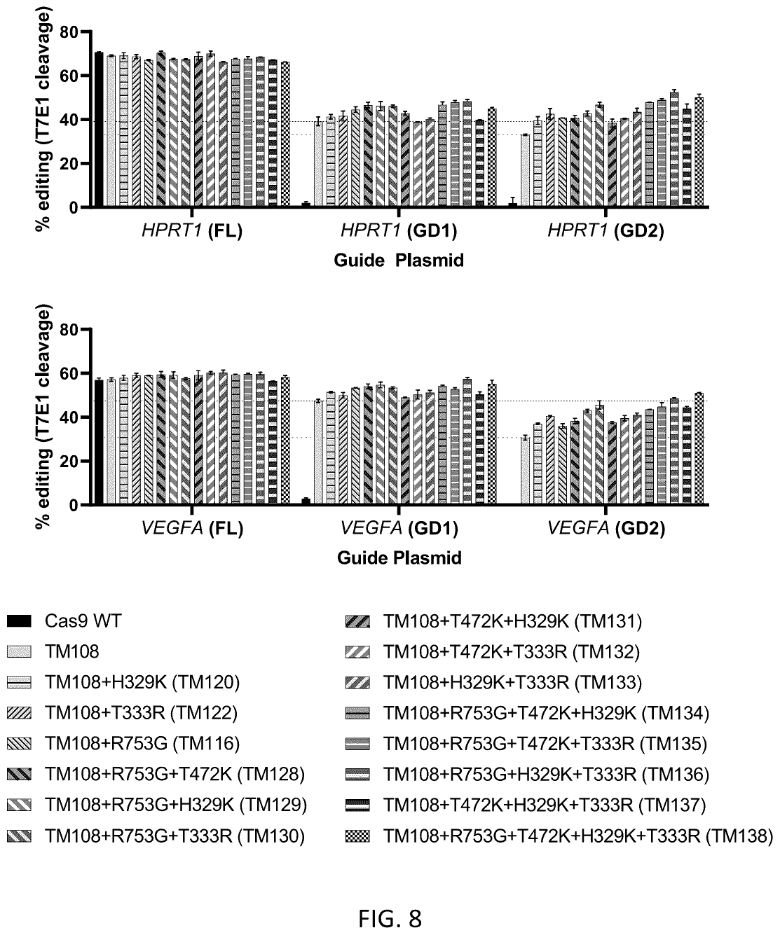

All combinations of the top four amino acid substitutions (R753G, T472K, H329K, and T333R) were then tested in HEK293 cells, with the results shown in FIG. 8. We found that combining R753G, H329K, and T333R with TM108 resulted in the highest activity Cas9 variant TM136 (K31E, G56A, E57K, A68K, H329K, T333R, T474R, R753G, S1106Y), shown in FIG. 8.

Example 4. Engineered Cas9 variants are compatible with other short guide designs.

Cas9 variants with combinations of amino acid substitutions identified from the bacterial screen were able to improve activity with GD1 and GD2 guide expression plasmids corresponding to the guides used in the screen. In order to test if these Cas9 variants were compatible with other, shorter, guide designs, we tested Cas9 TM108 and TM110 for activity in combination GD1-based guide plasmids with either 3 (GD3), 6 (GD4), or 9 (GD5) additional nucleotides removed from the 3′ end of the guide expression sequence. These designs results in guides that match the full length guide up to position 67, 64, or 61 respectively, with additional U's added as part of the transcription termination sequence. We find that both TM108 and TM110 have dramatically improved activity over Cas9 with guides as short as 64 nucleotides (GD4) (excluding the terminal U's, 44 nucleotide scaffold), data shown in FIG. 9. A slightly more extensive set of set of guide designs was then tested with Cas9 TM136, demonstrating that terminating at position 64 of the full-length guide, which when expressed off of a plasmid requiring the addition of terminal U's results in the equivalent of a 66-nucleotide guide still maintained activity. Decent activity was still maintained when the internal deletion was included resulting in the equivalent of a 58 nucleotide guide (64ID) if it was made as a synthetic guide (see FIG. 13).

Example 5. Engineered Cas9 variants are able to achieve high levels of editing in human cells using short guides when delivered as a ribonucleoprotein (RNP) complex.

Shorting the guide RNA and thus removing some of the 3′ secondary structure may cause decreased stability of the guide in cells or decrease the stability of the RNP complex and may more severely affect editing when Cas9 and guide RNA are delivered as RNP rather than plasmid. In order to test this, we purified wild-type Cas9 as well as Cas9 TM9, TM108, and TM136 variants from E. coli. The different variants were complexed with synthetic guides targeting either HPRT1 or VEGFA of either the full length (100 nt), GD1 (70 nt), or GD2 (65 nt) designs, where GD1 guides terminate at position 70 and GD2 guides terminate at position 71 but also have two internal deletions within the repeat-anti-repeat region resulting in 65 nucleotide guides (see Table 2). As previous testing was done with plasmid expressed guide, we also compared editing using either end modified or unmodified guides (see Table 2). The RNP was formed by mixing a 1:1.2 ratio of protein: guide and incubating the mix at room temperature for 10 min. RNP was delivered into HEK293 cells by Lonza nucleofection and genomic DNA was isolated after 48 hours. Editing was assessed by T7E1 assay with the results shown in FIG. 10.

Cas9 wild-type RNP was able to cause high levels of editing at both VEGFA and HPRT1 when paired with either unmodified or modified full length guide, however when paired with short scaffold guides no editing was detected at VEGFA and editing at HPRT1 was indistinguishable from the background (no Cas9/Guide) signal of the T7E1 assay for editing at HPRT1. In contrast, inclusion of only three amino acid substitutions from our screen as part of Cas9 TM9 (S1106Y, A68K, T474R) dramatically improved the editing of RNP formed with Cas9 TM9 and the short guides. Inclusion of additional amino acid substitutions as part of Cas9 TM108 (S1106Y, A68K, T474R, K31E, G56A, E57K) or Cas9 TM136 (S1106Y, A68K, T474R, K31E, G56A, E57K, R753G, H329K, T333R) further improved activity, with editing levels using both GD1 and GD2 approaching the level of editing seen with Cas9 wild-type paired with the full length guide at the relatively high dose tested (2 μM). Comparison of editing with modified vs unmodified GD1 and GD2 guides paired with Cas9 TM9, Cas9 TM108, and Cas9 TM136 revealed no decrease in editing with modified guides vs unmodified guides, indicating that the commonly used guide modification pattern (see Table 2) is well tolerated in the context of short scaffold guides paired with our Cas9 variants. We also tested if any of the amino acid substitutions impaired Cas9 variant activity with full length guide by testing a range of RNP concentrations (2, 0.25, and 0.0625 μM RNP) using the modified full length HPRT1 guide. We found that Cas9 TM9, TM108, and TM136 achieved editing comparable to Cas9 WT at all RNP concentrations tested, indicating that editing activity with the full-length guide was not impaired by the amino acid substitutions that were added.

Table 8 presents nucleotide and amino acid sequences of select Cas9 proteins described herein.

TABLE 8
Protein and DNA sequences

| | Amino | |
|---|---|---|
| | Acid | |
| Protein | Changes | Sequence |
| Cas9 | N/A | MDKKYSIGLDIGTNSVGWAVITDEYKVPSKKFKVLGNTDRHSIKKNLIGALLF |
| wild-type | | DSGETAEATRLKRTARRRYTRRKNRICYLQEIFSNEMAKVDDSFFHRLEESF |
| (Cas9 | | LVEEDKKHERHPIFGNIVDEVAYHEKYPTIYHLRKKLVDSTDKADLRLIYLALA |
| WT, | | HMIKFRGHFLIEGDLNPDNSDVDKLFIQLVQTYNQLFEENPINASGVDAKAI |
| Bacterial | | LSARLSKSRRLENLIAQLPGEKKNGLFGNLIALSLGLTPNFKSNFDLAEDAK |
| Screen) | | LQLSKDTYDDDLDNLLAQIGDQYADLFLAAKNLSDAILLSDILRVNTEITKAP |
| | | LSASMIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGELHA |
| | | ILRRQEDFYPFLKDNREKIEKILTFRIPYYVGPLARGNSRFAWMTRKSEETITP |
| | | WNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSPAIKK |
| | | GILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQLL |
| | | NAKLITQRKFDNLTKAERGGLSELDKAGFIKRQLVETRQITKHVAQILDSRM |
| | | NTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKK |
| | | TEVQTGGFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKYSLF |
| | | ELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLS |
| | | QLGGD (SEQ ID NO: 133) |
| Cas9 | N/A | ATGGACAAAAAGTACTCTATTGGCCTGGATATCGGGACCAACAGCGTCG |
| wild-type | | GGTGGGCTGTTATCACCGACGAGTATAAAGTACCTTCGAAAAAGTTCAAA |
| (Cas9 | | GTGCTGGGCAACACCGATCGCCATTCAATCAAAAAGAACTTGATTGGTG |
| WT, | | CGCTGTTGTTTGACTCCGGGGAAACCGCCGAGGCGACTCGCCTTAAAC |
| Bacterial | | GTACAGCACGTCGCCGGTACACTCGGCGTAAGAATCGCATTTGCTATTT |
| Screen) | | GCAGGAAATCTTTAGCAACGAGATGGCAAAAGTCGATGACTCGTTTTTCC |
| | | ACCGCCTCGAGGAAAGCTTTCTGGTGGAGGAAGACAAAAAGCATGAGC |
| | | GTCACCCGATCTTCGGCAACATTGTCGATGAAGTAGCGTATCATGAAAAA |
| | | TACCCAACCATTTACCACTTACGCAAAAAGCTGGTGGACAGCACTGACA |
| | | AAGCTGATTTGCGCCTTATCTATTTAGCCCTGGCACATATGATTAAGTTTCG |
| | | TGGTCACTTCCTGATCGAAGGAGACTTAAATCCCGACAACAGTGATGTTG |
| | | ATAAATTGTTTATTCAGCTTGTCCAAACTTACAATCAACTGTTCGAGGAAAA |
| | | CCCGATCAATGCCTCCGGTGTGGATGCAAAAGCCATTTTAAGTGCACGC |
| | | CTTAGCAAGTCCCGTCGCTTAGAAAACCTTATCGCGCAGCTGCCCGGC |
| | | GAGAAAAAGAATGGTTTGTTTGGGAACCTTATTGCCTTGAGCTTAGGCCT |
| | | CACCCCGAATTTCAAAAGTAATTTCGATCTTGCAGAAGACGCCAAATTAC |
| | | AACTGTCGAAGGATACTTATGATGACGATCTCGATAATCTGTTAGCGCAGA |
| | | TTGGTGACCAATACGCCGATCTTTTTCTGGCGGCTAAAAATCTGAGCGAC |
| | | GCCATCTTGCTTTCGGATATTCTCCGCGTTAACACCGAAATCACGAAAGC |
| | | GCCTCTTAGTGCCAGCATGATTAAACGTTATGATGAACACCACCAGGACC |
| | | TGACCTTACTCAAAGCGTTGGTTCGCCAGCAACTGCCAGAGAAGTACAA |
| | | AGAAATCTTCTTTGATCAGTCAAAGAATGGTTATGCCGGCTATATTGACGG |
| | | GGGTGCAAGCCAAGAGGAATTCTACAAATTTATCAAGCCTATTCTGGAGA |
| | | AAATGGATGGTACCGAAGAGTTATTGGTGAAGCTTAACCGTGAAGACCTC |
| | | CTGCGGAAACAGCGCACATTCGATAATGGTTCGATCCCACACCAAATCC |
| | | ATTTGGGGGAGTTACACGCTATTTTGCGTCGCCAGGAAGACTTTTACCCT |
| | | TTCCTGAAGGATAACCGGGAGAAAATTGAGAAGATCCTTACCTTTCGTATT |
| | | CCGTATTACGTAGGCCCCTTAGCACGGGGTAATAGCCGTTTCGCGTGGA |
| | | TGACACGGAAGTCGGAAGAGACGATCACCCCGTGGAACTTCGAAGAGG |
| | | TAGTCGACAAGGGCGCATCAGCGCAGTCTTTTATTGAACGTATGACGAAT |
| | | TTCGATAAAAACTTGCCCAATGAGAAGGTGCTTCCGAAACATTCCTTGTTA |
| | | TATGAATATTTTACAGTTTACAACGAGCTGACCAAGGTTAAATACGTGACG |
| | | GAAGGAATGCGCAAGCCCGCTTTTCTTAGCGGTGAGCAAAAAAAGGCG |
| | | ATCGTCGACCTGTTATTCAAAACGAATCGTAAGGTGACTGTAAAGCAACTC |
| | | AAAGAAGATTACTTCAAAAAGATTGAGTGCTTCGACAGCGTCGAAATCTCT |
| | | GGGGTAGAGGATCGGTTTAACGCAAGTTTAGGTACCTACCATGACCTGCT |
| | | TAAAATCATTAAGGATAAAGACTTCTTAGATAATGAAGAGAACGAAGATATT |
| | | CTCGAGGACATCGTCTTGACGTTAACCTTATTTGAGGATCGTGAAATGATT |
| | | GAGGAACGCCTCAAAACTTATGCCCACCTGTTCGACGATAAGGTGATGA |
| | | AGCAGCTGAAACGTCGGCGCTACACAGGATGGGGCCGCTTGAGTCGC |
| | | AAACTTATTAACGGAATCCGTGACAAGCAATCCGGCAAAACGATTCTGGA |
| | | TTTCTTGAAGTCGGACGGATTTGCTAATCGCAACTTCATGCAGTTGATCCA |
| | | TGATGACTCCCTGACTITTAAAGAGGATATTCAAAAGGCGCAGGTTAGTG |
| | | GTCAAGGCGACAGCTTACACGAACACATCGCAAATTTGGCTGGTTCGCC |
| | | GGCCATTAAAAAGGGGATCCTCCAGACCGTGAAAGTTGTAGATGAGCTT |
| | | GTTAAGGTCATGGGTCGTCATAAGCCCGAAAACATCGTGATTGAAATGGC |
| | | GCGGGAGAATCAAACGACCCAGAAAGGACAAAAGAATAGCCGTGAAC |
| | | GGATGAAGCGGATCGAGGAAGGCATTAAAGAGCTGGGGTCTCAAATCTT |
| | | GAAGGAACACCCTGTGGAGAACACTCAGCTCCAAAATGAAAAACTTTAC |
| | | CTGTACTATTTGCAGAACGGACGCGATATGTACGTGGACCAAGAGTTGGA |
| | | TATTAATCGGCTGAGTGACTACGACGTTGATCATATCGTCCCGCAGAGCTT |
| | | CCTCAAAGACGATTCTATTGACAATAAGGTACTGACGCGCTCTGATAAAAA |
| | | CCGTGGTAAGTCGGACAACGTGCCCTCCGAAGAGGTTGTGAAAAAGAT |
| | | GAAAAATTATTGGCGCCAGCTTTTAAACGCGAAGCTGATCACACAACGTA |
| | | AATTCGATAATTTGACCAAGGCTGAACGGGGTGGCCTGAGCGAGTTAGAT |
| | | AAGGCAGGATTTATTAAACGCCAGTTAGTGGAGACTCGTCAAATCACCAA |
| | | ACATGTCGCGCAGATTTTGGACAGCCGGATGAACACCAAGTACGATGAA |
| | | AATGACAAACTGATCCGTGAGGTGAAAGTCATTACTCTGAAGTCCAAATTA |
| | | GTTAGTGATTTCCGGAAGGACTTTCAATTCTACAAAGTCCGTGAAATTAATA |
| | | ACTATCATCACGCACATGACGCGTACCTGAATGCAGTGGTTGGGACCGC |
| | | CCTTATCAAGAAATATCCTAAGCTGGAGTCGGAGTTTGTCTATGGCGACTA |
| | | TAAGGTATACGATGTTCGCAAAATGATTGCGAAATCTGAGCAGGAGATCG |
| | | GTAAGGCAACCGCAAAATATTTCTTTTACTCAAACATTATGAATTTCTTTAAG |
| | | ACAGAAATCACTCTGGCCAACGGGGAGATTCGCAAACGTCCGTTGATC |
| | | GAAACAAACGGCGAGACTGGCGAAATTGTTTGGGACAAAGGGCGTGAT |
| | | TTCGCGACGGTGCGCAAGGTACTGAGCATGCCTCAAGTCAATATTGTTAA |
| | | GAAAACCGAAGTGCAGACGGGCGGGTTTTCCAAGGAAAGCATCTTACC |
| | | CAAACGTAATTCAGATAAACTTATTGCACGCAAAAAGGACTGGGATCCGA |
| | | AAAAGTATGGAGGCTTCGACAGTCCAACCGTAGCCTACTCTGTTCTCGTT |
| | | GTAGCGAAAGTAGAAAAGGGTAAATCCAAGAAACTGAAATCTGTCAAGGA |
| | | GTTGCTTGGAATCACCATTATGGAGCGTAGCTCCTTCGAGAAGAACCCG |
| | | ATTGACTTTCTGGAAGCCAAAGGATATAAAGAGGTCAAGAAAGATCTTATC |
| | | ATTAAGCTGCCTAAGTATTCACTCTTCGAGCTGGAAAATGGTCGTAAACG |
| | | CATGCTCGCTTCTGCCGGCGAGTTGCAGAAGGGCAATGAATTAGCACTT |
| | | CCATCAAAGTACGTTAACTTCCTGTATTTGGCCAGCCATTACGAGAAACTG |
| | | AAGGGGTCTCCAGAGGACAACGAACAGAAACAATTATTTGTAGAGCAGC |
| | | ACAAGCATTATCTTGATGAAATCATTGAGCAAATTTCCGAATTCAGTAAACG |
| | | CGTAATCCTGGCCGATGCAAACCTCGACAAGGTGCTGAGCGCTTACAAT |
| | | AAGCATCGCGACAAACCTATCCGTGAGCAGGCTGAAAATATCATTCACCT |
| | | GTTCACATTAACGAACCTGGGCGCTCCGGCCGCTITTAAATATTTCGACA |
| | | CGACAATCGACCGTAAGCGCTATACCAGTACGAAAGAAGTGTTGGATGC |
| | | GACCCTTATTCACCAGTCAATTACAGGATTATATGAGACCCGTATCGACCT |
| | | TAGCCAATTAGGTGGGGAT (SEQ ID NO: 134) |
| Cas9 | K31E, | MDKKYSIGLDIGTNSVGWAVITDEYKVPSKEFKVLGNTDRHSIKKNLIGALLF |
| TM136 | G56A, | DSAKTAEATRLKRTKRRRYTRRKNRICYLQEIFSNEMAKVDDSFFHRLEESFL |
| | E57K, | VEEDKKHERHPIFGNIVDEVAYHEKYPTIYHLRKKLVDSTDKADLRLIYLALA |
| | A68K, | HMIKFRGHFLIEGDLNPDNSDVDKLFIQLVQTYNQLFEENPINASGVDAKAI |
| | H329K, | LSARLSKSRRLENLIAQLPGEKKNGLFGNLIALSLGLTPNFKSNFDLAEDAK |
| | T333R, | LQLSKDTYDDDLDNLLAQIGDQYADLFLAAKNLSDAILLSDILRVNTEITKAP |
| | T474R, | LSASMIKRYDEHKQDLRLLKALVRQQLPEKYKEIFFDQSKNGYAGYIDGGA |
| | R753G, | SQEEFYKFIKPILEKMDGTEELLVKLNREDLLRKQRTFDNGSIPHQIHLGELH |
| | S1106Y | AILRRQEDFYPFLKDNREKIEKILTFRIPYYVGPLARGNSRFAWMTRKSEETIR |
| | | PWNFEEVVDKGASAQSFIERMTNFDKNLPNEKVLPKHSLLYEYFTVYNELT |
| | | KVKYVTEGMRKPAFLSGEQKKAIVDLLFKTNRKVTVKQLKEDYFKKIECFDS |
| | | VEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFL |
| | | KSDGFANRNFMQLIHDDSLTFKEDIQKAQVSGQGDSLHEHIANLAGSPAIK |
| | | KGILQTVKVVDELVKVMGGHKPENIVIEMARENQTTQKGQKNSRERMKRIE |
| | | EGIKELGSQILKEHPVENTQLQNEKLYLYYLQNGRDMYVDQELDINRLSDY |
| | | DVDHIVPQSFLKDDSIDNKVLTRSDKNRGKSDNVPSEEVVKKMKNYWRQL |
| | | LNAKLITQRKFDNLTKAERGGLSELDKAGFIKRQLVETRQITKHVAQILDSRM |
| | | NTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKK |
| | | TEVQTGGFYKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKYSLF |
| | | ELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLS |
| | | QLGGD (SEQ ID NO: 135) |
| Cas9 | K31E, | ATGGACAAGAAGTACAGCATCGGCCTGGACATCGGCACCAATTCTGTTG |
| TM136 | G56A, | GCTGGGCCGTGATCACCGACGAGTACAAGGTGCCCAGCAAGGAGTTC |
| | E57K, | AAGGTGCTGGGCAACACCGACCGGCACAGCATCAAGAAGAATCTGATC |
| | A68K, | GGCGCCCTGCTGTTCGACTCTGCCAAGACAGCCGAAGCCACCAGACT |
| | H329K, | GAAGAGAACCAAGAGACGGCGGTACACCAGAAGAAAGAACCGGATCT |
| | T333R, | GCTACCTGCAAGAGATCTTCAGCAACGAGATGGCCAAGGTGGACGACA |
| | T474R, | GCTTCTTCCACAGACTGGAAGAGTCCTTCCTGGTGGAAGAGGATAAGAA |
| | R753G, | GCACGAGCGGCACCCCATCTTCGGCAACATCGTGGATGAGGTGGCCTA |
| | S1106Y | CCACGAGAAGTACCCCACCATCTACCACCTGAGAAAGAAACTGGTGGA |
| | | CAGCACCGACAAGGCCGACCTGAGACTGATCTATCTGGCCCTGGCTCA |
| | | CATGATCAAGTTCCGGGGCCACTTCCTGATCGAGGGCGACCTGAATCCT |
| | | GACAACAGCGACGTGGACAAGCTGTTCATCCAGCTGGTGCAGACCTAC |
| | | AACCAGCTGTTCGAGGAAAACCCCATCAACGCCAGCGGAGTGGATGC |
| | | CAAGGCCATCCTGTCTGCCAGACTGAGCAAGAGCAGACGGCTGGAAAA |
| | | CCTGATCGCTCAGCTGCCCGGCGAGAAGAAGAATGGCCTGTTCGGCAA |
| | | CCTGATTGCCCTGAGCCTGGGCCTGACACCTAACTTCAAGAGCAACTTC |
| | | GACCTGGCCGAGGACGCCAAACTGCAGCTGTCCAAGGACACCTACGA |
| | | CGACGACCTGGACAATCTGCTGGCCCAGATCGGCGATCAGTACGCCG |
| | | ACTTGTTTCTGGCCGCCAAGAACCTGTCCGACGCCATCCTGCTGAGCG |
| | | ACATCCTGAGAGTGAACACCGAGATCACAAAGGCCCCTCTGAGCGCCT |
| | | CTATGATCAAGAGATACGACGAGCACAAGCAGGATCTGCGGCTGCTGAA |
| | | GGCCCTCGTTAGACAGCAGCTGCCTGAGAAGTACAAAGAGATTTTCTTC |
| | | GACCAGAGCAAGAACGGCTACGCCGGCTACATTGATGGCGGAGCCAG |
| | | CCAAGAGGAATTCTACAAGTTCATCAAGCCCATCCTCGAGAAGATGGAC |
| | | GGCACCGAGGAACTGCTGGTCAAGCTGAACAGAGAGGACCTGCTGCG |
| | | GAAGCAGCGGACCTTCGACAATGGCTCTATCCCTCACCAGATCCACCT |
| | | GGGAGAGCTGCACGCCATTCTGCGGAGACAAGAGGACTTTTACCCATT |
| | | CCTGAAGGACAACCGGGAAAAGATTGAGAAGATCCTGACCTTCAGGATC |
| | | CCCTACTACGTGGGACCACTGGCCAGAGGCAATAGCAGATTCGCCTGG |
| | | ATGACCAGAAAGAGCGAGGAAACCATCCGGCCCTGGAACTTCGAGGAA |
| | | GTGGTGGACAAGGGCGCCAGCGCTCAGTCCTTCATCGAGCGGATGAC |
| | | CAACTTCGATAAGAACCTGCCTAACGAGAAGGTGCTGCCCAAGCACAG |
| | | CCTGCTGTACGAGTACTTCACCGTGTACAACGAGCTGACCAAAGTGAAAT |
| | | ACGTGACCGAGGGAATGAGAAAGCCCGCCTTTCTGAGCGGCGAGCAG |
| | | AAAAAGGCCATTGTGGATCTGCTGTTCAAGACCAACCGGAAAGTGACCG |
| | | TGAAGCAGCTGAAAGAGGACTACTTCAAGAAAATCGAGTGCTTCGACAG |
| | | CGTGGAAATCAGCGGCGTGGAAGATCGGTTCAATGCCAGCCTGGGCAC |
| | | ATACCACGACCTGCTGAAAATTATCAAGGACAAGGACTTCCTGGACAAC |
| | | GAAGAGAACGAGGACATCCTGGAAGATATCGTGCTGACCCTGACACTGT |
| | | TTGAGGACAGAGAGATGATCGAGGAACGGCTGAAAACATACGCCCACC |
| | | TGTTCGACGACAAAGTGATGAAGCAACTGAAGCGGCGGAGATACACCG |
| | | GCTGGGGCAGACTGTCTCGGAAGCTGATCAACGGCATCCGGGATAAGC |
| | | AGTCCGGCAAGACCATCCTGGACTTTCTGAAGTCCGACGGCTTCGCCA |
| | | ATCGGAACTTCATGCAGCTGATCCACGACGACAGCCTGACCTTTAAAGA |
| | | GGATATCCAGAAAGCCCAGGTGTCCGGCCAGGGCGATTCTCTGCATGA |
| | | GCACATTGCCAACCTGGCCGGCTCTCCCGCCATTAAGAAGGGCATTCT |
| | | GCAGACAGTGAAGGTGGTGGACGAGCTGGTCAAAGTCATGGGCGGCC |
| | | ACAAGCCCGAGAACATCGTGATCGAAATGGCCAGAGAGAACCAGACCA |
| | | CACAGAAGGGCCAGAAGAACAGCCGCGAGAGAATGAAGCGGATCGAA |
| | | GAGGGCATCAAAGAGCTGGGCAGCCAGATCCTGAAAGAACACCCCGT |
| | | GGAAAACACCCAGCTGCAGAACGAGAAGCTGTACCTGTACTACCTCCA |
| | | GAACGGCCGGGATATGTACGTGGACCAAGAGCTGGACATCAACCGGCT |
| | | GTCCGACTACGATGTGGACCATATCGTGCCCCAGTCTTTTCTGAAGGAC |
| | | GACTCCATCGACAACAAGGTCCTGACCAGAAGCGACAAGAACCGGGG |
| | | CAAGAGCGATAACGTGCCCTCCGAAGAGGTGGTCAAGAAGATGAAGAA |
| | | CTACTGGCGACAGCTGCTGAACGCCAAGCTGATTACCCAGCGGAAGTT |
| | | CGATAACCTGACCAAGGCCGAGAGAGGCGGCCTGTCTGAACTGGATAA |
| | | GGCCGGCTTCATCAAGAGACAGCTGGTGGAAACCCGGCAGATCACCA |
| | | AACACGTGGCACAGATTCTGGACTCCCGGATGAACACTAAGTACGACGA |
| | | GAATGACAAGCTGATCCGGGAAGTGAAAGTGATCACCCTGAAGTCCAAG |
| | | CTGGTGTCCGATTTCCGGAAGGATTTCCAGTTCTACAAAGTGCGCGAGAT |
| | | CAACAACTACCATCACGCCCACGACGCCTACCTGAATGCCGTTGTTGG |
| | | AACAGCCCTGATCAAGAAGTATCCCAAGCTGGAAAGCGAGTTCGTGTAC |
| | | GGCGACTACAAGGTGTACGACGTGCGGAAGATGATCGCCAAGAGCGAG |
| | | CAAGAGATTGGCAAGGCTACCGCCAAGTACTTCTTCTACAGCAACATCAT |
| | | GAACTTTTTCAAGACCGAGATTACCCTGGCCAACGGCGAGATCAGAAAG |
| | | CGGCCTCTGATCGAGACAAACGGCGAAACCGGCGAGATTGTGTGGGAT |
| | | AAGGGCAGAGACTTTGCCACAGTGCGGAAAGTGCTGAGCATGCCCCAA |
| | | GTGAATATCGTGAAGAAAACCGAGGTGCAGACAGGCGGCTTCTACAAAG |
| | | AGTCCATTCTGCCCAAGAGAAACAGCGATAAGCTGATCGCCCGGAAGA |
| | | AGGACTGGGACCCTAAGAAGTACGGCGGCTTCGATAGCCCTACCGTGG |
| | | CCTATTCTGTGCTGGTGGTGGCCAAAGTGGAAAAGGGCAAGTCCAAGAA |
| | | ACTCAAGAGCGTGAAAGAGCTGCTGGGGATCACCATCATGGAAAGAAG |
| | | CAGCTTCGAGAAGAATCCTATCGATTTCCTCGAGGCCAAGGGCTACAAA |
| | | GAAGTGAAAAAGGACCTGATCATCAAGCTCCCCAAGTACTCCCTGTTCG |
| | | AGCTGGAAAATGGCCGGAAGCGGATGCTGGCTTCTGCTGGCGAACTGC |
| | | AGAAGGGAAACGAACTGGCCCTGCCTAGCAAATATGTGAACTTCCTGTA |
| | | CCTGGCCAGCCACTATGAGAAGCTGAAGGGCAGCCCCGAGGACAATG |
| | | AGCAAAAGCAGCTGTTTGTGGAACAGCACAAGCACTACCTGGACGAGAT |
| | | CATCGAGCAGATCAGCGAGTTCTCCAAGAGAGTGATCCTGGCCGACGC |
| | | TAATCTGGACAAAGTGCTGTCCGCCTACAACAAGCACCGGGACAAGCC |
| | | TATCAGAGAGCAGGCCGAGAATATCATCCACCTGTITACCCTGACCAATC |
| | | TGGGAGCCCCTGCCGCCTTCAAGTACTTTGACACCACCATCGACCGGA |
| | | AGCGCTACACCAGCACCAAAGAGGTGCTGGACGCCACACTGATCCAC |
| | | CAGTCTATCACCGGCCTGTACGAGACACGGATCGACCTGTCTCAGCTTG |
| | | GAGGCGAC (SEQ ID NO: 136) |
| TM9 | S1106Y, | MDKKYSIGLDIGTNSVGWAVITDEYKVPSKKFKVLGNTDRHSIKKNLIGALLF |
| | A68K, | DSGETAEATRLKRTKRRRYTRRKNRICYLQEIFSNEMAKVDDSFFHRLEESF |
| | T474R | LVEEDKKHERHPIFGNIVDEVAYHEKYPTIYHLRKKLVDSTDKADLRLIYLALA |
| | | HMIKFRGHFLIEGDLNPDNSDVDKLFIQLVQTYNQLFEENPINASGVDAKAI |
| | | LSARLSKSRRLENLIAQLPGEKKNGLFGNLIALSLGLTPNFKSNFDLAEDAK |
| | | LQLSKDTYDDDLDNLLAQIGDQYADLFLAAKNLSDAILLSDILRVNTEITKAP |
| | | LSASMIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGELHA |
| | | ILRRQEDFYPFLKDNREKIEKILTFRIPYYVGPLARGNSRFAWMTRKSEETIRP |
| | | WNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSPAIKK |
| | | GILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQLL |
| | | NAKLITQRKFDNLTKAERGGLSELDKAGFIKRQLVETRQITKHVAQILDSRM |
| | | NTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKK |
| | | TEVQTGGFYKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKYSLF |
| | | ELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLS |
| | | QLGGD (SEQ ID NO: 137) |
| TM9 | S1106Y, | ATGGACAAGAAGTACAGCATCGGCCTGGACATCGGCACCAATTCTGTTG |
| | A68K, | GCTGGGCCGTGATCACCGACGAGTACAAGGTGCCCAGCAAGAAATTCA |
| | T474R | AGGTGCTGGGCAACACCGACCGGCACAGCATCAAGAAGAATCTGATCG |
| | | GCGCCCTGCTGTTCGACTCTGGCGAAACAGCCGAAGCCACCAGACTG |
| | | AAGAGAACCAAGAGACGGCGGTACACCAGAAGAAAGAACCGGATCTG |
| | | CTACCTGCAAGAGATCTTCAGCAACGAGATGGCCAAGGTGGACGACAG |
| | | CTTCTTCCACAGACTGGAAGAGTCCTTCCTGGTGGAAGAGGATAAGAAG |
| | | CACGAGCGGCACCCCATCTTCGGCAACATCGTGGATGAGGTGGCCTAC |
| | | CACGAGAAGTACCCCACCATCTACCACCTGAGAAAGAAACTGGTGGAC |
| | | AGCACCGACAAGGCCGACCTGAGACTGATCTATCTGGCCCTGGCTCAC |
| | | ATGATCAAGTTCCGGGGCCACTTCCTGATCGAGGGCGACCTGAATCCTG |
| | | ACAACAGCGACGTGGACAAGCTGTTCATCCAGCTGGTGCAGACCTACA |
| | | ACCAGCTGTTCGAGGAAAACCCCATCAACGCCAGCGGAGTGGATGCC |
| | | AAGGCCATCCTGTCTGCCAGACTGAGCAAGAGCAGACGGCTGGAAAAC |
| | | CTGATCGCTCAGCTGCCCGGCGAGAAGAAGAATGGCCTGTTCGGCAAC |
| | | CTGATTGCCCTGAGCCTGGGCCTGACACCTAACTTCAAGAGCAACTTCG |
| | | ACCTGGCCGAGGACGCCAAACTGCAGCTGTCCAAGGACACCTACGAC |
| | | GACGACCTGGACAATCTGCTGGCCCAGATCGGCGATCAGTACGCCGA |
| | | CTTGTTTCTGGCCGCCAAGAACCTGTCCGACGCCATCCTGCTGAGCGA |
| | | CATCCTGAGAGTGAACACCGAGATCACAAAGGCCCCTCTGAGCGCCTC |
| | | TATGATCAAGAGATACGACGAGCACCACCAGGATCTGACCCTGCTGAAG |
| | | GCCCTCGTTAGACAGCAGCTGCCTGAGAAGTACAAAGAGATTTTCTTCGA |
| | | CCAGAGCAAGAACGGCTACGCCGGCTACATTGATGGCGGAGCCAGCC |
| | | AAGAGGAATTCTACAAGTTCATCAAGCCCATCCTCGAGAAGATGGACGG |
| | | CACCGAGGAACTGCTGGTCAAGCTGAACAGAGAGGACCTGCTGCGGA |
| | | AGCAGCGGACCTTCGACAATGGCTCTATCCCTCACCAGATCCACCTGG |
| | | GAGAGCTGCACGCCATTCTGCGGAGACAAGAGGACTTTTACCCATTCCT |
| | | GAAGGACAACCGGGAAAAGATTGAGAAGATCCTGACCTTCAGGATCCC |
| | | CTACTACGTGGGACCACTGGCCAGAGGCAATAGCAGATTCGCCTGGAT |
| | | GACCAGAAAGAGCGAGGAAACCATCCGGCCCTGGAACTTCGAGGAAG |
| | | TGGTGGACAAGGGCGCCAGCGCTCAGTCCTTCATCGAGCGGATGACC |
| | | AACTTCGATAAGAACCTGCCTAACGAGAAGGTGCTGCCCAAGCACAGC |
| | | CTGCTGTACGAGTACTTCACCGTGTACAACGAGCTGACCAAAGTGAAATA |
| | | CGTGACCGAGGGAATGAGAAAGCCCGCCTTTCTGAGCGGCGAGCAGA |
| | | AAAAGGCCATTGTGGATCTGCTGTTCAAGACCAACCGGAAAGTGACCGT |
| | | GAAGCAGCTGAAAGAGGACTACTTCAAGAAAATCGAGTGCTTCGACAGC |
| | | GTGGAAATCAGCGGCGTGGAAGATCGGTTCAATGCCAGCCTGGGCACA |
| | | TACCACGACCTGCTGAAAATTATCAAGGACAAGGACTTCCTGGACAACG |
| | | AAGAGAACGAGGACATCCTGGAAGATATCGTGCTGACCCTGACACTGTTT |
| | | GAGGACAGAGAGATGATCGAGGAACGGCTGAAAACATACGCCCACCTG |
| | | TTCGACGACAAAGTGATGAAGCAACTGAAGCGGCGGAGATACACCGGC |
| | | TGGGGCAGACTGTCTCGGAAGCTGATCAACGGCATCCGGGATAAGCAG |
| | | TCCGGCAAGACCATCCTGGACTTTCTGAAGTCCGACGGCTTCGCCAATC |
| | | GGAACTTCATGCAGCTGATCCACGACGACAGCCTGACCTTTAAAGAGGA |
| | | TATCCAGAAAGCCCAGGTGTCCGGCCAGGGCGATTCTCTGCATGAGCA |
| | | CATTGCCAACCTGGCCGGCTCTCCCGCCATTAAGAAGGGCATTCTGCA |
| | | GACAGTGAAGGTGGTGGACGAGCTGGTCAAAGTCATGGGCAGACACAA |
| | | GCCCGAGAACATCGTGATCGAAATGGCCAGAGAGAACCAGACCACACA |
| | | GAAGGGCCAGAAGAACAGCCGCGAGAGAATGAAGCGGATCGAAGAGG |
| | | GCATCAAAGAGCTGGGCAGCCAGATCCTGAAAGAACACCCCGTGGAAA |
| | | ACACCCAGCTGCAGAACGAGAAGCTGTACCTGTACTACCTCCAGAACG |
| | | GCCGGGATATGTACGTGGACCAAGAGCTGGACATCAACCGGCTGTCCG |
| | | ACTACGATGTGGACCATATCGTGCCCCAGTCTTTTCTGAAGGACGACTCC |
| | | ATCGACAACAAGGTCCTGACCAGAAGCGACAAGAACCGGGGCAAGAG |
| | | CGATAACGTGCCCTCCGAAGAGGTGGTCAAGAAGATGAAGAACTACTGG |
| | | CGACAGCTGCTGAACGCCAAGCTGATTACCCAGCGGAAGTTCGATAAC |
| | | CTGACCAAGGCCGAGAGAGGCGGCCTGTCTGAACTGGATAAGGCCGG |
| | | CTTCATCAAGAGACAGCTGGTGGAAACCCGGCAGATCACCAAACACGT |
| | | GGCACAGATTCTGGACTCCCGGATGAACACTAAGTACGACGAGAATGAC |
| | | AAGCTGATCCGGGAAGTGAAAGTGATCACCCTGAAGTCCAAGCTGGTGT |
| | | CCGATTTCCGGAAGGATTTCCAGTTCTACAAAGTGCGCGAGATCAACAA |
| | | CTACCATCACGCCCACGACGCCTACCTGAATGCCGTTGTTGGAACAGC |
| | | CCTGATCAAGAAGTATCCCAAGCTGGAAAGCGAGTTCGTGTACGGCGAC |
| | | TACAAGGTGTACGACGTGCGGAAGATGATCGCCAAGAGCGAGCAAGAG |
| | | ATTGGCAAGGCTACCGCCAAGTACTTCTTCTACAGCAACATCATGAACTT |
| | | TTTCAAGACCGAGATTACCCTGGCCAACGGCGAGATCAGAAAGCGGCC |
| | | TCTGATCGAGACAAACGGCGAAACCGGCGAGATTGTGTGGGATAAGGG |
| | | CAGAGACTTTGCCACAGTGCGGAAAGTGCTGAGCATGCCCCAAGTGAAT |
| | | ATCGTGAAGAAAACCGAGGTGCAGACAGGCGGCTTCTACAAAGAGTCC |
| | | ATTCTGCCCAAGAGAAACAGCGATAAGCTGATCGCCCGGAAGAAGGAC |
| | | TGGGACCCTAAGAAGTACGGCGGCTTCGATAGCCCTACCGTGGCCTATT |
| | | CTGTGCTGGTGGTGGCCAAAGTGGAAAAGGGCAAGTCCAAGAAACTCA |
| | | AGAGCGTGAAAGAGCTGCTGGGGATCACCATCATGGAAAGAAGCAGCT |
| | | TCGAGAAGAATCCTATCGATTTCCTCGAGGCCAAGGGCTACAAAGAAGT |
| | | GAAAAAGGACCTGATCATCAAGCTCCCCAAGTACTCCCTGTTCGAGCTG |
| | | GAAAATGGCCGGAAGCGGATGCTGGCTTCTGCTGGCGAACTGCAGAAG |
| | | GGAAACGAACTGGCCCTGCCTAGCAAATATGTGAACTTCCTGTACCTGG |
| | | CCAGCCACTATGAGAAGCTGAAGGGCAGCCCCGAGGACAATGAGCAA |
| | | AAGCAGCTGTTTGTGGAACAGCACAAGCACTACCTGGACGAGATCATCG |
| | | AGCAGATCAGCGAGTTCTCCAAGAGAGTGATCCTGGCCGACGCTAATCT |
| | | GGACAAAGTGCTGTCCGCCTACAACAAGCACCGGGACAAGCCTATCAG |
| | | AGAGCAGGCCGAGAATATCATCCACCTGTTTACCCTGACCAATCTGGGA |
| | | GCCCCTGCCGCCTTCAAGTACTTTGACACCACCATCGACCGGAAGCGC |
| | | TACACCAGCACCAAAGAGGTGCTGGACGCCACACTGATCCACCAGTCT |
| | | ATCACCGGCCTGTACGAGACACGGATCGACCTGTCTCAGCTTGGAGGC |
| | | GAC (SEQ ID NO: 138) |
| TM108 | S1106Y, | MDKKYSIGLDIGTNSVGWAVITDEYKVPSKEFKVLGNTDRHSIKKNLIGALLF |
| | A68K, | DSAKTAEATRLKRTKRRRYTRRKNRICYLQEIFSNEMAKVDDSFFHRLEESFL |
| | T474R, | VEEDKKHERHPIFGNIVDEVAYHEKYPTIYHLRKKLVDSTDKADLRLIYLALA |
| | K31E, | HMIKFRGHFLIEGDLNPDNSDVDKLFIQLVQTYNQLFEENPINASGVDAKAI |
| | G56A, | LSARLSKSRRLENLIAQLPGEKKNGLFGNLIALSLGLTPNFKSNFDLAEDAK |
| | E57K | LQLSKDTYDDDLDNLLAQIGDQYADLFLAAKNLSDAILLSDILRVNTEITKAP |
| | | LSASMIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGELHA |
| | | ILRRQEDFYPFLKDNREKIEKILTFRIPYYVGPLARGNSRFAWMTRKSEETIRP |
| | | WNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSPAIKK |
| | | GILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQLL |
| | | NAKLITQRKFDNLTKAERGGLSELDKAGFIKRQLVETRQITKHVAQILDSRM |
| | | NTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKK |
| | | TEVQTGGFYKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKYSLF |
| | | ELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLS |
| | | QLGGD (SEQ ID NO: 139) |
| TM108 | S1106Y, | ATGGACAAGAAGTACAGCATCGGCCTGGACATCGGCACCAATTCTGTTG |
| | A68K, | GCTGGGCCGTGATCACCGACGAGTACAAGGTGCCCAGCAAGGAGTTC |
| | T474R, | AAGGTGCTGGGCAACACCGACCGGCACAGCATCAAGAAGAATCTGATC |
| | K31E, | GGCGCCCTGCTGTTCGACTCTGCCAAGACAGCCGAAGCCACCAGACT |
| | G56A, | GAAGAGAACCAAGAGACGGCGGTACACCAGAAGAAAGAACCGGATCT |
| | | GCTACCTGCAAGAGATCTTCAGCAACGAGATGGCCAAGGTGGACGACA |
| | | GCTTCTTCCACAGACTGGAAGAGTCCTTCCTGGTGGAAGAGGATAAGAA |
| | | GCACGAGCGGCACCCCATCTTCGGCAACATCGTGGATGAGGTGGCCTA |
| | | CCACGAGAAGTACCCCACCATCTACCACCTGAGAAAGAAACTGGTGGA |
| | | CAGCACCGACAAGGCCGACCTGAGACTGATCTATCTGGCCCTGGCTCA |
| | | CATGATCAAGTTCCGGGGCCACTTCCTGATCGAGGGCGACCTGAATCCT |
| | | GACAACAGCGACGTGGACAAGCTGTTCATCCAGCTGGTGCAGACCTAC |
| | | AACCAGCTGTTCGAGGAAAACCCCATCAACGCCAGCGGAGTGGATGC |
| | | CAAGGCCATCCTGTCTGCCAGACTGAGCAAGAGCAGACGGCTGGAAAA |
| | | CCTGATCGCTCAGCTGCCCGGCGAGAAGAAGAATGGCCTGTTCGGCAA |
| | | CCTGATTGCCCTGAGCCTGGGCCTGACACCTAACTTCAAGAGCAACTTC |
| | | GACCTGGCCGAGGACGCCAAACTGCAGCTGTCCAAGGACACCTACGA |
| | | CGACGACCTGGACAATCTGCTGGCCCAGATCGGCGATCAGTACGCCG |
| | | ACTTGTTTCTGGCCGCCAAGAACCTGTCCGACGCCATCCTGCTGAGCG |
| | | ACATCCTGAGAGTGAACACCGAGATCACAAAGGCCCCTCTGAGCGCCT |
| | | CTATGATCAAGAGATACGACGAGCACCACCAGGATCTGACCCTGCTGAA |
| | | GGCCCTCGTTAGACAGCAGCTGCCTGAGAAGTACAAAGAGATTTTCTTC |
| | | GACCAGAGCAAGAACGGCTACGCCGGCTACATTGATGGCGGAGCCAG |
| | | CCAAGAGGAATTCTACAAGTTCATCAAGCCCATCCTCGAGAAGATGGAC |
| | | GGCACCGAGGAACTGCTGGTCAAGCTGAACAGAGAGGACCTGCTGCG |
| | | GAAGCAGCGGACCTTCGACAATGGCTCTATCCCTCACCAGATCCACCT |
| | | GGGAGAGCTGCACGCCATTCTGCGGAGACAAGAGGACTTTTACCCATT |
| | | CCTGAAGGACAACCGGGAAAAGATTGAGAAGATCCTGACCTTCAGGATC |
| | | CCCTACTACGTGGGACCACTGGCCAGAGGCAATAGCAGATTCGCCTGG |
| | | ATGACCAGAAAGAGCGAGGAAACCATCCGGCCCTGGAACTTCGAGGAA |
| | | GTGGTGGACAAGGGCGCCAGCGCTCAGTCCTTCATCGAGCGGATGAC |
| | | CAACTTCGATAAGAACCTGCCTAACGAGAAGGTGCTGCCCAAGCACAG |
| | | CCTGCTGTACGAGTACTTCACCGTGTACAACGAGCTGACCAAAGTGAAAT |
| | | ACGTGACCGAGGGAATGAGAAAGCCCGCCTTTCTGAGCGGCGAGCAG |
| | | AAAAAGGCCATTGTGGATCTGCTGTTCAAGACCAACCGGAAAGTGACCG |
| | | TGAAGCAGCTGAAAGAGGACTACTTCAAGAAAATCGAGTGCTTCGACAG |
| | | CGTGGAAATCAGCGGCGTGGAAGATCGGTTCAATGCCAGCCTGGGCAC |
| | | ATACCACGACCTGCTGAAAATTATCAAGGACAAGGACTTCCTGGACAAC |
| | | GAAGAGAACGAGGACATCCTGGAAGATATCGTGCTGACCCTGACACTGT |
| | | TTGAGGACAGAGAGATGATCGAGGAACGGCTGAAAACATACGCCCACC |
| | | TGTTCGACGACAAAGTGATGAAGCAACTGAAGCGGCGGAGATACACCG |
| | | GCTGGGGCAGACTGTCTCGGAAGCTGATCAACGGCATCCGGGATAAGC |
| | | AGTCCGGCAAGACCATCCTGGACTTTCTGAAGTCCGACGGCTTCGCCA |
| | | ATCGGAACTTCATGCAGCTGATCCACGACGACAGCCTGACCTTTAAAGA |
| | | GGATATCCAGAAAGCCCAGGTGTCCGGCCAGGGCGATTCTCTGCATGA |
| | | GCACATTGCCAACCTGGCCGGCTCTCCCGCCATTAAGAAGGGCATTCT |
| | | GCAGACAGTGAAGGTGGTGGACGAGCTGGTCAAAGTCATGGGCAGACA |
| | | CAAGCCCGAGAACATCGTGATCGAAATGGCCAGAGAGAACCAGACCAC |
| | | ACAGAAGGGCCAGAAGAACAGCCGCGAGAGAATGAAGCGGATCGAAG |
| | | AGGGCATCAAAGAGCTGGGCAGCCAGATCCTGAAAGAACACCCCGTG |
| | | GAAAACACCCAGCTGCAGAACGAGAAGCTGTACCTGTACTACCTCCAG |
| | | AACGGCCGGGATATGTACGTGGACCAAGAGCTGGACATCAACCGGCTG |
| | | TCCGACTACGATGTGGACCATATCGTGCCCCAGTCTTTTCTGAAGGACGA |
| | | CTCCATCGACAACAAGGTCCTGACCAGAAGCGACAAGAACCGGGGCA |
| | | AGAGCGATAACGTGCCCTCCGAAGAGGTGGTCAAGAAGATGAAGAACTA |
| | | CTGGCGACAGCTGCTGAACGCCAAGCTGATTACCCAGCGGAAGTTCGA |
| | | TAACCTGACCAAGGCCGAGAGAGGCGGCCTGTCTGAACTGGATAAGGC |
| | | CGGCTTCATCAAGAGACAGCTGGTGGAAACCCGGCAGATCACCAAACA |
| | | CGTGGCACAGATTCTGGACTCCCGGATGAACACTAAGTACGACGAGAAT |
| | | GACAAGCTGATCCGGGAAGTGAAAGTGATCACCCTGAAGTCCAAGCTG |
| | | GTGTCCGATTTCCGGAAGGATTTCCAGTTCTACAAAGTGCGCGAGATCAA |
| | | CAACTACCATCACGCCCACGACGCCTACCTGAATGCCGTTGTTGGAAC |
| | | AGCCCTGATCAAGAAGTATCCCAAGCTGGAAAGCGAGTTCGTGTACGGC |
| | | GACTACAAGGTGTACGACGTGCGGAAGATGATCGCCAAGAGCGAGCAA |
| | | GAGATTGGCAAGGCTACCGCCAAGTACTTCTTCTACAGCAACATCATGAA |
| | | CTTTTTCAAGACCGAGATTACCCTGGCCAACGGCGAGATCAGAAAGCG |
| | | GCCTCTGATCGAGACAAACGGCGAAACCGGCGAGATTGTGTGGGATAA |
| | | GGGCAGAGACTTTGCCACAGTGCGGAAAGTGCTGAGCATGCCCCAAGT |
| | | GAATATCGTGAAGAAAACCGAGGTGCAGACAGGCGGCTTCTACAAAGA |
| | | GTCCATTCTGCCCAAGAGAAACAGCGATAAGCTGATCGCCCGGAAGAA |
| | | GGACTGGGACCCTAAGAAGTACGGCGGCTTCGATAGCCCTACCGTGGC |
| | | CTATTCTGTGCTGGTGGTGGCCAAAGTGGAAAAGGGCAAGTCCAAGAAA |
| | | CTCAAGAGCGTGAAAGAGCTGCTGGGGATCACCATCATGGAAAGAAGC |
| | | AGCTTCGAGAAGAATCCTATCGATTTCCTCGAGGCCAAGGGCTACAAAG |
| | | AAGTGAAAAAGGACCTGATCATCAAGCTCCCCAAGTACTCCCTGTTCGA |
| | | GCTGGAAAATGGCCGGAAGCGGATGCTGGCTTCTGCTGGCGAACTGCA |
| | | GAAGGGAAACGAACTGGCCCTGCCTAGCAAATATGTGAACTTCCTGTAC |
| | | CTGGCCAGCCACTATGAGAAGCTGAAGGGCAGCCCCGAGGACAATGA |
| | | GCAAAAGCAGCTGTTTGTGGAACAGCACAAGCACTACCTGGACGAGAT |
| | | CATCGAGCAGATCAGCGAGTTCTCCAAGAGAGTGATCCTGGCCGACGC |
| | | TAATCTGGACAAAGTGCTGTCCGCCTACAACAAGCACCGGGACAAGCC |
| | | TATCAGAGAGCAGGCCGAGAATATCATCCACCTGTITACCCTGACCAATC |
| | | TGGGAGCCCCTGCCGCCTTCAAGTACTTTGACACCACCATCGACCGGA |
| | | AGCGCTACACCAGCACCAAAGAGGTGCTGGACGCCACACTGATCCAC |
| | | CAGTCTATCACCGGCCTGTACGAGACACGGATCGACCTGTCTCAGCTTG |
| | | GAGGCGAC (SEQ ID NO: 140) |
| TM110 | S1106Y, | MDKKYSIGLDIGTNSVGWAVITDEYKVPSKEFKVLGNTDRHSIKKNLIGALLF |
| | A68K, | DSAKTAEATRLKRTKRRRYTRRKNRICYLQEIFSNEMAKVDDSFFHRLEESFL |
| | T474R, | VEEDKKHERHPIFGNIVDEVAYHEKYPTIYHLRKKLVDSTDKADLRLIYLALA |
| | K31E, | HMIKFRGHFLIEGDLNPDNSDVDKLFIQLVQTYNQLFEENPINASGVDAKAI |
| | G56A, | LSARLSKSRRLENLIAQLPGEKKNGLFGNLIALSLGLTPNFKSNFDLAEDAK |
| | E57K, | LQLSKDTYDDDLDNLLAQIGDQYADLFLAAKNLSDAILLSDILRVNTEITKAP |
| | K1085E | LSASMIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGELHA |
| | | ILRRQEDFYPFLKDNREKIEKILTFRIPYYVGPLARGNSRFAWMTRKSEETIRP |
| | | WNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSPAIKK |
| | | GILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQLL |
| | | NAKLITQRKFDNLTKAERGGLSELDKAGFIKRQLVETRQITKHVAQILDSRM |
| | | NTKYDENDKLIREVKVITLKSKLVSDFRKDFQFYKVREINNYHHAHDAYLNA |
| | | VVGTALIKKYPKLESEFVYGDYKVYDVRKMIAKSEQEIGKATAKYFFYSNIMN |
| | | FFKTEITLANGEIRKRPLIETNGETGEIVWDKGRDFATVREVLSMPQVNIVKK |
| | | TEVQTGGFYKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKYSLF |
| | | ELENGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYETRIDLS |
| | | QLGGD (SEQ ID NO: 141) |
| TM110 | S1106Y, | ATGGACAAGAAGTACAGCATCGGCCTGGACATCGGCACCAATTCTGTTG |
| | A68K, | GCTGGGCCGTGATCACCGACGAGTACAAGGTGCCCAGCAAGGAGTTC |
| | T474R, | AAGGTGCTGGGCAACACCGACCGGCACAGCATCAAGAAGAATCTGATC |
| | K31E, | GGCGCCCTGCTGTTCGACTCTGCCAAGACAGCCGAAGCCACCAGACT |
| | G56A, | GAAGAGAACCAAGAGACGGCGGTACACCAGAAGAAAGAACCGGATCT |
| | E57K, | GCTACCTGCAAGAGATCTTCAGCAACGAGATGGCCAAGGTGGACGACA |
| | K1085E | GCTTCTTCCACAGACTGGAAGAGTCCTTCCTGGTGGAAGAGGATAAGAA |
| | | GCACGAGCGGCACCCCATCTTCGGCAACATCGTGGATGAGGTGGCCTA |
| | | CCACGAGAAGTACCCCACCATCTACCACCTGAGAAAGAAACTGGTGGA |
| | | CAGCACCGACAAGGCCGACCTGAGACTGATCTATCTGGCCCTGGCTCA |
| | | CATGATCAAGTTCCGGGGCCACTTCCTGATCGAGGGCGACCTGAATCCT |
| | | GACAACAGCGACGTGGACAAGCTGTTCATCCAGCTGGTGCAGACCTAC |
| | | AACCAGCTGTTCGAGGAAAACCCCATCAACGCCAGCGGAGTGGATGC |
| | | CAAGGCCATCCTGTCTGCCAGACTGAGCAAGAGCAGACGGCTGGAAAA |
| | | CCTGATCGCTCAGCTGCCCGGCGAGAAGAAGAATGGCCTGTTCGGCAA |
| | | CCTGATTGCCCTGAGCCTGGGCCTGACACCTAACTTCAAGAGCAACTTC |
| | | GACCTGGCCGAGGACGCCAAACTGCAGCTGTCCAAGGACACCTACGA |
| | | CGACGACCTGGACAATCTGCTGGCCCAGATCGGCGATCAGTACGCCG |
| | | ACTTGTTTCTGGCCGCCAAGAACCTGTCCGACGCCATCCTGCTGAGCG |
| | | ACATCCTGAGAGTGAACACCGAGATCACAAAGGCCCCTCTGAGCGCCT |
| | | CTATGATCAAGAGATACGACGAGCACCACCAGGATCTGACCCTGCTGAA |
| | | GGCCCTCGTTAGACAGCAGCTGCCTGAGAAGTACAAAGAGATTTTCTTC |
| | | GACCAGAGCAAGAACGGCTACGCCGGCTACATTGATGGCGGAGCCAG |
| | | CCAAGAGGAATTCTACAAGTTCATCAAGCCCATCCTCGAGAAGATGGAC |
| | | GGCACCGAGGAACTGCTGGTCAAGCTGAACAGAGAGGACCTGCTGCG |
| | | GAAGCAGCGGACCTTCGACAATGGCTCTATCCCTCACCAGATCCACCT |
| | | GGGAGAGCTGCACGCCATTCTGCGGAGACAAGAGGACTTTTACCCATT |
| | | CCTGAAGGACAACCGGGAAAAGATTGAGAAGATCCTGACCTTCAGGATC |
| | | CCCTACTACGTGGGACCACTGGCCAGAGGCAATAGCAGATTCGCCTGG |
| | | ATGACCAGAAAGAGCGAGGAAACCATCCGGCCCTGGAACTTCGAGGAA |
| | | GTGGTGGACAAGGGCGCCAGCGCTCAGTCCTTCATCGAGCGGATGAC |
| | | CAACTTCGATAAGAACCTGCCTAACGAGAAGGTGCTGCCCAAGCACAG |
| | | CCTGCTGTACGAGTACTTCACCGTGTACAACGAGCTGACCAAAGTGAAAT |
| | | ACGTGACCGAGGGAATGAGAAAGCCCGCCTTTCTGAGCGGCGAGCAG |
| | | AAAAAGGCCATTGTGGATCTGCTGTTCAAGACCAACCGGAAAGTGACCG |
| | | TGAAGCAGCTGAAAGAGGACTACTTCAAGAAAATCGAGTGCTTCGACAG |
| | | CGTGGAAATCAGCGGCGTGGAAGATCGGTTCAATGCCAGCCTGGGCAC |
| | | ATACCACGACCTGCTGAAAATTATCAAGGACAAGGACTTCCTGGACAAC |
| | | GAAGAGAACGAGGACATCCTGGAAGATATCGTGCTGACCCTGACACTGT |
| | | TTGAGGACAGAGAGATGATCGAGGAACGGCTGAAAACATACGCCCACC |
| | | TGTTCGACGACAAAGTGATGAAGCAACTGAAGCGGCGGAGATACACCG |
| | | GCTGGGGCAGACTGTCTCGGAAGCTGATCAACGGCATCCGGGATAAGC |
| | | AGTCCGGCAAGACCATCCTGGACTTTCTGAAGTCCGACGGCTTCGCCA |
| | | ATCGGAACTTCATGCAGCTGATCCACGACGACAGCCTGACCTTTAAAGA |
| | | GGATATCCAGAAAGCCCAGGTGTCCGGCCAGGGCGATTCTCTGCATGA |
| | | GCACATTGCCAACCTGGCCGGCTCTCCCGCCATTAAGAAGGGCATTCT |
| | | GCAGACAGTGAAGGTGGTGGACGAGCTGGTCAAAGTCATGGGCAGACA |
| | | CAAGCCCGAGAACATCGTGATCGAAATGGCCAGAGAGAACCAGACCAC |
| | | ACAGAAGGGCCAGAAGAACAGCCGCGAGAGAATGAAGCGGATCGAAG |
| | | AGGGCATCAAAGAGCTGGGCAGCCAGATCCTGAAAGAACACCCCGTG |
| | | GAAAACACCCAGCTGCAGAACGAGAAGCTGTACCTGTACTACCTCCAG |
| | | AACGGCCGGGATATGTACGTGGACCAAGAGCTGGACATCAACCGGCTG |
| | | TCCGACTACGATGTGGACCATATCGTGCCCCAGTCTTTTCTGAAGGACGA |
| | | CTCCATCGACAACAAGGTCCTGACCAGAAGCGACAAGAACCGGGGCA |
| | | AGAGCGATAACGTGCCCTCCGAAGAGGTGGTCAAGAAGATGAAGAACTA |
| | | CTGGCGACAGCTGCTGAACGCCAAGCTGATTACCCAGCGGAAGTTCGA |
| | | TAACCTGACCAAGGCCGAGAGAGGCGGCCTGTCTGAACTGGATAAGGC |
| | | CGGCTTCATCAAGAGACAGCTGGTGGAAACCCGGCAGATCACCAAACA |
| | | CGTGGCACAGATTCTGGACTCCCGGATGAACACTAAGTACGACGAGAAT |
| | | GACAAGCTGATCCGGGAAGTGAAAGTGATCACCCTGAAGTCCAAGCTG |
| | | GTGTCCGATTTCCGGAAGGATTTCCAGTTCTACAAAGTGCGCGAGATCAA |
| | | CAACTACCATCACGCCCACGACGCCTACCTGAATGCCGTTGTTGGAAC |
| | | AGCCCTGATCAAGAAGTATCCCAAGCTGGAAAGCGAGTTCGTGTACGGC |
| | | GACTACAAGGTGTACGACGTGCGGAAGATGATCGCCAAGAGCGAGCAA |
| | | GAGATTGGCAAGGCTACCGCCAAGTACTTCTTCTACAGCAACATCATGAA |
| | | CTTTTTCAAGACCGAGATTACCCTGGCCAACGGCGAGATCAGAAAGCG |
| | | GCCTCTGATCGAGACAAACGGCGAAACCGGCGAGATTGTGTGGGATAA |
| | | GGGCAGAGACTTTGCCACAGTGCGGGAGGTGCTGAGCATGCCCCAAG |
| | | TGAATATCGTGAAGAAAACCGAGGTGCAGACAGGCGGCTTCTACAAAGA |
| | | GTCCATTCTGCCCAAGAGAAACAGCGATAAGCTGATCGCCCGGAAGAA |
| | | GGACTGGGACCCTAAGAAGTACGGCGGCTTCGATAGCCCTACCGTGGC |
| | | CTATTCTGTGCTGGTGGTGGCCAAAGTGGAAAAGGGCAAGTCCAAGAAA |
| | | CTCAAGAGCGTGAAAGAGCTGCTGGGGATCACCATCATGGAAAGAAGC |
| | | AGCTTCGAGAAGAATCCTATCGATTTCCTCGAGGCCAAGGGCTACAAAG |
| | | AAGTGAAAAAGGACCTGATCATCAAGCTCCCCAAGTACTCCCTGTTCGA |
| | | GCTGGAAAATGGCCGGAAGCGGATGCTGGCTTCTGCTGGCGAACTGCA |
| | | GAAGGGAAACGAACTGGCCCTGCCTAGCAAATATGTGAACTTCCTGTAC |
| | | CTGGCCAGCCACTATGAGAAGCTGAAGGGCAGCCCCGAGGACAATGA |
| | | GCAAAAGCAGCTGTTTGTGGAACAGCACAAGCACTACCTGGACGAGAT |
| | | CATCGAGCAGATCAGCGAGTTCTCCAAGAGAGTGATCCTGGCCGACGC |
| | | TAATCTGGACAAAGTGCTGTCCGCCTACAACAAGCACCGGGACAAGCC |
| | | TATCAGAGAGCAGGCCGAGAATATCATCCACCTGTITACCCTGACCAATC |
| | | TGGGAGCCCCTGCCGCCTTCAAGTACTTTGACACCACCATCGACCGGA |
| | | AGCGCTACACCAGCACCAAAGAGGTGCTGGACGCCACACTGATCCAC |
| | | CAGTCTATCACCGGCCTGTACGAGACACGGATCGACCTGTCTCAGCTTG |
| | | GAGGCGAC (SEQ ID NO: 142) |

TABLE 9
Synthetic short guide designs

| | |
|---|---|
| 100 (full | NNNNNNNNNNNNNNNNNNNNGUUUUAGAGCUAGAAAUAGCAAGUUAAAAUAAGGCUA |
| length) | GUCCGUUAUCAACUUGAAAAAGUGGCACCGAGUCGGUGCUUUU (SEQ ID NO: 1) |
| 70 | NNNNNNNNNNNNNNNNNNNNGUUUUAGAGCUAGAAAUAGCAAGUUAAAAUAAGGCUAGUC |
| | CGUUAUCAAC (SEQ ID NO: 143) |
| 69 | NNNNNNNNNNNNNNNNNNNNGUUUUAGAGCUAGAAAUAGCAAGUUAAAAUAAGGCUAGUC |
| | CGUUAUCAA (SEQ ID NO: 144) |
| 68 | NNNNNNNNNNNNNNNNNNNNGUUUUAGAGCUAGAAAUAGCAAGUUAAAAUAAGGCUAGUC |
| | CGUUAUCA (SEQ ID NO: 145) |
| 67 | NNNNNNNNNNNNNNNNNNNNGUUUUAGAGCUAGAAAUAGCAAGUUAAAAUAAGGCUAGUC |
| | CGUUAUC (SEQ ID NO: 146) |
| 66 | NNNNNNNNNNNNNNNNNNNNGUUUUAGAGCUAGAAAUAGCAAGUUAAAAUAAGGCUAGUC |
| | CGUUAU (SEQ ID NO: 147) |
| 65 | NNNNNNNNNNNNNNNNNNNNGUUUUAGAGCUAGAAAUAGCAAGUUAAAAUAAGGCUAGUC |
| | CGUUA (SEQ ID NO: 148) |
| 64 | NNNNNNNNNNNNNNNNNNNNGUUUUAGAGCUAGAAAUAGCAAGUUAAAAUAAGGCUAGUC |
| | CGUU (SEQ ID NO: 149) |
| 63 | NNNNNNNNNNNNNNNNNNNNGUUUUAGAGCUAGAAAUAGCAAGUUAAAAUAAGGCUAGUC |
| | CGU (SEQ ID NO: 150) |
| 62 | NNNNNNNNNNNNNNNNNNNNGUUUUAGAGCUAGAAAUAGCAAGUUAAAAUAAGGCUAGUC |
| | CG (SEQ ID NO: 151) |
| 61 | NNNNNNNNNNNNNNNNNNNNGUUUUAGAGCUAGAAAUAGCAAGUUAAAAUAAGGCUAGUC |
| | C (SEQ ID NO: 152) |
| 60 | NNNNNNNNNNNNNNNNNNNNGUUUUAGAGCUAGAAAUAGCAAGUUAAAAUAAGGCUAGUC |
| | (SEQ ID NO: 153) |
| 59 | NNNNNNNNNNNNNNNNNNNNGUUUUAGAGCUAGAAAUAGCAAGUUAAAAUAAGGCUAGU |
| | (SEQ ID NO: 154) |
| 58 | NNNNNNNNNNNNNNNNNNNNGUUUUAGAGCUAGAAAUAGCAAGUUAAAAUAAGGCUAG |
| | (SEQ ID NO: 155) |

TABLE 10
Synthetic short guide designs with internal deletions (ID)

| | |
|---|---|
| 100 (full | NNNNNNNNNNNNNNNNNNNNGUUUUAGAGCUAGAAAUAGCAAGUUAAAAUAAGGCUA |
| length) | GUCCGUUAUCAACUUGAAAAAGUGGCACCGAGUCGGUGCUUUU (SEQ ID NO: 1) |
| 71ID | NNNNNNNNNNNNNNNNNNNNGUUUUAGAGGAAACAAGUUAAAAUAAGGCUAGUCCGUUAU |
| | CAACU (SEQ ID NO: 156) |
| 70ID | NNNNNNNNNNNNNNNNNNNNGUUUUAGAGGAAACAAGUUAAAAUAAGGCUAGUCCGUUAU |
| | CAAC (SEQ ID NO: 157) |
| 69ID | NNNNNNNNNNNNNNNNNNNNGUUUUAGAGGAAACAAGUUAAAAUAAGGCUAGUCCGUUAU |
| | CAA (SEQ ID NO: 158) |
| 68ID | NNNNNNNNNNNNNNNNNNNNGUUUUAGAGGAAACAAGUUAAAAUAAGGCUAGUCCGUUAU |
| | CA (SEQ ID NO: 159) |
| 67ID | NNNNNNNNNNNNNNNNNNNNGUUUUAGAGGAAACAAGUUAAAAUAAGGCUAGUCCGUUAU |
| | C (SEQ ID NO: 160) |
| 66ID | NNNNNNNNNNNNNNNNNNNNGUUUUAGAGGAAACAAGUUAAAAUAAGGCUAGUCCGUUAU |
| | (SEQ ID NO: 161) |
| 65ID | NNNNNNNNNNNNNNNNNNNNGUUUUAGAGGAAACAAGUUAAAAUAAGGCUAGUCCGUUA |
| | (SEQ ID NO: 162) |
| 64ID | NNNNNNNNNNNNNNNNNNNNGUUUUAGAGGAAACAAGUUAAAAUAAGGCUAGUCCGUU |
| | (SEQ ID NO: 163) |
| 63ID | NNNNNNNNNNNNNNNNNNNNGUUUUAGAGGAAACAAGUUAAAAUAAGGCUAGUCCGU |
| | (SEQ ID NO: 164) |
| 62ID | NNNNNNNNNNNNNNNNNNNNGUUUUAGAGGAAACAAGUUAAAAUAAGGCUAGUCCG (SEQ |
| | ID NO: 165) |
| 61ID | NNNNNNNNNNNNNNNNNNNNGUUUUAGAGGAAACAAGUUAAAAUAAGGCUAGUCC (SEQ |
| | ID NO: 166) |
| 60ID | NNNNNNNNNNNNNNNNNNNNGUUUUAGAGGAAACAAGUUAAAAUAAGGCUAGUC (SEQ ID |
| | NO: 167) |
| 59ID | NNNNNNNNNNNNNNNNNNNNGUUUUAGAGGAAACAAGUUAAAAUAAGGCUAGU (SEQ ID |
| | NO: 168) |

Transcription termination of the plasmid expressed guides from a U6 promoter results in the unavoidable addition of 4 U's. Table 9 and Table 10 provides the sequences of the synthetic short guides produced without the need for transcription termination. N represents a region directed to a target-specific spacer sequence within a synthetic guide and can comprise any nucleotide sequence that is complimentary to the protospacer of a target. In some embodiments, N can be at least 15, 16, 17, 18, 19, or 20nt long.

### Definitions

To aid in understanding the invention, several terms are defined below.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “Alt-R,” as that term may modify a Cas reagent, such as a Cas protein, a Cas guide RNA, including a crRNA, tracrRNA, or sgRNA, or a Cas RNP complex refers to a trademarked (™ or®) IDT product that is a purified molecule of defined sequence composition and length and often include chemical modifications to help increase activity, nuclease resistance, and reduce innate immune responses compared to other Cas reagents. Alt-R Cas proteins and enzymes include nuclear localization sequences (NLSs) and a terminal affinity tag, such as a one or more C-terminal 6-His tags.

The terms “target DNA,” “DNA target,” and “DNA target site” refers to the specific DNA site at which a targeting DNA endonuclease contacts and initiates cleavage.

The term “CRISPR” refers to Clustered Regularly Interspaced Short Palindromic Repeat bacterial adaptive immune system.

The terms “Cas” and “Cas endonuclease” generally refers to a CRISPR-associated endonuclease.

The term “Cas protein” generally refers to a wild-type protein, including a variant thereof, of a CRISPR-associated endonuclease (including the interchangeable terms Cas and Cas endonuclease).

The term “Cas nucleic acid” generally refers to a nucleic acid of a CRISPR-associated endonuclease, including a guide RNA, sgRNA, crRNA, or tracrRNA.

The terms “Cas9,” “Cas9” and “CRISPR/Cas9” refer to the CRISPR-associated bacterial adaptive immune system of, for example, Streptococcus pyogenes. Examples of this system are disclosed in U.S. Pat. Nos. 10,717,978 and 11,242,542, filed Oct. 10, 2017 and Apr. 26, 2018, respectively, the contents of which are incorporated by reference herein. The term “Cas9” includes a Cas9 protein amino acid sequence, a Cas9 protein amino acid sequence that includes additional amino acids (for example, such as an affinity tag or nuclear localization signal), or an amino acid sequence having at least 80% homology to a Cas9 protein amino acid sequence, including 80%, 85%, 90%, 95%, and 99% homology to a Cas9 protein amino acid sequence.

The terms “scaffold sequence” and “scaffold region” refers to positions 21-96 of a standard 100 nt Cas9 sgRNA.

The term “variant,” as that term modifies a reference protein amino acid sequence (for example, Cas9 protein), refers to a protein that includes at least one amino acid substitution of the reference protein amino acid sequence.

The term “polypeptide” refers to any linear or branched peptide comprising more than one amino acid. Polypeptide includes protein or fragment thereof or fusion thereof, provided such protein, fragment or fusion retains a useful biochemical or biological activity.

A fusion protein typically includes extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin (GLNDIFEAQKIEWHE (SEQ ID NO: 170)); Calmodulin-tag, which protein calmodulin is a peptide bound by the (KRRWKKNFIAVSAANRFKKISSSGAL (SEQ ID NO: 169)); polyglutamate tag, which is a peptide binding efficiently to anion-exchange resin such as Mono-Q (EEEEEE) (SEQ ID NO: 171); E-tag, which is a peptide recognized by an antibody (GAPVPYPDPLEPR) (SEQ ID NO: 172); FLAG-tag, which is a peptide recognized by an antibody (DYKDDDDK) (SEQ ID NO: 173); HA-tag, which is a peptide from hemagglutinin recognized by an antibody (YPYDVPDYA) (SEQ ID NO: 174); His-tag, which is typically 5-10 histidines bound by a nickel or cobalt chelate (HHHHHH) (SEQ ID NO: 175); Myc-tag, which is a peptide derived from c-myc recognized by an antibody (EQKLISEEDL) (SEQ ID NO: 176); NE-tag, which is a novel 18-amino-acid synthetic peptide (TKENPRSNQEESYDDNES) (SEQ ID NO: 177) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A (KETAAAKFERQHMDS) (SEQ ID NO: 178); SBP-tag, which is a peptide which binds to streptavidin; (MDEKTTGWRGGHVVEGLAGELEQLRARLEHHPQGQREP) (SEQ ID NO: 179); Softag 1, which is intended for mammalian expression (SLAELLNAGLGGS) (SEQ ID NO: 180); Softag 3, which is intended for prokaryotic expression (TQDPSRVG) (SEQ ID NO: 181); Strep-tag, which is a peptide which binds to streptavidin or the modified streptavidin called streptactin (Strep-tag II: WSHPQFEK) (SEQ ID NO: 182); TC tag, which is a tetracysteine tag that is recognized by FLASH and ReAsH biarsenical compounds (CCPGCC) V5 tag (SEQ ID NO: 183), which is a peptide recognized by an antibody (GKPIPNPLLGLDST) (SEQ ID NO: 184); VSV-tag, a peptide recognized by an antibody (YTDIEMNRLGK) (SEQ ID NO: 185); Xpress tag (DLYDDDDK) (SEQ ID NO: 186); Isopeptag, which is a peptide which binds covalently to pilin-C protein (TDKDMTITFTNKKDAE) (SEQ ID NO: 187); SpyTag, which is a peptide which binds covalently to SpyCatcher protein (AHIVMVDAYKPTK) (SEQ ID NO: 188); SnoopTag, a peptide which binds covalently to SnoopCatcher protein (KLGDIEFIKVNK) (SEQ ID NO: 189); BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein which is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nus-tag; Thioredoxin-tag; and Fc-tag, derived from immunoglobulin Fc domain, which allows dimerization and solubilization and can be used for purification on Protein-A Sepharose. Nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity when used in eukaryotic cells where the target genomic DNA substrate resides in the nucleus. One skilled in the art would appreciate these various fusion tag technologies, as well as how to make and use fusion proteins that include them.

### REFERENCE

1. Jinek, M. et al. A programmable dual-RNA-guided DNA endonuclease in adaptive bacterial immunity. Science 337, 816-821 (2012).

2. Cong, L. et al. Multiplex genome engineering using CRISPR/Cas systems. Science 339, 819-823 (2013).

3. Mali, P. et al. RNA-guided human genome engineering via Cas9. Science 339, 823-826 (2013).

4. Gao, Z., Herrera-Carrillo, E. & Berkhout, B. Delineation of the Exact Transcription Termination Signal for Type 3 Polymerase III. Mol Ther Nucleic Acids 10, 36-44 (2018).

5. Nishimasu, H. et al. Crystal structure of Cas9 in complex with guide RNA and target DNA. 5. Cell 156, 935-949 (2014).

6. Kim, S., Kim, D., Cho, S.W., Kim, J. & Kim, J. S. Highly efficient RNA-guided genome editing in human cells via delivery of purified Cas9 ribonucleoproteins. Genome Res 24, 1012-1019 (2014).

7. Liang, X. et al. Rapid and highly efficient mammalian cell engineering via Cas9 protein transfection. J Biotechnol 208, 44-53 (2015).

8. Hendel, A. et al. Chemically modified guide RNAs enhance CRISPR-Cas genome editing in human primary cells. Nature biotechnology 33, 985-989 (2015).

9. Vakulskas, C. A. et al. A high-fidelity Cas9 mutant delivered as a ribonucleoprotein complex enables efficient gene editing in human hematopoietic stem and progenitor cells. Nat Med 24, 1216-1224 (2018).

10. Anzalone, A. V. et al. Search-and-replace genome editing without double-strand breaks or donor DNA. Nature 576, 149-157 (2019).

11. Bravo, J. P. K. et al. Structural basis for mismatch surveillance by CRISPR-Cas9. Nature 603, 343-347 (2022).

All references, including publications, patent applications, and patents, as well as Appendices, cited herein are hereby incorporated by reference to the same extent as if each reference were individually and specifically indicated to be incorporated by reference and were set forth in its entirety herein. Nucleotide and amino acid sequences form part of this disclosure and is filed as a companion supplemental Sequence Listing with this disclosure.

Preferred embodiments of this invention are described herein, including the best mode known to the inventors for carrying out the invention. Variations of those preferred embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated variant Cas9 protein, comprising an amino acid sequence selected from the group consisting of the following relative to the wild-type Cas9 amino sequence of SEQ ID NO: 133:
a single amino acid substitution-comprising at least one substitution selected from Table 5 or Table 6;
a double amino acid substitution comprising S1106Y substitution and an additional substitution selected from the group consisting of E60K, A68K, T474R, A725R, A728W, H99A, E108P, and E130K;
a triple amino acid substitution comprising A68K/S1106Y and an additional substitution selected from the group consisting of T474R, E60K, A725R, A728W, H99A, E108P, E130K, and T333K;
a 4-amino acid substitution comprising A68K/T474R/S1106Y and an additional substitution selected from the group consisting of E60K, A725R, A728W, E108P, H99A, E108V, E114S, D124E, 1322V, K323L, Q330E, Q330V, L332K, P344R, E345T, E345Y, E349T, Q354S, D364G, D364V, G1104P, G1104A, S1109K, S1109R, S1109A, K1113A, R1114G, D1117G, K1118S, T445S, Y451W, R457A, M465L, T466V, T472K, T472R, L51R, D54K, D54R, G56A, E57K, T58S, T58G, L64K, Y72L, K65Y, K65F, T67S, N77K, C80R, D718K, S719N, E722K, H723M, N726R, L727V, A728G, S730G, A732S, I737V, L738R, L738Y, L738W, T740A, R753G, R1084E, K1085E, K1096V, T1098K, T1098R, E1099V, G1104A, L1198A, K26S, K30L, K31E, F32T, K33D, H1349R, H1349Y, and I1352P;
a 5-amino acid substitution comprising A68K/T474R/S1106Y and an additional substitution selected from the group consisting of G56A/E57K, K31E/G56A, K31E/E57K, K1085E/G56A, K1085E/E57K, and K1085E/K31E;
a 6-amino acid substitution comprising A68K/T474R/S1106Y and an additional substitution selected from the group consisting of K31E/K1085E/G56A, K31E/K1085E/E57K, K31E/G56A/E57K, and G56A/K1085E/E57K;
a 7-amino acid substitution comprising K31E/G56A/E57K/A68K/T474R/S1106Y and an additional substitution selected from the group consisting of K1085E, G1104A, M465L, T472K, R1084E, H1349Y, R753G, E108V, E130N, H329K, Q330V, T333R, S355C, A50T, I733Y, R753S, and P1090Y;
an 8-amino acid substitution comprising K31E/G56A/E57K/A68K/T474R/S1106Y and an additional substitution selected from the group consisting of H1349Y/R753G, T472K/R753G, H329K/R753G, T333R/R753G, H329K/T472K, T333R/T472K, and H329K/T333R;
a 9-amino acid substitution comprising K31E/G56A/E57K/A68K/T474R/S1106Y and an additional substitution selected from the group consisting of H329K/T472K/R753G, T333R/T472K/R753G, H329K/T333R/R753G, and H329K/T333R/T472K; and
a 10-amino acid substitution comprising K31E, G56A, E57K, A68K, H329K, T333R, T472K, T474R, R753G, and S1106Y.

2. The isolated variant Cas9 protein of claim 1, wherein the isolated variant is selected from the group consisting of a Cas9 variant with the following substitutions S1106Y, A68K, T474R with SEQ ID NO: 137; a Cas9 variant with the following substitutions S1106Y, A68K, T474R, K31E, G56A, E57K with SEQ ID NO: 139; a Cas9 variant with the following substitutions S1106Y, A68K, T474R, K31E, G56A, E57K, K1085E with SEQ ID NO: 141; and a Cas9 variant with the following substitutions K31E, G56A, E57K, A68K, H329K, T333R, T474R, R753G, S1106Y with SEQ ID NO: 135, wherein the substitutions are relative to the wild-type Cas9 amino acid sequence of SEQ ID NO: 133.

3. An isolated nucleic acid encoding the variant Cas9 protein of claim 1.

4. An mRNA encoding the variant Cas9 protein of claim 1.

5. A host cell comprising a nucleic acid encoding the modified Cas9 protein of claim 1.

6. The host cell of claim 5, wherein the host cell is selected from the group consisting of bacterial cells, insect cells, plant cells, mammalian cells, an immortalized cell, a HEK293 kidney cell, a Jurkat T cell, a primary human T cell, and HSPCs, an induced pluripotent stem cell.

7. A gene editing system, comprising:
a. at least one of the variant Cas9 proteins, or a nucleic acid encoding at least one of the variant Cas9 proteins, of claim 1; and
b. at least one of a single guide RNA (sgRNA) which has a scaffold sequence shorter than 76 nucleotides,
wherein the gene editing system exhibits enhanced editing activity relative to the gene editing activity of a wild-type Cas9 having the sequence of SEQ ID NO: 133 in the presence of the sgRNA.

8. The gene editing system of claim 7, wherein the sgRNA comprises a tetraloop.

9. The gene editing system of claim 7, wherein the sgRNA comprises a target-specific spacer sequence, a repeat sequence, a tetraloop region, an anti-repeat region, a stem loop 1 region, linker, a stem loop 2 region, a stem loop 3 region, and a terminal region.

10. The gene editing system of claim 7, wherein the sgRNA comprises a target-specific spacer sequence, a repeat sequence, a tetraloop region, an anti-repeat region, a stem loop 1 region, a linker region, and a stem loop 2 region, wherein the stem loop 3 region has been deleted.

11. The gene editing system of claim 7, wherein the sgRNA comprises a 20 nt target-specific spacer sequence, a 12 nt repeat region, a 4 nt tetraloop, and a 26 nt region comprising an anti-repeat region, a stem loop 1 region, a linker, a stem loop 2 region, a stem loop 3 region, and a terminal region.

12. The gene editing system of claim 7, wherein the sgRNA further comprises a poly-U terminator region.

13. The gene editing system of claim 7, wherein the sgRNA has a number of deletions from the 3′ end selected from the group consisting of 24 nt, 28 nt, 33 nt, 34 nt, 36 nt, 39 nt, and 41 nt.

14. The gene editing system of claim 7, wherein the sgRNA has a length selected from the group consisting of 76 nt, 72 nt, 66 nt, 67 nt, 64 nt, 61 nt, and 59 nt.

15. The gene editing system of claim 7 wherein the Cas9 variant has the same gene editing activity with the sgRNA as the gene editing activity of wild-type Cas9 paired with full length guides.

16. A kit comprising the gene editing system of claim 7 and instructions for use.

17. A composition comprising the variant Cas9 protein of claim 1, formulated for use in biochemical assays, industrial processes, or therapeutic applications.

18-26. (canceled)

27. A method of delivering the gene editing system of claim 7 to a cell, the method comprising the steps of:
(a) providing a first viral vector component encoding a sgRNA that hybridizes with a target sequence;
(b) providing a second viral vector component encoding the variant Cas9 protein;
wherein components (a) and (b) are located on same or different vectors of the system; and
(c) transducing the cell with the viral vector(s) under conditions sufficient to express the variant Cas9 protein and the sgRNA,
wherein the Cas9 and the sgRNA form a complex that binds to and edits the target sequence.

28. A method for delivering the gene editing system of claim 7 to a cell, comprising:
(a) providing lipid nanoparticles encapsulating a sgRNA that hybridizes with a target sequence;
(b) providing lipid nanoparticles encapsulating an mRNA encoding the variant Cas9 protein;
wherein components (a) and (b) are located on same or different vectors of the system,
(c) transducing the cell with the lipid nanoparticles under conditions sufficient to express the variant Cas9 protein and the sgRNA,
wherein the variant Cas9 protein and the sgRNA are expressed and form a complex to edit a specific target sequence in the cell's genome, and wherein the Cas9 variant has the same gene editing activity with the sgRNA as the gene editing activity of wild-type Cas9 paired with full length guides.

29. A method for delivering the gene editing system of claim 7 to a cell, comprising:
(a) providing a ribonucleoprotein (RNP) complex comprising the gene editing system; and
(b) introducing the RNP complex into the cell using electroporation,
wherein the gene editing system binds to and edits a target sequence within the genome of the cell, and wherein the Cas9 variant has the same gene editing activity with the sgRNA as the gene editing activity of wild-type Cas9 paired with full length guides.

30-34. (canceled)
